(19) United States
(12) Patent Application Publication
(10) Pub. No.: US 2009/0030024 A1
(43) Pub. Date: Jan. 29, 2009

(54) THIAZOLES AS FUNGICIDES

(75) Inventors: Jorg Nico Greul, Leichlingen (DE); Oliver Gaertzen, Koln (DE); Ralf Dunkel, Lyon (FR); Oliver Guth, Leverkusen (DE); Stefan Hillebrand, Neuss (DE); Kerstin Ilg, Koln (DE); Peter Schreier, Koln (DE); Ulrike Wachendorff-Neumann, Neuwied (DE); Peter Dahmen, Neuss (DE); Arnd Voerste, Koln (DE); Hiroyuki Hadano, Tochigi (JP); Samir Bennabi, Caluire-et-Cuire (FR)

Correspondence Address: WOMBLE CARLYLE SANDRIDGE & RICE, PLLC, ATTN: PATENT DOCKETING 32ND FLOOR, P.O. BOX 7037, ATLANTA, GA, 30357-0037, US

(73) Assignee: Bayer CropScience AG, Monheim (DE)

(21) Appl. No.: 12/067,634

(22) PCT Filed: Sep. 12, 2006
(86) PCT No.: PCT/EP2006/008861

§ 371 Date: Jul. 16, 2008

(30) Foreign Application Priority Data
Sep. 24, 2005 (DE) 10 2005 045 722.3
Oct. 7, 2005 (DE) 10 2005 048 072.1

(51) Int. Cl. A01N 43/54; C07D 417/04; A01P 3/00; C07D 417/14

(52) U.S. Cl. 514/275; 544/284; 544/328

(57) ABSTRACT

Use of compounds of the formula (I),

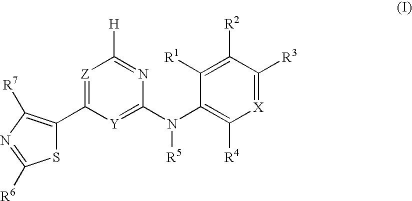

in which
R¹, R², R³, R⁴, R⁵, R⁶, R⁷, R⁸ and X, Y, Z are as defined in the description as fungicides. Compounds of the formula (Ia),

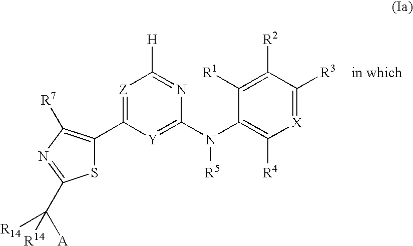

R¹, R², R³, R⁴, R⁵, R⁶, R⁷, R⁸, R¹⁴, A, X, Y and Z are as defined in the description, process for preparing these compounds and their use for controlling unwanted microorganisms.

The invention relates to thiazoles and their use for controlling unwanted microorganisms.

It is already known that certain thiazoles can be used as crop protection agents (see WO-A 2003/029249).

However, since the ecological and economical demands made on modern fungicides are increasing constantly, for example with respect to activity spectrum, toxicity, selectivity, application rate, formation of residues and favourable manufacture, and there can furthermore be problems, for example, with resistances, there is a constant need to develop novel fungicides which, at least in some areas, have advantages over those of the prior art.

Surprisingly, it has now been found that the present thiazoles achieve the objects mentioned at least in some aspects and are therefore suitable as fungicides.

Some of these thiazoles are already known as pharmaceutically active compounds (see, for example, WO-A 2005/012298, WO-A 2005/005438 or WO-A 2004/078682), but not their surprising fungicidal activity.

The invention provides the use of compounds of the formula (I) (thiazoles of the formula (I))

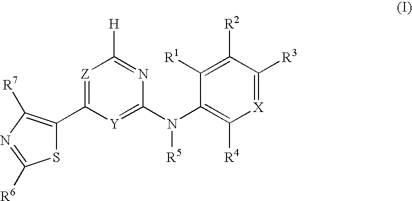

in which the symbols are as defined below:
R¹ to R⁴ independently of one another are hydrogen, halogen, cyano, hydroxyl, nitro, OR¹¹, SR¹¹, SOR¹¹, SO₂R¹¹, SO₂N(R¹¹)₂, C═OR¹¹, NR¹¹COOR¹², N(R¹¹)₂, NR¹¹COR¹¹, NR¹¹COR¹², NR¹¹SO₂R¹², OCON(R¹¹)₂, OC═OR¹¹, CON(R¹¹)₂, COOR¹¹, (CH₂)ₘOR¹¹, (CH₂)ₘSR¹¹, (CH₂)ₘN(R¹¹)₂, (CH₂)ₘCOOR¹², (CH₂)ₘNR¹¹COOR¹¹, unsubstituted or substituted C₁-C₈-alkyl, C₁-C₈-haloalkyl, C₃-C₈-cycloalkyl; where m=1-8
   or in each case two adjacent radicals R¹, R² or R², R³ or R³, R⁸ or R⁸, R⁴ together form an unsubstituted or substituted heterocyclic five- or six-membered ring,
R⁵ is hydrogen, unsubstituted or substituted C₁-C₈-alkyl, unsubstituted or substituted C₁-C₄-alkylC(═O), C₁-C₄-alkylOC(═O), unsubstituted or substituted C₁-C₄-alkoxy(C₁-C₄)alkyl, unsubstituted or substituted C₂-C₆-alkenyl, unsubstituted or substituted C₂-C₆-alkynyl, C₁-C₆-alkylsulphinyl, C₁-C₆-alkylsulphonyl, C₃-C₈-cycloalkyl; C₁-C₆-haloalkyl, C₁-C₄-haloalkylsulphinyl, C₁-C₄-haloalkylsulphonyl, halo-C₁-C₄-alkoxy-C₁-C₄-alkyl, C₃-C₈-halocycloalkyl having in each case 1 to 9 fluorine, chlorine and/or bromine atoms; formyl, formyl-C₁-C₃-alkyl, (C₁-C₃-alkyl)carbonyl-C₁-C₃-alkyl, (C₁-C₃-alkoxy)carbonyl-C₁-C₃-alkyl; halo-(C₁-C₃-alkyl)carbonyl-C₁-C₃-alkyl, halo-(C₁-C₃-alkoxy)carbonyl-C₁-C₃-alkyl having in each case 1 to 13 fluorine, chlorine and/or bromine atoms; (C₁-C₈-alkyl)carbonyl, (C₁-C₈-alkoxy)carbonyl, (C₁-C₈-alkylthio)carbonyl, (C₁-C₄-alkoxy-C₁-C₄-alkyl)carbonyl, (C₃-C₆-alkenyloxy)carbonyl, (C₃-C₆-alkynyloxy)carbonyl, (C₃-C₈-cycloalkyl)carbonyl; (C₁-C₆-haloalkyl)carbonyl, (C₁-C₆-haloalkylthio)carbonyl, (C₁-C₆-haloalkoxy)carbonyl, (C₃-C₆-haloalkenyloxy)carbonyl, (C₃-C₆-haloalkynyloxy)carbonyl, (halo-C₁-C₄-alkoxy-C₁-C₄-alkyl)carbonyl, (C₃-C₈-halocycloalkyl)carbonyl having in each case 1 to 9 fluorine, chlorine and/or bromine atoms, or —CH₂—C≡C—R^{1-A}, —CH₂—CH═CH—R^{1-A}, —CH═C═CH—R^{1-A}, —C(═O)C(═O)R², —CONR³R⁴, —CH₂NR⁵R⁶, C₁-C₄-trialkylsilyl or C₁-C₄-dialkylmonophenylsilyl,
R^{1-A} represents hydrogen, C₁-C₆-alkyl, C₁-C₆-haloalkyl, C₂-C₆-alkenyl, C₂-C₆-alkynyl, C₃-C₇-cycloalkyl, (C₁-C₄-alkoxy)carbonyl, (C₃-C₆-alkenyloxy)carbonyl, (C₃-C₆-alkynyloxy)-carbonyl or cyano,
X is nitrogen or CR⁸,
Y is nitrogen or CR⁹,
Z is nitrogen or CR¹⁰,
R⁶ is hydrogen, halogen, cyano, hydroxyl, OR¹¹, SR¹¹, COR¹¹, CO₂R¹¹, unsubstituted or substituted C₁-C₈-alkyl, C₁-C₈-haloalkyl, C₁-C₄-trialkylsilyl, N(R¹¹)₂, NR¹¹COR¹¹, NR¹¹COR¹², NR¹¹SOₙR¹¹, CON(R¹¹)₂, C₃-C₈-cycloalkyl, aryl, hetaryl; where n=0-2,
R⁷ is hydrogen, halogen, cyano, hydroxyl, amino, N(R¹¹)₂, nitro, OR¹¹, SR¹¹, unsubstituted or substituted C₁-C₈-alkyl, unsubstituted or substituted C₃-C₆-cycloalkyl, C₁-C₄-trialkylsilyl, COOR¹¹, CON(R¹¹)₂, COR¹¹ or
R⁷ and R¹⁰ together form a saturated or unsaturated bridge of the following structure:

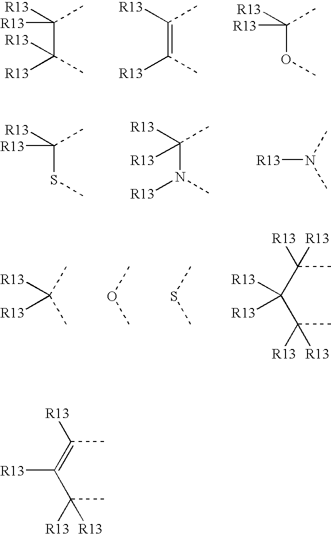

where R¹³=independently of one another hydrogen, halogen, unsubstituted or substituted C₁-C₃-alkyl, unsubstituted or substituted C₁-C₃-alkoxy or
   two geminal radicals R¹³ represent doubly attached oxygen or sulphur,
R⁸ is hydrogen, halogen, cyano, hydroxyl, nitro, OR¹¹, SR¹¹, SOR¹¹, SO₂R¹¹, SO₂N(R¹¹)₂, C═OR¹¹, NR¹¹COOR², N(R¹¹)₂, NR¹¹COR¹¹, NR¹¹COR², NRIISO₂R², OCON(R¹¹)₂, OC═OR¹¹, CON(R¹¹)₂, COOR¹¹, (CH₂)ₘOR¹¹, (CH₂)ₘSR¹¹, (CH₂)ₘN(R¹¹)₂, (CH₂)ₘCOOR², (CH₂)ₘNR¹¹COOR¹¹, unsubstituted or substituted C₁-C₈-alkyl, C₁-C₈-haloalkyl, C₃-C₈-cycloalkyl; where m=1-8,
R⁹ is hydrogen, halogen, N(R¹¹)₂, cyano, hydroxyl, OR¹¹, SR¹¹, COR¹¹, unsubstituted or substituted C₁-C₈-alkyl or unsubstituted or substituted C₃-C₈-cycloalkyl,
R¹⁰ is hydrogen, halogen, cyano, hydroxyl, amino, N(R¹¹)₂, nitro, OR¹¹, SR¹¹, unsubstituted or substituted C₁-C₈-alkyl, C₁-C₄-trialkylsilyl, COOR¹¹, CON(R¹¹)₂,
R¹¹ are identical or different and are hydrogen, unsubstituted or substituted C₁-C₈-alkyl, C₁-C₈-haloalkyl, C₁-C₄-trialkylsilyl, aryl, or
   if two radicals R¹¹ are attached to one nitrogen atom, two radicals R¹¹ may form a 3- to 7-membered unsubstituted or substituted saturated or unsaturated cycle which may contain up to five further heteroatoms selected from the group consisting of N, O and S, where two oxygen atoms are not adjacent, or
   if two radicals R¹¹ are adjacent in the grouping NR¹¹COR¹¹, two radicals R¹¹ may form a 3- to 7-membered unsubstituted or substituted saturated or unsaturated cycle which may contain up to five further heteroatoms selected from the group consisting of N, O and S, where two oxygen atoms are not adjacent,
R¹² are identical or different and are unsubstituted or substituted C₁-C₈-alkyl, C₁-C₈-haloalkyl, (CH₂)ₚOR¹¹, C₁-C₄-trialkylsilyl; where p=1-4
   and agrochemically active salts thereof as fungicides.

Compounds of the formula (I) are highly suitable for controlling unwanted microorganisms. Especially, they have strong fungicidal activity and can be used both in crop protection and in the protection of materials.

The formula (I) provides a general definition of the compounds according to the invention.

Preference is given to compounds of the formula (I) in which one or more of the symbols have one of the preferred meanings given below, i.e.,
R¹ to R⁴ independently of one another are hydrogen, fluorine, chlorine, bromine, iodine, cyano, hydroxyl, nitro, OR¹¹, O-Me, O-Et, O—Pr, O-iPr, O-Bu, O-secBu, O-isoBu, O-tBu, O—(CH₂)₂OH, O—(CH₂)₂OCH₃, O—(CH₂)₃OH, O—(CH₂)₃OCH₃, OCF₃, SR¹¹, SH, S-Me, S-Et, S—Pr, S-iPr, S-Bu, S-secBu, S-isoBu, S-tBu, SCF₃, SOR¹¹, SO-Me, SO-Et, SO—Pr, SO-iPr, SO-Bu, SO-secBu, SO-isoBu, SO-tBu, SO₂R¹¹, SO₂-Me, SO₂-Et, SO₂—Pr, SO₂-iPr, SO₂—Bu, SO₂-secBu, SO₂-isoBu, SO₂-tBu, SON(R¹¹)₂, SONHMe, SONMe₂, SONHEt, SONEt₂, SONHPr, SONPr₂, SONHBu, SONBu₂, SONHCF₃, SON(CF₃)₂, SO₂N(R¹¹)₂, SO₂NHMe, SO₂NMe₂, SO₂NEt₂, SO₂NHEt, SO₂NPr₂, SO₂NHPr, SO₂NHCF₃, SO₂N(CF₃)₂, COR¹¹, COMe, COEt, COPr, COiPr, COBu, COsecBu, CO-isoBu, COtBu, COCF₃, NR¹¹CO₂R¹¹, NR¹¹CO₂Me, NR¹¹ CO₂Et, NR¹¹ CO₂Pr, NR¹¹ CO₂iPr, NR¹¹ CO₂Bu, NR¹¹CO₂secBu, NR¹¹CO₂ isoBu, NR¹¹CO₂tBu, NR¹¹COR¹¹, NHCOMe, NHCOEt, NHCOPr, NHCOiPr, NHCOBu, NHCOiBu, NHCOsecBu, NHCOisoBu, NHCOtBu, NHCO(CH₂)₂OH, NHCO(CH₂)₂OCH₃, NHCO(CH₂)₃OH, NHCO(CH₂)₃OCH₃, NR¹¹COMe, NR¹¹ COEt, NR¹¹ COPr, NR¹¹ COiPr, NR¹¹ COBu, NR¹¹ COsecBu, NR¹¹ COtBu, NR¹¹ CO(CH₂)₂OH, NR¹¹CO(CH₂)₂OCH₃, NR¹¹CO(CH₂)₃OH, NR¹¹CO(CH₂)₃OCH₃, N(R¹¹)₂, NMe₂, NEt₂, NHMe, NH₂, NHtBu, NHEt, NHPr, NHiPr, NHBu, NHiBu, NHsecBu, piperazin-1-yl, 4-methylpiperazin-1-yl, morpholin-1-yl, NR¹¹SO₂R¹², NHSOR¹¹, NR¹¹SOR¹¹, NHSOMe, NHSO₂Me, NHSOEt, NHSO₂Et, NMeSOMe, NHSO₂R¹¹, NR¹¹SO₂R¹¹, NMeSO₂Me, NMeSOEt, NMeSO₂Et, NHSOCF₃, NHSO₂CF₃, OCON(R¹¹)₂, OCONHMe, OCONHEt, OCONHPr, OCONHiPr, OCONHBu, OCONHsecBu, OCONHisoBu, OCONHtBu, OCONMe₂, OCONEt₂, OCONPr₂, OCONiPr₂, OCONBu₂, OCONsecBu₂, OCONisoBu₂, OCONHtBu₂, OCOR¹¹, OCOMe, OCOEt, OCOPr, OCOiPr, OCOBu, OCOsecBu, OCOisoBu, OCOtBu, CON(R¹¹)₂, CONHEt, CONEt₂, CONHMe, CONMe₂, CONHPr, CONPr₂, CONHBu, CONHsecBu, CONHisoBu, CONHtBu, CONHCH(CH₃)CH₂OH, CONHCH(CH₃)CH₂OCH₃, CONHCH(C₂H₅)CH₂OH, CONHCH(C₂H₅)CH₂OCH₃, CONH(CH₂)₂OCH₃, CONH(CH₂)₂OH, CONH(CH₂)₃OCH₃, CONH(CH₂)₃OH, CONR¹¹CH(CH₃)CH₂OH, CONR¹¹CH(CH₃)CH₂OCH₃, CONR¹¹CH(C₂H₅)CH₂OH, CONR¹¹CH(C₂H₅)CH₂OCH₃, CONR¹¹ (CH₂)₂OCH₃, CONR¹¹ (CH₂)₂OH, CONR¹¹(CH₂)₃OCH₃, CONR¹¹(CH₂)₃OH, CO₂R¹¹, CO₂Me, CO₂Et, CO₂Pr, CO₂iPr, CO₂Bu, CO₂secBu, CO₂ isoBu, CO₂tBu, CO₂(CH₂)₂OH, CO₂(CH₂)₂OCH₃, CO₂(CH₂)₃OH, CO₂(CH₂)₃OCH₃, (CH₂)ₘOR¹¹, CH₂OH, (CH₂)₂OH, (CH₂)₃OH, (CH₂)₄OH, CH₂OMe, (CH₂)₂OMe, (CH₂)₃OMe, (CH₂)₄OMe, (CH₂)ₘSR¹¹, CH₂SH, (CH₂)₂SH, (CH₂)₃SH, (CH₂)₄SH, CH₂SMe, (CH₂)₂SMe, (CH₂)₃SMe, (CH₂)₄SMe, (CH₂)ₘN(R¹¹)₂, CH₂NH₂, CH₂NAc₂, CH₂N(COCF₃)₂, CH₂NHAc, CH₂NHCOCF₃, (CH₂)₂NH₂, (CH₂)₃NH₂, (CH₂)₄NH₂, CH₂NMe₂, (CH₂)₂NHMe, (CH₂)₂NMe₂, (CH₂)₃NHMe, (CH₂)₃NMe₂, (CH₂)₄NHMe, (CH₂)₄NMe₂, (CH₂)ₘCOOR¹², CH₂COOMe, (CH₂)₂COOMe, (CH₂)₃COOMe, CH₂COOEt, (CH₂)₂COOEt, (CH₂)₃COOEt, CH₂COOPr, (CH₂)₂COOPr, (CH₂)₃COOPr, CH₂COOiPr, (CH₂)₂COOiPr, (CH₂)₃COOiPr, CH₂COOtBu, (CH₂)₂COOtBu, (CH₂)₃COOtBu, CH₂COO(CH₂)₂OH CH₂COO(CH₂)₂OCH₃, CH₂COO(CH₂)₃OH, CH₂COO(CH₂)₃OCH₃, CH₂NHCOOR¹¹, CH₂NR¹¹COOR¹¹, CH₂NHCOOMe, CH₂NHCOOtBu, CH₂NHCOOEt, CH₂NHCOOPr, CH₂NHCOOiPr, CH₂NHCOOBu, CH₂NHCOOtBu, CH₂NHCOOsecBu, CH₂NHCOOisoBu, CH₂NR¹¹COOEt, CH₂NR¹¹COOPr, CH₂NR¹¹COOiPr, CH₂NR¹¹COOBu, CH₂NR¹¹COOtBu, CH₂NR¹¹COOsecBu, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; cyclopropyl, cyclobutyl, cyclopentyl, cyclohexyl; CF₃, CF₂H, CCl₃, C₂F₅, C₃F₇, CF(CF₃)₂
   or in each case two adjacent radicals R¹, R² or R², R³ or R³, R³ or R⁸, R⁴ together form a heterocyclic five- or six-membered ring which is unsubstituted or substituted by 1 to 4 halogen atoms or by 1 to 4 C₁-C₃-alkyl groups and which contains in each case up to two oxygen atoms,
R⁵ is hydrogen, methyl, ethyl, propyl, 1-methylethyl, butyl, 1-methylpropyl, 2-methylpropyl, 1,1-dimethylethyl, CF₃, CF₂H, CCl₃, C₂F₅, C₃F₇, CF(CF₃)₂;
   acetyl, C₂H₅C(═O), C₃H₇C(═O), C₄H₉C(═O), CF₃C(═O), C₂F₅C(═O), CH₃C(═O), C₂H₅OC (═O), C₃H₇C(═O), C₃H₇C(═O), C₄H₉C(═O), CF₃C(═O), CCl₃OC(═O), C₂F₅C(═O), CH₂OCH₃; C₂H₄OCH₃, CH═CH₂, CH₂CH═CH₂, C≡CH, CH₂C≡CH, SOCH₃, SOC₂H₅, SOC₃H₇ SO₂CH₃, SO₂C₂H₅, SO₂C₃H₇, cyclopropyl, cyclobutyl, cyclopentyl, cyclohexyl; CH₂OCF₃; C₂H₄OCF₃, C═ONH₂, SiMe₃, SiMe₂tBu, SiMe₂Ph,
X is nitrogen or CR⁸
Y is nitrogen or CR⁹,
Z is nitrogen or CR¹⁰,
   where either Y and Z are nitrogen,
or Y is nitrogen and Z is CR¹⁰,
or Y is CR⁹ and Z is nitrogen,
R⁶ is hydrogen, fluorine, chlorine, bromine, iodine, cyano, hydroxyl, OR¹¹, O-Me, O-Et, O—Pr, O-iPr, O-Bu, O-secBu, O-isoBu, O-tBu, OCH(CH₃)CH₂OH, OCH(CH₃)CH₂OCH₃, OCH(C₂H₅)CH₂OH, OCH(C₂H₅)CH₂OCH₃, O(CH₂)₂OCH₃, O(CH₂)₂OH, O(CH₂)₃OCH₃, O(CH₂)₃OH, OCF₃, SR¹¹, S-Me, S-Et, S—Pr, S-iPr, S-Bu, S-secBu, S-isoBu, S-tBu, SCF₃, SOR¹¹, SO-Me, SO-Et, SO—Pr, SO-iPr, SO-Bu, SO-secBu, SO-isoBu, SO-tBu, SO₂R¹¹, SO₂-Me, SO₂-Et, SO₂—Pr, SO₂-iPr, SO₂—Bu, SO₂-secBu, SO₂-isoBu, SO₂-tBu, COR¹¹, COMe, COEt, COPr, COiPr, COBu, COsecBu, COisoBu, COtBu, COCF₃, CO₂R¹¹, CO₂Me, CO₂Et, CO₂Pr, CO₂iPr, CO₂Bu, CO₂secBu, CO₂ isoBu, CO₂tBu, CO₂CH(CH₃)CH₂OH, CO₂CH(CH₃)CH₂OCH₃, CO₂CH(C₂H₅)CH₂OH, CO₂CH(C₂H₅)CH₂OCH₃, CO₂(CH₂)₂OCH₃, CO₂(CH₂)₂OH, CO₂(CH₂)₃OCH₃, CO₂(CH₂)₃OH, NR¹¹CO₂R¹¹, NR¹¹CO₂Me, NR¹¹CO₂Et, NR¹¹ CO₂Pr, NR¹¹CO₂iPr, NR¹¹CO₂Bu, NR¹¹CO₂secBu, NR¹¹CO₂ isoBu, NR¹¹CO₂tBu, NR¹¹COR¹¹, NR¹¹COMe, NR¹¹COEt, NR¹¹ COPr, NR¹¹COiPr, NR¹¹COBu, NR¹¹COsecBu, NR¹¹COisoBu, NR¹¹COtBu, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methyl-pentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; CH₂OCH₃, (CH₂)₂OCH₃, CH(CH₃)CH₂OCH₃, (CH₂)₂OH, CH(CH₃)CH₂OH, CF₃, CF₂H, CCl₃, C₂F₅, C₃F₇, CF(CF₃)₂,
   SiMe₃, SiMe₂tBu, SiMe₂Ph, NHR¹¹, N(R¹¹)₂, NMe₂, NEt₂, NHMe, NH₂, NHtBu, NHEt, NHPr, NHiPr, NHBu, NHiBu, NHsecBu, piperazin-1-yl, 4-methylpiperazin-1-yl, morpholin-1-yl, NHCH(CH₃)CH₂OH, NHCH(CH₃)CH₂OCH₃, NHCH(C₂H₅)CH₂OH, NHCH(C₂H₅)CH₂OCH₃, NH(CH₂)₂OCH₃, NH(CH₂)₂OH, NH(CH₂)₃OCH₃, NH(CH₂)₃OH, NHAc, NR¹¹ CH(CH₃)CH₂OH, NR¹¹CH(CH₃)CH₂OCH₃, NR¹¹CH(C₂H₅)CH₂OH, NR¹¹CH(C₂H₅)CH₂OCH₃, NR¹¹(CH₂)₂OCH₃, NR¹¹(CH₂)₂OH, NR¹¹ (CH₂)₃OCH₃, NR¹¹(CH₂)₃OH, NR¹¹Ac, NHCOEt, NHCOPr, NHCOiPr, NHCOBu, NHCOsecBu, NHCOisoBu, NHCOtBu, NHCOCF₃, NHCOC₂F₅, N(Me)tBu, N(Me)Et, N(Me)Pr, N(Me)iPr, N(Me)Bu, N(Me)secBu, N(Me)isoBu, N(CH₃)CH(CH₃)CH₂OH, N(CH₃)CH(CH₃)CH₂OCH₃, N(CH₃)CH(C₂H₅)CH₂OH, N(CH₃)CH(C₂H₃)CH₂OCH₃, N(CH₃)(CH₂)₂OCH₃, N(CH₃)(CH₂)₂OH, N(CH₃)(CH₂)₃OCH₃, N(CH₃)(CH₂)₃OH, NHCOR¹¹, NR¹¹COR¹¹, NMeAc, NMeCOEt, NMeCOPr, NMeCOiPr, NMeCOBu, NMeCOsecBu, NMeCOisoBu, NMeCOtBu, NMeCOCF₃, NMeCOC₂F₅, NHCO₂R¹¹, NR¹¹CO₂R¹¹, NHCO₂Me, NHCO₂Et, NHCO₂Pr, NHCO₂iPr, NHCO₂Bu, NHCO₂secBu, NHCO₂ isoBu, NHCO₂tBu, NHCO₂CF₃, NHCO₂C₂F₅, NHCO₂CH(CH₃)CH₂OH, NHCO₂CH(CH₃)CH₂OCH₃, NHCO₂CH(C₂H₅)CH₂OH, NHCO₂CH(C₂H₅)CH₂OCH₃, NHCO₂(CH₂)₂OCH₃, NHCO₂(CH₂)₂OH, NHCO₂(CH₂)₃OCH₃, NHCO₂(CH₂)₃OH, NMeCO₂Me, NMeCO₂Et, NMeCO₂Pr, NMeCO₂iPr, NMeCO₂Bu, NMeCO₂secBu, NMeCO₂ isoBu, NMeCO₂tBu, NMeCO₂CF₃, NMeCO₂C₂F₅, NEtCO₂Me, NEtCO₂Et, NEtCO₂Pr, NEtCO₂iPr, NEtCO₂Bu, NEtCO₂secBu, NEtCO₂ isoBu, NEtCO₂tBu, NEtCO₂CF₃, NEtCO₂C₂F₅, SON(R¹¹)₂, SONHMe, SONMe₂, SONHEt, SONEt₂, SONHPr, SONPr₂, SONHBu, SONBu₂, SONHCF₃, SON(CF₃)₂, SO₂N(R¹¹)₂, SO₂NHMe, SO₂NMe₂, SO₂NEt₂, SO₂NHEt, SO₂NPr₂, SO₂NHPr, SO₂NHCF₃, SO₂N(CF₃)₂, SO₂NHCH(CH₃)CH₂OH, SO₂NHCH(CH₃)CH₂OCH₃, SO₂NHCH(C₂H₅)CH₂OH, SO₂NHCH(C₂H₅)CH₂OCH₃, SO₂NH(CH₂)₂OCH₃, SO₂NH(CH₂)₂OH, SO₂NH(CH₂)₃OCH₃, SO₂NH(CH₂)₃OH, NHSOR¹¹, NR¹¹SOR¹¹, NHSOMe, NHSO₂Me, NHSOEt, NHSO₂Et, NMeSOMe, NHSO₂R¹¹, NR¹¹SO₂R¹¹, NMeSO₂Me, NMeSOEt, NMeSO₂Et, NHSOCF₃, NHSO₂CF₃, CON(R¹¹)₂, CONH₂, CONHMe, CONMe₂, CONHEt, CONEt₂, CO-morpholine, CO-piperidine, CO-piperazine, CO-(4-methylpiperazine), CONHCH(CH₃)CH₂OH, CONHCH(CH₃)CH₂OCH₃, CONHCH(C₂H₅)CH₂OH, CONHCH(C₂H₅)CH₂OCH₃, CONH(CH₂)₂OCH₃, CONH(CH₂)₂OH, CONH(CH₂)₃OCH₃, CONH(CH₂)₃OH, cyclopropyl, cyclopentyl, cyclohexyl, phenyl, pyridin-4-yl, pyridin-3-yl, pyridin-2-yl, thien-2-yl, thien-3-yl, furan-2-yl, furan-3-yl, N-1,3-oxazolidin-2-one, N-4-methyl-1,3-oxazolidin-2-one, N-4,4-dimethyl-1,3-oxazolidin-2-one, N-4,5-dimethyl-1,3-oxazolidin-2-one, N-5,5-dimethyl-1,3-oxazolidin-2-one, N-4-ethyl-1,3-oxazolidin-2-one, N-4,4-ethyl-1,3-oxazolidin-2-one, NHCH₂-tetrahydrofuran-2-yl,
R⁷ is hydrogen, fluorine, chlorine, bromine, iodine, cyano, hydroxyl, amino, N(R¹¹)₂, nitro, OR¹¹, SR¹¹, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methyl-pentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; CF₃, CF₂H, CCl₃, C₂F₅, C₃F₇, CF(CF₃)₂, cyclopropyl, cyclobutyl, cyclopentyl, cyclohexyl, SiMe₃, COOR¹¹, CON(R¹¹)₂, COR¹¹, or
R⁷ and R¹⁰ together form a saturated or unsaturated bridge of the following structure:

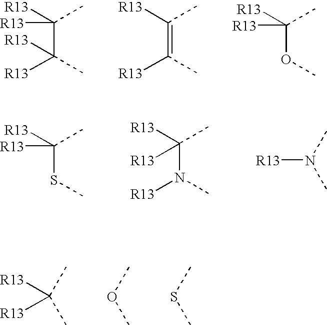

where R¹³=independently of one another hydrogen, fluorine, chlorine, bromine, iodine, methyl, ethyl, propyl, 1-methylethyl, CF₂H, CF₃, C₂F₅, OCH₃, OC₂H₅, OCF₃, OC₂F₅ or
   two geminal radicals R¹³ represent doubly attached oxygen or sulphur,
R⁸ is hydrogen, fluorine, chlorine, bromine, iodine, cyano, hydroxyl, nitro, OR¹¹, O-Me, O-Et, O—Pr, O-iPr, O-Bu, O-secBu, O-isoBu, O-tBu, O—(CH₂)₂OH, O—(CH₂)₂OCH₃, O—(CH₂)₃OH, O—(CH₂)₃OCH₃, OCF₃, SR¹¹, SH, S-Me, S-Et, S—Pr, S-iPr, S-Bu, S-secBu, S-isoBu, S-tBu, SCF₃, SOR¹¹, SO-Me, SO-Et, SO—Pr, SO-iPr, SO-Bu, SO-secBu, SO-isoBu, SO-tBu, SO₂R¹¹, SO₂-Me, SO₂-Et, SO₂—Pr, SO₂-iPr, SO₂—Bu, SO₂-secBu, SO₂-isoBu, SO₂-tBu, SON(R¹¹)₂, SONHMe, SONMe₂, SONHEt, SONEt₂, SONHPr, SONPr₂, SONHBu, SONBu₂, SONHCF₃, SON(CF₃)₂, SO₂N(R¹¹)₂, SO₂NHMe, SO₂NMe₂, SO₂NEt₂, SO₂NHEt, SO₂NPr₂, SO₂NHPr, SO₂NHCF₃, SO₂N(CF₃)₂, COR¹¹, COMe, COEt, COPr, COiPr, COBu, COsecBu, COisoBu, COtBu, COCF₃, NR¹¹CO₂R¹¹, NR¹¹CO₂Me, NR¹¹CO₂Et, NR¹¹CO₂Pr, NR¹¹CO₂iPr, NR¹¹CO₂Bu, NR¹¹CO₂secBu, NR¹¹CO₂ isoBu, NR¹¹CO₂tBu, NR¹¹COR¹¹, NHCOMe, NHCOEt, NHCOPr, NHCOiPr, NHCOBu, NHCOiBu, NHCOsecBu, NHCOisoBu, NHCOtBu, NHCO(CH₂)₂OH, NHCO(CH₂)₂OCH₃, NHCO(CH₂)₃OH, NHCO(CH₂)₃OCH₃, NR¹¹COMe, NR¹¹ COEt, NR¹¹ COPr, NR¹¹COiPr, NR¹¹COBu, NR¹¹COsecBu, NR¹¹COisoBu, NR¹¹COtBu, NR¹¹CO(CH₂)₂OH, NR¹¹CO(CH₂)₂OCH₃, NR¹¹CO(CH₂)₃OH, NR¹¹CO(CH₂)₃OCH₃, N(R¹¹)₂, NMe₂, NEt₂, NHMe, NH₂, NHtBu, NHEt, NHPr, NHiPr, NHBu, NHsecBu, NHisoBu, 4-methylpiperazin-1-yl, piperazin-1-yl, morpholin-1-yl, NR¹¹ SO₂R¹², NHSOR¹¹, NR¹¹SOR¹¹, NHSOMe, NHSO₂Me, NHSOEt, NHSO₂Et, NMeSOMe, NHSO₂R¹¹, NR¹¹SO₂R¹¹, NMeSO₂Me, NMeSOEt, NMeSO₂Et, NHSOCF₃, NHSO₂CF₃, OCON(R¹¹)₂, OCONHMe, OCONHEt, OCONHPr, OCONHiPr, OCONHBu, OCONHsecBu, OCONHisoBu, OCONHtBu, OCONMe₂, OCONEt₂, OCONPr₂, OCONiPr₂, OCONBu₂, OCONsecBu₂, OCONisoBu₂, OCONtBu₂, OCOR¹¹, OCOMe, OCOEt, OCOPr, OCOiPr, OCOBu, OCOsecBu, OCOisoBu, OCOtBu, CON(R¹¹)₂, CONHEt, CONEt₂, CONHMe, CONMe₂, CONHPr, CONPr₂, CONHBu, OCONHBu, CONHtBu, CONHCH(CH₃)CH₂OH, CONHCH(CH₃)CH₂OCH₃, CONHCH(C₂H₅)CH₂OH, CONHCH(C₂Hs)CH₂OCH₃, CONH(CH₂)₂OCH₃, CONH(CH₂)₂OH, CONH(CH₂)₃OCH₃, CONH(CH₂)₃OH, CONR¹¹CH(CH₃)CH₂OH, CONR¹¹ CH(CH₃)CH₂OCH₃, CONR¹¹CH(C₂H₅)CH₂OH, CONR¹¹CH(C₂H₅)CH₂OCH₃, CONR¹¹ (CH₂)₂OCH₃, CONR¹¹(CH₂)₂OH, CONR¹¹(CH₂)₃OCH₃, CONR¹¹(CH₂)₃OH, CO₂R¹¹, CO₂Me, CO₂Et, CO₂Pr, CO₂iPr, CO₂Bu, CO₂secBu, CO₂ isoBu, CO₂tBu, CO₂(CH₂)₂OH, CO₂(CH₂)₂OCH₃, CO₂(CH₂)₃OH, CO₂(CH₂)₃OCH₃, (CH₂)ₘOR¹¹, CH₂OH, (CH₂)₂OH, (CH₂)₃OH, (CH₂)₄OH, CH₂OMe, (CH₂)₂OMe, (CH₂)₃OMe, (CH₂)₄OMe, (CH₂)ₘSR¹¹, CH₂SH, (CH₂)₂SH, (CH₂)₃SH, (CH₂)₄SH, CH₂SMe, (CH₂)₂SMe, (CH₂)₃SMe, (CH₂)₄SMe, (CH₂)ₘN(R¹¹)₂, CH₂NH₂, CH₂NAc₂, CH₂N(COCF₃)₂, CH₂NHAc, CH₂NHCOCF₃, (CH₂)₂NH₂, (CH₂)₃NH₂, (CH₂)₄NH₂, CH₂NMe₂, (CH₂)₂NHMe, (CH₂)₂NMe₂, (CH₂)₃NHMe, (CH₂)₃NMe₂, (CH₂)₄NHMe, (CH₂)₄NMe₂, (CH₂)ₘCOOR¹², CH₂COOMe, (CH₂)₂COOMe, (CH₂)₃COOMe, CH₂COOEt, (CH₂)₂COOEt, (CH₂)₃COOEt, CH₂COOPr, (CH₂)₂COOPr, (CH₂)₃COOPr, CH₂COOiPr, (CH₂)₂COOiPr, (CH₂)₃COOiPr, CH₂COOtBu, (CH₂)₂COOtBu, (CH₂)₃COOtBu, CH₂COO(CH₂)₂OH, CH₂COO(CH₂)₂OCH₃, CH₂COO(CH₂)₃OH, CH₂COO(CH₂)₃OCH₃, CH₂NHCOOR¹¹, CH₂NR¹¹COOR¹¹, CH₂NHCOOMe, CH₂NHCOOtBu, CH₂NHCOOEt, CH₂NHCOOPr, CH₂NHCOOiPr, CH₂NHCOOBu, CH₂NHCOOtBu, CH₂NHCOOsecBu, CH₂NHCOOisoBu, CH₂NR¹¹COOEt, CH₂NR¹¹COOPr, CH₂NR¹¹COOiPr, CH₂NR¹¹ COOBu, CH₂NR¹¹ COOtBu, CH₂NR¹¹ COOsecBu, CH₂NR¹¹COOisoBu, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methyl-propyl; cyclopropyl, cyclobutyl, cyclopentyl, cyclohexyl; CF₃, CF₂H, CCl₃, C₂F₅, C₃F₇, CF(CF₃)₂,
R⁹ is hydrogen, fluorine, chlorine, bromine, iodine, N(R¹¹)₂, NMe₂, NEt₂, NHMe, NH₂, NHtBu, NHEt, NHPr, NHiPr, NHBu, NHsecBu, NHisoBu, 4-methylpiperazin-1-yl, piperazin-1-yl, morpholin-1-yl, cyano, hydroxyl, OR¹¹, O-Me, O-Et, O—Pr, O-iPr, O-Bu, O-secBu, O-isoBu, O-tBu, O—(CH₂)₂OH, O—(CH₂)₂OCH₃, O—(CH₂)₃OH, O—(CH₂)₃OCH₃, OCF₃, SR¹¹, SH, S-Me, S-Et, S—Pr, S-iPr, S-Bu, S-secBu, S-isoBu, S-tBu, SCF₃, COR¹¹, COMe, COEt, COPr, COiPr, COBu, COsecBu, COisoBu, COtBu, COCF₃, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methyl-propyl; CF₃, CF₂H, CCl₃, C₂F₅, C₃F₇, CF(CF₃)₂, cyclopropyl, cyclobutyl, cyclopentyl, cyclohexyl,
R¹⁰ is hydrogen, fluorine, chlorine, bromine, iodine, cyano, hydroxyl, amino, N(R¹¹)₂, nitro, OR¹¹, SR¹¹, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methyl-pentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; CF₃, CF₂H, CCl₃, C₂F₅, C₃F₇, CF(CF₃)₂, cyclopropyl, cyclobutyl, cyclopentyl, cyclohexyl, SiMe₃, COOR¹¹, CON(R¹¹)₂,
R¹¹ are identical or different and are hydrogen, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, CH₂CH₂OMe, CH(CH₃)CH₂OMe, CH₂CH(CH₃)OMe, CH₂CH₂OEt, CH(CH₃)CH₂OEt, CH₂CH(CH₃)OEt, CF₃, CF₂H, CCl₃, C₂F₅, C₃F₇, CF(CF₃)₂, cyclopropyl, cyclobutyl, cyclopentyl, cyclohexyl, SiMe₃, phenyl or
   if two radicals R¹¹ are attached to one nitrogen atom, two radicals R¹¹ may form a 3- to 7-membered, unsubstituted or substituted saturated or unsaturated cycle which may contain up to five further heteroatoms from the group consisting of N, O and S, where two oxygen atoms are not adjacent, or
   if two radicals R¹¹ are adjacent in the grouping NR¹¹COR¹¹, two radicals R¹¹ may form a 3- to 7-membered unsubstituted or substituted saturated or unsaturated cycle which may contain up to five further heteroatoms selected from the group consisting of N, O and S, where two oxygen atoms are not adjacent,
R¹² is methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; CF₃, CF₂H, CCl₃, C₂F₅, C₃F₇, CF(CF₃)₂, cyclopropyl, cyclobutyl, cyclopentyl, cyclohexyl, CH₂OR¹¹, (CH₂)₂OR¹¹, (CH₂)₃OR¹¹, (CH₂)₄OR¹¹, SiMe₃.

Particular preference is given to compounds of the formula (I) in which one or more of the symbols have one of the following meanings:
R¹ to R⁴ independently of one another are hydrogen, fluorine, chlorine, bromine, iodine, cyano, hydroxyl, nitro, OR¹¹, O-Me, O-Et, O—Pr, O-iPr, O-Bu, O-secBu, O-isoBu, O-tBu, O—(CH₂)₂OH, O—(CH₂)₂OCH₃, O—(CH₂)₃OH, O—(CH₂)₃OCH₃, OCF₃, SR¹¹, SH, S-Me, S-Et, S—Pr, S-iPr, S-Bu, S-secBu, S-isoBu, S-tBu, SCF₃, SOR¹¹, SO-Me, SO-Et, SO—Pr, SO-iPr, SO-Bu, SO-secBu, SO-isoBu, SO-tBu, SO₂R¹¹, SO₂-Me, SO₂-Et, SO₂—Pr, SO₂-iPr, SO₂—Bu, SO₂-secBu, SO₂-isoBu, SO₂-tBu, SON(R¹¹)₂, SONHMe, SONMe₂, SONHEt, SONEt₂, SONHPr, SONPr₂, SONHBu, SONBu₂, SONHCF₃, SON(CF₃)₂, SO₂N(R¹¹)₂, SO₂NHMe, SO₂NMe₂, SO₂NEt₂, SO₂NHEt, SO₂NPr₂, SO₂NHPr, SO₂NHCF₃, SO₂N(CF₃)₂, COR¹¹, COMe, COEt, COPr, COiPr, COBu, COsecBu, CO-isoBu, COtBu, COCF₃, NR¹¹CO₂R¹¹, NR¹¹CO₂Me, NR¹¹CO₂Et, NR¹¹CO₂Pr, NR¹¹CO₂iPr, NR¹¹CO₂Bu, NR¹¹CO₂secBu, NR¹¹CO₂ isoBu, NR¹¹CO₂tBu, NR¹¹COR¹¹, NHCOMe, NHCOEt, NHCOPr, NHCOiPr, NHCOBu, NHCOiBu, NHCOsecBu, NHCOisoBu, NHCOtBu, NHCO(CH₂)₂OH, NHCO(CH₂)₂OCH₃, NHCO(CH₂)₃OH, NHCO(CH₂)₃OCH₃, NR¹¹COMe, NR¹¹COEt, NR¹¹ COPr, NR¹¹COiPr, NR¹¹COBu, NR¹¹COsecBu, NR¹¹COtBu, NR¹¹CO(CH₂)₂OH, NR¹¹CO(CH₂)₂OCH₃, NR¹¹CO(CH₂)₃OH, NR¹¹CO(CH₂)₃OCH₃, N(R¹¹)₂, NMe₂, NEt₂, NHMe, NH₂, NHtBu, NHEt, NHPr, NHiPr, NHBu, NHiBu, NHsecBu, piperazin-1-yl, 4-methylpiperazin-1-yl, morpholin-1-yl, NR¹¹SO₂R¹², NHSOR¹¹, NR¹¹SOR¹¹, NHSOMe, NHSO₂Me, NHSOEt, NHSO₂Et, NMeSOMe, NHSO₂R¹¹, NR¹¹SO₂R¹¹, NMeSO₂Me, NMeSOEt, NMeSO₂Et, NHSOCF₃, NHSO₂CF₃, OCON(R¹¹)₂, OCONHMe, OCONHEt, OCONHPr, OCONHiPr, OCONHBu, OCONHsecBu, OCONHisoBu, OCONHtBu, OCONMe₂, OCONEt₂, OCONPr₂, OCONiPr₂, OCONBu₂, OCONsecBu₂, OCONisoBu₂, OCONHtBu₂, OCOR¹¹, OCOMe, OCOEt, OCOPr, OCOiPr, OCOBu, OCOsecBu, OCOisoBu, OCOtBu, CON(R¹¹)₂, CONHEt, CONEt₂, CONHMe, CONMe₂, CONHPr, CONPr₂, CONHBu, CONHsecBu, CONHisoBu, CONHtBu, CONHCH(CH₃)CH₂OH, CONHCH(CH₃)CH₂OCH₃, CONHCH(C₂H₅)CH₂OH, CONHCH(C₂H₅)CH₂OCH₃, CONH(CH₂)₂OCH₃, CONH(CH₂)₂OH, CONH(CH₂)₃OCH₃, CONH(CH₂)₃OH, CONR¹¹CH(CH₃)CH₂OH, CONR¹¹CH(CH₃)CH₂OCH₃, CONR¹¹CH(C₂H₅)CH₂OH, CONR¹¹CH(C₂Hs)CH₂OCH₃, CONR¹¹ (CH₂)₂OCH₃, CONR¹¹(CH₂)₂OH, CONR¹¹(CH₂)₃OCH₃, CONR¹¹(CH₂)₃OH, CO₂R¹¹, CO₂Me, CO₂Et, CO₂Pr, CO₂iPr, CO₂Bu, CO₂secBu, CO₂ isoBu, CO₂tBu, CO₂(CH₂)₂OH, CO₂(CH₂)₂OCH₃, CO₂(CH₂)₃OH, CO₂(CH₂)₃OCH₃, (CH₂)ₘOR¹¹, CH₂OH, (CH₂)₂OH, (CH₂)₃OH, (CH₂)₄OH, CH₂OMe, (CH₂)₂OMe, (CH₂)₃OMe, (CH₂)₄OMe, (CH₂)₂SR¹¹, CH₂SH, (CH₂)₂SH, (CH₂)₃SH, (CH₂)₄SH, CH₂SMe, (CH₂)₂SMe, (CH₂)₃SMe, (CH₂)₄SMe, (CH₂)ₘN(R¹¹)₂, CH₂NH₂, CH₂NAC₂, CH₂N(COCF₃)₂, CH₂NHAc, CH₂NHCOCF₃, (CH₂)₂NH₂, (CH₂)₃NH₂, (CH₂)₄NH₂, CH₂NMe₂, (CH₂)₂NHMe, (CH₂)₂NMe₂, (CH₂)₃NHMe, (CH₂)₃NMe₂, (CH₂)₄NHMe, (CH₂)₄NMe₂, (CH₂)ₘCOOR², CH₂COOMe, (CH₂)₂COOMe, (CH₂)₃COOMe, CH₂COOEt, (CH₂)₂COOEt, (CH₂)₃COOEt, CH₂COOPr, (CH₂)₂COOPr, (CH₂)₃COOPr, CH₂COOiPr, (CH₂)₂COOiPr, (CH₂)₃COOiPr, CH₂COOtBu, (CH₂)₂COOtBu, (CH₂)₃COOtBu, CH₂COO(CH₂)₂OH CH₂COO(CH₂)₂OCH₃, CH₂COO(CH₂)₃OH, CH₂COO(CH₂)₃OCH₃, CH₂NHCOOR¹¹, CH₂NR¹¹COORt, CH₂NHCOOMe, CH₂NHCOOtBu, CH₂NHCOOEt, CH₂NHCOOPr, CH₂NHCOOiPr, CH₂NHCOOBu, CH₂NHCOOtBu, CH₂NHCOOsecBu, CH₂NHCOOisoBu, CH₂NR¹¹COOEt, CH₂NR¹¹COOPr, CH₂NR¹¹COOiPr, CH₂NR¹¹COOBu, CH₂NR¹¹COOtBu, CH₂NR¹¹ COOsecBu, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; cyclopropyl, cyclobutyl, cyclopentyl, cyclohexyl; CF₃, CF₂H, CCl₃, C₂F₅, C₃F₇, CF(CF₃)₂,
   or in each case two adjacent radicals R¹, R² or R², R³ or R³, R⁸ or R⁸, R⁴ together form a 2,3-dihydro-1,4-dioxin, a 2,2,3,3-tetrafluoro-2,3-dihydro-1,4-dioxin, a 1,3-dioxole or a 2,2-difluoro-1,3-dioxole ring,
R⁵ is hydrogen, methyl, ethyl, propyl, 1-methylethyl, butyl, 1-methylpropyl, 2-methylpropyl, 1,1-dimethylethyl, CF₃, CF₂H, CCl₃, C₂F₅, C₃F₇, CF(CF₃)₂; acetyl, C₂H₅C(═O), C₃H₇C(═O), C₄H₉C(═O), CF₃C(═O), C₂F₅C(═O), CH₂OCH₃; C₂H₄OCH₃, CH═CH₂, CH₂CH═CH₂, C≡CH, CH₂C≡CH, SOCH₃, SOC₂H₅, SOC₃H₇ SO₂CH₃, SO₂C₂H₅, SO₂C₃H₇, cyclopropyl, CH₂OCF₃; C₂H₄OCF₃, C═ONH₂, SiMe₃, SiMe₂tBu, SiMe₂Ph,
X is nitrogen or CR⁸,
Y is nitrogen,
Z is nitrogen or CR¹⁰,
R⁶ is hydrogen, fluorine, chlorine, bromine, iodine, cyano, hydroxyl, OR¹¹, O-Me, O-Et, O—Pr, O-iPr, O-Bu, O-secBu, O-isoBu, O-tBu, OCH(CH₃)CH₂OH, OCH(CH₃)CH₂OCH₃, OCH(C₂H₅)CH₂OH, OCH(C₂H₅)CH₂OCH₃, O(CH₂)₂OCH₃, O(CH₂)₂OH, O(CH₂)₃OCH₃, O(CH₂)₃OH, OCF₃, SR¹¹, S-Me, S-Et, S—Pr, S-iPr, S-Bu, S-secBu, S-isoBu, S-tBu, SCF₃, SOR¹¹, SO-Me, SO-Et, SO—Pr, SO-iPr, SO-Bu, SO-secBu, SO-isoBu, SO-tBu, SO₂R¹¹, SO₂-Me, SO₂-Et, SO₂—Pr, SO₂-iPr, SO₂—Bu, SO₂-secBu, SO₂-isoBu, SO₂-tBu, COR¹¹, COMe, COEt, COPr, COiPr, COBu, COsecBu, COisoBu, COtBu, COCF₃, CO₂R¹¹, CO₂Me, CO₂Et, CO₂Pr, CO₂iPr, CO₂Bu, CO₂secBu, CO₂ isoBu, CO₂tBu, CO₂CH(CH₃)CH₂OH, CO₂CH(CH₃)CH₂OCH₃, CO₂CH(C₂H₅)CH₂OH, CO₂CH(C₂H₅)CH₂OCH₃, CO₂(CH₂)₂OCH₃, CO₂(CH₂)₂OH, CO₂(CH₂)₃OCH₃, CO₂(CH₂)₃OH, NR¹¹CO₂R¹¹, NR¹¹CO₂Me, NR¹¹CO₂Et, NR¹¹CO₂Pr, NR¹¹CO₂iPr, NR¹¹CO₂Bu, NR¹¹CO₂secBu, NR¹¹CO₂ isoBu, NR¹¹CO₂tBu, NR¹¹COR¹¹, NR¹¹COMe, NR¹¹COEt, NR¹¹COPr, NR¹¹COiPr, NR¹¹COBu, NR¹¹COsecBu, NR¹¹COisoBu, NR¹¹COtBu, Me, Et, Pr, iPr, Bu, secBu, isoBu, tBu, CH₂OCH₃, (CH₂)₂OCH₃, CH(CH₃)CH₂OCH₃, (CH₂)₂OH, CH(CH₃)CH₂OH, CF₃, CF₂H, CCl₃, C₂F₅, C₃F₇, CF(CF₃)₂,
   SiMe₃, SiMe₂tBu, SiMe₂Ph, NHR¹¹, N(R¹¹)₂, NMe₂, NEt₂, NHMe, NH₂, NHtBu, NHEt, NHPr, NHiPr, NHBu, NHsecBu, NHisoBu, NHCH(CH₃)CH₂OH, NHCH(CH₃)CH₂OCH₃, NHCH(C₂H₅)CH₂OH, NHCH(C₂H₅)CH₂OCH₃, NH(CH₂)₂OCH₃, NH(CH₂)₂OH, NH(CH₂)₃OCH₃, NH(CH₂)₃OH, NHAc, NR¹¹CH(CH₃)CH₂OH, NR¹¹CH(CH₃)CH₂OCH₃, NR¹¹CH(C₂H₅)CH₂OH, NR¹¹CH(C₂H₅)CH₂OCH₃, NR¹¹(CH₂)₂OCH₃, NR¹¹(CH₂)₂OH, NR¹¹(CH₂)₃OCH₃, NR¹¹(CH₂)₃OH, NR¹¹Ac, NHCOEt, NHCOPr, NHCOiPr, NHCOBu, NHCOsecBu, NHCOisoBu, NHCOtBu, NHCOCF₃, NHCOC₂F₅, N(Me)tBu, N(Me)Et, N(Me)Pr, N(Me)iPr, N(Me)Bu, N(Me)secBu, N(Me)isoBu, N(CH₃)CH(CH₃)CH₂OH, N(CH₃)CH(CH₃)CH₂OCH₃, N(CH₃)CH(C₂H₅)CH₂OH, N(CH₃)CH(C₂H₅)CH₂OCH₃, N(CH₃)(CH₂)₂OCH₃, N(CH₃)(CH₂)₂OH, N(CH₃)(CH₂)₃OCH₃, N(CH₃)(CH₂)₃OH, NHCOR¹¹, NR¹¹COR¹¹, NMeAc, NMeCOEt, NMeCOPr, NMeCOiPr, NMeCOBu, NMeCOsecBu, NMeCOisoBu, NMeCOtBu, NMeCOCF₃, NMeCOC₂F₅, NHCO₂R¹¹, NR¹¹CO₂R¹¹, NHCO₂Me, NHCO₂Et, NHCO₂Pr, NHCO₂iPr, NHCO₂Bu, NHCO₂secBu, NHCO₂ isoBu, NHCO₂tBu, NHCO₂CF₃, NHCO₂C₂F₅, NHCO₂CH(CH₃)CH₂OH, NHCO₂CH(CH₃)CH₂OCH₃, NHCO₂CH(C₂H₅)CH₂OH, NHCO₂CH(C₂H₅)CH₂OCH₃, NHCO₂(CH₂)₂OCH₃, NHCO₂(CH₂)₂OH, NHCO₂(CH₂)₃OCH₃, NHCO₂(CH₂)₃OH, NMeCO₂Me, NMeCO₂Et, NMeCO₂Pr, NMeCO₂iPr, NMeCO₂Bu, NMeCO₂secBu, NMeCO₂ isoBu, NMeCO₂tBu, NMeCO₂CF₃, NMeCO₂C₂F₅, NEtCO₂Me, NEtCO₂Et, NEtCO₂Pr, NEtCO₂iPr, NEtCO₂Bu, NEtCO₂secBu, NEtCO₂ isoBu, NEtCO₂tBu, NEtCO₂CF₃, NEtCO₂C₂F₅, SON(R¹¹)₂, SONHMe, SONMe₂, SONHEt, SONEt₂, SONHPr, SONPr₂, SONHBu, SONBu₂, SONHCF₃, SON(CF₃)₂, SO₂N(R¹¹)₂, SO₂NHMe, SO₂NMe₂, SO₂NEt₂, SO₂NHEt, SO₂NPr₂, SO₂NHPr, SO₂NHCF₃, SO₂N(CF₃)₂, SO₂NHCH(CH₃)CH₂OH, SO₂NHCH(CH₃)CH₂OCH₃, SO₂NHCH(C₂H₅)CH₂OH, SO₂NHCH(C₂H₅)CH₂OCH₃, SO₂NH(CH₂)₂OCH₃, SO₂NH(CH₂)₂OH, SO₂NH(CH₂)₃OCH₃, SO₂NH(CH₂)₃OH, NHSOR¹¹, NR¹¹SOR¹¹, NHSOMe, NHSO₂Me, NHSOEt, NHSO₂Et, NMeSOMe, NHSO₂R¹¹, NR¹¹ SO₂R¹¹, NMeSO₂Me, NMeSOEt, NMeSO₂Et, NHSOCF₃, NHSO₂CF₃, CON(R¹¹)₂, CONH₂, CONHMe, CONMe₂, CONHEt, CONEt₂, CO-morpholine, CO-piperidine, CO-piperazine, CO-(4-methylpiperazine), CONHCH(CH₃)CH₂OH, CONHCH(CH₃)CH₂OCH₃, CONHCH(C₂H₅)CH₂OH, CONHCH(C₂H₅)CH₂OCH₃, CONH(CH₂)₂OCH₃, CONH(CH₂)₂OH, CONH(CH₂)₃OCH₃, CONH(CH₂)₃OH, cyclopropyl, cyclopentyl, cyclohexyl, phenyl, pyridin-4-yl, pyridin-3-yl, pyridin-2-yl, thien-2-yl, thien-3-yl, furan-2-yl, furan-3-yl, N-1,3-oxazolidin-2-one, N-4-methyl-1,3-oxazolidin-2-one, N4,4-dimethyl-1,3-oxazolidin-2-one, N-4,5-dimethyl-1,3-oxazolidin-2-one, N-5,5-dimethyl-1,3-oxazolidin-2-one, N-4-ethyl-1,3-oxazolidin-2-one, N-4,4-ethyl-1,3-oxazolidin-2-one, NHCH₂-tetrahydrofuran-2-yl,
R⁷ is hydrogen, fluorine, chlorine, bromine, iodine, cyano, hydroxyl, amino, N(R¹¹)₂, nitro, OR¹¹, SR¹¹, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methyl-pentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; CF₃, CF₂H, CCl₃, C₂F₅, C₃F₇, CF(CF₃)₂, cyclopropyl, cyclobutyl, cyclopentyl, cyclohexyl, SiMe₃, COOR¹¹, CON(R¹¹)₂, COR¹¹, or
R⁷ and R¹⁰ together form a saturated or unsaturated bridge of the following structure:

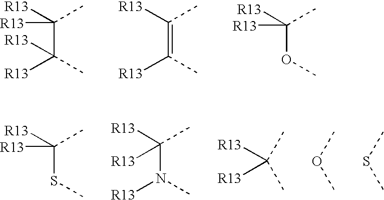

where R¹³=independently of one another hydrogen, fluorine, chlorine, bromine, iodine, methyl, ethyl, propyl, 1-methylethyl, CF₂H, CF₃, C₂F₅, OCH₃; OC₂H₅, OCF₃, OC₂F₅ or
   two geminal radicals R¹³ represent doubly attached oxygen or sulphur,
R⁸ is hydrogen, fluorine, chlorine, bromine, iodine, cyano, hydroxyl, nitro, OR¹¹, O-Me, O-Et, O—Pr, O-iPr, O-Bu, O-secBu, O-isoBu, O-tBu, O—(CH₂)₂OH, O—(CH₂)₂OCH₃, O—(CH₂)₃OH, O—(CH₂)₃OCH₃, OCF₃, SR¹¹, SH, S-Me, S-Et, S—Pr, S-iPr, S-Bu, S-secBu, S-isoBu, S-tBu, SCF₃, SOR¹¹, SO-Me, SO-Et, SO—Pr, SO-iPr, SO-Bu, SO-secBu, SO-isoBu, SO-tBu, SO₂R¹¹, SO₂-Me, SO₂-Et, SO₂—Pr, SO₂-iPr, SO₂—Bu, SO₂-secBu, SO₂-isoBu, SO₂-tBu, SON(R¹¹)₂, SONHMe, SONMe₂, SONHEt, SONEt₂, SONHPr, SONPr₂, SONHBu, SONBu₂, SONHCF₃, SON(CF₃)₂, SO₂N(R¹¹)₂, SO₂NHMe, SO₂NMe₂, SO₂NEt₂, SO₂NHEt, SO₂NPr₂, SO₂NHPr, SO₂NHCF₃, SO₂N(CF₃)₂, COR¹¹, COMe, COEt, COPr, COiPr, COBu, COsecBu, COisoBu, COtBu, COCF₃, NR¹¹CO₂R¹¹, NR¹¹CO₂Me, NR¹¹CO₂Et, NR¹¹CO₂Pr, NR¹¹CO₂iPr, NR¹¹ CO₂Bu, NR¹¹CO₂secBu, NR¹¹CO₂ isoBu, NR¹¹ CO₂tBu, NR¹¹ COR¹¹, NHCOMe, NHCOEt, NHCOPr, NHCOiPr, NHCOBu, NHCOiBu, NHCOsecBu, NHCOisoBu, NHCOtBu, NHCO(CH₂)₂OH, NHCO(CH₂)₂OCH₃, NHCO(CH₂)₃OH, NHCO(CH₂)₃OCH₃, NR¹¹COMe, NR¹¹ COEt, NR¹¹ COPr, NR¹¹ COiPr, NR¹¹COBu, NR¹¹ COsecBu, NR¹¹COisoBu, NR¹¹COtBu, NR¹¹CO(CH₂)₂OH, NR¹¹CO(CH₂)₂OCH₃, NR¹¹CO(CH₂)₃OH, NR¹¹CO(CH₂)₃OCH₃, N(R¹¹)₂, NMe₂, NEt₂, NHMe, NH₂, NHtBu, NHEt, NHPr, NHiPr, NHBu, NHsecBu, NHisoBu, 4-methylpiperazin-1-yl, piperazin-1-yl, morpholin-1-yl, NR¹¹SO₂R¹², NHSOR¹¹, NR¹¹SOR¹¹, NHSOMe, NHSO₂Me, NHSOEt, NHSO₂Et, NMeSOMe, NHSO₂R¹¹, NR¹¹SO₂R¹¹, NMeSO₂Me, NMeSOEt, NMeSO₂Et, NHSOCF₃, NHSO₂CF₃, OCON(R¹¹)₂, OCONHMe, OCONHEt, OCONHPr, OCONHiPr, OCONHBu, OCONHsecBu, OCONHisoBu, OCONHtBu, OCONMe₂, OCONEt₂, OCONPr₂, OCONiPr₂, OCONBu₂, OCONsecBu₂, OCONisoBu₂, OCONtBu₂, OCOR¹¹, OCOMe, OCOEt, OCOPr, OCOiPr, OCOBu, OCOsecBu, OCOisoBu, OCOtBu, CON(R¹¹)₂, CONHEt, CONEt₂, CONHMe, CONMe₂, CONHPr, CONPr₂, CONHBu, OCONHBu, CONHtBu, CONHCH(CH₃)CH₂OH, CONHCH(CH₃)CH₂OCH₃, CONHCH(C₂H₅)CH₂OH, CONHCH(C₂H₅)CH₂OCH₃, CONH(CH₂)₂OCH₃, CONH(CH₂)₂OH, CONH(CH₂)₃OCH₃, CONH(CH₂)₃OH, CONR¹¹CH(CH₃)CH₂OH, CONR¹¹CH(CH₃)CH₂OCH₃, CONR¹¹CH(C₂H₅)CH₂OH, CONR¹¹ CH(C₂H₅)CH₂OCH₃, CONR¹¹(CH₂)₂OCH₃, CONR¹¹(CH₂)₂OH, CONR¹¹(CH₂)₃OCH₃, CONR¹¹(CH₂)₃OH, CO₂R¹¹, CO₂Me, CO₂Et, CO₂Pr, CO₂iPr, CO₂Bu, CO₂secBu, CO₂ isoBu, CO₂tBu, CO₂(CH₂)₂OH, CO₂(CH₂)₂OCH₃, CO₂(CH₂)₃OH, CO₂(CH₂)₃OCH₃, (CH₂)ₘOR¹¹, CH₂OH, (CH₂)₂OH, (CH₂)₃OH, (CH₂)₄OH, CH₂OMe, (CH₂)₂OMe, (CH₂)₃OMe, (CH₂)₄OMe, (CH₂)ₘSR¹¹, CH₂SH, (CH₂)₂SH, (CH₂)₃SH, (CH₂)₄SH, CH₂SMe, (CH₂)₂SMe, (CH₂)₃SMe, (CH₂)₄SMe, (CH₂)ₘN(R¹¹)₂, CH₂NH₂, CH₂NAc₂, CH₂N(COCF₃)₂, CH₂NHAc, CH₂NHCOCF₃, (CH₂)₂NH₂, (CH₂)₃NH₂, (CH₂)₄NH₂, CH₂NMe₂, (CH₂)₂NHMe, (CH₂)₂NMe₂, (CH₂)₃NHMe, (CH₂)₃NMe₂, (CH₂)₄NHMe, (CH₂)₄NMe₂, (CH₂)ₘCOOR¹², CH₂COOMe, (CH₂)₂COOMe, (CH₂)₃COOMe, CH₂COOEt, (CH₂)₂COOEt, (CH₂)₃COOEt, CH₂COOPr, (CH₂)₂COOPr, (CH₂)₃COOPr, CH₂COOiPr, (CH₂)₂COOiPr, (CH₂)₃COOiPr, CH₂COOtBu, (CH₂)₂COOtBu, (CH₂)₃COOtBu, CH₂COO(CH₂)₂OH CH₂COO(CH₂)₂OCH₃, CH₂COO(CH₂)₃OH, CH₂COO(CH₂)₃OCH₃, CH₂NHCOOR¹¹, CH₂NR¹¹COOR¹¹, CH₂NHCOOMe, CH₂NHCOOtBu, CH₂NHCOOEt, CH₂NHCOOPr, CH₂NHCOOiPr, CH₂NHCOOBu, CH₂NHCOOtBu, CH₂NHCOOsecBu, CH₂NHCOOisoBu, CH₂NR¹¹COOEt, CH₂NR¹¹COOPr, CH₂NR¹¹ COOiPr, CH₂NR¹¹COOBu, CH₂NR¹¹COOtBu, CH₂NR¹¹COOsecBu, CH₂NR¹¹COOisoBu, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methyl-propyl; cyclopropyl, cyclobutyl, cyclopentyl, cyclohexyl; CF₃, CF₂H, CCl₃, C₂F₅, C₃F₇, CF(CF₃)₂,
R¹⁰ is hydrogen, fluorine, chlorine, bromine, iodine, cyano, hydroxyl, amino, N(R¹¹)₂, nitro, OR¹¹, SR¹¹, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methyl-pentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; CF₃, CF₂H, CCl₃, C₂F₅, C₃F₇, CF(CF₃)₂, cyclopropyl, cyclobutyl, cyclopentyl, cyclohexyl, SiMe₃, COOR¹¹, CON(R¹¹)₂,
R¹¹ are identical or different and are hydrogen, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, CH₂CH₂OMe, CH(CH₃)CH₂OMe, CH₂CH(CH₃)OMe, CH₂CH₂OEt, CH(CH₃)CH₂OEt, CH₂CH(CH₃)OEt, CF₃, CF₂H, CCl₃, C₂F₅, C₃F₇, CF(CF₃)₂, cyclopropyl, cyclobutyl, cyclopentyl, cyclohexyl, SiMe₃, phenyl or
   if two radicals R¹¹ are attached to one nitrogen atom, these radicals together may represent piperidin-1-yl, piperazin-1-yl, 4-methylpiperazin-1-yl or morpholin-1-yl, or,
   if two radicals R¹¹ are adjacent in the grouping NR¹¹COR¹¹, two radicals R¹¹ may form a 3- to 7-membered, unsubstituted or substituted saturated or unsaturated cycle which may contain up to five further heteroatoms selected from the group consisting of N, O and S, where two oxygen atoms are not adjacent,
R¹² is methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; CF₃, CF₂H, CCl₃, C₂F₅, C₃F₇, CF(CF₃)₂, cyclopropyl, cyclobutyl, cyclopentyl, cyclohexyl, CH₂OR¹¹, (CH₂)₂OR¹¹, (CH₂)₃OR¹¹, (CH₂)₄OR¹¹, SiMe₃.

Very particular preference is given to compounds of the formula (I) in which one or more of the symbols have one of the following meanings:
R¹ to R⁴ independently of one another are hydrogen, fluorine, chlorine, bromine, iodine, cyano, hydroxyl, nitro, OR¹¹, O-Me, O-Et, O—Pr, O-iPr, O-Bu, O-secBu, O-isoBu, O-tBu, O—(CH₂)₂OH, O—(CH₂)₂OCH₃, O—(CH₂)₃OH, O—(CH₂)₃OCH₃, OCF₃, SR¹¹, SH, S-Me, S-Et, S—Pr, S-iPr, S-Bu, S-secBu, S-isoBu, S-tBu, SCF₃, SOR¹¹, SO-Me, SO-Et, SO—Pr, SO-iPr, SO-Bu, SO-secBu, SO-isoBu, SO-tBu, SO₂R¹¹, SO₂-Me, SO₂-Et, SO₂—Pr, SO₂-iPr, SO₂—Bu, SO₂-secBu, SO₂-isoBu, SO₂-tBu, SON(R¹¹)₂, SONHMe, SONMe₂, SONHEt, SONEt₂, SONHPr, SONPr₂, SONHBu, SONBu₂, SONHCF₃, SON(CF₃)₂, SO₂N(R¹¹)₂, SO₂NHMe, SO₂NMe₂, SO₂NEt₂, SO₂NHEt, SO₂NPr₂, SO₂NHPr, SO₂NHCF₃, SO₂N(CF₃)₂, COR¹¹, COMe, COEt, COPr, COiPr, COBu, COsecBu, CO-isoBu, COtBu, COCF₃, NR¹¹CO₂R¹¹, NR¹¹CO₂Me, NR¹¹CO₂Et, NR¹¹CO₂Pr, NR¹¹CO₂iPr, NR¹¹CO₂Bu, NR¹¹CO₂secBu, NR¹¹CO₂ isoBu, NR¹¹CO₂tBu, NR¹¹COR¹¹, NHCOMe, NHCOEt, NHCOPr, NHCOiPr, NHCOBu, NHCOiBu, NHCOsecBu, NHCOisoBu, NHCOtBu, NHCO(CH₂)₂OH, NHCO(CH₂)₂OCH₃, NHCO(CH₂)₃OH, NHCO(CH₂)₃OCH₃, NR¹¹COMe, NR¹¹COEt, NR¹¹COPr, NR¹¹COiPr, NR¹¹COBu, NR¹¹COsecBu, NR¹¹COtBu, NR¹¹CO(CH₂)₂OH, NR¹¹CO(CH₂)₂OCH₃, NR¹¹CO(CH₂)₃OH, NR¹¹ CO(CH₂)₃OCH₃, N(R¹¹)₂, NMe₂, NEt₂, NHMe, NH₂, NHtBu, NHEt, NHPr, NHiPr, NHBu, NHiBu, NHsecBu, piperazin-1-yl, 4-methylpiperazin-1-yl, morpholin-1-yl, NR¹¹SO₂R¹², NHSOR¹¹, NR¹¹SOR¹¹, NHSOMe, NHSO₂Me, NHSOEt, NHSO₂Et, NMeSOMe, NHSO₂R¹¹, NR¹¹SO₂R¹¹, NMeSO₂Me, NMeSOEt, NMeSO₂Et, NHSOCF₃, NHSO₂CF₃, OCON(R¹¹)₂, OCONHMe, OCONHEt, OCONHPr, OCONHiPr, OCONHBu, OCONHsecBu, OCONHisoBu, OCONHtBu, OCONMe₂, OCONEt₂, OCONPr₂, OCONiPr₂, OCONBu₂, OCONsecBu₂, OCONisoBu₂, OCONHtBu₂, OCOR¹¹, OCOMe, OCOEt, OCOPr, OCOiPr, OCOBu, OCOsecBu, OCOisoBu, OCOtBu, CON(R¹¹)₂, CONHEt, CONEt₂, CONHMe, CONMe₂, CONHPr, CONPr₂, CONHBu, CONHsecBu, CONHisoBu, CONHtBu, CONHCH(CH₃)CH₂OH, CONHCH(CH₃)CH₂OCH₃, CONHCH(C₂H₅)CH₂OH, CONHCH(C₂H₅)CH₂OCH₃, CONH(CH₂)₂OCH₃, CONH(CH₂)₂OH, CONH(CH₂)₃OCH₃, CONH(CH₂)₃OH, CONR¹¹CH(CH₃)CH₂OH, CONR¹¹CH(CH₃)CH₂OCH₃, CONR¹¹CH(C₂H₅)CH₂OH, CONR¹¹CH(C₂H₅)CH₂OCH₃, CONRt¹¹(CH₂)₂OCH₃, CONR¹¹(CH₂)₂OH, CONR¹¹(CH₂)₃OCH₃, CONR¹¹(CH₂)₃OH, CO₂R¹¹, CO₂Me, CO₂Et, CO₂Pr, CO₂iPr, CO₂Bu, CO₂secBu, CO₂ isoBu, CO₂tBu, CO₂(CH₂)₂OH, CO₂(CH₂)₂OCH₃, CO₂(CH₂)₃OH, CO₂(CH₂)₃OCH₃, (CH₂)ₘOR¹¹, CH₂OH, (CH₂)₂OH, (CH₂)₃OH, (CH₂)₄OH, CH₂OMe, (CH₂)₂OMe, (CH₂)₃OMe, (CH₂)₄OMe, (CH₂)ₘSR¹¹, CH₂SH, (CH₂)₂SH, (CH₂)₃SH, (CH₂)₄SH, CH₂SMe, (CH₂)₂SMe, (CH₂)₃SMe, (CH₂)₄SMe, (CH₂)ₘN(R¹¹)₂, CH₂NH₂, CH₂NAc₂, CH₂N(COCF₃)₂, CH₂NHAc, CH₂NHCOCF₃, (CH₂)₂NH₂, (CH₂)₃NH₂, (CH₂)₄NH₂, CH₂NMe₂, (CH₂)₂NHMe, (CH₂)₂NMe₂, (CH₂)₃NHMe, (CH₂)₃NMe₂, (CH₂)₄NHMe, (CH₂)₄NMe₂, (CH₂)ₘCOOR², CH₂COOMe, (CH₂)₂COOMe, (CH₂)₃COOMe, CH₂COOEt, (CH₂)₂COOEt, (CH₂)₃COOEt, CH₂COOPr, (CH₂)₂COOPr, (CH₂)₃COOPr, CH₂COOiPr, (CH₂)₂COOiPr, (CH₂)₃COOiPr, CH₂COOtBu, (CH₂)₂COOtBu, (CH₂)₃COOtBu, CH₂COO(CH₂)₂OH CH₂COO(CH₂)₂OCH₃, CH₂COO(CH₂)₃OH, CH₂COO(CH₂)₃OCH₃, CH₂NHCOOR¹¹, CH₂NR¹¹COOR¹¹, CH₂NHCOOMe, CH₂NHCOOtBu, CH₂NHCOOEt, CH₂NHCOOPr, CH₂NHCOOiPr, CH₂NHCOOBu, CH₂NHCOOtBu, CH₂NHCOOsecBu, CH₂NHCOOisoBu, CH₂NR¹¹COOEt, CH₂NR¹¹COOPr, CH₂NR¹¹COOiPr, CH₂NR¹¹COOBu, CH₂NR¹¹COOtBu, CH₂NR¹¹COOsecBu, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; cyclopropyl, cyclobutyl, cyclopentyl, cyclohexyl; CF₃, CF₂H, CCl₃, C₂F₅, C₃F₇, CF(CF₃)₂
   or in each case two adjacent radicals R¹, R² or R², R³ or R³, R⁸ or R⁸, R⁴ together form a 2,3-dihydro-1,4-dioxin, a 2,2,3,3-tetrafluoro-2,3-dihydro-1,4-dioxin, a 1,3-dioxole or a 2,2-difluoro-1,3-dioxole ring,
R⁵ is hydrogen, acetyl, trifluoroacetyl,
X is nitrogen or CR⁸,
Y is nitrogen,
Z is nitrogen or CR¹⁰,
R⁶ is hydrogen, fluorine, chlorine, bromine, iodine, cyano, hydroxyl, OR¹¹, O-Me, O-Et, O—Pr, O-iPr, O-Bu, O-secBu, O-isoBu, O-tBu, OCH(CH₃)CH₂OH, OCH(CH₃)CH₂OCH₃, OCH(C₂H₅)CH₂OH, OCH(C₂H₅)CH₂OCH₃, O(CH₂)₂OCH₃, O(CH₂)₂OH, O(CH₂)₃OCH₃, O(CH₂)₃OH, OCF₃, SR¹¹, S-Me, S-Et, S—Pr, S-iPr, S-Bu, S-secBu, S-isoBu, S-tBu, SCF₃, SOR¹¹, SO-Me, SO-Et, SO—Pr, SO-iPr, SO-Bu, SO-secBu, SO-isoBu, SO-tBu, SO₂R¹¹, SO₂-Me, SO₂-Et, SO₂—Pr, SO₂-iPr, SO₂—Bu, SO₂-secBu, SO₂-isoBu, SO₂-tBu, COR¹¹, COMe, COEt, COPr, COiPr, COBu, COsecBu, COisoBu, COtBu, COCF₃, CO₂R¹¹, CO₂Me, CO₂Et, CO₂Pr, CO₂iPr, CO₂Bu, CO₂secBu, CO₂ isoBu, CO₂tBu, CO₂CH(CH₃)CH₂OH, CO₂CH(CH₃)CH₂OCH₃, CO₂CH(C₂H₅)CH₂OH, CO₂CH(C₂H₅)CH₂OCH₃, CO₂(CH₂)₂OCH₃, CO₂(CH₂)₂OH, CO₂(CH₂)₃OCH₃, CO₂(CH₂)₃OH, NR¹¹CO₂R¹¹, NR¹¹CO₂Me, NR¹¹CO₂Et, NR¹¹CO₂Pr, NR¹¹CO₂iPr, NR¹¹CO₂Bu, NR¹¹CO₂secBu, NR¹¹CO₂ isoBu, NR¹¹CO₂tBu, NR¹¹ COR¹¹, NR¹¹COMe, NR¹¹COEt, NR¹¹COPr, NR¹¹ COiPr, NR¹¹ COBu, NR¹¹COsecBu, NR¹¹COisoBu, NR¹¹COtBu, Me, Et, Pr, iPr, Bu, secBu, isoBu, tBu, CH₂OCH₃, (CH₂)₂OCH₃, CH(CH₃)CH₂OCH₃, (CH₂)₂OH, CH(CH₃)CH₂OH, CF₃, CF₂H, CCl₃, C₂F₅, C₃F₇, CF(CF₃)₂,
   SiMe₃, SiMe₂tBu, SiMe₂Ph, NHR¹¹, N(R¹¹)₂, NMe₂, NEt₂, NHMe, NH₂, NHtBu, NHEt, NHPr, NHiPr, NHBu, NHsecBu, NHisoBu, NHCH(CH₃)CH₂OH, NHCH(CH₃)CH₂OCH₃, NHCH(C₂H₅)CH₂OH, NHCH(C₂H₅)CH₂OCH₃, NH(CH₂)₂OCH₃, NH(CH₂)₂OH, NH(CH₂)₃OCH₃, NH(CH₂)₃OH, NHAc, NR¹¹CH(CH₃)CH₂OH, NR¹¹CH(CH₃)CH₂OCH₃, NR¹¹CH(C₂H₅)CH₂OH, NR¹¹CH(C₂H₅)CH₂OCH₃, NR¹¹ (CH₂)₂OCH₃, NR¹¹(CH₂)₂OH, NR¹¹(CH₂)₃OCH₃, NR¹¹(CH₂)₃OH, NR¹¹Ac, NHCOEt, NHCOPr, NHCOiPr, NHCOBu, NHCOsecBu, NHCOisoBu, NHCOtBu, NHCOCF₃, NHCOC₂F₅, N(Me)tBu, N(Me)Et, N(Me)Pr, N(Me)iPr, N(Me)Bu, N(Me)secBu, N(Me)isoBu, N(CH₃)CH(CH₃)CH₂OH, N(CH₃)CH(CH₃)CH₂OCH₃, N(CH₃)CH(C₂H₅)CH₂OH, N(CH₃)CH(C₂H₅)CH₂OCH₃, N(CH₃)(CH₂)₂OCH₃, N(CH₃)(CH₂)₂OH, N(CH₃)(CH₂)₃OCH₃, N(CH₃)(CH₂)₃OH, NHCOR¹¹, NR¹¹COR¹¹, NMeAc, NMeCOEt, NMeCOPr, NMeCOiPr, NMeCOBu, NMeCOsecBu, NMeCOisoBu, NMeCOtBu, NMeCOCF₃, NMeCOC₂F₅, NHCO₂R¹¹, NR¹¹CO₂R¹¹, NHCO₂Me, NHCO₂Et, NHCO₂Pr, NHCO₂iPr, NHCO₂Bu, NHCO₂secBu, NHCO₂ isoBu, NHCO₂tBu, NHCO₂CF₃, NHCO₂C₂F₅, NHCO₂CH(CH₃)CH₂OH, NHCO₂CH(CH₃)CH₂OCH₃, NHCO₂CH(C₂H₅)CH₂OH, NHCO₂CH(C₂H₅)CH₂OCH₃, NHCO₂(CH₂)₂OCH₃, NHCO₂(CH₂)₂OH, NHCO₂(CH₂)₃OCH₃, NHCO₂(CH₂)₃OH, NMeCO₂Me, NMeCO₂Et, NMeCO₂Pr, NMeCO₂iPr, NMeCO₂Bu, NMeCO₂secBu, NMeCO₂ isoBu, NMeCO₂tBu, NMeCO₂CF₃, NMeCO₂C₂F₅, NEtCO₂Me, NEtCO₂Et, NEtCO₂Pr, NEtCO₂iPr, NEtCO₂Bu, NEtCO₂secBu, NEtCO₂ isoBu, NEtCO₂tBu, NEtCO₂CF₃, NEtCO₂C₂F₅, SON(R¹¹)₂, SONHMe, SONMe₂, SONHEt, SONEt₂, SONHPr, SONPr₂, SONHBu, SONBu₂, SONHCF₃, SON(CF₃)₂, SO₂N(R¹¹)₂, SO₂NHMe, SO₂NMe₂, SO₂NEt₂, SO₂NHEt, SO₂NPr₂, SO₂NHPr, SO₂NHCF₃, SO₂N(CF₃)₂, SO₂NHCH(CH₃)CH₂OH, SO₂NHCH(CH₃)CH₂OCH₃, SO₂NHCH(C₂H₅)CH₂OH, SO₂NHCH(C₂H₅)CH₂OCH₃, SO₂NH(CH₂)₂OCH₃, SO₂NH(CH₂)₂OH, SO₂NH(CH₂)₃OCH₃, SO₂NH(CH₂)₃OH, NHSOR¹¹, NR¹¹SOR¹¹, NHSOMe, NHSO₂Me, NHSOEt, NHSO₂Et, NMeSOMe, NHSO₂R¹¹, NR¹¹SO₂R¹¹, NMeSO₂Me, NMeSOEt, NMeSO₂Et, NHSOCF₃, NHSO₂CF₃, CON(R¹¹)₂, CONH₂, CONHMe, CONMe₂, CONHEt, CONEt₂, CO-morpholine, CO-piperidine, CO-piperazine, CO-(4-methylpiperazine), CONHCH(CH₃)CH₂OH, CONHCH(CH₃)CH₂OCH₃, CONHCH(C₂H₅)CH₂OH, CONHCH(C₂H₅)CH₂OCH₃, CONH(CH₂)₂OCH₃, CONH(CH₂)₂OH, CONH(CH₂)₃OCH₃, CONH(CH₂)₃OH, cyclopropyl, cyclopentyl, cyclohexyl, phenyl, pyridin-4-yl, pyridin-3-yl, pyridin-2-yl, thien-2-yl, thien-3-yl, furan-2-yl, furan-3-yl, N-1,3-oxazolidin-2-one, N-4-methyl-1,3-oxazolidin-2-one, N-4,4-dimethyl-1,3-oxazolidin-2-one, N4,5-dimethyl-1,3-oxazolidin-2-one, N-5,5-dimethyl-1,3-oxazolidin-2-one, N-4-ethyl-1,3-oxazolidin-2-one, N-4,4-ethyl-1,3-oxazolidin-2-one, NHCH₂-tetrahydrofuran-2-yl,
R⁷ is hydrogen, fluorine, chlorine, bromine, iodine, cyano, hydroxyl, amino, N(R¹¹)₂, NMe₂, NEt₂, NHMe, NH₂, NHtBu, NHEt, NHPr, NHiPr, NHBu, NHiBu, NHsecBu, piperazin-1-yl, 4-methylpiperazin-1-yl, morpholin-1-yl, nitro, OR¹¹, O-Me, O-Et, O—Pr, O-iPr, O-Bu, O-secBu, O-isoBu, O-tBu, OCH(CH₃)CH₂OH, OCH(CH₃)CH₂OCH₃, OCH(C₂H₅)CH₂OH, OCH(C₂H₅)CH₂OCH₃, O(CH₂)₂OCH₃, O(CH₂)₂OH, O(CH₂)₃OCH₃, O(CH₂)₃OH, OCF₃, SR¹¹, S-Me, S-Et, S—Pr, S-iPr, S-Bu, S-secBu, S-isoBu, S-tBu, SCF₃, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; CF₃, CF₂H, CCl₃, C₂F₅, C₃F₇, CF(CF₃)₂, cyclopropyl, cyclobutyl, cyclopentyl, cyclohexyl, SiMe₃, COOR¹¹, CO₂Me, CO₂Et, CO₂Pr, CO₂iPr, CO₂Bu, CO₂secBu, CO₂ isoBu, CO₂tBu, CON(R¹¹)₂, CONH₂, CONHMe, CONMe₂, CONHEt, CONEt₂, CO-morpholine, CO-piperidine, CO-piperazine, CO-(4-methylpiperazine), CONHCH(CH₃)CH₂OH, CONHCH(CH₃)CH₂OCH₃, CONHCH(C₂H₅)CH₂OH, CONHCH(C₂H₅)CH₂OCH₃, CONH(CH₂)₂OCH₃, CONH(CH₂)₂OH, CONH(CH₂)₃OCH₃, CONH(CH₂)₃OH, COR¹¹, COMe, COEt, COPr, COiPr, COBu, COsecBu, COisoBu, COtBu, COF₃ or
R⁷ and R¹⁰ together form a saturated or unsaturated bridge of the following structure:

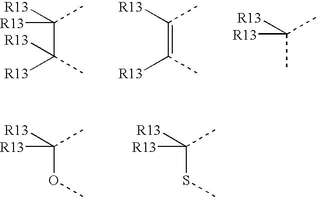

where R¹³=independently of one another hydrogen, fluorine, chlorine, bromine, iodine, methyl, ethyl, propyl, 1-methylethyl, CF₂H, CF₃, C₂F₅, OCH₃; OC₂H₅, OCF₃, OC₂F₅ or
   two geminal radicals R¹³ represent doubly attached oxygen or sulphur,
R⁸ is hydrogen, fluorine, chlorine, bromine, iodine, cyano, hydroxyl, nitro, OR¹¹, O-Me, O-Et, O—Pr, O-iPr, O-Bu, O-secBu, O-isoBu, O-tBu, O—(CH₂)₂OH, O—(CH₂)₂OCH₃, O—(CH₂)₃OH, O—(CH₂)₃OCH₃, OCF₃, SR¹¹, SH, S-Me, S-Et, S—Pr, S-iPr, S-Bu, S-secBu, S-isoBu, S-tBu, SCF₃, SOR¹¹, SO-Me, SO-Et, SO—Pr, SO-iPr, SO-Bu, SO-secBu, SO-isoBu, SO-tBu, SO₂R¹¹, SO₂-Me, SO₂-Et, SO₂—Pr, SO₂-iPr, SO₂—Bu, SO₂-secBu, SO₂-isoBu, SO₂-tBu, SON(R¹¹)₂, SONHMe, SONMe₂, SONHEt, SONEt₂, SONHPr, SONPr₂, SONHBu, SONBu₂, SONHCF₃, SON(CF₃)₂, SO₂N(R¹¹)₂, SO₂NHMe, SO₂NMe₂, SO₂NEt₂, SO₂NHEt, SO₂NPr₂, SO₂NHPr, SO₂NHCF₃, SO₂N(CF₃)₂, COR¹¹, COMe, COEt, COPr, COiPr, COBu, COsecBu, COisoBu, COtBu, COCF₃, NR¹¹CO₂R¹¹, NR¹¹CO₂Me, NR¹¹CO₂Et, NR¹¹CO₂Pr, NR¹¹CO₂iPr, NR¹¹CO₂Bu, NR¹¹CO₂secBu, NR¹¹CO₂ isoBu, NR¹¹CO₂tBu, NR¹¹COR¹¹, NHCOMe, NHCOEt, NHCOPr, NHCOiPr, NHCOBu, NHCOiBu, NHCOsecBu, NHCOisoBu, NHCOtBu, NHCO(CH₂)₂OH, NHCO(CH₂)₂OCH₃, NHCO(CH₂)₃OH, NHCO(CH₂)₃OCH₃, NR¹¹COMe, NR¹¹COEt, NR¹¹ COPr, NR¹¹ COiPr, NR¹¹COBu, NR¹¹COsecBu, NR¹¹COisoBu, NR¹¹COtBu, NR¹¹CO(CH₂)₂OH, NR¹¹CO(CH₂)₂OCH₃, NR¹¹CO(CH₂)₃OH, NR¹¹CO(CH₂)₃OCH₃, N(R¹¹)₂, NMe₂, NEt₂, NHMe, NH₂, NHtBu, NHEt, NHPr, NHiPr, NHBu, NHsecBu, NHisoBu, 4-methylpiperazin-1-yl, piperazin-1-yl, morpholin-1-yl, NR¹¹SO₂R¹², NHSOR¹¹, NR¹¹SOR¹¹, NHSOMe, NHSO₂Me, NHSOEt, NHSO₂Et, NMeSOMe, NHSO₂R¹¹, NR¹¹SO₂R¹¹, NMeSO₂Me, NMeSOEt, NMeSO₂Et, NHSOCF₃, NHSO₂CF₃, OCON(R¹¹)₂, OCONHMe, OCONHEt, OCONHPr, OCONHiPr, OCONHBu, OCONHsecBu, OCONHisoBu, OCONHtBu, OCONMe₂, OCONEt₂, OCONPr₂, OCONiPr₂, OCONBu₂, OCONsecBu₂, OCONisoBu₂, OCONtBu₂, OCOR¹¹, OCOMe, OCOEt, OCOPr, OCOiPr, OCOBu, OCOsecBu, OCOisoBu, OCOtBu, CON(R¹¹)₂, CONHEt, CONEt₂, CONHMe, CONMe₂, CONHPr, CONPr₂, CONHBu, OCONHBu, CONHtBu, CONHCH(CH₃)CH₂OH, CONHCH(CH₃)CH₂OCH₃, CONHCH(C₂H₅)CH₂OH, CONHCH(C₂H₅)CH₂OCH₃, CONH(CH₂)₂OCH₃, CONH(CH₂)₂OH, CONH(CH₂)₃OCH₃, CONH(CH₂)₃OH, CONR¹¹CH(CH₃)CH₂OH, CONR¹¹ CH(CH₃)CH₂OCH₃, CONR¹¹CH(C₂H₅)CH₂OH, CONR¹¹CH(C₂H₅)CH₂OCH₃, CONR¹¹ (CH₂)₂OCH₃, CONR¹¹(CH₂)₂OH, CONR¹¹ (CH₂)₃OCH₃, CONR¹¹ (CH₂)₃OH, CO₂R¹¹, CO₂Me, CO₂Et, CO₂Pr, CO₂iPr, CO₂Bu, CO₂secBu, CO₂ isoBu, CO₂tBu, CO₂(CH₂)₂OH, CO₂(CH₂)₂OCH₃, CO₂(CH₂)₃OH, CO₂(CH₂)₃OCH₃, (CH₂)ₘOR¹¹, CH₂OH, (CH₂)₂OH, (CH₂)₃OH, (CH₂)₄OH, CH₂OMe, (CH₂)₂OMe, (CH₂)₃OMe, (CH₂)₄OMe, (CH₂)ₘSR¹¹, CH₂SH, (CH₂)₂SH, (CH₂)₃SH, (CH₂)₄SH, CH₂SMe, (CH₂)₂SMe, (CH₂)₃SMe, (CH₂)₄SMe, (CH₂)ₘN(R¹¹)₂, CH₂NH₂, CH₂NAc₂, CH₂N(COCF₃)₂, CH₂NHAc, CH₂NHCOCF₃, (CH₂)₂NH₂, (CH₂)₃NH₂, (CH₂)₄NH₂, CH₂NMe₂, (CH₂)₂NHMe, (CH₂)₂NMe₂, (CH₂)₃NHMe, (CH₂)₃NMe₂, (CH₂)₄NHMe, (CH₂)₄NMe₂, (CH₂)ₘCOOR¹², CH₂COOMe, (CH₂)₂COOMe, (CH₂)₃COOMe, CH₂COOEt, (CH₂)₂COOEt, (CH₂)₃COOEt, CH₂COOPr, (CH₂)₂COOPr, (CH₂)₃COOPr, CH₂COOiPr, (CH₂)₂COOiPr, (CH₂)₃COOiPr, CH₂COOtBu, (CH₂)₂COOtBu, (CH₂)₃COOtBu, CH₂COO(CH₂)₂OH, CH₂COO(CH₂)₂OCH₃, CH₂COO(CH₂)₃OH, CH₂COO(CH₂)₃OCH₃, CH₂NHCOOR¹¹, CH₂NR¹¹COOR¹¹, CH₂NHCOOMe, CH₂NHCOOtBu, CH₂NHCOOEt, CH₂NHCOOPr, CH₂NHCOOiPr, CH₂NHCOOBu, CH₂NHCOOtBu, CH₂NHCOOsecBu, CH₂NHCOOisoBu, CH₂NR¹¹COOEt, CH₂NR¹¹COOPr, CH₂NR¹¹ COOiPr, CH₂NR¹¹COOBu, CH₂NR¹¹COOtBu, CH₂NR¹¹COOsecBu, CH₂NR¹¹COOisoBu, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methyl-propyl; cyclopropyl, cyclobutyl, cyclopentyl, cyclohexyl; CF₃, CF₂H, CCl₃, C₂F₅, C₃F₇, CF(CF₃)₂,
R¹⁰ is hydrogen, fluorine, chlorine, bromine, iodine, cyano, hydroxyl, amino, N(R¹¹)₂, NMe₂, NEt₂, NHMe, NH₂, NHtBu, NHEt, NHPr, NHiPr, NHBu, NHsecBu, NHisoBu, 4-methylpiperazin-1-yl, piperazin-1-yl, morpholin-1-yl, nitro, OR¹¹, O-Me, O-Et, O—Pr, O-iPr, O-Bu, O-secBu, O-isoBu, O-tBu, OCH(CH₃)CH₂OH, OCH(CH₃)CH₂OCH₃, OCH(C₂H₅)CH₂OH, OCH(C₂H₅)CH₂OCH₃, O(CH₂)₂OCH₃, O(CH₂)₂OH, O(CH₂)₃OCH₃, O(CH₂)₃OH, OCF₃, SR¹¹, S-Me, S-Et, S—Pr, S-iPr, S-Bu, S-secBu, S-isoBu, S-tBu, SCF₃, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; CF₃, CF₂H, CCl₃, C₂F₅, C₃F₇, CF(CF₃)₂, cyclopropyl, cyclobutyl, cyclopentyl, cyclohexyl, SiMe₃, COOR¹¹, CO₂Me, CO₂Et, CO₂Pr, CO₂iPr, CO₂Bu, CO₂secBu, CO₂ isoBu, CO₂tBu, CON(R¹¹)₂ CONH₂, CONHMe, CONMe₂, CONHEt, CONEt₂, CO-morpholine, CO-piperidine, CO-piperazine, CO-(4-methylpiperazine), CONHCH(CH₃)CH₂OH, CONHCH(CH₃)CH₂OCH₃, CONHCH(C₂H₅)CH₂OH, CONHCH(C₂H₅)CH₂OCH₃, CONH(CH₂)₂OCH₃, CONH(CH₂)₂OH, CONH(CH₂)₃OCH₃, CONH(CH₂)₃OH,
R¹¹ are identical or different and are hydrogen, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, CH₂CH₂OMe, CH(CH₃)CH₂OMe, CH₂CH(CH₃)OMe, CH₂CH₂OEt, CH(CH₃)CH₂OEt, CH₂CH(CH₃)OEt, CF₃, CF₂H, CCl₃, C₂F₅, C₃F₇, CF(CF₃)₂, cyclopropyl, cyclobutyl, cyclopentyl, cyclohexyl, SiMe₃, phenyl or
   if two radicals R¹¹ are attached to one nitrogen atom, these radicals together may represent piperidin-1-yl, piperazin-1-yl, 4-methylpiperazin-1-yl or morpholin-1-yl, or
   if two radicals R¹¹ are adjacent in the grouping NR¹¹COR¹¹, two radicals R¹¹ may form a 3- to 7-membered, unsubstituted or substituted saturated or unsaturated cycle which may contain up to five further heteroatoms selected from the group consisting of N, O and S, where two oxygen atoms are not adjacent,
R¹² is methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; CF₃, CF₂H, CCl₃, C₂F₅, C₃F₇, CF(CF₃)₂, cyclopropyl, cyclobutyl, cyclopentyl, cyclohexyl, CH₂OR¹¹, (CH₂)₂OR¹¹, (CH₂)₃OR¹¹, (CH₂)₄OR¹¹, SiMe₃.

Furthermore, very particular preference is given to compounds of the formula (I) in which one or more of the symbols have one of the following meanings:
R¹ to R⁴ independently of one another are hydrogen, fluorine, chlorine, cyano, hydroxyl, nitro, OMe, CF₃, COCH₃, COOCH₃, COOH, N(Me)₂, NHCOCH₃, NHCOCF₃, NHSO₂CH₃, NHCOOCH₃, NHCOO(CH₂CH₂)OCH₃, 4-N-methylpiperazin-1-yl, CH₂NH₂, CH₂NHCOOCH₃, CH₂NHCOOtBu, methyl,
   or in each case two adjacent radicals R¹, R² or R², R³ or R³, R⁸ or R⁸, R⁴ together form a 2,3-dihydro-1,4-dioxin or a 1,3-dioxole ring,
R⁵ is hydrogen, COCH₃,
X is nitrogen or CR⁸,
Y is nitrogen,
Z is nitrogen or CR¹⁰,
R⁶ is hydrogen, S-Me, NHCOMe, NHCOCF₃, NMe₂, NHMe, NH₂, NHtBu, NHCH(CH₃)CH₂OH, NHCH(CH₃)CH₂OCH₃, CONMe₂, cyclopropyl, cyclohexyl, phenyl, N-1,3-oxazolidin-2-one, N-4-methyl-1,3-oxazolidin-2-one, NHCH₂-tetrahydrofuran-2-yl,
R⁷ is hydrogen, methyl, 1-methylpropyl, CF₃, CF₂H, cyclopropyl, or
R⁷ and R¹⁰ together form a saturated or unsaturated bridge of the following structure:

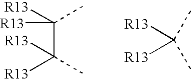

where R¹³=hydrogen,
R⁸ is hydrogen, fluorine, chlorine, cyano, hydroxyl, nitro, OMe, CF₃, COCH₃, COOCH₃, COOH, N(Me)₂, NHCOCH₃, NHCOCF₃, NHSO₂CH₃, NHCOOCH₃, NHCOO(CH₂CH₂)OCH₃, 4-N-methylpiperazin-1-yl, CH₂NH₂, CH₂NHCOOCH₃, CH₂NHCOOtBu, methyl,
R¹⁰ is hydrogen or
R⁷ and R¹⁰ together form a saturated or unsaturated bridge of the following structure:

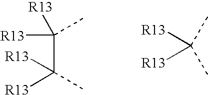

where R¹³=hydrogen.

Furthermore, very particular preference is given to compounds of the formula (I) in which one or more of the symbols have one of the following meanings:
R¹ to R⁴ independently of one another are hydrogen, fluorine, chlorine, iodine, cyano, hydroxyl, nitro, OMe, OCH₂tBu, OCOCH₃, SO₂NH₂, SO₂N(CH₃)₂, COCH₃, COCH₂CH₂CH₃, COOCH₂CH₂OCH₃, COOCH₂CH₂CH₂OCH₃, CONHCH₂CH₂OCH₃, NH₂, N(Me)₂, NHCOCH₃, NHSO₂CH₃, NHCOOCH₃, NHCOOCH₂CH₃, NHCOOtBu, NHCOOCH₂CH₂OCH₃, 4-N-acetylpiperazin-1-yl, N-pyrrolidin-2-on-1-yl, CH₂NHCOOCH₃, CH₂NHCOOtBu, methyl, CF₃
   or in each case two adjacent radicals R¹, R² or R², R³ or R³, R⁸ or R⁸, R⁴ together form a 2,3-dihydrofuran-2-one, 2,3-dihydro-1,4-dioxin, a 2,2,3,3-tetrafluoro-2,3-dihydro-1,4-dioxin, a 1,3-dioxole or a 2,2-difluoro-1,3-dioxole ring,
R⁵ is hydrogen, COCH₃, COCF₃, COOCH₃,
X is nitrogen or CR⁸,
R⁸ is hydrogen, O-Me,
Y is nitrogen,
Z is CR¹⁰,
   R¹⁰ is hydrogen, fluorine, chlorine, bromine, cyano, CF₃ or
R⁷ and R¹⁰ together form a CH₂ or a CH₂CH₂ bridge,
R⁶ is methyl, ethyl, isopropyl, cyclopropyl, phenyl, 4-chlorophenyl, 3-pyridyl, CH₂OCH₃, O(CH₂)₂OCH₃, S-Me, NH₂, NHMe, NMe₂, NHtBu, NHBu, NHiBu, N-morpholinyl, NH(CH₂)₂OH, NH(CH₂)₂OCH₃, NH(CH₂)₃OCH₃, NHCH(CH₃)CH₂OCH₃, NCH₃(CH₂)₂OCH₃, NCH₃(CH₂)₃OCH₃, NHCH₂-tetrahydrofuran-2-yl, N-1,3-oxazolidin-2-one,
   R⁷ is hydrogen, methyl, CF₃, CF₂H, or
R⁷ and R¹⁰ together form a CH₂ or a CH₂CH₂ bridge.

Moreover, very particular preference is given compounds of the formula (I) in which one or more of the symbols have one of the following meanings:
R¹ to R⁴ independently of one another are hydrogen, fluorine, chlorine, iodine, cyano, hydroxyl, nitro, OMe, OCH₂tBu, OCOCH₃, SO₂NH₂, SO₂N(CH₃)₂, COCH₃, COCH₂CH₂CH₃, COOCH₂CH₂OCH₃, COOCH₂CH₂CH₂OCH₃, CONHCH₂CH₂OCH₃, NH₂, N(Me)₂, NHCOCH₃, NHSO₂CH₃, NHCOOCH₃, NHCOOCH₂CH₃, NHCOOtBu, NHCOOCH₂CH₂OCH₃, CH₂NHCOOCH₃, CH₂NHCOOtBu, methyl, CF₃
   or in each case two adjacent radicals R¹, R² or R², R³ or R³, R⁸ or R⁸, R⁴ together form a 2,3-dihydro-1,4-dioxin, a 2,2,3,3-tetrafluoro-2,3-dihydro-1,4-dioxin, a 1,3-dioxole or 2,2-difluoro-1,3-dioxole ring,
R⁵ is hydrogen, COCH₃, COCF₃, COOCH₃,
X is nitrogen or CR⁸,
R⁸ is hydrogen, O-Me,
Y is nitrogen,
Z is CR¹⁰,
   R¹⁰ is hydrogen, fluorine, chlorine, bromine, cyano, CF₃, or
R⁷ and R¹⁰ together form a CH₂— or a CH₂CH₂ bridge,
R⁶ is methyl, ethyl, isopropyl, cyclopropyl, phenyl, 3-pyridyl, CH₂OCH₃, O(CH₂)₂OCH₃, S-Me, NH₂, NHMe, NMe₂, NHtBu, NHBu, NHiBu, N-morpholinyl, NH(CH₂)₂OH, NH(CH₂)₂OCH₃, NH(CH₂)₃OCH₃, NHCH(CH₃)CH₂OCH₃, NCH₃(CH₂)₂OCH₃, NCH₃(CH₂)₃OCH₃, NHCH₂-tetrahydrofuran-2-yl, N-1,3-oxazolidin-2-one,
R⁷ is hydrogen, methyl, CF₃, CF₂H, or
R⁷ and R¹⁰ together form a CH₂ or a CH₂CH₂ bridge.

The radical definitions mentioned above can be combined with one another as desired. Moreover, individual definitions may not apply.

The compounds of the formula (I) furthermore comprise the compounds of the formula (Ia). Not known and thus also part of the subject-matter of the invention are the compounds of the formula (Ia)

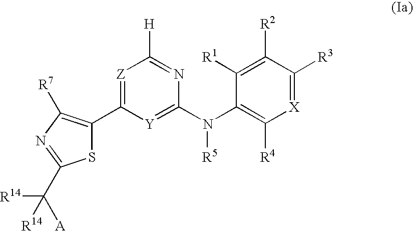

in which the symbols are as defined below:
R¹ to R⁴ independently of one another are hydrogen, halogen, cyano, hydroxyl, nitro, OR¹¹, SR¹¹, SOR¹¹, SO₂R¹¹, SO₂N(R¹¹)₂, C═OR¹¹, NR¹¹COOR¹², N(R¹¹)₂, NR¹¹COR¹¹, NR¹¹COR², NR¹¹SO₂R², OCON(R¹¹)₂, OC═OR¹¹, CON(R¹¹)₂, COOR¹¹, (CH₂)ₘOR¹¹, (CH₂)ₘSR¹¹, (CH₂)ₘN(R¹¹)₂, (CH₂)ₘCOOR¹², (CH₂)ₘNR¹¹COOR¹¹, unsubstituted or substituted C₁-C₈-alkyl, C₁-C₈-haloalkyl, C₃-C₈-cycloalkyl; where m=1-8
   or in each case two adjacent radicals R¹, R² or R², R³ or R³, R⁸ or R⁸, R⁴ together form an unsubstituted or substituted heterocyclic five- or six-membered ring,
R⁵ is hydrogen, unsubstituted or substituted C₁-C₈-alkyl, unsubstituted or substituted C₁-C₄-alkylC(═O), C₁-C₄-alkylOC(═O), unsubstituted or substituted C₁-C₄-alkoxy(C₁-C₄)alkyl, unsubstituted or substituted C₂-C₆-alkenyl, unsubstituted or substituted C₂-C₆-alkynyl, C₁-C₆-alkylsulphinyl, C₁-C₆-alkylsulphonyl, C₃-C₈-cycloalkyl; C₁-C₆-haloalkyl, C₁-C₄-haloalkylsulphinyl, C₁-C₄-haloalkylsulphonyl, halo-C₁-C₄-alkoxy-C₁-C₄-alkyl, C₃-C₈-halocycloalkyl having in each case 1 to 9 fluorine, chlorine and/or bromine atoms; formyl, formyl-C₁-C₃-alkyl, (C₁-C₃-alkyl)carbonyl-C₁-C₃-alkyl, (C₁-C₃-alkoxy)carbonyl-C₁-C₃-alkyl; halo-(C₁-C₃-alkyl)carbonyl-C₁-C₃-alkyl, halo-(C₁-C₃-alkoxy)carbonyl-C₁-C₃-alkyl having in each case 1 to 13 fluorine, chlorine and/or bromine atoms; (C₁-C₈-alkyl)carbonyl, (C₁-C₈-alkoxy)carbonyl, (C₁-C₈-alkylthio)carbonyl, (C₁-C₄-alkoxy-C₁-C₄-alkyl)carbonyl, (C₃-C₆-alkenyloxy)carbonyl, (C₃-C₆-alkynyloxy)carbonyl, (C₃-C₈-cycloalkyl)carbonyl; (C₁-C₆-haloalkyl)carbonyl, (C₁-C₆-haloalkylthio)carbonyl, (C₁-C₆-haloalkoxy)carbonyl, (C₃-C₆-haloalkenyloxy)carbonyl, (C₃-C₆-haloalkynyloxy)carbonyl, (halo-C₁-C₄-alkoxy-C₁-C₄-alkyl)carbonyl, (C₃-C₈-halocycloalkyl)carbonyl having in each case 1 to 9 fluorine, chlorine and/or bromine atoms, or —CH₂—C═C—R^{1-A}, —CH₂—CH═CH—R^{1-A}, —CH═C═CH—R^{1-A}, —C(═O)C(═O)R², —CONR³R⁴, —CH₂NR⁵R⁶, C₁-C₄-trialkylsilyl or C₁-C₄-dialkylmonophenylsilyl,
R^{1-A} represents hydrogen, C₁-C₆-alkyl, C₁-C₆-haloalkyl, C₂-C₆-alkenyl, C₂-C₆-alkynyl, C₃-C₇-cycloalkyl, (C₁-C₄-alkoxy)carbonyl, (C₃-C₆-alkenyloxy)carbonyl, (C₃-C₆-alkynyloxy)-carbonyl or cyano,
X is nitrogen or CR⁸,
Y is nitrogen or CR⁹,
Z is nitrogen or CR¹⁰,
R⁷ is hydrogen, halogen, cyano, hydroxyl, amino, N(R¹¹)₂, nitro, OR¹¹, SR¹¹, unsubstituted or substituted C₁-C₈-alkyl, unsubstituted or substituted C₃-C₆-cycloalkyl, C₁-C₄-trialkylsilyl, unsubstituted or substituted aryl or hetaryl, COOR¹¹, CON(R¹¹)₂, COR¹¹or
R⁷ and R¹⁰ together form a saturated or unsaturated bridge of the following structure:

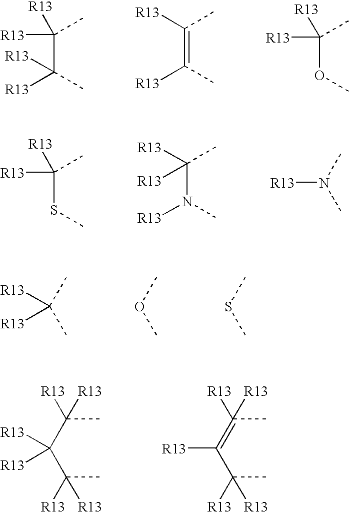

where R¹³=independently of one another hydrogen, halogen, unsubstituted or substituted C₁-C₃-alkyl, unsubstituted or substituted C₁-C₃-alkoxy or
   two geminal radicals R¹³ represent doubly attached oxygen or sulphur,
R⁸ is hydrogen, halogen, cyano, hydroxyl, nitro, OR¹¹, SR¹¹, SOR¹¹, SO₂R¹¹, SO₂N(R¹¹)₂, C═OR¹¹, NR¹¹COOR², N(R¹¹)₂, NR¹¹COR¹¹, NR¹¹COR², NR¹¹SO₂R², OCON(R¹¹)₂, OC═OR¹¹, CON(R¹¹)₂, COOR¹¹, (CH₂)ₘOR¹¹, (CH₂)ₘSR¹¹, (CH₂)ₘN(R¹¹)₂, (CH₂)ₘCOOR², (CH₂)ₘNR¹¹COOR¹¹, unsubstituted or substituted C₁-C₈-alkyl, C₁-C₈-haloalkyl, C₃-C₈-cycloalkyl; where m=1-8,
R⁹ is hydrogen, halogen, N(R¹¹)₂, cyano, hydroxyl, OR¹¹, SR¹¹, COR¹¹, unsubstituted or substituted C₁-C₈-alkyl or unsubstituted or substituted C₃-C₈-cycloalkyl,
R¹⁰ is hydrogen, halogen, cyano, hydroxyl, amino, N(R¹¹)₂, nitro, OR¹¹, SR¹¹, unsubstituted or substituted C₁-C₈-alkyl, C₁-C₄-trialkylsilyl, COOR¹¹, CON(R¹¹)₂,
R¹¹ are identical or different and are hydrogen, unsubstituted or substituted C₁-C₈-alkyl, C₁-C₈-haloalkyl, C₁-C₄-trialkylsilyl, aryl, or
   if two radicals R¹¹ are attached to one nitrogen atom, two radicals R¹¹ may form a 3- to 7-membered unsubstituted or substituted saturated or unsaturated cycle which may contain up to five further heteroatoms selected from the group consisting of N, O and S, where two oxygen atoms are not adjacent, or
   if two radicals R¹¹ are adjacent in the grouping NR¹¹COR¹¹, two radicals R¹¹ may form a 3- to 7-membered unsubstituted or substituted saturated or unsaturated cycle which may contain up to five further heteroatoms selected from the group consisting of N, O and S, where two oxygen atoms are not adjacent,
R¹² are identical or different and are unsubstituted or substituted C₁-C₈-alkyl, C₁-C₈-haloalkyl, (CH₂)ₜR¹¹, C₁-C₄-trialkylsilyl; where t=1-4,
R¹⁴ independently of one another are hydrogen, halogen, cyano, hydroxyl, OR¹¹, SR¹¹, COR¹¹, unsubstituted or substituted C₁-C₈-alkyl, C₁-C₈-haloalkyl, C₁-C₄-trialkylsilyl or unsubstituted or substituted C₃-C₈-cycloalkyl,
A is OR¹⁵, SR¹⁵, N(R¹⁵)₂ or C(R¹⁶)₃,
R¹⁵ independently of one another are COR⁷, unsubstituted or substituted C₂-C₈-alkyl, C₁-C₈-haloalkyl, unsubstituted or substituted C₃-C₈-cycloalkyl, (CH₂)ᵤOR¹¹; where u=1-4,
R¹⁶ independently of one another are hydrogen, halogen, cyano, hydroxyl, OR¹¹, SR¹¹, COR¹¹, N(R¹¹)₂, unsubstituted or substituted C₁-C₈-alkyl, or unsubstituted or substituted C₃-C₈-cycloalkyl, or
   in each case two radicals R¹⁶ represent doubly attached oxygen or doubly attached sulphur, or
   in each case two radicals R¹⁴, R¹⁴ or R¹⁵, R¹⁵ or R¹⁶, R¹⁶ or R¹⁴, R¹⁶ together form a saturated or unsaturated carbocyclic 3- to 7-membered ring or an optionally subtituted 4-pyridyl,
R¹⁷ is unsubstituted or substituted C₁-C₃-alkyl or C₁-C₃-haloalkyl
   and also agrochemically active salts thereof.

Compounds of the formula (Ia) are highly suitable for controlling unwanted microorganisms. Especially, they have strong fungicidal activity and can be used both in crop protection and in the protection of materials.

The formula (Ia) provides a general definition of the compounds according to the invention.

Preference is given to compounds of the formula (Ia) in which one or more of the symbols have one of the preferred meanings given below, i.e.,
R¹ to R⁴ independently of one another are hydrogen, fluorine, chlorine, bromine, iodine, cyano, hydroxyl, nitro, OR¹¹, O-Me, O-Et, O—Pr, O-iPr, O-Bu, O-secBu, O-isoBu, O-tBu, O—(CH₂)₂OH, O—(CH₂)₂OCH₃, O—(CH₂)₃OH, O—(CH₂)₃OCH₃, OCF₃, SR¹¹, SH, S-Me, S-Et, S—Pr, S-iPr, S-Bu, S-secBu, S-isoBu, S-tBu, SCF₃, SOR¹¹, SO-Me, SO-Et, SO—Pr, SO-iPr, SO-Bu, SO-secBu, SO-isoBu, SO-tBu, SO₂R¹¹, SO₂-Me, SO₂-Et, SO₂—Pr, SO₂-iPr, SO₂—Bu, SO₂-secBu, SO₂-isoBu, SO₂-tBu, SON(R¹¹)₂, SONHMe, SONMe₂, SONHEt, SONEt₂, SONHPr, SONPr₂, SONHBu, SONBu₂, SONHCF₃, SON(CF₃)₂, SO₂N(R¹¹)₂, SO₂NHMe, SO₂NMe₂, SO₂NEt₂, SO₂NHEt, SO₂NPr₂, SO₂NHPr, SO₂NHCF₃, SO₂N(CF₃)₂, COR¹¹, COMe, COEt, COPr, COiPr, COBu, COsecBu, CO-isoBu, COtBu, COCF₃, NR¹¹CO₂R¹¹, NR¹¹CO₂Me, NR¹¹CO₂Et, NR¹¹CO₂Pr, NR¹¹CO₂iPr, NR¹¹CO₂Bu, NR¹¹CO₂secBu, NR¹¹CO₂ isoBu, NR¹¹CO₂tBu, NR¹¹COR¹¹, NHCOMe, NHCOEt, NHCOPr, NHCOiPr, NHCOBu, NHCOiBu, NHCOsecBu, NHCOisoBu, NHCOtBu, NHCO(CH₂)₂OH, NHCO(CH₂)₂OCH₃, NHCO(CH₂)₃OH, NHCO(CH₂)₃OCH₃, NR¹¹COMe, NR¹¹COEt, NR¹¹COPr, NR¹¹COiPr, NR¹¹COBu, NR¹¹COsecBu, NR¹¹COtBu, NR¹¹CO(CH₂)₂OH, NR¹¹CO(CH₂)₂OCH₃, NR¹¹CO(CH₂)₃OH, NR¹¹CO(CH₂)₃OCH₃, N(R¹¹)₂, NMe₂, NEt₂, NHMe, NH₂, NHtBu, NHEt, NHPr, NHiPr, NHBu, NHiBu, NHsecBu, piperazin-1-yl, 4-methylpiperazin-1-yl, morpholin-1-yl, NR¹¹SO₂R¹², NHSOR¹¹, NR¹¹SOR¹¹, NHSOMe, NHSO₂Me, NHSOEt, NHSO₂Et, NMeSOMe, NHSO₂R¹¹, NR¹¹SO₂R¹¹, NMeSO₂Me, NMeSOEt, NMeSO₂Et, NHSOCF₃, NHSO₂CF₃, OCON(R¹¹)₂, OCONHMe, OCONHEt, OCONHPr, OCONHiPr, OCONHBu, OCONHsecBu, OCONHisoBu, OCONHtBu, OCONMe₂, OCONEt₂, OCONPr₂, OCONiPr₂, OCONBu₂, OCONsecBu₂, OCONisoBu₂, OCONHtBu₂, OCOR¹¹, OCOMe, OCOEt, OCOPr, OCOiPr, OCOBu, OCOsecBu, OCOisoBu, OCOtBu, CON(R¹¹)₂, CONHEt, CONEt₂, CONHMe, CONMe₂, CONHPr, CONPr₂, CONHBu, CONHsecBu, CONHisoBu, CONHtBu, CONHCH(CH₃)CH₂OH, CONHCH(CH₃)CH₂OCH₃, CONHCH(C₂H₅)CH₂OH, CONHCH(C₂H₅)CH₂OCH₃, CONH(CH₂)₂OCH₃, CONH(CH₂)₂OH, CONH(CH₂)₃OCH₃, CONH(CH₂)₃OH, CONR¹¹CH(CH₃)CH₂OH, CONR¹¹CH(CH₃)CH₂OCH₃, CONR¹¹CH(C₂H₅)CH₂OH, CONR¹¹CH(C₂H₅)CH₂OCH₃, CONR¹¹(CH₂)₂OCH₃, CONR¹¹(CH₂)₂OH, —CONR¹¹(CH₂)₃OCH₃, CONR¹¹(CH₂)₃OH, CO₂R¹¹, CO₂Me, CO₂Et, CO₂Pr, CO₂iPr, CO₂Bu, CO₂secBu, CO₂ isoBu, CO₂tBu, CO₂(CH₂)₂OH, CO₂(CH₂)₂OCH₃, CO₂(CH₂)₃OH, CO₂(CH₂)₃OCH₃, (CH₂)ₘOR¹¹, CH₂OH, (CH₂)₂OH, (CH₂)₃OH, (CH₂)₄OH, CH₂OMe, (CH₂)₂OMe, (CH₂)₃OMe, (CH₂)₄OMe, (CH₂)ₘSR¹¹, CH₂SH, (CH₂)₂SH, (CH₂)₃SH, (CH₂)₄SH, CH₂SMe, (CH₂)₂SMe, (CH₂)₃SMe, (CH₂)₄SMe, (CH₂)ₘN(R¹¹)₂, CH₂NH₂, CH₂NAc₂, CH₂N(COCF₃)₂, CH₂NHAc, CH₂NHCOCF₃, (CH₂)₂NH₂, (CH₂)₃NH₂, (CH₂)₄NH₂, CH₂NMe₂, (CH₂)₂NHMe, (CH₂)₂NMe₂, (CH₂)₃NHMe, (CH₂)₃NMe₂, (CH₂)₄NHMe, (CH₂)₄NMe₂, (CH₂)ₘCOOR¹², CH₂COOMe, (CH₂)₂COOMe, (CH₂)₃COOMe, CH₂COOEt, (CH₂)₂COOEt, (CH₂)₃COOEt, CH₂COOPr, (CH₂)₂COOPr, (CH₂)₃COOPr, CH₂COOiPr, (CH₂)₂COOiPr, (CH₂)₃COOiPr, CH₂COOtBu, (CH₂)₂COOtBu, (CH₂)₃COOtBu, CH₂COO(CH₂)₂OH CH₂COO(CH₂)₂OCH₃, CH₂COO(CH₂)₃OH, CH₂COO(CH₂)₃OCH₃, CH₂NHCOOR¹¹, CH₂NR¹¹COOR¹¹, CH₂NHCOOMe, CH₂NHCOOtBu, CH₂NHCOOEt, CH₂NHCOOPr, CH₂NHCOOiPr, CH₂NHCOOBu, CH₂NHCOOtBu, CH₂NHCOOsecBu, CH₂NHCOOisoBu, CH₂NR¹¹COOEt, CH₂NR¹¹COOPr, CH₂NR¹¹COOiPr, CH₂NR¹¹COOBu, CH₂NR¹¹COOtBu, CH₂NR¹¹COOsecBu, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; cyclopropyl, cyclobutyl, cyclopentyl, cyclohexyl; CF₃, CF₂H, CCl₃, C₂F₅, C₃F₇, CF(CF₃)₂
   or in each case two adjacent radicals R¹, R² or R², R³ or R³, R⁸ or R⁸, R⁴ together form a heterocyclic five- or six-membered ring which is unsubstituted or substituted by 1 to 4 halogen atoms or by 1 to 4 C₁-C₃-alkyl groups and which contains in each case up to two oxygen atoms,
R⁵ is hydrogen, methyl, ethyl, propyl, 1-methylethyl, butyl, 1-methylpropyl, 2-methylpropyl, 1,1-dimethylethyl, CF₃, CF₂H, CCl₃, C₂F₅, C₃F₇, CF(CF₃)₂,
   acetyl, C₂H₅C(═O), C₃H₇C(═O), C₄H₉C(═O), CF₃C(═O), C₂F₅C(═O), CH₃C(═O), C₂H₅OC (═O), C₃H₇C(═O), C₃H₇C(═O), C₄H₉C(═O), CF₃C(═O), CCl₃C(═O), C₂F₅C(═O), CH₂OCH₃; C₂H₄OCH₃, CH═CH₂, CH₂CH═CH₂, C═CH, CH₂C≡CH, SOCH₃, SOC₂H₅, SOC₃H₇ SO₂CH₃, SO₂C₂H₅, SO₂C₃H₇, cyclopropyl, cyclobutyl, cyclopentyl, cyclohexyl; CH₂OCF₃; C₂H₄OCF₃, C═ONH₂, SiMe₃, SiMe₂tBu, SiMe₂Ph,
X is nitrogen or CR⁸,
Y is nitrogen or CR⁹,
Z is nitrogen or CR¹⁰,
   where either Y and Z are nitrogen,
or Y is nitrogen and Z is CR¹⁰,
or Y is CR⁹ and Z is nitrogen,
R⁷ is hydrogen, fluorine, chlorine, bromine, iodine, cyano, hydroxyl, amino, N(R¹¹)₂, nitro, OR¹¹, SR¹¹, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methyl-pentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; CF₃, CF₂H, CCl₃, C₂F₅, C₃F₇, CF(CF₃)₂, cyclopropyl, cyclobutyl, cyclopentyl, cyclohexyl, phenyl, p-Cl-phenyl, p-F-phenyl, p-Br-phenyl, p-I-phenyl, p-methoxyphenyl, p-trifluoromethoxyphenyl, 2-pyridyl, 3-pyridyl, 4-pyridyl, 2-thienyl, 3-thienyl, SiMe₃, COOR¹¹, CON(R¹¹)₂, COR¹¹, or
R⁷ and R¹⁰ together form a saturated or unsaturated bridge of the following structure:

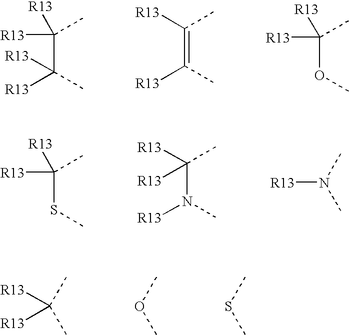

where R¹³=independently of one another hydrogen, fluorine, chlorine, bromine, iodine, methyl, ethyl, propyl, 1-methylethyl, CF₂H, CF₃, C₂F₅, OCH₃, OC₂H₅, OCF₃, OC₂F₅ or
   two geminal radicals R¹³ represent doubly attached oxygen or sulphur,
R⁸ is hydrogen, fluorine, chlorine, bromine, iodine, cyano, hydroxyl, nitro, OR¹¹, O-Me, O-Et, O—Pr, O-iPr, O-Bu, O-secBu, O-isoBu, O-tBu, O—(CH₂)₂OH, O—(CH₂)₂OCH₃, O—(CH₂)₃OH, O—(CH₂)₃OCH₃, OCF₃, SR¹¹, SH, S-Me, S-Et, S—Pr, S-iPr, S-Bu, S-secBu, S-isoBu, S-tBu, SCF₃, SOR¹¹, SO-Me, SO-Et, SO—Pr, SO-iPr, SO-Bu, SO-secBu, SO-isoBu, SO-tBu, SO₂R¹¹, SO₂-Me, SO₂-Et, SO₂—Pr, SO₂-iPr, SO₂—Bu, SO₂-secBu, SO₂-isoBu, SO₂-tBu, SON(R¹¹)₂, SONHMe, SONMe₂, SONHEt, SONEt₂, SONHPr, SONPr₂, SONHBu, SONBu₂, SONHCF₃, SON(CF₃)₂, SO₂N(R¹¹)₂, SO₂NHMe, SO₂NMe₂, SO₂NEt₂, SO₂NHEt, SO₂NPr₂, SO₂NHPr, SO₂NHCF₃, SO₂N(CF₃)₂, COR¹¹, COMe, COEt, COPr, COiPr, COBu, COsecBu, COisoBu, COtBu, COCF₃, NR¹¹CO₂R¹¹, NR¹¹CO₂Me, NR¹¹CO₂Et, NR¹¹CO₂Pr, NR¹¹CO₂iPr, NR¹¹CO₂Bu, NR¹¹CO₂secBu, NR¹¹CO₂ isoBu, NR¹¹ CO₂tBu, NR¹¹COR¹¹, NHCOMe, NHCOEt, NHCOPr, NHCOiPr, NHCOBu, NHCOiBu, NHCOsecBu, NHCOisoBu, NHCOtBu, NHCO(CH₂)₂OH, NHCO(CH₂)₂OCH₃, NHCO(CH₂)₃OH, NHCO(CH₂)₃OCH₃, NR¹¹COMe, NR¹¹COEt, NR¹¹COPr, NR¹¹COiPr, NR¹¹COBu, NR¹¹ COsecBu, NR¹¹ COisoBu, NR¹¹ COtBu, NR¹¹ CO(CH₂)₂OH, NR¹¹ CO(CH₂)₂OCH₃, NR¹¹CO(CH₂)₃OH, NR¹¹CO(CH₂)₃OCH₃, N(R¹¹)₂, NMe₂, NEt₂, NHMe, NH₂, NHtBu, NHEt, NHPr, NHiPr, NHBu, NHsecBu, NHisoBu, 4-methylpiperazin-1-yl, piperazin-1-yl, morpholin-1-yl, NR¹¹SO₂R¹², NHSOR¹¹, NR¹¹SOR¹¹, NHSOMe, NHSO₂Me, NHSOEt, NHSO₂Et, NMeSOMe, NHSO₂R¹¹, NR¹¹SO₂R¹¹, NMeSO₂Me, NMeSOEt, NMeSO₂Et, NHSOCF₃, NHSO₂CF₃, OCON(R¹¹)₂, OCONHMe, OCONHEt, OCONHPr, OCONHiPr, OCONHBu, OCONHsecBu, OCONHisoBu, OCONHtBu, OCONMe₂, OCONEt₂, OCONPr₂, OCONiPr₂, OCONBu₂, OCONsecBu₂, OCONisoBu₂, OCONtBu₂, OCOR¹¹, OCOMe, OCOEt, OCOPr, OCOiPr, OCOBu, OCOsecBu, OCOisoBu, OCOtBu, CON(R¹¹)₂, CONHEt, CONEt₂, CONHMe, CONMe₂, CONHPr, CONPr₂, CONHBu, OCONHBu, CONHtBu, CONHCH(CH₃)CH₂OH, CONHCH(CH₃)CH₂OCH₃, CONHCH(C₂H₅)CH₂OH, CONHCH(C₂H₅)CH₂OCH₃, CONH(CH₂)₂OCH₃, CONH(CH₂)₂OH, CONH(CH₂)₃OCH₃, CONH(CH₂)₃OH, CONR¹¹CH(CH₃)CH₂OH, CONR¹¹CH(CH₃)CH₂OCH₃, CONR¹¹CH(C₂H₅)CH₂OH, CONR¹¹CH(C₂H₅)CH₂OCH₃, CONR¹¹ (CH₂)₂OCH₃, CONR¹¹(CH₂)₂OH, CONR¹¹(CH₂)₃OCH₃, CONR¹¹ (CH₂)₃OH, CO₂R¹¹, CO₂Me, CO₂Et, CO₂Pr, CO₂iPr, CO₂Bu, CO₂secBu, CO₂ isoBu, CO₂tBu, CO₂(CH₂)₂OH, CO₂(CH₂)₂OCH₃, CO₂(CH₂)₃OH, CO₂(CH₂)₃OCH₃, (CH₂)ₘOR¹¹, CH₂OH, (CH₂)₂OH, (CH₂)₃OH, (CH₂)₄OH, CH₂OMe, (CH₂)₂OMe, (CH₂)₃OMe, (CH₂)₄OMe, (CH₂)ₘSR¹¹, CH₂SH, (CH₂)₂SH, (CH₂)₃SH, (CH₂)₄SH, CH₂SMe, (CH₂)₂SMe, (CH₂)₃SMe, (CH₂)₄SMe, (CH₂)ₘN(R¹¹)₂, CH₂NH₂, CH₂NAC₂, CH₂N(COCF₃)₂, CH₂NHAc, CH₂NHCOCF₃, (CH₂)₂NH₂, (CH₂)₃NH₂, (CH₂)₄NH₂, CH₂NMe₂, (CH₂)₂NHMe, (CH₂)₂NMe₂, (CH₂)₃NHMe, (CH₂)₃NMe₂, (CH₂)₄NHMe, (CH₂)₄NMe₂, (CH₂)ₘCOOR¹², CH₂COOMe, (CH₂)₂COOMe, (CH₂)₃COOMe, CH₂COOEt, (CH₂)₂COOEt, (CH₂)₃COOEt, CH₂COOPr, (CH₂)₂COOPr, (CH₂)₃COOPr, CH₂COOiPr, (CH₂)₂COOiPr, (CH₂)₃COOiPr, CH₂COOtBu, (CH₂)₂COOtBu, (CH₂)₃COOtBu, CH₂COO(CH₂)₂OH, CH₂COO(CH₂)₂OCH₃, CH₂COO(CH₂)₃OH, CH₂COO(CH₂)₃OCH₃, CH₂NHCOOR¹¹, CH₂NR¹¹COOR¹¹, CH₂NHCOOMe, CH₂NHCOOtBu, CH₂NHCOOEt, CH₂NHCOOPr, CH₂NHCOOiPr, CH₂NHCOOBu, CH₂NHCOOtBu, CH₂NHCOOsecBu, CH₂NHCOOisoBu, CH₂NR¹¹COOEt, CH₂NR¹¹ COOPr, CH₂NR¹¹ COOiPr, CH₂NR¹¹COOBu, CH₂NR¹¹COOtBu, CH₂NR¹¹COOsecBu, CH₂NR¹¹COOisoBu, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methyl-propyl; cyclopropyl, cyclobutyl, cyclopentyl, cyclohexyl; CF₃, CF₂H, CCl₃, C₂F₅, C₃F₇, CF(CF₃)₂,
R⁹ is hydrogen, fluorine, chlorine, bromine, iodine, N(R¹¹)₂, NMe₂, NEt₂, NHMe, NH₂, NHtBu, NHEt, NHPr, NHiPr, NHBu, NHsecBu, NHisoBu, 4-methylpiperazin-1-yl, piperazin-1-yl, morpholin-1-yl, cyano, hydroxyl, OR¹¹, O-Me, O-Et, O—Pr, O-iPr, O-Bu, O-secBu, O-isoBu, O-tBu, O—(CH₂)₂OH, O—(CH₂)₂OCH₃, O—(CH₂)₃OH, O—(CH₂)₃OCH₃, OCF₃, SR¹¹, SH, S-Me, S-Et, S—Pr, S-iPr, S-Bu, S-secBu, S-isoBu, S-tBu, SCF₃, COR¹¹, COMe, COEt, COPr, COiPr, COBu, COsecBu, COisoBu, COtBu, COCF₃, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methyl-propyl; CF₃, CF₂H, CCl₃, C₂F₅, C₃F₇, CF(CF₃)₂, cyclopropyl, cyclobutyl, cyclopentyl, cyclohexyl,
R¹⁰ is hydrogen, fluorine, chlorine, bromine, iodine, cyano, hydroxyl, amino, N(R¹¹)₂, nitro, OR¹¹, SR¹¹, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methyl-pentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; CF₃, CF₂H, CCl₃, C₂F₅, C₃F₇, CF(CF₃)₂, cyclopropyl, cyclobutyl, cyclopentyl, cyclohexyl, SiMe₃, COOR¹¹, CON(R¹¹)₂,
R¹¹ are identical or different and are hydrogen, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, CH₂CH₂OMe, CH(CH₃)CH₂OMe, CH₂CH(CH₃)OMe, CH₂CH₂OEt, CH(CH₃)CH₂OEt, CH₂CH(CH₃)OEt, CF₃, CF₂H, CCl₃, C₂F₅, C₃F₇, CF(CF₃)₂, cyclopropyl, cyclobutyl, cyclopentyl, cyclohexyl, SiMe₃, COOR¹¹, CON(R¹¹)₂, phenyl or
   if two radicals R¹¹ are attached to one nitrogen atom, two radicals R¹¹ may form a 3- to 7-membered, unsubstituted or substituted saturated or unsaturated cycle which may contain up to five further heteroatoms from the group consisting of N, O and S, where two oxygen atoms are not adjacent, or
   if two radicals R¹¹ are adjacent in the grouping NR¹¹COR¹¹, two radicals R¹¹ may form a 3- to 7-membered unsubstituted or substituted saturated or unsaturated cycle which may contain up to five further heteroatoms selected from the group consisting of N, O and S, where two oxygen atoms are not adjacent,
R¹² is methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; CF₃, CF₂H, CCl₃, C₂F₅, C₃F₇, CF(CF₃)₂, cyclopropyl, cyclobutyl, cyclopentyl, cyclohexyl, CH₂OR¹¹, (CH₂)₂OR¹¹, (CH₂)₃OR¹¹, (CH₂)₄OR¹¹, SiMe₃,
R¹⁴ independently of one another are hydrogen, fluorine, chlorine, bromine, iodine, cyano, hydroxyl, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; CF₃, CF₂H, CCl₃, C₂F₅, C₃F₇, CF(CF₃)₂, or cyclopropyl, cyclohexyl, cyclopentyl, SiMe₃,
A is OR¹⁵, SR¹⁵, N(R¹⁵)₂ or C(R¹⁶)₃,
R¹⁵ independently of one another are COR¹⁷, COCH₃, COCF₃, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, CH₂CH₂OMe, CH(CH₃)CH₂OMe, CH₂CH(CH₃)OMe, CH₂CH₂OEt, CH(CH₃)CH₂OEt, CH₂CH(CH₃)OEt, CF₃, CF₂H, CCl₃, C₂F₅, C₃F₇, CF(CF₃)₂, cyclopropyl, cyclobutyl, cyclohexyl, CH₂OR¹¹, (CH₂)₂OR¹¹, (CH₂)₃OR¹¹, (CH₂)₄OR¹¹,
R¹⁶ independently of one another are hydrogen, halogen, cyano, hydroxyl, OR¹¹, SR¹¹, COR¹¹, N(R¹¹)₂, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methyl-pentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; CF₃, CF₂H, CCl₃, C₂F₅, C₃F₇, CF(CF₃)₂, cyclopropyl, cyclobutyl, cyclopentyl, cyclohexyl, CH₂OR¹¹, (CH₂)₂OR¹¹, (CH₂)₃OR¹¹ (CH₂)₄OR¹¹, SiMe₃ or
   in each case two radicals R¹⁶ represent doubly attached oxygen or doubly attached sulphur, or
   in each case two radicals R¹⁴, R¹⁴ or R¹⁵, R¹⁵ or R¹⁶, R¹⁶ or R¹⁴, R¹⁵ or R¹⁴, R¹⁶ together form a cyclopentyl, cyclohexyl, cyclopentenyl, cyclohexenyl, cyclopentadienyl, cyclohexadienyl, 4-pyridyl,
R¹⁷ is methyl, ethyl, propyl, 1-methylethyl, CF₃, CF₂H, CCl₃, C₂F₅, C₃F₇, CF(CF₃)₂.

Particular preference is given to compounds of the formula (Ia) in which one or more of the symbols have one of the following meanings:
R¹ to R⁴ independently of one another are hydrogen, fluorine, chlorine, bromine, iodine, cyano, hydroxyl, nitro, OR¹¹, O-Me, O-Et, O—Pr, O-iPr, O-Bu, O-secBu, O-isoBu, O-tBu, O—(CH₂)₂OH, O—(CH₂)₂OCH₃, O—(CH₂)₃OH, O—(CH₂)₃OCH₃, OCF₃, SR¹¹, SH, S-Me, S-Et, S—Pr, S-iPr, S-Bu, S-secBu, S-isoBu, S-tBu, SCF₃, SOR¹¹, SO-Me, SO-Et, SO—Pr, SO-iPr, SO-Bu, SO-secBu, SO-isoBu, SO-tBu, SO₂R¹¹, SO₂-Me, SO₂-Et, SO₂—Pr, SO₂-iPr, SO₂—Bu, SO₂-secBu, SO₂-isoBu, SO₂-tBu, SON(R¹¹)₂, SONHMe, SONMe₂, SONHEt, SONEt₂, SONHPr, SONPr₂, SONHBu, SONBu₂, SONHCF₃, SON(CF₃)₂, SO₂N(R¹¹)₂, SO₂NHMe, SO₂NMe₂, SO₂NEt₂, SO₂NHEt, SO₂NPr₂, SO₂NHPr, SO₂NHCF₃, SO₂N(CF₃)₂, COR¹¹, COMe, COEt, COPr, COiPr, COBu, COsecBu, CO-isoBu, COtBu, COCF₃, NR¹¹CO₂R¹¹, NR¹¹CO₂Me, NR¹¹ CO₂Et, NR¹¹ CO₂Pr, NR¹¹ CO₂iPr, NR¹¹ CO₂Bu, NR¹¹CO₂secBu, NR¹¹CO₂ isoBu, NR¹¹CO₂tBu, NR¹¹COR¹¹, NHCOMe, NHCOEt, NHCOPr, NHCOiPr, NHCOBu, NHCOiBu, NHCOsecBu, NHCOisoBu, NHCOtBu, NHCO(CH₂)₂OH, NHCO(CH₂)₂OCH₃, NHCO(CH₂)₃OH, NHCO(CH₂)₃OCH₃, NR¹¹COMe, NR¹¹COEt, NR¹¹COPr, NR¹¹COiPr, NR¹¹COBu, NR¹¹COsecBu, NR¹¹COtBu, NR¹¹CO(CH₂)₂OH, NR¹¹CO(CH₂)₂OCH₃, NR¹¹ CO(CH₂)₃OH, NR¹¹CO(CH₂)₃OCH₃, N(R¹¹)₂, NMe₂, NEt₂, NHMe, NH₂, NHtBu, NHEt, NHPr, NHiPr, NHBu, NHiBu, NHsecBu, piperazin-1-yl, 4-methylpiperazin-1-yl, morpholin-1-yl, NRlISO₂R¹², NHSOR¹¹, NR¹¹SOR¹¹, NHSOMe, NHSO₂Me, NHSOEt, NHSO₂Et, NMeSOMe, NHSO₂R¹¹, NR¹¹SO₂R¹¹, NMeSO₂Me, NMeSOEt, NMeSO₂Et, NHSOCF₃, NHSO₂CF₃, OCON(R¹¹)₂, OCONHMe, OCONHEt, OCONHPr, OCONHiPr, OCONHBu, OCONHsecBu, OCONHisoBu, OCONHtBu, OCONMe₂, OCONEt₂, OCONPr₂, OCONiPr₂, OCONBu₂, OCONsecBu₂, OCONisoBu₂, OCONHtBu₂, OCOR¹¹, OCOMe, OCOEt, OCOPr, OCOiPr, OCOBu, OCOsecBu, OCOisoBu, OCOtBu, CON(R¹¹)₂, CONHEt, CONEt₂, CONHMe, CONMe₂, CONHPr, CONPr₂, CONHBu, CONHsecBu, CONHisoBu, CONHtBu, CONHCH(CH₃)CH₂OH, CONHCH(CH₃)CH₂OCH₃, CONHCH(C₂H₅)CH₂OH, CONHCH(C₂H₅)CH₂OCH₃, CONH(CH₂)₂OCH₃, CONH(CH₂)₂OH, CONH(CH₂)₃OCH₃, CONH(CH₂)₃OH, CONR¹¹CH(CH₃)CH₂OH, CONR¹¹CH(CH₃)CH₂OCH₃, CONR¹¹CH(C₂H₅)CH₂OH, CONR¹¹CH(C₂H₅)CH₂OCH₃, CONR¹¹(CH₂)₂OCH₃, CONR¹¹(CH₂)₂OH, CONR¹¹(CH₂)₃OCH₃, CONR¹¹(CH₂)₃OH, CO₂R¹¹, CO₂Me, CO₂Et, CO₂Pr, CO₂iPr, CO₂Bu, CO₂secBu, CO₂isoBu, CO₂tBu, CO₂(CH₂)₂OH, CO₂(CH₂)₂OCH₃, CO₂(CH₂)₃OH, CO₂(CH₂)₃OCH₃, (CH₂)ₘOR¹¹, CH₂OH, (CH₂)₂OH, (CH₂)₃OH, (CH₂)₄OH, CH₂OMe, (CH₂)₂OMe, (CH₂)₃OMe, (CH₂)₄OMe, (CH₂)ₘSR¹¹, CH₂SH, (CH₂)₂SH, (CH₂)₃SH, (CH₂)₄SH, CH₂SMe, (CH₂)₂SMe, (CH₂)₃SMe, (CH₂)₄SMe, (CH₂)ₘN(R¹¹)₂, CH₂NH₂, CH₂NAc₂, CH₂N(COCF₃)₂, CH₂NHAc, CH₂NHCOCF₃, (CH₂)₂NH₂, (CH₂)₃NH₂, (CH₂)₄NH₂, CH₂NMe₂, (CH₂)₂NHMe, (CH₂)₂NMe₂, (CH₂)₃NHMe, (CH₂)₃NMe₂, (CH₂)₄NHMe, (CH₂)₄NMe₂, (CH₂)ₘCOOR¹², CH₂COOMe, (CH₂)₂COOMe, (CH₂)₃COOMe, CH₂COOEt, (CH₂)₂COOEt, (CH₂)₃COOEt, CH₂COOPr, (CH₂)₂COOPr, (CH₂)₃COOPr, CH₂COOiPr, (CH₂)₂COOiPr, (CH₂)₃COOiPr, CH₂COOtBu, (CH₂)₂COOtBu, (CH₂)₃COOtBu, CH₂COO(CH₂)₂OH CH₂COO(CH₂)₂OCH₃, CH₂COO(CH₂)₃OH, CH₂COO(CH₂)₃OCH₃, CH₂NHCOOR¹¹, CH₂NR¹¹COOR¹¹, CH₂NHCOOMe, CH₂NHCOOtBu, CH₂NHCOOEt, CH₂NHCOOPr, CH₂NHCOOiPr, CH₂NHCOOBu, CH₂NHCOOtBu, CH₂NHCOOsecBu, CH₂NHCOOisoBu, CH₂NR¹¹COOEt, CH₂NR¹¹COOPr, CH₂NR¹¹COOiPr, CH₂NR¹¹COOBu, CH₂NR¹¹COOtBu, CH₂NR¹¹COOsecBu, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; cyclopropyl, cyclobutyl, cyclopentyl, cyclohexyl; CF₃, CF₂H, CCl₃, C₂F₅, C₃F₇, CF(CF₃)₂,
   or in each case two adjacent radicals R¹, R² or R², R³ or R³, R³ or R⁸, R⁴ together form a 2,3-dihydro-1,4-dioxin, a 2,2,3,3-tetrafluoro-2,3-dihydro-1,4-dioxin, a 1,3-dioxole or a 2,2-difluoro-1,3-dioxole ring,
R⁵ is hydrogen, methyl, ethyl, propyl, 1-methylethyl, butyl, 1-methylpropyl, 2-methylpropyl, 1,1-dimethylethyl, CF₃, CF₂H, CCl₃, C₂F₅, C₃F₇, CF(CF₃)₂; acetyl, C₂H₅C(═O), C₃H₇C(═O), C₄H₉C(═O), CF₃C(═O), C₂F₅C(═O), CH₂OCH₃; C₂H₄OCH₃, CH═CH₂, CH₂CH═CH₂, C≡CH, CH₂C≡CH, SOCH₃, SOC₂H₅, SOC₃H₇ SO₂CH₃, SO₂C₂H₅, SO₂C₃H₇, cyclopropyl, CH₂OCF₃; C₂H₄OCF₃, C═ONH₂, SiMe₃, SiMe₂tBu, SiMe₂Ph,
X is nitrogen or CR⁸,
Y is nitrogen,
Z is nitrogen or CR¹⁰,
R⁷ is hydrogen, fluorine, chlorine, bromine, iodine, cyano, hydroxyl, amino, N(R¹¹)₂, nitro, OR¹¹, SR¹¹, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methyl-pentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; CF₃, CF₂H, CCl₃, C₂F₅, C₃F₇, CF(CF₃)₂, cyclopropyl, cyclobutyl, cyclopentyl, cyclohexyl, phenyl, p-Cl-phenyl, p-F-phenyl, p-Br-phenyl, p-I-phenyl, p-methoxyphenyl, p-trifluoromethoxyphenyl, 2-pyridyl, 3-pyridyl, 4-pyridyl, 2-thienyl, 3-thienyl, SiMe₃, COOR¹¹, CON(R¹¹)₂, COR¹¹, or
R⁷ and R¹⁰ together form a saturated or unsaturated bridge of the following structure:

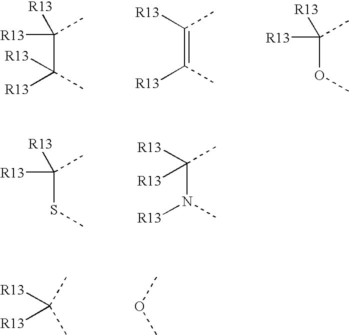

where R¹³=independently of one another hydrogen, fluorine, chlorine, bromine, iodine, methyl, ethyl, propyl, 1-methylethyl, CF₂H, CF₃, C₂F₅, OCH₃; OC₂H₅, OCF₃, OC₂F₅ or
   two geminal radicals R¹³ represent doubly attached oxygen or sulphur,
R⁸ is hydrogen, fluorine, chlorine, bromine, iodine, cyano, hydroxyl, nitro, OR¹¹, O-Me, O-Et, O—Pr, O-iPr, O-Bu, O-secBu, O-isoBu, O-tBu, O—(CH₂)₂OH, O—(CH₂)₂OCH₃, O—(CH₂)₃OH, O—(CH₂)₃OCH₃, OCF₃, SR¹¹, SH, S-Me, S-Et, S—Pr, S-iPr, S-Bu, S-secBu, S-isoBu, S-tBu, SCF₃, SOR¹¹, SO-Me, SO-Et, SO—Pr, SO-iPr, SO-Bu, SO-secBu, SO-isoBu, SO-tBu, SO₂R¹¹, SO₂-Me, SO₂-Et, SO₂—Pr, SO₂-iPr, SO₂—Bu, SO₂-secBu, SO₂-isoBu, SO₂-tBu, SON(R¹¹)₂, SONHMe, SONMe₂, SONHEt, SONEt₂, SONHPr, SONPr₂, SONHBu, SONBu₂, SONHCF₃, SON(CF₃)₂, SO₂N(R¹¹)₂, SO₂NHMe, SO₂NMe₂, SO₂NEt₂, SO₂NHEt, SO₂NPr₂, SO₂NHPr, SO₂NHCF₃, SO₂N(CF₃)₂, COR¹¹, COMe, COEt, COPr, COiPr, COBu, COsecBu, COisoBu, COtBu, COCF₃, NR¹¹CO₂R¹¹, NR¹¹CO₂Me, NR¹¹CO₂Et, NR¹¹CO₂Pr, NR¹¹CO₂iPr, NR¹¹CO₂Bu, NR¹¹CO₂secBu, NR¹¹CO₂ isoBu, NR¹¹CO₂tBu, NR¹¹ COR¹¹, NHCOMe, NHCOEt, NHCOPr, NHCOiPr, NHCOBu, NHCOiBu, NHCOsecBu, NHCOisoBu, NHCOtBu, NHCO(CH₂)₂OH, NHCO(CH₂)₂OCH₃, NHCO(CH₂)₃OH, NHCO(CH₂)₃OCH₃, NR¹¹COMe, NR¹¹COEt, NR¹¹COPr, NR¹¹COiPr, NR¹¹COBu, NR¹¹COsecBu, NR¹¹COisoBu, NR¹¹COtBu, NR¹¹CO(CH₂)₂OH, NR¹¹CO(CH₂)₂OCH₃, NR¹¹CO(CH₂)₃OH, NR¹¹CO(CH₂)₃OCH₃, N(R¹¹)₂, NMe₂, NEt₂, NHMe, NH₂, NHtBu, —NHEt, NHPr, NHiPr, NHBu, NHsecBu, NHisoBu, 4-methylpiperazin-1-yl, piperazin-1-yl, morpholin-1-yl, NR¹¹SO₂R¹², NHSOR¹¹, NR¹¹SOR¹¹, NHSOMe, NHSO₂Me, NHSOEt, NHSO₂Et, NMeSOMe, NHSO₂R¹¹, NR¹¹SO₂R¹¹, NMeSO₂Me, NMeSOEt, NMeSO₂Et, NHSOCF₃, NHSO₂CF₃, OCON(R¹¹)₂, OCONHMe, OCONHEt, OCONHPr, OCONHiPr, OCONHBu, OCONHsecBu, OCONHisoBu, OCONHtBu, OCONMe₂, OCONEt₂, OCONPr₂, OCONiPr₂, OCONBu₂, OCONsecBu₂, OCONisoBu₂, OCONtBu₂, OCOR¹¹, OCOMe, OCOEt, OCOPr, OCOiPr, OCOBu, OCOsecBu, OCOisoBu, OCOtBu, CON(R¹¹)₂, CONHEt, CONEt₂, CONHMe, CONMe₂, CONHPr, CONPr₂, CONHBu, OCONHBu, CONHtBu, CONHCH(CH₃)CH₂OH, CONHCH(CH₃)CH₂OCH₃, CONHCH(C₂H₅)CH₂OH, CONHCH(C₂H₅)CH₂OCH₃, CONH(CH₂)₂OCH₃, CONH(CH₂)₂OH, CONH(CH₂)₃OCH₃, CONH(CH₂)₃OH, CONR¹¹CH(CH₃)CH₂OH, CONR¹¹CH(CH₃)CH₂OCH₃, CONR¹¹CH(C₂H₅)CH₂OH, CONR¹¹CH(C₂H₅)CH₂OCH₃, CONR¹¹ (CH₂)₂OCH₃, CONR¹¹(CH₂)₂OH, CONR¹¹(CH₂)₃OCH₃, CONR¹¹(CH₂)₃OH, CO₂R¹¹, CO₂Me, CO₂Et, CO₂Pr, CO₂iPr, CO₂Bu, CO₂secBu, CO₂ isoBu, CO₂tBu, CO₂(CH₂)₂OH, CO₂(CH₂)₂OCH₃, CO₂(CH₂)₃OH, CO₂(CH₂)₃OCH₃, (CH₂)ₘOR¹¹, CH₂OH, (CH₂)₂OH, (CH₂)₃OH, (CH₂)₄OH, CH₂OMe, (CH₂)₂OMe, (CH₂)₃OMe, (CH₂)₄OMe, (CH₂)ₘSR¹¹, CH₂SH, (CH₂)₂SH, (CH₂)₃SH, (CH₂)₄SH, CH₂SMe, (CH₂)₂SMe, (CH₂)₃SMe, (CH₂)₄SMe, (CH₂)ₘN(R¹¹)₂, CH₂NH₂, CH₂NAc₂, CH₂N(COCF₃)₂, CH₂NHAc, CH₂NHCOCF₃, (CH₂)₂NH₂, (CH₂)₃NH₂, (CH₂)₄NH₂, CH₂NMe₂, (CH₂)₂NHMe, (CH₂)₂NMe₂, (CH₂)₃NHMe, (CH₂)₃NMe₂, (CH₂)₄NHMe, (CH₂)₄NMe₂, (CH₂)ₘCOOR¹², CH₂COOMe, (CH₂)₂COOMe, (CH₂)₃COOMe, CH₂COOEt, (CH₂)₂COOEt, (CH₂)₃COOEt, CH₂COOPr, (CH₂)₂COOPr, (CH₂)₃COOPr, CH₂COOiPr, (CH₂)₂COOiPr, (CH₂)₃COOiPr, CH₂COOtBu, (CH₂)₂COOtBu, (CH₂)₃COOtBu, CH₂COO(CH₂)₂OH, CH₂COO(CH₂)₂OCH₃, CH₂COO(CH₂)₃OH, CH₂COO(CH₂)₃OCH₃, CH₂NHCOOR¹¹, CH₂NR¹¹COOR¹¹, CH₂NHCOOMe, CH₂NHCOOtBu, CH₂NHCOOEt, CH₂NHCOOPr, CH₂NHCOOiPr, CH₂NHCOOBu, CH₂NHCOOtBu, CH₂NHCOOsecBu, CH₂NHCOOisoBu, CH₂NR¹¹COOEt, CH₂NR¹¹ COOPr, CH₂NR¹¹ COOiPr, CH₂NR¹¹COOBu, CH₂NR¹¹COOtBu, CH₂NR¹¹COOsecBu, CH₂NR¹¹COOisoBu, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methyl-propyl; cyclopropyl, cyclobutyl, cyclopentyl, cyclohexyl; CF₃, CF₂H, CCl₃, C₂F₅, C₃F₇, CF(CF₃)₂,
R¹⁰ is hydrogen, fluorine, chlorine, bromine, iodine, cyano, hydroxyl, amino, N(R¹¹)₂, nitro, OR¹¹, SR¹¹, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methyl-pentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; CF₃, CF₂H, OCF₃, OCF₂H, CCl₃, C₂F₅, C₃F₇, CF(CF₃)₂, cyclopropyl, cyclobutyl, cyclopentyl, cyclohexyl, SiMe₃, COOR¹¹, CON(R¹¹)₂,
R¹¹ are identical or different and are hydrogen, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, CH₂CH₂OMe, CH(CH₃)CH₂OMe, CH₂CH(CH₃)OMe, CH₂CH₂OEt, CH(CH₃)CH₂OEt, CH₂CH(CH₃)OEt, CF₃, CF₂H, CCl₃, C₂F₅, C₃F₇, CF(CF₃)₂, cyclopropyl, cyclobutyl, cyclopentyl, cyclohexyl, SiMe₃, COOR¹¹, CON(R¹¹)₂, phenyl or
   if two radicals R¹¹ are attached to one nitrogen atom, these radicals together may represent piperidin-1-yl, piperazin-1-yl, 4-methylpiperazin-1-yl or morpholin-1-yl, or
   if two radicals R¹¹ are adjacent in the grouping NR¹¹COR¹¹, two radicals R¹¹ may form a 3- to 7-membered, unsubstituted or substituted saturated or unsaturated cycle which may contain up to five further heteroatoms selected from the group consisting of N, O and S, where two oxygen atoms are not adjacent,
R¹² is methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; CF₃, CF₂H, CCl₃, C₂F₅, C₃F₇, CF(CF₃)₂, cyclopropyl, cyclobutyl, cyclopentyl, cyclohexyl, CH₂OR¹¹, (CH₂)₂OR¹¹, (CH₂)₃OR¹¹, (CH₂)₄OR¹¹, SiMe₃,
R¹⁴ independently of one another are hydrogen, fluorine, chlorine, bromine, iodine, cyano, hydroxyl, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; CF₃, CF₂H, CCl₃, C₂F₅, C₃F₇, CF(CF₃)₂, or cyclopropyl, cyclohexyl, cyclopentyl, SiMe₃,
A is OR¹⁵, N(R¹⁵)₂ or C(R¹⁶)₃,
R¹⁵ independently of one another are COR⁷, COCH₃, COCF₃, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, CH₂CH₂OMe, CH(CH₃)CH₂OMe, CH₂CH(CH₃)OMe, CH₂CH₂OEt, CH(CH₃)CH₂OEt, CH₂CH(CH₃)OEt, CF₃, CF₂H, CCl₃, C₂F₅, C₃F₇, CF(CF₃)₂, cyclopropyl, cyclobutyl, cyclohexyl, CH₂OR¹¹, (CH₂)₂OR¹¹, (CH₂)₃OR¹¹, (CH₂)₄OR¹¹,
R¹⁶ independently of one another are hydrogen, halogen, cyano, hydroxyl, OR¹¹, SR¹¹, COR¹¹, N(R¹¹)₂, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; CF₃, CF₂H, CCl₃, C₂F₅, C₃F₇, CF(CF₃)₂, cyclopropyl, cyclobutyl, cyclopentyl, cyclohexyl, CH₂OR¹¹, (CH₂)₂OR¹¹, (CH₂)₃OR¹¹, (CH₂)₄OR¹¹, SiMe₃ or
   in each case two radicals R¹⁶ represent doubly attached oxygen or doubly attached sulphur, or
   in each case two radicals R⁴, R¹⁴ or R¹¹, R¹¹ or R¹⁶, R¹⁶ or R¹⁴, R¹¹ or R¹⁴, R¹⁶ together form a cyclopentyl, cyclohexyl, cyclopentenyl, cyclohexenyl, cyclopentadienyl, cyclohexadienyl, 4-pyridyl,
R¹⁷ is methyl, ethyl, propyl, 1-methylethyl, CF₃, CF₂H, CCl₃, C₂F₅, C₃F₇, CF(CF₃)₂.

Very particular preference is given to compounds of the formula (Ia) in which one or more of the symbols have one of the following meanings:
R¹ to R⁴ independently of one another are hydrogen, fluorine, chlorine, bromine, iodine, cyano, hydroxyl, nitro, OR¹¹, O-Me, O-Et, O—Pr, O-iPr, O-Bu, O-secBu, O-isoBu, O-tBu, O—(CH₂)₂OH, O—(CH₂)₂OCH₃, O—(CH₂)₃OH, O—(CH₂)₃OCH₃, OCF₃, SR¹¹, SH, S-Me, S-Et, S—Pr, S-iPr, S-Bu, S-secBu, S-isoBu, S-tBu, SCF₃, SOR¹¹, SO-Me, SO-Et, SO—Pr, SO-iPr, SO-Bu, SO-secBu, SO-isoBu, SO-tBu, SO₂R¹¹, SO₂-Me, SO₂-Et, SO₂—Pr, SO₂-iPr, SO₂—Bu, SO₂-secBu, SO₂-isoBu, SO₂-tBu, SON(R¹¹)₂, SONHMe, SONMe₂, SONHEt, SONEt₂, SONHPr, SONPr₂, SONHBu, SONBu₂, SONHCF₃, SON(CF₃)₂, SO₂N(R¹¹)₂, SO₂NHMe, SO₂NMe₂, SO₂NEt₂, SO₂NHEt, SO₂NPr₂, SO₂NHPr, SO₂NHCF₃, SO₂N(CF₃)₂, COR¹¹, COMe, COEt, COPr, COiPr, COBu, COsecBu, CO-isoBu, COtBu, COCF₃, NR¹¹CO₂R¹¹, NR¹¹CO₂Me, NR¹¹CO₂Et, NR¹¹CO₂Pr, NR¹¹CO₂iPr, NR¹¹CO₂Bu, NR¹¹CO₂secBu, NR¹¹ CO₂ isoBu, NR¹¹ CO₂tBu, NR¹¹ COR¹¹, NHCOMe, NHCOEt, NHCOPr, NHCOiPr, NHCOBu, NHCOiBu, NHCOsecBu, NHCOisoBu, NHCOtBu, NHCO(CH₂)₂OH, NHCO(CH₂)₂OCH₃, NHCO(CH₂)₃OH, NHCO(CH₂)₃OCH₃, NR¹¹COMe, NR¹¹COEt, NR¹¹COPr, NR¹¹COiPr, NR¹¹COBu, NR¹¹COsecBu, NR¹¹COtBu, NR¹¹CO(CH₂)₂OH, NR¹¹CO(CH₂)₂OCH₃, NR¹¹CO(CH₂)₃OH, NR¹¹CO(CH₂)₃OCH₃, N(R¹¹)₂, NMe₂, NEt₂, NHMe, NH₂, NHtBu, NHEt, NHPr, NHiPr, NHBu, NHiBu, NHsecBu, piperazin-1-yl, 4-methylpiperazin-1-yl, morpholin-1-yl, NR¹¹SO₂R¹², NHSOR¹¹, NR¹¹SOR¹¹, NHSOMe, NHSO₂Me, NHSOEt, NHSO₂Et, NMeSOMe, NHSO₂R¹¹, NR¹¹SO₂R¹¹, NMeSO₂Me, NMeSOEt, NMeSO₂Et, NHSOCF₃, NHSO₂CF₃, OCON(R¹¹)₂, OCONHMe, OCONHEt, OCONHPr, OCONHiPr, OCONHBu, OCONHsecBu, OCONHisoBu, OCONHtBu, OCONMe₂, OCONEt₂, OCONPr₂, OCONiPr₂, OCONBu₂, OCONsecBu₂, OCONisoBu₂, OCONHtBu₂, OCOR¹¹, OCOMe, OCOEt, OCOPr, OCOiPr, OCOBu, OCOsecBu, OCOisoBu, OCOtBu, CON(R¹¹)₂, CONHEt, CONEt₂, CONHMe, CONMe₂, CONHPr, CONPr₂, CONHBu, CONHsecBu, CONHisoBu, CONHtBu, CONHCH(CH₃)CH₂OH, CONHCH(CH₃)CH₂OCH₃, CONHCH(C₂H₅)CH₂OH, CONHCH(C₂H₅)CH₂OCH₃, CONH(CH₂)₂OCH₃, CONH(CH₂)₂OH, CONH(CH₂)₃OCH₃, CONH(CH₂)₃OH, CONR¹¹CH(CH₃)CH₂OH, CONR¹¹CH(CH₃)CH₂OCH₃, CONR¹¹CH(C₂H₅)CH₂OH, CONR¹¹CH(C₂H₅)CH₂OCH₃, CONR¹¹(CH₂)₂OCH₃, CONR¹¹(CH₂)₂OH, CONR¹¹(CH₂)₃OCH₃, CONR¹¹(CH₂)₃OH, CO₂R¹¹, CO₂Me, CO₂Et, CO₂Pr, CO₂iPr, CO₂Bu, CO₂secBu, CO₂ isoBu, CO₂tBu, CO₂(CH₂)₂OH, CO₂(CH₂)₂OCH₃, CO₂(CH₂)₃OH, CO₂(CH₂)₃OCH₃, (CH₂)ₘOR¹¹, CH₂OH, (CH₂)₂OH, (CH₂)₃OH, (CH₂)₄OH, CH₂OMe, (CH₂)₂OMe, (CH₂)₃OMe, (CH₂)₄OMe, (CH₂)ₘSR¹¹, CH₂SH, (CH₂)₂SH, (CH₂)₃SH, (CH₂)₄SH, CH₂SMe, (CH₂)₂SMe, (CH₂)₃SMe, (CH₂)₄SMe, (CH₂)ₘN(R¹¹)₂, CH₂NH₂, CH₂NAc₂, CH₂N(COCF₃)₂, CH₂NHAc, CH₂NHCOCF₃, (CH₂)₂NH₂, (CH₂)₃NH₂, (CH₂)₄NH₂, CH₂NMe₂, (CH₂)₂NHMe, (CH₂)₂NMe₂, (CH₂)₃NHMe, (CH₂)₃NMe₂, (CH₂)₄NHMe, (CH₂)₄NMe₂, (CH₂)ₘCOOR¹², CH₂COOMe, (CH₂)₂COOMe, (CH₂)₃COOMe, CH₂COOEt, (CH₂)₂COOEt, (CH₂)₃COOEt, CH₂COOPr, (CH₂)₂COOPr, (CH₂)₃COOPr, CH₂COOiPr, (CH₂)₂COOiPr, (CH₂)₃COOiPr, CH₂COOtBu, (CH₂)₂COOtBu, (CH₂)₃COOtBu, CH₂COO(CH₂)₂OH CH₂COO(CH₂)₂OCH₃ CH₂COO(CH₂)₃OH, CH₂COO(CH₂)₃OCH₃ CH₂NHCOOR¹¹, CH₂NR¹¹COOR¹¹, CH₂NHCOOMe, CH₂NHCOOtBu, CH₂NHCOOEt, CH₂NHCOOPr, CH₂NHCOOiPr, CH₂NHCOOBu, CH₂NHCOOtBu, CH₂NHCOOsecBu, CH₂NHCOOisoBu, CH₂NR¹¹COOEt, CH₂NR¹¹COOPr, CH₂NR¹¹COOiPr, CH₂NR¹¹COOBu, CH₂NR¹¹ COOtBu, CH₂NR¹¹ COOsecBu, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; cyclopropyl, cyclobutyl, cyclopentyl, cyclohexyl; CF₃, CF₂H, CCl₃, C₂F₅, C₃F₇, CF(CF₃)₂,
   or in each case two adjacent radicals R¹, R² or R², R³ or R³, R⁸ or R⁸, R⁴ together form a 2,3-dihydro-1,4-dioxin, a 2,2,3,3-tetrafluoro-2,3-dihydro-1,4-dioxin, a 1,3-dioxole or a 2,2-difluoro-1,3-dioxole ring,
R⁵ is hydrogen, acetyl, trifluoroacetyl,
X is nitrogen or CR⁸,
Y is nitrogen,
Z is nitrogen or CR¹⁰,
R⁷ is hydrogen, fluorine, chlorine, bromine, iodine, cyano, hydroxyl, amino, N(R¹¹)₂, nitro, OR¹¹, SR¹¹, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methyl-pentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; CF₃, CF₂H, CCl₃, C₂F₅, C₃F₇, CF(CF₃)₂, cyclopropyl, cyclobutyl, cyclopentyl, cyclohexyl, phenyl, p-Cl-phenyl, p-F-phenyl, p-Br-phenyl, p-I-phenyl, p-methoxyphenyl, p-trifluoromethoxyphenyl, 2-pyridyl, 3-pyridyl, 4-pyridyl, 2-thienyl, 3-thienyl, SiMe₃, COOR¹¹, CO₂Me, CO₂Et, CO₂Pr, CO₂iPr, CO₂Bu, CO₂secBu, CO₂ isoBu, CO₂tBu, CO₂(CH₂)₂OH, CO₂(CH₂)₂OCH₃, CO₂(CH₂)₃OH, CO₂(CH₂)₃OCH₃, CON(R¹¹)₂, CONHEt, CONEt₂, CONHMe, CONMe₂, CONHPr, CONPr₂, CONHBu, CONHsecBu, CONHisoBu, CONHtBu, CONHCH(CH₃)CH₂OH, CONHCH(CH₃)CH₂OCH₃, CONHCH(C₂H₅)CH₂OH, CONHCH(C₂H₅)CH₂OCH₃, CONH(CH₂)₂OCH₃, CONH(CH₂)₂OH, CONH(CH₂)₃OCH₃, CONH(CH₂)₃OH, CONR¹¹CH(CH₃)CH₂OH, COR¹¹COMe, COEt, COPr, COiPr, COBu, COsecBu, CO-isoBu, COtBu, COCF₃ or
R⁷ and R¹⁰ together form a saturated or unsaturated bridge of the following structure:

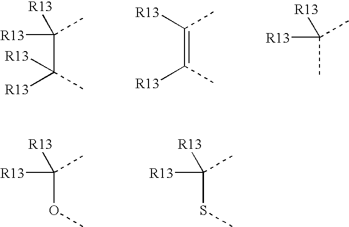

where R¹³=independently of one another hydrogen, fluorine, chlorine, bromine, iodine, methyl, ethyl, propyl, 1-methylethyl, CF₂H, CF₃, C₂F₅, OCH₃; OC₂H₅, OCF₃, OC₂F₅ or
   two geminal radicals R¹³ represent doubly attached oxygen or sulphur,
R⁸ is hydrogen, fluorine, chlorine, bromine, iodine, cyano, hydroxyl, nitro, OR¹¹, O-Me, O-Et, O—Pr, O-iPr, O-Bu, O-secBu, O-isoBu, O-tBu, O—(CH₂)₂OH, O—(CH₂)₂OCH₃, O—(CH₂)₃OH, O—(CH₂)₃OCH₃, OCF₃, SR¹¹, SH, S-Me, S-Et, S—Pr, S-iPr, S-Bu, S-secBu, S-isoBu, S-tBu, SCF₃, SOR¹¹, SO-Me, SO-Et, SO—Pr, SO-iPr, SO-Bu, SO-secBu, SO-isoBu, SO-tBu, SO₂R¹¹, SO₂-Me, SO₂-Et, SO₂—Pr, SO₂-iPr, SO₂—Bu, SO₂-secBu, SO₂-isoBu, SO₂-tBu, SON(R¹¹)₂, SONHMe, SONMe₂, SONHEt, SONEt₂, SONHPr, SONPr₂, SONHBu, SONBu₂, SONHCF₃, SON(CF₃)₂, SO₂N(R¹¹)₂, SO₂NHMe, SO₂NMe₂, SO₂NEt₂, SO₂NHEt, SO₂NPr₂, SO₂NHPr, SO₂NHCF₃, SO₂N(CF₃)₂, COR¹¹, COMe, COEt, COPr, COiPr, COBu, COsecBu, COisoBu, COtBu, COCF₃, NR¹¹CO₂R¹¹, NR¹¹CO₂Me, NR¹¹CO₂Et, NR¹¹CO₂Pr, NR¹¹CO₂iPr, NR¹¹CO₂Bu, NR¹¹CO₂secBu, NR¹¹CO₂ isoBu, NR¹¹CO₂tBu, NR¹¹ COR¹¹, NHCOMe, NHCOEt, NHCOPr, NHCOiPr, NHCOBu, NHCOiBu, NHCOsecBu, NHCOisoBu, NHCOtBu, NHCO(CH₂)₂OH, NHCO(CH₂)₂OCH₃, NHCO(CH₂)₃OH, NHCO(CH₂)₃OCH₃, NR¹¹COMe, NR¹¹COEt, NR¹¹COPr, NR¹¹COiPr, NR¹¹COBu, NR¹¹COsecBu, NR¹¹COisoBu, NR¹¹COtBu, NR¹¹CO(CH₂)₂OH, NR¹¹CO(CH₂)₂OCH₃, NR¹¹CO(CH₂)₃OH, NR¹¹CO(CH₂)₃OCH₃, N(R¹¹)₂, NMe₂, NEt₂, NHMe, NH₂, NHtBu, NHEt, NHPr, NHiPr, NHBu, NHsecBu, NHisoBu, 4-methylpiperazin-1-yl, piperazin-1-yl, morpholin-1-yl, NR¹¹SO₂R¹², NHSOR¹¹, NR¹¹SOR¹¹, NHSOMe, NHSO₂Me, NHSOEt, NHSO₂Et, NMeSOMe, NHSO₂R¹¹, NR¹¹SO₂R¹¹, NMeSO₂Me, NMeSOEt, NMeSO₂Et, NHSOCF₃, NHSO₂CF₃, OCON(R¹¹)₂, OCONHMe, OCONHEt, OCONHPr, OCONHiPr, OCONHBu, OCONHsecBu, OCONHisoBu, OCONHtBu, OCONMe₂, OCONEt₂, OCONPr₂, OCONiPr₂, OCONBu₂, OCONsecBu₂, OCONisoBu₂, OCONtBu₂, OCOR¹¹, OCOMe, OCOEt, OCOPr, OCOiPr, OCOBu, OCOsecBu, OCOisoBu, OCOtBu, CON(R¹¹)₂, CONHEt, CONEt₂, CONHMe, CONMe₂, CONHPr, CONPr₂, CONHBu, OCONHBu, CONHtBu, CONHCH(CH₃)CH₂OH, CONHCH(CH₃)CH₂OCH₃, CONHCH(C₂H₅)CH₂OH, CONHCH(C₂H₅)CH₂OCH₃, CONH(CH₂)₂OCH₃, CONH(CH₂)₂OH, CONH(CH₂)₃OCH₃, CONH(CH₂)₃OH, CONR¹¹CH(CH₃)CH₂OH, CONR¹¹CH(CH₃)CH₂OCH₃, CONR¹¹CH(C₂H₅)CH₂OH, CONR¹¹CH(C₂H₅)CH₂OCH₃, CONR¹¹(CH₂)₂OCH₃, CONR¹¹(CH₂)₂OH, CONR¹¹(CH₂)₃OCH₃, CONR¹¹(CH₂)₃OH, CO₂R¹¹, CO₂Me, CO₂Et, CO₂Pr, CO₂iPr, CO₂Bu, CO₂secBu, CO₂ isoBu, CO₂tBu, CO₂(CH₂)₂OH, CO₂(CH₂)₂OCH₃, CO₂(CH₂)₃OH, CO₂(CH₂)₃OCH₃, (CH₂)ₘOR¹¹, CH₂OH, (CH₂)₂OH, (CH₂)₃OH, (CH₂)₄OH, CH₂OMe, (CH₂)₂OMe, (CH₂)₃OMe, (CH₂)₄OMe, (CH₂)ₘSR¹¹, CH₂SH, (CH₂)₂SH, (CH₂)₃SH, (CH₂)₄SH, CH₂SMe, (CH₂)₂SMe, (CH₂)₃SMe, (CH₂)₄SMe, (CH₂)ₘN(R¹¹)₂, CH₂NH₂, CH₂NAc₂, CH₂N(COCF₃)₂, CH₂NHAc, CH₂NHCOCF₃, (CH₂)₂NH₂, (CH₂)₃NH₂, (CH₂)₄NH₂, CH₂NMe₂, (CH₂)₂NHMe, (CH₂)₂NMe₂, (CH₂)₃NHMe, (CH₂)₃NMe₂, (CH₂)₄NHMe, (CH₂)₄NMe₂, (CH₂)ₘCOOR¹², CH₂COOMe, (CH₂)₂COOMe, (CH₂)₃COOMe, CH₂COOEt, (CH₂)₂COOEt, (CH₂)₃COOEt, CH₂COOPr, (CH₂)₂COOPr, (CH₂)₃COOPr, CH₂COOiPr, (CH₂)₂COOiPr, (CH₂)₃COOiPr, CH₂COOtBu, (CH₂)₂COOtBu, (CH₂)₃COOtBu, CH₂COO(CH₂)₂OH, CH₂COO(CH₂)₂OCH₃, CH₂COO(CH₂)₃OH, CH₂COO(CH₂)₃OCH₃, CH₂NHCOOR¹¹, CH₂NR¹¹COOR¹¹, CH₂NHCOOMe, CH₂NHCOOtBu, CH₂NHCOOEt, CH₂NHCOOPr, CH₂NHCOOiPr, CH₂NHCOOBu, CH₂NHCOOtBu, CH₂NHCOOsecBu, CH₂NHCOOisoBu, CH₂NR¹¹COOEt, CH₂NR¹¹COOPr, CH₂NR¹¹ COOiPr, CH₂NR¹¹COOBu, CH₂NR¹¹COOtBu, CH₂NR¹¹COOsecBu, CH₂NR¹¹COOisoBu, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methyl-propyl; cyclopropyl, cyclobutyl, cyclopentyl, cyclohexyl; CF₃, CF₂H, CCl₃, C₂F₅, C₃F₇, CF(CF₃)₂,
R¹⁰ is hydrogen, fluorine, chlorine, bromine, iodine, cyano, hydroxyl, amino, N(R¹¹)₂, NMe₂, NEt₂, NHMe, NH₂, NHtBu, NHEt, NHPr, NHiPr, NHBu, NHsecBu, NHisoBu, 4-methylpiperazin-1-yl, piperazin-1-yl, morpholin-1-yl, nitro, OR¹¹, O-Me, O-Et, O—Pr, O-iPr, O-Bu, O-secBu, O-isoBu, O-tBu, OCH(CH₃)CH₂OH, OCH(CH₃)CH₂OCH₃, OCH(C₂H₅)CH₂OH, OCH(C₂H₅)CH₂OCH₃, O(CH₂)₂OCH₃, O(CH₂)₂OH, O(CH₂)₃OCH₃, O(CH₂)₃OH, OCF₃, SR¹¹, S-Me, S-Et, S—Pr, S-iPr, S-Bu, S-secBu, S-isoBu, S-tBu, SCF₃, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; CF₃, CF₂H, CCl₃, C₂F₅, C₃F₇, CF(CF₃)₂, cyclopropyl, cyclobutyl, cyclopentyl, cyclohexyl, SiMe₃, COOR¹¹, CO₂Me, CO₂Et, CO₂Pr, CO₂iPr, CO₂Bu, CO₂secBu, CO₂ isoBu, CO₂tBu, CON(R¹¹)₂, CONH₂, CONHMe, CONMe₂, CONHEt, CONEt₂, CO-morpholine, CO-piperidine, CO-piperazine, CO-(4-methylpiperazine), CONHCH(CH₃)CH₂OH, CONHCH(CH₃)CH₂OCH₃, CONHCH(C₂H₅)CH₂OH, CONHCH(C₂H₅)CH₂OCH₃, CONH(CH₂)₂OCH₃, CONH(CH₂)₂OH, CONH(CH₂)₃OCH₃, CONH(CH₂)₃OH,
R¹¹ are identical or different and are hydrogen, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, CH₂CH₂OMe, CH(CH₃)CH₂OMe, CH₂CH(CH₃)OMe, CH₂CH₂OEt, CH(CH₃)CH₂OEt, CH₂CH(CH₃)OEt, CF₃, CF₂H, CCl₃, C₂F₅, C₃F₇, CF(CF₃)₂, cyclopropyl, cyclobutyl, cyclopentyl, cyclohexyl, SiMe₃, COOR¹¹, CON(R¹¹)₂, phenyl or
   if two radicals R¹¹ are attached to one nitrogen atom, these radicals together may represent piperidin-1-yl, piperazin-1-yl, 4-methylpiperazin-1-yl or morpholin-1-yl, or
   if two radicals R¹¹ are adjacent in the grouping NR¹¹COR¹¹, two radicals R¹¹ may form a 3- to 7-membered, unsubstituted or substituted saturated or unsaturated cycle which may contain up to five further heteroatoms selected from the group consisting of N, O and S, where two oxygen atoms are not adjacent,
R¹² is methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; CF₃, CF₂H, CCl₃, C₂F₅, C₃F₇, CF(CF₃)₂, cyclopropyl, cyclobutyl, cyclopentyl, cyclohexyl, CH₂OR¹¹, (CH₂)₂OR¹¹, (CH₂)₃OR¹¹, (CH₂)₄OR¹¹, SiMe₃,
R¹⁴ independently of one another are hydrogen, fluorine, chlorine, bromine, iodine, cyano, hydroxyl, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; CF₃, CF₂H, CCl₃, C₂F₅, C₃F₇, CF(CF₃)₂, or cyclopropyl, cyclohexyl, cyclopentyl, SiMe₃,
A is OR¹⁵, N(R⁵)₂ or C(R¹⁶)₃,
R¹⁵ independently of one another are COR¹⁷, COCH₃, COCF₃, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, CH₂CH₂OMe, CH(CH₃)CH₂OMe, CH₂CH(CH₃)OMe, CH₂CH₂OEt, CH(CH₃)CH₂OEt, CH₂CH(CH₃)OEt, CF₃, CF₂H, CCl₃, C₂F₅, C₃F₇, CF(CF₃)₂, cyclopropyl, cyclobutyl, cyclohexyl, CH₂OR¹¹, (CH₂)₂OR¹¹, (CH₂)₃OR¹¹, (CH₂)₄OR¹¹,
R¹⁶ independently of one another are hydrogen, halogen, cyano, hydroxyl, OR¹¹, SR¹¹, COR¹¹, N(R¹¹)₂, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; CF₃, CF₂H, CCl₃, C₂F₅, C₃F₇, CF(CF₃)₂, cyclopropyl, cyclobutyl, cyclopentyl, cyclohexyl, CH₂OR¹¹, (CH₂)₂OR¹¹, (CH₂)₃OR¹¹, (CH₂)₄OR¹¹, SiMe₃ or
   in each case two radicals R¹⁶ represent doubly attached oxygen or doubly attached sulphur, or
   in each case two radicals R¹⁴, R¹⁴ or R¹⁵, R¹⁵ or R¹⁶, R¹⁶ or R¹⁴, R¹⁵ or R¹⁴, R¹⁶ together form a cyclopentyl, cyclohexyl, cyclopentenyl, cyclohexenyl, cyclopentadienyl, cyclohexadienyl, 4-pyridyl,
R¹⁷ is methyl, ethyl, propyl, 1-methylethyl, CF₃, CF₂H, CCl₃, C₂F₅, C₃F₇, CF(CF₃)₂.

Furthermore, very particular preference is given to compounds of the formula (Ia) in which one or more of the symbols have one of the following meanings:
R¹ to R⁴ independently of one another are hydrogen, fluorine, chlorine, cyano, hydroxyl, nitro, OMe, CF₃, COCH₃, COOCH₃, COOH, N(Me)₂, NHCOCH₃, NHCOCF₃, NHSO₂CH₃, NHCOOCH₃, NHCOO(CH₂CH₂)OCH₃, 4-N-methylpiperazin-1-yl, CH₂NH₂, CH₂NHCOOCH₃, CH₂NHCOOtBu, methyl,
   or in each case two adjacent radicals R¹, R² or R², R³ or R³, R⁸ or R⁸, R⁴ together form a 2,3-dihydro-1,4-dioxin or a 1,3-dioxole ring,
R⁵ is hydrogen, COCH₃,
X is nitrogen or CR⁸,
Y is nitrogen,
Z is nitrogen or CR¹¹,
R⁷ is hydrogen, methyl, 1-methylpropyl, CF₃, CF₂H, cyclopropyl, or
R⁷ and R¹⁰ together form a saturated or unsaturated bridge of the following structure:

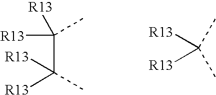

where R¹³=hydrogen,
R⁸ is hydrogen, fluorine, chlorine, cyano, hydroxyl, nitro, OMe, CF₃, COCH₃, COOCH₃, COOH, N(Me)₂, NHCOCH₃, NHCOCF₃, NHSO₂CH₃, NHCOOCH₃, NHCOO(CH₂CH₂)OCH₃, 4-N-methylpiperazin-1-yl, CH₂NH₂, CH₂NHCOOCH₃, CH₂NHCOOtBu, methyl,
R¹⁰ is hydrogen, or
R⁷ and R¹⁰ together form a saturated or unsaturated bridge of the following structure:

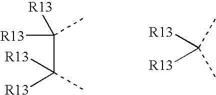

where R¹³=hydrogen
R¹¹ are identical or different and are hydrogen, methyl, ethyl, CH(CH₃)CH₂OMe, CH₂CH(CH₃)OMe, CH₂CH₂OEt, CH(CH₃)CH₂OEt, CH₂CH(CH₃)OEt, cyclopropyl, cyclohexyl, or
   if two radicals R¹¹ are attached to one nitrogen atom, these radicals together may represent 4-methylpiperazin-1-yl,
R¹² is methyl, ethyl, propyl, 1-methylethyl, CF₃, cyclopropyl, cyclohexyl,
R¹⁴ independently of one another are hydrogen, methyl, cyclopropyl,
A is OR¹⁵ or C(R¹⁶)₃,
R¹⁵ is ethyl,
R¹⁶ is hydrogen,
R¹⁷ is methyl, ethyl, propyl, 1-methylethyl, CF₃, CF₂H, CCl₃, C₂F₅, C₃F₇, CF(CF₃)₂.

Furthermore, very particular preference is given to compounds of the formula (Ia) in which one or more of the symbols have one of the following meanings:
R¹ to R⁴ independently of one another are hydrogen, fluorine, chlorine, iodine, cyano, hydroxyl, nitro, OMe, OCH₂tBu, OCOCH₃, SO₂NH₂, SO₂N(CH₃)₂, COCH₃, COCH₂CH₂CH₃, COOCH₂CH₂OCH₃, COOCH₂CH₂CH₂OCH₃, CONHCH₂CH₂OCH₃, NH₂, N(Me)₂, NHCOCH₃, NHSO₂CH₃, NHCOOCH₃, NHCOOCH₂CH₃, NHCOOtBu, NHCOOCH₂CH₂OCH₃, 4-N-acetylpiperazin-1-yl, N-pyrrolidin-2-on-1-yl, CH₂NHCOOCH₃, CH₂NHCOOtBu, methyl, CF₃
   or in each case two adjacent radicals R¹, R² or R², R³ or R³, R⁸ or R⁸, R⁴ together form a 2,3-dihydrofuran-2-one, 2,3-dihydro-1,4-dioxin, a 2,2,3,3-tetrafluoro-2,3-dihydro-1,4-dioxin, a 1,3-dioxole or a 2,2-difluoro-1,3-dioxole ring,
R⁵ is hydrogen, COCH₃, COCF₃, COOCH₃,
X is nitrogen or CR⁸,
R⁸ is hydrogen, O-Me,
Y is nitrogen,
Z is CR¹⁰,
R¹⁰ is hydrogen, fluorine, chlorine, bromine, cyano, CF₃ or
R⁷ and R¹⁰ together form a CH₂ or a CH₂CH₂ bridge,
R⁷ is hydrogen, methyl, CF₃, CF₂H, or
R⁷ and R¹⁰ together form a CH₂ or a CH₂CH₂ bridge,
R¹¹ are identical or different and are hydrogen, methyl, ethyl, CH(CH₃)CH₂OMe, CH₂CH(CH₃)OMe, CH₂CH₂OEt, CH(CH₃)CH₂OEt, CH₂CH(CH₃)OEt, cyclopropyl, cyclohexyl, or
   if two radicals R¹¹ are attached to one nitrogen atom, these radicals together may represent 4-methylpiperazin-1-yl,
R¹² is methyl, ethyl, propyl, 1-methylethyl, CF₃, cyclopropyl, cyclohexyl,
R¹⁴ independently of one another are hydrogen, methyl, cyclopropyl,
A is OR¹⁵ or C(R¹⁶)₃,
R¹⁵ is ethyl,
R¹⁶ is hydrogen,
R¹⁷ is methyl, ethyl, propyl, 1-methylethyl, CF₃, CF₂H, CCl₃, C₂F₅, C₃F₇, CF(CF₃)₂.

Moreover, very particular preference is given to compounds of the formula (Ia) in which one or more of the symbols have one of the following meanings:
R¹ to R⁴ independently of one another are hydrogen, fluorine, chlorine, iodine, cyano, hydroxyl, nitro, OMe, OCH₂tBu, OCOCH₃, SO₂NH₂, SO₂N(CH₃)₂, COCH₃, COCH₂CH₂CH₃, COOCH₂CH₂OCH₃, COOCH₂CH₂CH₂OCH₃, CONHCH₂CH₂OCH₃, NH₂, N(Me)₂, NHCOCH₃, NHSO₂CH₃, NHCOOCH₃, NHCOOCH₂CH₃, NHCOOtBu, NHCOOCH₂CH₂OCH₃, CH₂NHCOOCH₃, CH₂NHCOOtBu, methyl, CF₃
   or in each case two adjacent radicals R¹, R² or R², R³ or R³, R⁸ or R⁸, R⁴ together form a 2,3-dihydro-1,4-dioxin, a 2,2,3,3-tetrafluoro-2,3-dihydro-1,4-dioxin, a 1,3-dioxole or 2,2-difluoro-1,3-dioxole ring,
R⁵ is hydrogen, COCH₃, COCF₃, COOCH₃,
X is nitrogen or CR⁸,
R⁸ is hydrogen, O-Me,
Y is nitrogen,
Z is CR¹⁰,
R¹⁰ is hydrogen, fluorine, chlorine, bromine, cyano, CF₃ or
R⁷ and R¹⁰ together form a CH₂ or a CH₂CH₂ bridge,
R⁶ is ethyl, isopropyl, CH₂OCH₃, cyclopropyl, 3-pyridyl,
R⁷ is hydrogen, methyl, CF₃, CF₂H, or
R⁷ and R¹⁰ together form a CH₂ or a CH₂CH₂ bridge,
R¹¹ are identical or different and are hydrogen, methyl, ethyl, CH(CH₃)CH₂OMe, CH₂CH(CH₃)OMe, CH₂CH₂OEt, CH(CH₃)CH₂OEt, CH₂CH(CH₃)OEt, cyclopropyl, cyclohexyl, or
   if two radicals R¹¹ are attached to one nitrogen atom, these radicals together may represent 4-methylpiperazin-1-yl,
R¹² is methyl, ethyl, propyl, 1-methylethyl, CF₃, cyclopropyl, cyclohexyl,
R¹⁴ independently of one another are hydrogen, methyl, cyclopropyl,
A is OR¹⁵ or C(R¹⁶)₃,
R¹⁵ is ethyl,
R¹⁶ is hydrogen,
R¹⁷ is methyl, ethyl, propyl, 1-methylethyl, CF₃, CF₂H, CCl₃, C₂F₅, C₃F₇, CF(CF₃)₂.

Furthermore, very particular preference is given to compounds of the formula (Ia) in which one or more of the symbols have one of the following meanings:
R¹ to R⁴ independently of one another are hydrogen, fluorine, chlorine, cyano, hydroxyl, nitro, OMe, CF₃, COCH₃, COOCH₃, COOH, N(Me)₂, NHCOCH₃, NHSO₂CH₃, NHCOOCH₃, NHCOO(CH₂CH₂)OCH₃, 4-N-methylpiperazin-1-yl, CH₂NH₂, CH₂NHCOOCH₃, CH₂NHCOOtBu
   or in each case two adjacent radicals R¹, R² or R², R³ or R³, R⁸ or R⁸, R⁴ together form a 2,3-dihydro-1,4-dioxin, or a 1,3-dioxole ring,
R⁵ is hydrogen,
X is nitrogen or CR⁸,
Y is nitrogen,
Z is nitrogen or CR¹⁰,
R⁷ is hydrogen, methyl, 1-methylpropyl, CF₃, cyclopropyl, or
R⁷ and R¹⁰ together form a saturated or unsaturated bridge of the following structure:

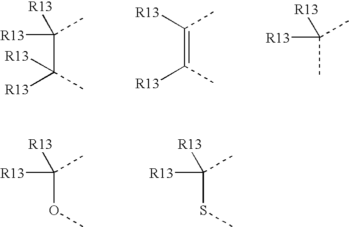

where R¹³=hydrogen,
R⁸ is hydrogen, fluorine, chlorine, cyano, hydroxyl, nitro, OMe, CF₃, COCH₃, COOCH₃, COOH, N(Me)₂, NHCOCH₃, NHSO₂CH₃, NHCOOCH₃, NHCOO(CH₂CH₂)OCH₃, 4-N-methylpiperazin-1-yl, CH₂NH₂, CH₂NHCOOCH₃, CH₂NHCOOtBu,
R¹⁰ is hydrogen or
R⁷ and R¹⁰ together form a saturated or unsaturated bridge of the following structure:

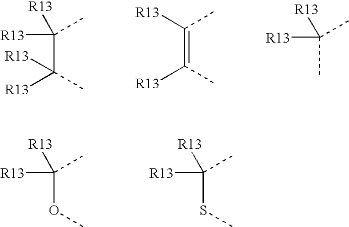

where R¹³=hydrogen,
R¹¹ are identical or different and are hydrogen, methyl, ethyl, CH(CH₃)CH₂OMe, CH₂CH(CH₃)OMe, CH₂CH₂OEt, CH(CH₃)CH₂OEt, CH₂CH(CH₃)OEt, cyclopropyl, cyclohexyl, or
   if two radicals R¹¹ are attached to one nitrogen atom, these radicals together may represent 4-methylpiperazin-1-yl,
R¹² is methyl, ethyl, propyl, 1-methylethyl, CF₃, cyclopropyl, cyclohexyl,
R¹⁴ independently of one another are hydrogen, methyl,
A is OR¹⁵ or C(R¹⁶)₃,
R¹⁵ is ethyl,
R¹⁶ is hydrogen,
R¹⁷ is methyl, ethyl, propyl, 1-methylethyl, CF₃, CF₂H, CCl₃, C₂F₅, C₃F₇, CF(CF₃)₂.

The radical definitions mentioned above may be combined with one another as desired. Moreover, individual definitions may not apply.

In the definitions of the symbols given in the formulae above, collective terms were used which are generally representative of the following substituents:

halogen: fluorine, chlorine, bromine and iodine;
alkyl: saturated straight-chain or branched hydrocarbon radicals having 1 to 4, 6 or 8 carbon atoms, for example C₁-C₆-alkyl, such as methyl, ethyl, propyl, 1-methylethyl, butyl, 1-methyl-propyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethyl-butyl, 1,1,2-trimethylpropyl, 1,2,2-trimethylpropyl, 1-ethyl-1-methylpropyl and 1-ethyl-2-methyl-propyl;
haloalkyl: straight-chain or branched alkyl groups having 1 to 8 carbon atoms (as mentioned above), where some or all of the hydrogen atoms in these groups may be replaced by halogen atoms as mentioned above, for example C₁-C₃-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl and 1,1,1-trifluoroprop-2-yl;
alkenyl: unsaturated straight-chain or branched hydrocarbon radicals having 2 to 4, 6 or 8 carbon atoms and a double bond in any position, for example C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;
alkynyl: straight-chain or branched hydrocarbon groups having 2 to 4, 6 or 8 carbon atoms and a triple bond in any position, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;
cycloalkyl: monocyclic saturated hydrocarbon groups having 3 to 6 carbon ring members, such as cyclopropyl, cyclobutyl, cyclopentyl and cyclohexyl;
cylcoalkenyl: monocyclic nonaromatic hydrocarbon groups having 3 to 8 carbon ring members and at least one double bond, such as cyclopenten-1-yl, cyclohexen-1-yl, cyclohepta-1,3-dien-1-yl;
alkoxycarbonyl: an alkoxy group having 1 to 6 carbon atoms (as mentioned above) which is attached to the skeleton via a carbonyl group (—CO—);
oxyalkyleneoxy: divalent unbranched chains of 1 to 3 CH₂ groups, where both valencies are attached to the skeleton via an oxygen atom, for example OCH₂O, OCH₂CH₂O and OCH₂CH₂CH₂O;
a five- to ten-membered saturated or partially unsaturated heterocycle which contains one to four heteroatoms from the group consisting of oxygen, nitrogen and sulphur: mono- or bicyclic heterocycles (heterocyclyl) which contain, in addition to carbon ring members, one to three nitrogen atoms and/or one oxygen or sulphur atom or one or two oxygen and/or sulphur atoms; if the ring contains a plurality of oxygen atoms, these are not directly adjacent; for example oxiranyl, aziridinyl, 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl and 1,2,4-hexahydrotriazin-3-yl;
a five- to ten-membered aromatic heterocycle which contains one to four heteroatoms from the group consisting of oxygen, nitrogen and sulphur: mono- or bicyclic heteroaryl, for example
5-membered heteroaryl which contains one to four nitrogen atoms or one to three nitrogen atoms and one sulphur or oxygen atom: 5-membered heteroaryl groups which, in addition to carbon atoms, may contain one to four nitrogen atoms or one to three nitrogen atoms and one sulphur or oxygen atom as ring members, for example 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl;
benzo-fused 5-membered heteroaryl which contains one to three nitrogen atoms or one nitrogen atom and one oxygen or sulphur atom: 5-membered heteroaryl groups which, in addition to carbon atoms, may contain one to four nitrogen atoms or one to three nitrogen atoms and one sulphur or oxygen atom as ring members and in which two adjacent carbon ring members or one nitrogen and one adjacent carbon ring member may be bridged by a buta-1,3-diene-1,4-diyl group, in which one or two carbon atoms may be replaced by nitrogen atoms;
   5-membered heteroaryl which is attached via nitrogen and contains one to four nitrogen atoms, or benzo-fused 5-membered heteroaryl which is attached via nitrogen and contains one to three nitrogen atoms: 5-membered heteroaryl groups which, in addition to carbon atoms, may contain one to four nitrogen atoms or one to three nitrogen atoms as ring members and in which two adjacent carbon ring members or one nitrogen and one adjacent carbon ring member may be bridged by a buta-1,3-diene-1,4-diyl group in which one or two carbon atoms may be replaced by nitrogen atoms, where these rings are attached to the skeleton via one of the nitrogen ring members, for example 1-pyrrolyl, 1-pyrazolyl, 1,2,4-triazol-1-yl, 1-imidazolyl, 1,2,3-triazol-1-yl, 1,3,4-triazol-1-yl;
6-membered heteroaryl which contains one to three or one to four nitrogen atoms: 6-membered heteroaryl groups which, in addition to carbon atoms, may contain one to three or one to four nitrogen atoms as ring members, for example 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl.

Compounds of the formulae I and Ia can be prepared by generally known methods. A possible route for the synthesis of compounds Ib in which Y═N and Z=CH is illustrated in scheme 1. Here, the symbols are as defined above. R⁶ is as defined above. In addition, R⁶ may also represent a group C(R⁴)₂A in which the symbols are as defined above.

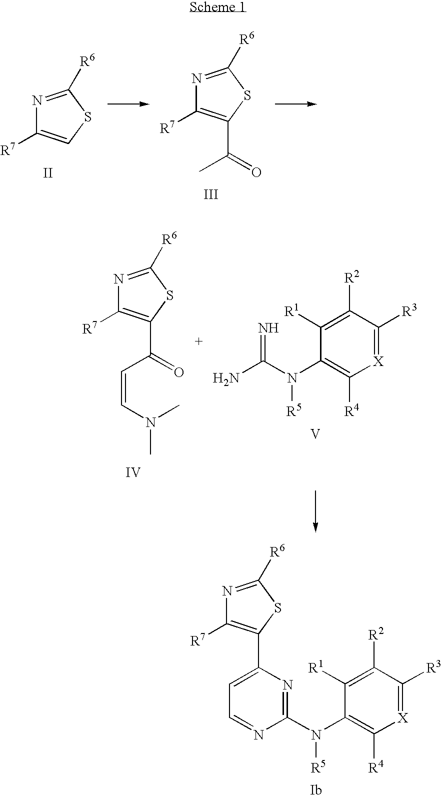

where R⁶ is C(R¹⁴)₂A for the synthesis of type Ia.

The synthesis of substituted thiazoles of type II and III is described in the literature (Houben-Weyl, Methoden der Organischen Chemie [Methods of Organic Chemistry], volume E6b, Hetarenes III/Part 2, Thieme Verlag 1994, pp. 1-361; see also, inter alia, WO-A 2005/012298; WO-A 2004/056368; WO-A 2001/072745). Thus, for example, thiazoles II can be synthesized according to the Hantzsch method by condensing appropriately substituted alpha-haloketones with appropriately substituted thioamides or (optionally N-substituted) thioureas. Alternatively, these thiazoles can be prepared by cyclocondensation of alpha-acylaminoketones using, for example, phosphorus pentasulphide (variant of the Robinson-Gabriel synthesis). Thiazol-5-yl methyl ketones III can be obtained, for example, by Friedel-Crafts acylation of 5-unsubstituierted thiazoles.

Alternatively, thiazol-5-yl methyl ketones of type III can be prepared from the thiazolyl-5-carboxylic acid derivatives described below; various routes to achieve this have been described in the literature (for example iron-catalysed addition of methyl Grignard to carbonyl chlorides: J. Org, Chem. 2004, 69, 3943; addition of methyl Grignard to Weinreb amides: Synlett 1999, 1091; addition of malonic esters to carbonyl chlorides followed by double decarboxylation: Tetrahedron 1992, 48, 9233; addition of methyl Grignard to carbonitriles: J. Am. Chem. Soc. 1956, 78, 2141).

In a condensation reaction, the thiazolyl ketones III are reacted with a methylene-activated compound to give enaminoketones IV (Chem. Ber. 1964, 97, 3397). 1,3-Dicarbonyl equivalents prepared in this manner can then be condensed with guanidines V or salts thereof to give pyrimidines Ib.

The substituted guanidines V or the corresponding guanidinium salts can be prepared by reacting suitable amines with cyanamide, for example by heating in a suitable solvent, for example ethanol, if appropriate in the presence of stoichiometric amounts of mineral acid, for example concentrated nitric acid or concentrated hydrochloric acid (US 1972/3681459; US 1975/3903159; US 1976/3076787).

Alternatively, compounds Ib can be synthesized from 2,4-disubstituted thiazole-5-carboxylic acid derivates VI (scheme 2).

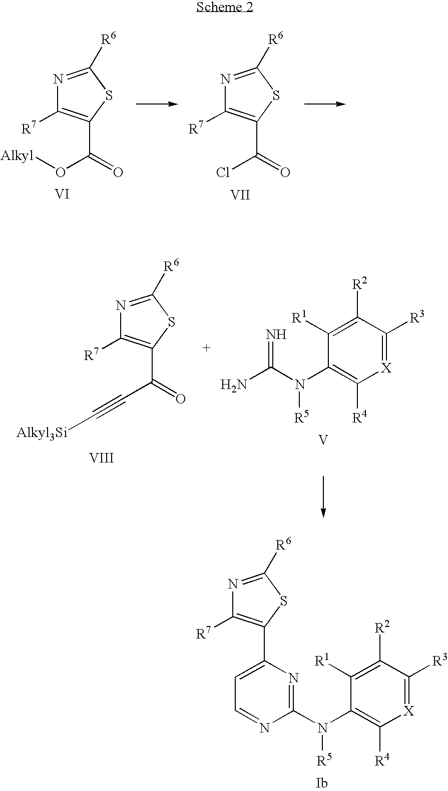

where R⁶ is C(R¹⁴)₂A for the synthesis of type Ia; Alkyl=independently of one another branched or unbranched C₁-C₄-alkyl.

The derivatives VI can be obtained by condensation reactions of 2-halo-1,3-dicarbonyl compounds, such as, for example, 2-halo-3-keto esters, 2-halo-3-ketonitriles or 2-halomalonic ester nitrile (Houben-Weyl, Methoden der Organischen Chemie, [Methods of Organic Chemistry] volume E6b, Hetarenes III/Part 2, Thieme Verlag 1994, pp. 1-361).

The present invention also provides intermediates of type VIII. Likewise, the present invention provides the conversion of the intermediates VIII into compounds Ib. The process is suitable in particular for preparing compounds Ia and I.

To this end, the thiazolecarboxylic acids are converted by known methods into the corresponding carbonyl chlorides VII (e.g. J. Chem. Soc. Perkin. Trans. I 1982, 159; J. Heterocycl. Chem. 1985, 22, 1621; J. Med. Chem. 1999, 42, 5064; J. Fluorine Chem. 2004, 125, 1287). These are then converted under Sonogashira conditions at room temperature into the corresponding trialkylsilylalkinones VIII (Org. Lett. 2003, 5, 3451; Synthesis 2003, 2815). The preferred palladium catalyst is (Ph₃P)₂PdCl₂; the preferred cocatalyst is CuI. The preferred stoichiometric auxiliary base is triethylamine. The Sonogashira reaction is preferably carried out in THF, but acetonitrile or other solvents, such as diethyl ether and the like, are also suitable as reaction medium.

Furthermore, alternative routes for preparing trialkylsilylalkinones are described in the literature (addition of lithium(trimethylsilyl)acetylide to Weinreb amides: Synth. Commun. 1993, 23, 487; Friedel-Crafts acylation of bis(trimethylsilyl)acetylene: J. Org. Chem. 1973, 38, 2254; Stille coupling with tributyl(trimethylsilylalkynyl)stannane: J. Org. Chem. 1982, 47, 2549).

The trialkylsilylalkinones VIII are then reacted in a condensation reaction with guanidines V or salts thereof to give the target compounds Ib. To this end, the starting materials, in the case of the guanidine salts with use of an auxiliary base, for example potassium carbonate, are reacted in a suitable solvent, for example DMF or 2-methoxyethanol, at 100° C. for 4-20 h. In addition, the use of other solvents (for example alcohols) and other auxiliary bases is conceivable.

Alternatively, the trialkylsilylalkinone can be prepared under Sonogashira conditions and the condensation reaction to give the aminopyrimidine can be carried out in a one-pot process; to this end, after the coupling reaction has been carried out, a cosolvent, for example methanol, is added if required and the mixture is reacted under reflux for a number of hours (Org. Lett. 2003, 5, 3451; Synthesis 2003, 2815).

This gives compounds (I) and (Ia), with the proviso that one of the two symbols Y or Z represents a nitrogen atom and the other remaining symbol represents an optionally substituted carbon atom. The thiazolecarboxylic esters VI required for carrying out the process according to the invention are known from the literature or can be obtained by processes analogous to the literature. The reaction to give the thiazolecarbonyl chlorides VII is carried out by literature methods. The trialkylsilanones VIII are obtained analogously to literature procedures. The guanidines V are likewise obtained by literature procedures.

The process according to the invention is preferably carried out using a noble metal catalyst customary for such reactions and a cocatalyst. The preferred palladium catalyst is (Ph₃P)₂PdCl₂; the preferred cocatalyst is CuI.

The process according to the invention is preferably carried out using one or more diluents. Suitable diluents are virtually all inert organic solvents. These preferably include aliphatic and aromatic, optionally halogenated hydrocarbons, such as pentane, hexane, heptane, cyclohexane, petroleum ether, benzine, ligroin, benzene, toluene, xylene, methylene chloride, ethylene chloride, chloroform, carbon tetrachloride, chlorobenzene and o-dichlorobenzene, ethers, such as diethyl ether and dibutyl ether, glycol dimethyl ether and diglycol dimethyl ether, tetrahydrofuran and dioxane, ketones, such as acetone, methyl ethyl ketone, methyl isopropyl ketone or methyl isobutyl ketone, esters, such as methyl acetate or ethyl acetate, nitriles, such as, for example, acetonitrile or propionitrile, amides, such as, for example, dimethylformamide, dimethylacetamide and N-methylpyrrolidone, and also dimethyl sulphoxide, tetramethylene sulphone and hexamethyl-phosphoric triamide. Preference is given to using tetrahydrofuran, diethyl ether or else acetonitrile.

The reaction temperatures in the process according to the invention can be varied within a relatively wide range. In general, the process is carried out at temperatures between 0° C. and 250° C., preferably at temperatures between 10° C. and 65° C.

If guanidinium salts are used, the process according to the invention for preparing the compounds of the formula Ib is preferably carried out using one or more reaction auxiliaries.

Suitable reaction auxiliaries are, if appropriate, the customary inorganic or organic bases or acid acceptors. These preferably include alkali metal or alkaline earth metal acetates, amides, carbonates, bicarbonates, hydrides, hydroxides or alkoxides, such as, for example, sodium acetate, potassium acetate or calcium acetate, lithium amide, sodium amide, potassium amide or calcium amide, sodium carbonate, potassium carbonate or calcium carbonate, sodium bicarbonate, potassium bicarbonate or calcium bicarbonate, lithium hydride, sodium hydride, potassium hydride or calcium hydride, lithium hydroxide, sodium hydroxide, potassium hydroxide or calcium hydroxide, sodium methoxide, ethoxide, n- or i-propoxide, n-, i-, s- or t-butoxide or potassium methoxide, ethoxide, n- or i-propoxide, n-, i-, s- or t-butoxide; furthermore also basic organic nitrogen compounds, such as, for example, trimethylamine, triethylamine, tripropylamine, tributylamine, ethyldiisopropylamine, N,N-dimethylcyclohexylamine, dicyclohexylamine, ethyldicyclohexylamine, N,N-dimethylaniline, N,N-dimethylbenzylamine, pyridine, 2-methyl-, 3-methyl-, 4-methyl-, 2,4-dimethyl-, 2,6-dimethyl-, 3,4-dimethyl- and 3,5-dimethylpyridine, 5-ethyl-2-methylpyridine, 4-dimethylaminopyridine, N-methylpiperidine, 1,4-diazabicyclo[2.2.2]-octane (DABCO), 1,5-diazabicyclo[4.3.0]non-5-ene (DBN) or 1,8 diazabicyclo[5.4.0]undec-7-ene (DBU). Preference is given to using potassium carbonate.

The process according to the invention is preferably carried out using one or more diluents. Suitable diluents are virtually all inert organic solvents. These preferably include aliphatic and aromatic, optionally halogenated hydrocarbons, such as pentane, hexane, heptane, cyclohexane, petroleum ether, benzine, ligroin, benzene, toluene, xylene, methylene chloride, ethylene chloride, chloroform, carbon tetrachloride, chlorobenzene and o-dichlorobenzene, ethers, such as diethyl ether and dibutyl ether, glycol dimethyl ether and diglycol dimethyl ether, tetrahydrofuran and dioxane, ketones, such as acetone, methyl ethyl ketone, methyl isopropyl ketone or methyl isobutyl ketone, esters, such as methyl acetate or ethyl acetate, nitriles, such as, for example, acetonitrile or propionitrile, amides, such as, for example, dimethylformamide, dimethylacetamide and N-methylpyrrolidone, and also dimethyl sulphoxide, tetramethylene sulphone and hexamethylphosphoric triamide. However, it is also possible to use alcohols, such as, for example, methanol, ethanol, propanol, i-propanol, butanol, i-butanol, 2-methoxyethanol. Preference is given to dimethylformamide and 2-methoxyethanol.

In the process according to the invention, the reaction temperatures can be varied within a relatively wide range. In general, the process is carried out at temperatures between 0° C. and 250° C., preferably at temperatures between 10° C. and 120° C.

The process according to the invention is generally carried out under atmospheric pressure. However, it is also possible to operate under elevated or reduced pressure.

For carrying out the process according to the invention, the starting materials required in each case are generally employed in approximately equimolar amounts. However, it is also possible to use a relatively large excess of one of the components employed in each case. Work-up in the processes according to the invention is in each case carried out by customary methods (cf. the Preparation Examples).

Analogous 2-4-disubstituted pyridines of type I or Ia (Z and Y are carbon) can be synthesized by methods known from the literature (J. Med. Chem. 2003, 46(15), 3230-3244; J. Med. Chem. 1985, 28(11), 1628-36; U.S. Pat. No. 6,218,537)

Analogous 2-4-disubstituted 1,3,5-triazines of type I or Ia (Z and Y are nitrogen) can also be synthesized by methods known from the literature (Heterocycles 1992, 34(5), 929-35; Austr. J. Chem. 1981, 34(3), 623-34).

Isomeric 4-6-disubstituted pyrimidines of type I or Ia (Y is carbon) can be synthesized by methods known from the literature, too (J. Heterocycl Chem. 1980, 17(7), 1385-7).

The synthesis of bridged compounds Ic is also achieved by known methods. A possible synthesis route is shown in scheme 3 (also described in WO 2005/005438):

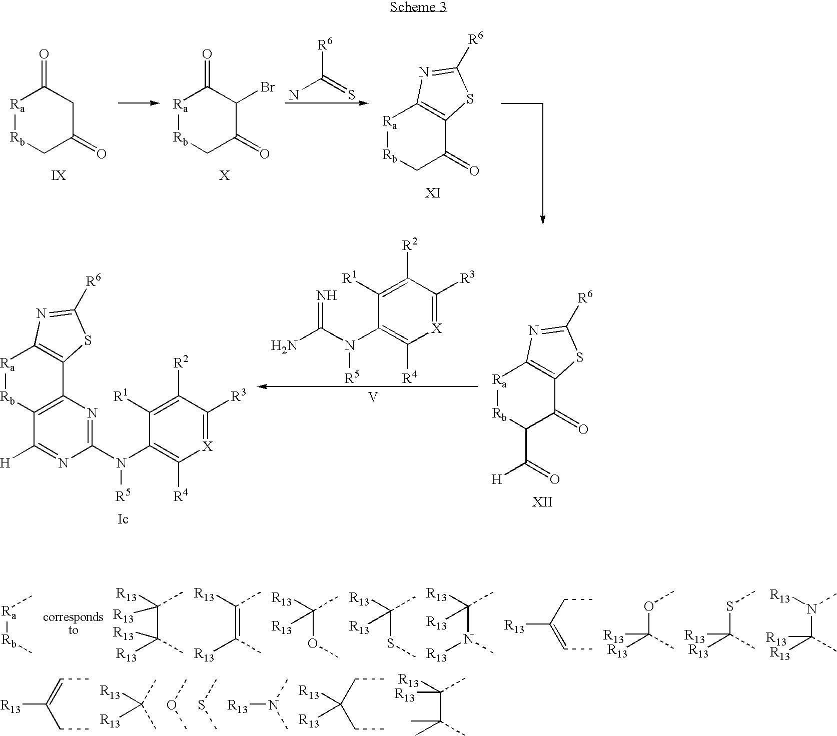

Cyclic 1,3-diketones IX are either commercially available or easily obtainable by methods described in the literature. The synthesis of heterocyclic 1,3-diketones is described, for example, in J. Org. Chem. 1977, 42, 1163 (Rₐ—R_{b}=—O—CH₂— or —S—CH₂—). The bromination of suitable cyclic 1,3-diketones is described in J. Chem. Soc. 1965, 353; J. Chem. Soc. Perkin Trans. I 1987, 2153; or Z. Chem. 1967, 7, 422 and affords the corresponding 2-brominated cyclic 1,3-diones X which are then reacted with thioamides or thiourea to give the corresponding thiazoles XI. Alternatively to the bromination, it is possible to carry out a chlorination with SOCl₂, described in J. Prakt. Chem. 1963, 20(5-6), 285 or Tetrahedron Lett. 1967, 34, 3331. The formyl group can be introduced by reaction with a formic acid derivative, for example ethyl formate, under basic conditions. The 1,3-dicarbonyl compounds XII obtained in this manner can be condensed directly with amidines or guanidines V or salts thereof; such reactions are described, for example, in Gazz. Chim. Ital. 1973, 103, 1063. Alternatively, the acyl compounds can initially be converted into enaminones (Chem. Ber. 1964, 97, 3397) which can then be condensed with amidines or guanidines V or salts thereof. Fully aromatized or unsaturated compounds can be obtained, for example, by oxidation.

Synthesis of precursors of thiazolyl ketones of type XI is described starting with 2-methyl-4-(1-hydroxycarbonylethyl)thiazolyl-5-carboxylic acid or 2-methyl-4-(B3-hydroxycarbonylbutyl)-thiazolyl-5-carboxylic acid XIII (J. Prakt. Chem. 1963, 20(5-6), 285-90). An overview is given in scheme 4.

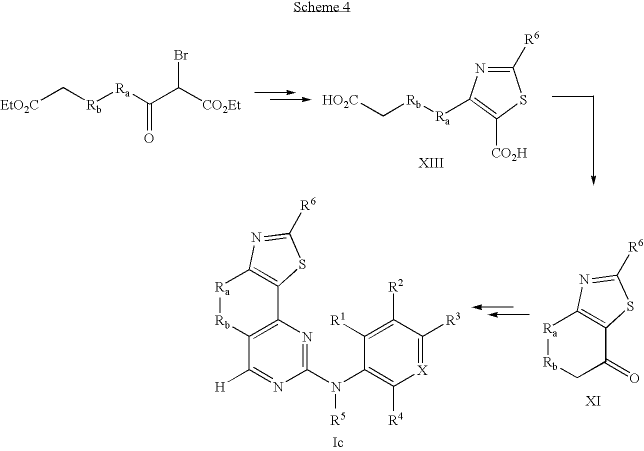

The processes according to the invention for preparing the compounds of the formula Ic are preferably carried out using one or more reaction auxiliaries.

Suitable reaction auxiliaries are, if appropriate, the customary inorganic or organic bases or acid acceptors. These preferably include alkali metal or alkaline earth metal acetates, amides, carbonates, bicarbonates, hydrides, hydroxides or alkoxides, such as, for example, sodium acetate, potassium acetate or calcium acetate, lithium amide, sodium amide, potassium amide or calcium amide, sodium carbonate, potassium carbonate or calcium carbonate, sodium bicarbonate, potassium bicarbonate or calcium bicarbonate, lithium hydride, sodium hydride, potassium hydride or calcium hydride, lithium hydroxide, sodium hydroxide, potassium hydroxide or calcium hydroxide, sodium methoxide, ethoxide, n- or i-propoxide, n-, i-, s- or t-butoxide or potassium methoxide, ethoxide, n- or i-propoxide, n-, i-, s- or t-butoxide; furthermore also basic organic nitrogen compounds, such as, for example, trimethylamine, triethylamine, tripropylamine, tributylamine, ethyldiisopropylamine, N,N-dimethylcyclohexylamine, dicyclohexyalmine, ethyldicyclohexylamine, N,N-dimethylaniline, N,N-dimethylbenzylamine, pyridine, 2-methyl-, 3-methyl-, 4-methyl-, 2,4-dimethyl-, 2,6-dimethyl-, 3,4-dimethyl- and 3,5-dimethylpyridine, 5-ethyl-2-methylpyridine, 4-dimethylaminopyridine, N-methylpiperidine, 1,4-diazabicyclo[2.2.2]-octane (DABCO), 1,5-diazabicyclo[4.3.0]non-5-ene (DBN) or 1,8 diazabicyclo[5.4.0]undec-7-ene (DBU).

The processes according to the invention are preferably carried out using one or more diluents. Suitable diluents are virtually all inert organic solvents. These preferably include aliphatic and aromatic, optionally halogenated hydrocarbons, such as pentane, hexane, heptane, cyclohexane, petroleum ether, benzine, ligroin, benzene, toluene, xylene, methylene chloride, ethylene chloride, chloroform, carbon tetrachloride, chlorobenzene and o-dichlorobenzene, ethers, such as diethyl ether and dibutyl ether, glycol dimethyl ether and diglycol dimethyl ether, tetrahydrofuran and dioxane, ketones, such as acetone, methyl ethyl ketone, methyl isopropyl ketone or methyl isobutyl ketone, esters, such as methyl acetate or ethyl acetate, nitriles, such as, for example, acetonitrile or propionitrile, amides, such as, for example, dimethylformamide, dimethylacetamide and N-methylpyrrolidone, and also dimethyl sulphoxide, tetramethylene sulphone and hexamethylphosphoric triamide.

In the processes according to the invention, the reaction temperatures can be varied within a relatively wide range. In general, the process is carried out at temperatures between 0° C. and 250° C., preferably at temperatures between 10° C. and 185° C.

The processes according to the invention are generally carried out under atmospheric pressure. However, it is also possible to operate under elevated or reduced pressure.

For carrying out the processes according to the invention, the starting materials required in each case are generally employed in approximately equimolar amounts. However, it is also possible to use a relatively large excess of one of the components employed in each case. Work-up in the processes according to the invention is in each case carried out by customary methods (cf. the Preparation Examples).

Similarly to known methods, compounds of type Id can also be prepared from substituted 5-bromothiazoles or 5-iodothiazoles XIV, here referred to as 5-halothiazoles, and optionally substituted pyrimidines XV. In the pyrimidines XV, R¹⁰ is as defined above. A possible synthesis route is illustrated in scheme 5.

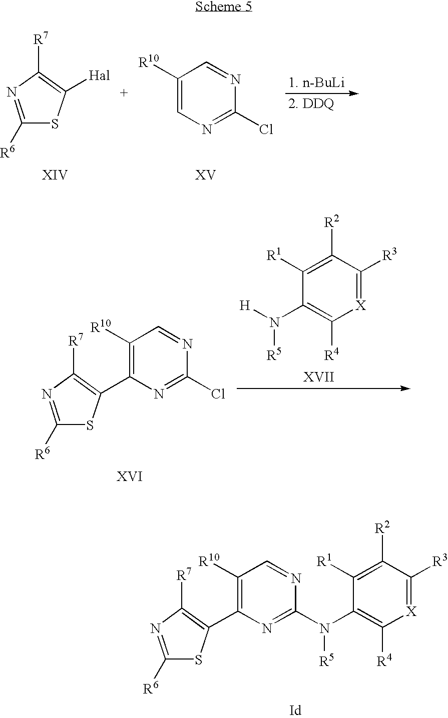

Some 5-halothiazoles XIV are known, or they are prepared by bromination or iodination of known thiazoles by customary methods (Organikum, 21st edition, Wiley-VCH, 2001). Initially, the 5-halothiazoles XIV are metallated using a metal or a metal organyl, such as, for example, n-butyllithium. The resulting organometallic compounds are then added to the corresponding 2-chloropyrimidines XV, with formation of dihydropyrimidines. By reaction with an oxidizing agent, such as, for example, 4,5-dichloro-3,6-dioxocyclohexa-1,4-diene-1,2-dicarbonitriles (DDQ), the resulting dihydropyrimidines are rearomatized to give the pyrimidines XVI (J. Heterocyclic Chem. 1990, 27, 1393; J. Org. Chem. 1988, 53, 4137). The pyrimidines XVI are then reacted by customary methods under acidic, basic or metal-catalysed conditions, if appropriate also in the absence of a catalyst, with the amines of the formula XVII to give the target compounds Id (Houben-Weyl, Methoden der organischen Chemie; J. Med. Chem. 2004, 47, 4716-4730; J. Med. Chem. 2004, 47, 2724-2727, Org. Lett. 2002, 4, 3481-3484; GB2369359; WO 2002/096888). Intermediates of type XVI are novel. The process is also provided by the present invention. In particular, the process is suitable for preparing compounds Ia and I.

2-Chloropyrimidines of the formula XV are known and/or can be prepared by known processes (Houben-Weyl, Methoden der organischen Chemie).

Suitable diluents for carrying out the first step of the process according to the invention are all inert organic solvents. These preferably include aliphatic, alicyclic or aromatic hydrocarbons, such as, for example, petroleum ether, hexane, hepane, cyclohexane, methylcyclohexane, benzene, toluene, xylene or decalin; halogenated hydrocarbons, such as, for example, chlorobenzene, dichlorobenzene, dichloromethane, chloroform, carbon tetrachloride, dichloroethane or trichloroethane; ethers, such as diethyl ether, diisopropyl ether, methyl t-butyl ether, methyl-t-amyl ether, dioxane, tetrahydrofuran, 1,2-dimethoxyethane, 1,2-diethoxyethane or anisole; ketones, such as acetone, butanone, methyl isobutyl ketone or cyclohexanone; nitriles, such as acetonitrile, propionitrile, n- or i-butyronitrile or benzonitrile; amides, such as N,N-dimethylformamide, N,N-dimethylacetamide, N-formanilide, N-methylpyrrolidone or hexamethylphosphoric triamide.

Suitable metal or metal organyls for metallating the 5-halothiazoles XIV in the first step of the process according to the invention are, in addition to n-butyllithium, all metals or metal organyls suitable for preparing metal organyls. These preferably include metals of the first and second main group of the Periodic Table, such as, for example, lithium or magnesium, or metal organyls thereof, such as, for example, methyllithium, phenyllithium, sec- or tert-butyllithium or methylmagnesium bromide.

Suitable for rearomatizing the dihydropyrimidines in the first step of the process according to the invention are, in addition to 4,5-dichloro-3,6-dioxocyclohexa-1,4-diene-1,2-dicarbonitriles (DDQ), also other oxidizing agents. These preferably include other quinones, such as, for example, 2,3,5,6-tetrachloro[1,4]benzoquinone (chloranil); N-haloimides, such as, for example, N-bromosuccinimide; sulphur or selenium; photochemically, radiochemically or thermally generated radicals, especially those at oxygen atoms, such as hydroxyl, hydroperoxy or alkoxy radicals.

Suitable diluents for carrying out the second step of the process according to the invention are all organic solvents. These preferably include aliphatic, alicyclic or aromatic hydrocarbons, such as, for example, petroleum ether, hexane, heptane, cyclohexane, methylcyclohexane, benzene, toluene, xylene or decalin; halogenated hydrocarbons, such as, for example, chlorobenzene, dichlorobenzene, dichloromethane, chloroform, carbon tetrachloride, dichloroethane or trichloroethane; alcohols, such as, for example, methanol, ethanol, n-propanol, isopropanol, n-butanol, tert-butanol; water; ethers, such as diethyl ether, diisopropyl ether, methyl t-butyl ether, methyl t-amyl ether, dioxane, tetrahydrofuran, 1,2-dimethoxyethane, 1,2-diethoxyethane or anisole; ketones, such as acetone, butanone, methyl isobutyl ketone or cyclohexanone; nitriles, such as acetonitrile, propionitrile, n- or i-butyronitrile or benzonitrile; amides, such as N,N-dimethylformamide, N,N-dimethylacetamide, N-methylformanilide, N-methylpyrrolidone or hexamethylphosphoric triamide, or mixtures thereof with water.

The second step of the process according to the invention is, if appropriate, carried out in the presence of a suitable acid acceptor. Suitable acid acceptors are all customary inorganic or organic bases. These preferably include alkaline earth metal or alkali metal hydrides, hydroxides, amides, alkoxides, acetates, carbonates or bicarbonates, such as, for example, sodium hydride, sodium amide, lithium diisopropylamide, sodium methoxide, sodium ethoxide, potassium tert-butoxide, sodium hydroxide, potassium hydroxide, sodium acetate, sodium carbonate, potassium carbonate, potassium bicarbonate, sodium bicarbonate or ammonium carbonate, and also tertiary amines, such as trimethylamine, triethylamine, tributylamine, N,N-dimethylaniline, N,N-dimethyl-benzylamine, pyridine, N-methylpiperidine, N-methylmorpholine, N,N-dimethylaminopyridine, diazabicyclooctane (DABCO), diazabicyclononene (DBN) or diazabicycloundecene (DBU).

If appropriate, the second step of the process according to the invention can also be carried out in the presence of a suitable acid. Suitable acids are, preferably, mineral acids, such as, for example, hydrochloric acid or sulphuric acid, but also organic acids, such as, for example, formic acid, acetic acid, 4-toluenesulphonic acid or ascorbic acid.

If appropriate, the second step of the process according to the invention can also be carried out in the presence of a suitable catalyst. Suitable catalysts are especially palladium salts or complexes. Preferred for this purpose are palladium chloride, palladium acetate, tetrakis(triphenylphosphine)-palladium or bis(triphenylphosphine)palladium dichloride. It is possible also for a palladium complex to be generated in the reaction mixture by adding a palladium salt and a complex ligand separately to the reaction. Preferred ligands are organophosphorus compounds. The following may be mentioned by way of example: triphenylphosphine, tri-o-tolylphosphine, 2,2′-bis(diphenylphosphino)-1,1-binaphthyl, dicyclohexylphosphinebiphenyl, 1,4-bis(diphenylphosphino)butane, bisdiphenylphosphinoferrocene, di(tert-butylphosphino)biphenyl, di(cyclohexylphosphino)-biphenyl, 2-dicyclohexylphosphino-2′-N,N-dimethylaminobiphenyl, tricyclohexylphosphine, tri-tert-butylphosphine, 2,2′-bisdiphenylphosphanyl-[1,1′]binaphthalenyl, 4,6-bisdiphenylphosphanyl-dibenzofuran. However, ligands may also be dispensed with.

The reaction temperatures for carrying out the process according to the invention may be varied in a relatively wide range. In general, the process is carried out at temperatures of from −100° C. to 150° C., preferably at temperatures of from −80° C. to 110° C., very particularly preferably at temperatures of from −80° C. to 70° C.

For carrying out the process according to the invention, the starting materials required in each case are generally employed in approximately equimolar amounts. However, it is also possible to use a relatively large excess of one of the components employed in each case. Work-up in the processes according to the invention is in each case carried out by customary methods (cf. the Preparation Examples).

The process according to the invention is generally carried out under atmospheric pressure. However, it is also possible to operate under elevated or reduced pressure—in general between 0.1 bar and 10 bar.

The compounds according to the invention have potent microbicidal activity and can be employed for controlling unwanted microorganisms, such as fungi and bacteria, in crop protection and in the protection of materials.

Fungicides can be employed in crop protection for controlling, for example, Plasmodiophoromycetes, Oomycetes, Chytridiomycetes, Zygomycetes, Ascomycetes, Basidiomycetes and Deuteromycetes.

Bactericides can be employed in crop protection for controlling, for example, Pseudomonadaceae, Rhizobiaceae, Enterobacteriaceae, Corynebacteriaceae and Streptomycetaceae.

The thiazoles according to the invention have very good fungicidal properties and can be used for controlling phytopathogenic fungi, such as Plasmodiophoromycetes, Oomycetes, Chytridiomycetes, Zygomycetes, Ascomycetes, Basidiomycetes and Deuteromycetes, etc.

Some pathogens causing fungal and bacterial diseases which come under the generic names listed above may be mentioned as examples, but not by way of limitation:

Xanthomonas species, such as, for example, Xanthomonas campestris pv. oryzae;
Pseudomonas species, such as, for example, Pseudomonas syringae pv. lachrymans;
Erwinia species, such as, for example, Erwinia amylovora;
diseases caused by powdery mildew pathogens, such as, for example
Blumeria species such as, for example, Blumeria graminis;
Podosphaera species such as, for example, Podosphaera leucotricha;
Sphaerotheca species such as, for example, Sphaerotheca fuliginea;
Uncinula species such as, for example, Uncinula necator;
diseases caused by rust pathogens such as, for example,
Gymnosporangium species such as, for example, Gymnosporangium sabinae
Hemileia species such as, for example, Hemileia vastatrix;
Phakopsora species such as, for example, Phakopsora pachyrhizi and Phakopsora meibomiae;
Puccinia species such as, for example, Puccinia recondita;
Uromyces species such as, for example, Uromyces appendiculatus;
diseases caused by pathogens from the Oomycetes group such as, for example,
Bremia species such as, for example, Bremia lactucae;
Peronospora species such as, for example, Peronospora pisi or P. brassicae;
Phytophthora species such as, for example, Phytophthora infestans;
Plasmopara species such as, for example, Plasmopara viticola;
Pseudoperonospora species such as, for example, Pseudoperonospora humuli or Pseudoperonospora cubensis;
Pythium species such as, for example, Pythium ultimum;
leaf spot diseases and leaf wilts caused by, for example,
Alternaria species such as, for example, Alternaria solani;
Cercospora species such as, for example, Cercospora beticola;
Cladiosporum species such as, for example, Cladiosporium cucumerinum;
Cochliobolus Species such as, for Example, Cochliobolus sativus
(conidia form: Drechslera, syn: Helminthosporium);
Colletotrichum species such as, for example, Colletotrichum lindemuthanium;
Cycloconium species such as, for example, Cycloconium oleaginum;
Diaporthe species such as, for example, Diaporthe citri;
Elsinoe species such as, for example, Elsinoe fawcettii;
Gloeosporium species such as, for example, Gloeosporium laeticolor;
Glomerella species such as, for example, Glomerella cingulata;
Guignardia species such as, for example, Guignardia bidwelli;
Leptosphaeria species such as, for example, Leptosphaeria maculans;
Magnaporthe species such as, for example, Magnaporthe grisea;
Mycosphaerella species such as, for example, Mycosphaerelle graminicola;
Phaeosphaeria species such as, for example, Phaeosphaeria nodorum;
Pyrenophora species such as, for example, Pyrenophora teres;
Ramularia species such as, for example, Ramularia collo-cygni;
Rhynchosporium species such as, for example, Rhynchosporium secalis;
Septoria species such as, for example, Septoria apii;
Typhula species such as, for example, Typhula incarnata;
Venturia species such as, for example, Venturia inaequalis;
root and stem diseases caused by, for example,
Corticium species such as, for example, Corticium graminearum;
Fusarium species such as, for example, Fusarium oxysporum;
Gaeumannomyces species such as, for example, Gaeumannomyces graminis;
Rhizoctonia species such as, for example, Rhizoctonia solani;
Tapesia species such as, for example, Tapesia acuformis;
Thielaviopsis species such as, for example, Thielaviopsis basicola;
ear and panicle diseases (including maize cobs), caused by, for example,
Alternaria species such as, for example, Alternaria spp.;
Aspergillus species such as, for example, Aspergillus flavus;
Cladosporium species such as, for example, Cladosporium spp.;
Claviceps species such as, for example, Claviceps purpurea;
Fusarium species such as, for example, Fusarium culmorum;
Gibberella species such as, for example, Gibberella zeae;
Monographella species such as, for example, Monographella nivalis;
diseases caused by smuts such as, for example,
Sphacelotheca species such as, for example, Sphacelotheca reiliana;
Tilletia species such as, for example, Tilletia caries;
Urocystis species such as, for example, Urocystis occulta;
Ustilago species such as, for example, Ustilago nuda;
fruit rots caused by, for example,
Aspergillus species such as, for example, Aspergillus flavus;
Botrytis species such as, for example, Botrytis cinerea;
Penicillium species such as, for example, Penicillium expansum;
Sclerotinia species such as, for example, Sclerotinia sclerotiorum;
Verticilium species such as, for example, Verticilium alboatrum;
seed- and soil-borne rots and wilts, and seedling diseases, caused by, for example,
Fusarium species such as, for example, Fusarium culmorum;
Phytophthora species such as, for example, Phytophthora cactorum;
Pythium species such as, for example, Pythium ultimum;
Rhizoctonia species such as, for example, Rhizoctonia solani;
Sclerotium species such as, for example, Sclerotium rolfsii;
cankers, galls and witches' broom disease, caused by, for example,
Nectria species such as, for example, Nectria galligena; wilts caused by, for example,
Monilinia species such as, for example, Monilinia laxa;
deformations of leaves, flowers and fruits, caused by, for example,
Taphrina species such as, for example, Taphrina deformans;
degenerative diseases of woody plants, caused by, for example,
Esca species such as, for example, Phaemoniella clamydospora;
diseases of inflorescences and seeds, caused by, for example,
Botrytis species such as, for example, Botrytis cinerea;
diseases of plant tubers, caused by, for example,
Rhizoctonia Species such as, for Example, Rhizoctonia solani.

The active compounds according to the invention also show a strong invigorating action in plants. Accordingly, they are suitable for mobilizing the internal defences of the plant against attack by unwanted microorganisms.

In the present context, plant-invigorating (resistance-inducing) compounds are to be understood as meaning those substances which are capable of stimulating the defence system of plants such that, when the treated plants are subsequently inoculated with unwanted microorganisms, they display substantial resistance to these microorganisms.

In the present case, unwanted microorganisms are to be understood as meaning phytopathogenic fungi and bacteria. The compounds according to the invention can thus be used to protect plants within a certain period of time after treatment against attack by the pathogens mentioned. The period of time for which this protection is achieved generally extends for 1 to 10 days, preferably 1 to 7 days, from the treatment of the plants with the active compounds.

The fact that the active compounds are well tolerated by plants at the concentrations required for controlling plant diseases permits the treatment of above-ground parts of plants, of propagation stock and seeds, and of the soil.

The active compounds according to the invention can be employed with particularly good results for controlling cereal diseases, such as, for example, against Erysiphe species, against Puccinia and against Fusaria species, of rice diseases, such as, for example, against, Pyricularia and Rhizoctonia, and of diseases in viticulture and in the cultivation of fruit and vegetables, such as, for example, against Botrytis, Venturia, Sphaerotheca and Podosphaera species.

The active compounds according to the invention are also suitable for increasing the yield of crops. In addition, they show reduced toxicity and are well tolerated by plants.

If appropriate, the active compounds according to the invention can, at certain concentrations and application rates, also be employed as herbicides, for regulating plant growth and for controlling animal pests. If appropriate, they can also be used as intermediates or precursors in the synthesis of other active compounds.

According to the invention, it is possible to treat all plants and parts of plants. Plants are to be understood here as meaning all plants and plant populations, such as desired and undesired wild plants or crop plants (including naturally occurring crop plants). Crop plants can be plants which can be obtained by conventional breeding and optimization methods or by biotechnological and genetic engineering methods or combinations of these methods, including the transgenic plants and including plant cultivars which can or cannot be protected by plant breeders' certificates. Parts of plants are to be understood as meaning all above-ground and below-ground parts and organs of plants, such as shoot, leaf, flower and root, examples which may be mentioned being leaves, needles, stems, trunks, flowers, fruit-bodies, fruits and seeds and also roots, tubers and rhizomes. Parts of plants also include harvested material and vegetative and generative propagation material, for example seedlings, tubers, rhizomes, cuttings and seeds.

The treatment of the plants and parts of plants according to the invention with the active compounds is carried out directly or by action on their environment, habitat or storage area according to customary treatment methods, for example by dipping, spraying, evaporating, atomizing, broadcasting, brushing-on and, in the case of propagation material, in particular in the case of seeds, furthermore by one- or multilayer coating.

In the protection of materials, the compounds according to the invention can be employed for protecting industrial materials against infection with, and destruction by, unwanted microorganisms.

Industrial materials in the present context are understood as meaning non-living materials which have been prepared for use in industry. For example, industrial materials which are intended to be protected by active compounds according to the invention from microbial change or destruction can be tackifiers, sizes, paper and board, textiles, leather, wood, paints and plastic articles, cooling lubricants and other materials which can be infected with, or destroyed by, microorganisms. Parts of production plants, for example cooling-water circuits, which may be impaired by the proliferation of microorganisms may also be mentioned within the scope of the materials to be protected. Industrial materials which may be mentioned within the scope of the present invention are preferably tackifiers, sizes, papers and boards, leather, wood, paints, cooling lubricants and heat-transfer liquids, particularly preferably wood.

Microorganisms capable of degrading or changing the industrial materials which may be mentioned are, for example, bacteria, fungi, yeasts, algae and slime organisms. The active compounds according to the invention preferably act against fungi, in particular moulds, wood-discolouring and wood-destroying fungi (Basidiomycetes) and against slime organisms and algae.

Microorganisms of the following genera may be mentioned as examples:

Alternaria, such as Alternaria tenuis,
Aspergillus, such as Aspergillus niger,
Chaetomium, such as Chaetomium globosum,
Coniophora, such as Coniophora puetana,
Lentinus, such as Lentinus tigrinus,
Penicillium, such as Penicillium glaucum,
Polyporus, such as Polyporus versicolor,
Aureobasidium, such as Aureobasidium pullulans,
Sclerophoma, such as Sclerophoma pityophila,
Trichoderma, such as Trichoderma viride,
Escherichia, such as Escherichia coli,
Pseudomonas, such as Pseudomonas aeruginosa, and
Staphylococcus, such as Staphylococcus aureus.

Depending on their particular physical and/or chemical properties, the active compounds can be converted into the customary formulations, such as solutions, emulsions, suspensions, powders, foams, pastes, granules, aerosols and microencapsulations in polymeric substances and in coating compositions for seeds, and ULV cool and warm fogging formulations.

These formulations are produced in a known manner, for example by mixing the active compounds with extenders, that is liquid solvents, liquefied gases under pressure, and/or solid carriers, optionally with the use of surfactants, that is emulsifiers and/or dispersants, and/or foam formers. If the extender used is water, it is also possible to employ, for example, organic solvents as auxiliary solvents. Essentially, suitable liquid solvents are: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example petroleum fractions, alcohols such as butanol or glycol and their ethers and esters, ketones such as acetone, methyl ethyl ketone, methyl isobutyl ketone or cyclohexanone, strongly polar solvents such as dimethylformamide or dimethyl sulphoxide, or else water. Liquefied gaseous extenders or carriers are to be understood as meaning liquids which are gaseous at standard temperature and under atmospheric pressure, for example aerosol propellants such as halogenated hydrocarbons, or else butane, propane, nitrogen and carbon dioxide. Suitable solid carriers are: for example ground natural minerals such as kaolins, clays, talc, chalk, quartz, attapulgite, montmorillonite or diatomaceous earth, and ground synthetic minerals such as finely divided silica, alumina and silicates. Suitable solid carriers for granules are: for example crushed and fractionated natural rocks such as calcite, pumice, marble, sepiolite and dolomite, or else synthetic granules of inorganic and organic meals, and granules of organic material such as sawdust, coconut shells, maize cobs and tobacco stalks. Suitable emulsifiers and/or foam formers are: for example nonionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates, or else protein hydrolysates. Suitable dispersants are: for example lignosulphite waste liquors and methylcellulose.

Tackifiers such as carboxymethylcellulose, natural and synthetic polymers in the form of powders, granules or latices, such as gum arabic, polyvinyl alcohol and polyvinyl acetate, or else natural phospholipids such as cephalins and lecithins and synthetic phospholipids can be used in the formulations. Other possible additives are mineral and vegetable oils.

It is possible to use colorants such as inorganic pigments, for example iron oxide, titanium oxide and Prussian Blue, and organic dyestuffs such as alizarin dyestuffs, azo dyestuffs and metal phthalocyanine dyestuffs, and trace nutrients such as salts of iron, manganese, boron, copper, cobalt, molybdenum and zinc.

The formulations generally comprise between 0.1 and 95 percent by weight of active compound, preferably between 0.5 and 90%.

The active compounds according to the invention can, as such or in their formulations, also be used in a mixture with known fungicides, bactericides, acaricides, nematicides or insecticides, to broaden, for example, the activity spectrum or to prevent development of resistance. In many cases, synergistic effects are obtained, i.e. the activity of the mixture is greater than the activity of the individual components.

Suitable mixing components are, for example, the following compounds:

### Fungicides:

### 1. Nucleic Acid Synthesis Inhibition

benalaxyl, benalaxyl-M, bupirimate, chiralaxyl, clozylacon, dimethirimol, ethirimol, furalaxyl, hymexazol, mefenoxam, metalaxyl, metalaxyl-M, ofurace, oxadixyl, oxolinic acid

### 2. Mitosis and Cell Division Inhibition

benomyl, carbendazim, diethofencarb, fuberidazole, pencycuron, thiabendazole, thiophanate-methyl, zoxamide

### 3. Inhibition of the Respiratory Chain

### 3.1 Complex I

diflumetorim

### 3.2 Complex II

boscalid, carboxin, fenfuram, flutolanil, furametpyr, furmecyclox, mepronil, oxycarboxin, penthiopyrad, thifluzamide

### 3.3 Complex III

amisulbrom, azoxystrobin, cyazofamid, dimoxystrobin, enestrobin, famoxadone, fenamidone, fluoxastrobin, kresoxim-methyl, metominostrobin, orysastrobin, picoxystrobin, pyraclostrobin, trifloxystrobin

### 3.4 Decouplers

dinocap, fluazinam, methyldinocap

### 3.5 ATP Production Inhibition

fentin acetate, fentin chloride, fentin hydroxide, silthiofam

### 4. Amino Acid and Protein Biosynthesis Inhibition

andoprim, blasticidin-S, cyprodinil, kasugamycin, kasugamycin hydrochloride hydrate, mepanipyrim, pyrimethanil

### 5. Signal Transduction Inhibition

fenpiclonil, fludioxonil, quinoxyfen

### 6. Lipid and Membrane Synthesis Inhibition

chlozolinate, iprodione, procymidone, vinclozolin pyrazophos, edifenphos, iprobenfos (IBP), isoprothiolane tolclofos-methyl, biphenyl iodocarb, propamocarb, propamocarb hydrochloride

### 7. Inhibition Ofergosterol Biosynthesis

fenhexamid,
azaconazole, bitertanol, bromuconazole, cyproconazole, diclobutrazole, difenoconazole, diniconazole, diniconazole-M, epoxiconazole, etaconazole, fenbuconazole, fluquinconazole, flurprimidol, flusilazole, flutriafol, furconazole, furconazole-cis, hexaconazole, imnibenconazole, ipconazole, metconazole, myclobutanil, paclobutrazol, penconazole, propiconazole, prothioconazole, pyrifenox, simeconazole, tebuconazole, tetraconazole, triadimefon, triadimenol, triticonazole, uniconazole, voriconazole,
imazalil, imazalil sulphate, oxpoconazole, fenarimol, flurprimidol, nuarimol, pyrifenox, triforine, pefurazoate, prochloraz, triflumizole, viniconazole,
aldimorph, dodemorph, dodemorph acetate, fenpropidin, fenpropimorph, tridemorph, spiroxamine,
naftifine, pyributicarb, terbinafine

### 8. Cell Wall Synthesis Inhibition

benthiavalicarb, dimethomorph, flumorph, iprovalicarb, polyoxins, polyoxorim, validamycin A

### 9. Melanin Biosynthesis Inhibition

capropamid, diclocymet, fenoxanil, phthalide, pyroquilon, tricyclazole

### 10. Resistance Induction

acibenzolar-5-methyl, probenazole, tiadinil
11. Compounds with Multisite Activity
Bordeaux mixture, captafol, captan, chlorothalonil, copper salts, such as: copper hydroxide, copper naphthenate, copper oxychloride, copper oxide, copper sulphate, oxine copper, dichlofluanid, dithianone, dodine, dodine free base, ferbam, fluorofolpet, folpet, guazatine, guazatine acetate, iminoctadine, iminoctadine albesilate, iminoctadine triacetate, mancopper, mancozeb, maneb, metiram, metiram zinc, propineb, sulphur and sulphur preparations comprising calcium polysulphide, thiram, tolylfluanid, zineb, ziram

### 12. Unknown

amibromdol, benthiazole, bethoxazin, capsimycin, carvon, chinomethionat, chloropicrin, cufraneb, cyflufenamid, cymoxanil, dazomet, debacarb, dichlorophen, diclomezin, dicloran, difenzoquat, difenzoquat methylsulphate, diphenylamine, ferimzone, flumetover, fluopicolide, fluoroimide, flusulphamide, fosetyl-aluminium, fosetyl-calcium, fosetyl-sodium, hexachlorobenzene, 8-hydroxyquinolinsulphate, irumamycin, methasulphocarb, methyl isothiocyanate, metrafenone, mildiomycin, natamycin, nickel dimethyldithiocarbamate, nitrothal-isopropyl, octhilinone, oxamocarb, oxyfenthiin, pentachlorophenol and its salts, 2-phenylphenol and its salts, phosphorous acid and its salts, piperalin, propamocarb fosetylate, propanosin-sodium, proquinazid, pyrroInitrin, quintozen, tecloftalam, tecnazen, triazoxid, trichlamid, zarilamid and 2-amino-4-methyl-N-phenyl-5-thiazolecarboxamide, 2-chloro-N-(2,3-dihydro-1,1,3-trimethyl-1H-inden-4-yl)-3-pyridinecarboxamide, 3-[5-(4-chlorophenyl)-2,3-dimethylisoxazolidin-3-yl]pyridine, cis-1-(4-chlorophenyl)-2-(1H-1,2,4-triazol-1-yl)cycloheptanol, 2,4-dihydro-5-methoxy-2-methyl-4-[[[[1-[3-(trifluoromethyl)phenyl]ethylidene]amino]oxy]methyl]phenyl]-3H-1,2,3-triazol-3-one, methyl 1-(2,3-dihydro-2,2-dimethyl-1H-inden-1-yl)-1H-imidazole-5-carboxylate, methyl 2-[[[cyclopropyl[(4-methoxyphenyl)imino]methyl]thio]methyl]phenyl-3-methoxyacrylate, methyl 3-(4-chlorophenyl)-3-{[N-(isopropoxycarbonyl)valyl]amino}propanoate, 4-chloro-alpha-propynyloxy-N-[2-[3-methoxy-4-(2-propynyloxy)phenyl]ethyl]benzacetamide, 2-(2-{[6-(3-chloro-2-methylphenoxy)-5-fluoropyrimidin-4-yl]oxy}phenyl)-2-(methoxyimino)-N-methylacetamide, (2S)—N-[2-[4-[[3-(4-chlorophenyl)-2-propynyl]oxy]-3-methoxyphenyl]ethyl]-3-methyl-2-[(methylsulphonyl)amino]butanamide, 5-chloro-7-(4-methylpiperidin-1-yl)-6-(2,4,6-trifluorophenyl)[1,2,4]triazolo[1,5-a]pyrimidine, 5-chloro-6-(2,4,6-trifluorophenyl)-N-[(1R)-1,2,2-trimethylpropyl][1,2,4]triazolo[1,5-a]pyrimidine-7-amine, 5-chloro-N-[(1R)-1,2-dimethylpropyl]-6-(2,4,6-trifluorophenyl)[1,2,4]triazolo[1,5-a]pyrimidine-7-amine, N-[1-(5-bromo-3-chloropyridin-2-yl)ethyl]-2,4-dichloronicotinamide, N-(5-bromo-3-chloropyridin-2-yl)methyl-2,4-dichloronicotinamide, N-[1-(5-bromo-3-chloropyridin-2-yl)ethyl]-2-fluoro-4-iodonicotinamide, 2-butoxy-6-iodo-3-propylbenzopyranon-4-one, N-[2-(4-{[3-(4-chlorophenyl)prop-2-yn-1-yl]oxy}-3-methoxyphenyl)ethyl]-N-2-(methylsulphonyl)valinamide, N-{(Z)-[(cyclopropylmethoxy)imino][6-(difluoromethoxy)-2,3-difluorophenyl]methyl}-2-phenylacetamide, N-(4-chloro-2-nitrophenyl)-N-ethyl-4-methylbenzenesulphonamide, N-(3-ethyl-3,5,5-trimethylcyclohexyl)-3-formylamino-2-hydroxybenzamide, 2-[[[[1-[3(1-fluoro-2-phenylethyl)oxy]phenyl]ethylidene]amino]oxy]methyl]-alpha-(methoxyimino)-N-methyl-alpha-benzacetamide, N-{2-[3-chloro-5-(trifluoromethyl)pyridin-2-yl]ethyl}-2-(trifluoromethyl)benzamide, N-(3′,4′-dichloro-5-fluorobiphenyl-2-yl)-3-(difluoromethyl)-1-methyl-1H-pyrazole-4-carboxamide, N-(6-methoxy-3-pyridinyl)cyclopropanecarboxamide, 1-[(4-methoxyphenoxy)methyl]-2,2-dimethylpropyl-1H-imidazole-1-carboxylic acid, O-[1-[(4-methoxyphenoxy)methyl]-2,2-dimethylpropyl]-1H-imidazole-1-carbothioic acid, 2,3,5,6-tetrachloro-4-(methylsulphonyl)pyridine, 3,4,5-trichloro-2,6-pyridinedicarbonitrile.

### Bactericides:

bronopol, dichlorophen, nitrapyrin, nickel dimethyldithiocarbamate, kasugamycin, octhilinone, furancarboxylic acid, oxytetracyclin, probenazole, streptomycin, tecloftalam, copper sulphate and other copper preparations.

### Insecticides/Acaricides/Nematicides:

### 1. Acetylcholinesterase (AChE) Inhibitors

1.1 carbamates (for example alanycarb, aldicarb, aldoxycarb, allyxycarb, aminocarb, azamethiphos, bendiocarb, benfuracarb, bufencarb, butacarb, butocarboxim, butoxycarboxim, carbaryl, carbofuran, carbosulfan, chloethocarb, coumaphos, cyanofenphos, cyanophos, dimetilan, ethiofencarb, fenobucarb, fenothiocarb, formetanate, furathiocarb, isoprocarb, metam-sodium, methiocarb, methomyl, metolcarb, oxamyl, pirimicarb, promecarb, propoxur, thiodicarb, thiofanox, triazamate, trimethacarb, XMC, xylylcarb)
1.2 organophosphates (for example acephate, azamethiphos, azinphos (-methyl, -ethyl), bromophos-ethyl, bromfenvinfos (-methyl), butathiofos, cadusafos, carbophenothion, chlorethoxyfos, chlorfenvinphos, chlormephos, chlorpyrifos (-methyl/-ethyl), coumaphos, cyanofenphos, cyanophos, chlorfenvinphos, demeton-s-methyl, demeton-s-methylsulphon, dialifos, diazinon, dichlofenthion, dichlorvos/DDVP, dicrotophos, dimethoate, dimethylvinphos, dioxabenzofos, disulfoton, EPN, ethion, ethoprophos, etrimfos, famphur, fenamiphos, fenitrothion, fensulfothion, fenthion, flupyrazofos, fonofos, formothion, fosmethilan, fosthiazate, heptenophos, iodofenphos, iprobenfos, isazofos, isofenphos, isopropyl o-salicylate, isoxathion, malathion, mecarbam, methacrifos, methamidophos, methidathion, mevinphos, monocrotophos, naled, omethoate, oxydemeton-methyl, parathion (-methyl/-ethyl), phenthoate, phorate, phosalone, phosmet, phosphamidon, phosphocarb, phoxim, pirimiphos (-methyl/-ethyl), profenofos, propaphos, propetamphos, prothiofos, prothoate, pyraclofos, pyridaphenthion, pyridathion, quinalphos, sebufos, sulfotep, sulprofos, tebupirimfos, temephos, terbufos, tetrachlorvinphos, thiometon, triazophos, triclorfon, vamidothion)

### 2. Sodium Channel Modulators/Blockers of Voltage-Gated Sodium Channels

2.1 pyrethroids (for example acrinathrin, allethrin (d-cis-trans, d-trans), beta-cyfluthrin, bifenthrin, bioallethrin, bioallethrin-5-cyclopentyl isomer, bioethanomethrin, biopermethrin, bioresmethrin, chlovaporthrin, cis-cypermethrin, cis-resmethrin, cis-permethrin, clocythrin, cycloprothrin, cyfluthrin, cyhalothrin, cypermethrin (alpha-, beta-, theta-, zeta-), cyphenothrin, DDT, deltamethrin, empenthrin (IR-isomer), esfenvalerate, etofenprox, fenfluthrin, fenpropathrin, fenpyrithrin, fenvalerate, flubrocythrinate, flucythrinate, flufenprox, flumethrin, fluvalinate, fubfenprox, gamma-cybalothrin, imiprothrin, kadethrin, lambda-cyhalothrin, metofluthrin, permethrin (cis-, trans-), phenothrin (lR-trans isomer), prallethrin, profluthrin, protrifenbute, pyresmethrin, resmethrin, RU 15525, silafluofen, tau-fluvalinate, tefluthrin, terallethrin, tetramethrin (IR-isomer), tralomethrin, transfluthrin, ZXI 8901, pyrethrins (pyrethrum))

### 2.2 Oxadiazines (for Example Indoxacarb)

### 3. Acetylcholine Receptor Agonists/Antagonists

### 3.1 Chloronicotinyls/Neonicotinoids (for Example Acetamiprid, Clothianidin, Dinotefuran, Imidacloprid, Nitenpyram, Nithiazine, Thiacloprid, Thiamethoxam)

### 3.2 Nicotine, Bensultap, Cartap

### 4. Acetylcholine Receptor Modulators

### 4.1 Spinosyns (for Example Spinosad)

### 5. Antagonists of GABA-Gated Chloride Channels

### 5.1 Cyclodiene Organochlorines (for Example Camphechlor, Chlordane, Endosulfan, Gamma-HCH, HCH, Heptachlor, Lindane, Methoxychlor)

### 5.2 Fiproles (for Example Acetoprole, Ethiprole, Fipronil, Vaniliprole)

### 6. Chloride Channel Activators

### 6.1 Mectins (for Example Abamectin, Avermectin, Emamectin, Emamectin-Benzoate, Ivermectin, Milbemectin, Milbemycin)

### 7. Juvenile Hormone Mimetics

### (for Example Diofenolan, Epofenonane, Fenoxycarb, Hydroprene, Kinoprene, Methoprene, Pyriproxifen, Triprene)

### 8. Ecdyson Agonists/Disruptors

### 8.1 Diacylhydrazines (for Example Chromafenozide, Halofenozide, Methoxyfenozide, Tebufenozide)

### 9. Chitin Biosynthesis Inhibitors

### 9.1 Benzoylureas (for Example Bistrifluoron, Chlofluazuron, Diflubenzuron, Fluazuron, Flucycloxuron, Flufenoxuron, Hexaflumuron, Lufenuron, Novaluron, Noviflumuron, Penfluoron, Teflubenzuron, Triflumuron)

### 9.2 Buprofezin

### 9.3 Cyromazine

### 10. Inhibitors of Oxidative Phosphorylation, ATP Disruptors

### 10.1 Diafenthiuron

### 10.2 Organotins (for Example Azocyclotin, Cyhexatin, Fenbutatin-Oxide)

### 11. Decouplers Ofoxidative Phosphorylation Acting by Interrupting the H-Proton Gradient

### 11.1 Pyrroles (for Example Chlorfenapyr)

### 11.2 Dinitrophenols (for Example Binapacryl, Dinobuton, Dinocap, DNOC)

### 12. Site-I Electron Transport Inhibitors

### 12.1 METIs (for Example Fenazaquin, Fenpyroximate, Pyrimidifen, Pyridaben, Tebufenpyrad, Tolfenpyrad)

### 12.2 Hydramethylnone

### 12.3 Dicofol

### 13. Site-II Electron Transport Inhibitors

### 13.1 Rotenone

### 14. Site-III Electron Transport Inhibitors

### 14.1 Acequinocyl, Fluacrypyrim

### 15. Microbial Disruptors of the Insect Gut Membrane

Bacillus thuringiensis Strains

### 16. Inhibitors of Fat Synthesis

### 16.1 Tetronic Acids (for Example Spirodiclofen, Spiromesifen)

16.2 tetramic acids [for example 3-(2,5-dimethylphenyl)-8-methoxy-2-oxo-1-azaspiro[4.5]dec-3-en-4-yl ethyl carbonate (alias: carbonic acid, 3-(2,5-dimethylphenyl)-8-methoxy-2-oxo-1-azaspiro[4.5]dec-3-en-4-yl ethyl ester, CAS Reg. No.: 382608-10-8) and carbonic acid, cis-3-(2,5-dimethylphenyl)-8-methoxy-2-oxo-1-azaspiro[4.5]dec-3-en-4-yl ethyl ester (CAS Reg. No.: 203313-25-1)]

### 17. Carboxamides

### (for Example Flonicamid)

### 18. Octopaminergic Agonists

### (for Example Amitraz)

### 19. Inhibitors of Magnesium-Stimulated A TPase

### (for Example Propargite)

### 20. Phthalamides

(for Example N²-[1,1-dimethyl-2-(methylsulphonyl)ethyl]-3-iodo-N′-[2-methyl-4-[1,2,2,2-tetrafluoro-1-(trifluoromethyl)ethyl]phenyl]-1,2-benzenedicarboxamide (CAS Reg. No.: 272451-65-7), flubendiamide)

### 21. Nereistoxin Analogues

### (for Example Thiocyclam Hydrogen Oxalate, Thiosultap-Sodium)

### 22. Biologicals, Hormones or Pheromones

(for Example Azadirachtin, Bacillus spec., Beauveria spec., Codlemone, Metarrhizium spec., Paecilomyces spec., Thuringiensin, Verticillium spec.)
23. Active Compounds with Unknown or Unspecific Mechanisms of Action

### 23.1 Fumigants (for Example Aluminium Phosphide, Methyl Bromide, Sulphuryl Fluoride)

### 23.2 Selective Antifeedants (for Example Cryolite, Flonicamid, Pymetrozine)

### 23.3 Mite Growth Inhibitors (for Example Clofentezine, Etoxazole, Hexythiazox)

23.4 Amidoflumet, Benclothiaz, Benzoximate, Bifenazate, Bromopropylate, Buprofezin, Chinomethionat, Chlordimeform, chlorobenzilate, chloropicrin, clothiazoben, cycloprene, cyflumetofen, dicyclanil, Fenoxacrim, Fentrifanil, Flubenzimine, Flufenerim, Flutenzin, Gossyplure, Hydramethyl-None, Japonilure, Metoxadiazone, Petroleum, Piperonyl Butoxide, Potassium Oleate, Pyrafluprole, Pyridalyl, Pyriprole, Sulfluramid, Tetradifon, Tetrasul, Triarathene, Verbutin,
furthermore the compound 3-methylphenyl propylcarbamate (Tsumacide Z), the compound 3-(5-chloro-3-pyridinyl)-8-(2,2,2-trifluoroethyl)-8-azabicyclo[3.2.1]octane-3-carbonitrile (CAS Reg. No. 185982-80-3) and the corresponding 3-endo-isomer (CAS Reg. No. 185984-60-5) (cf. WO 96/37494, WO 98/25923), and preparations which comprise insecticidally active plant extracts, nematodes, fungi or viruses.

A mixture with other known active compounds, such as herbicides, or with fertilizers and growth regulators, safeners and/or semiochemicals is also possible.

In addition, the compounds of the formula (I) according to the invention also have very good antimycotic activity. They have a very broad antimycotic activity spectrum in particular against dermatophytes and yeasts, moulds and diphasic fungi (for example against Candida species such as Candida albicans, Candida glabrata) and Epidermophyton floccosum, Aspergillus species such as Aspergillus niger and Aspergillus fumigatus, Trichophyton species such as Trichophyton mentagrophytes, Microsporon species such as Microsporon canis and audouinii. The list of these fungi does by no means limit the mycotic spectrum which can be covered, but is only for illustration.

The active compounds can be used as such, in the form of their formulations or the use forms prepared therefrom, such as ready-to-use solutions, suspensions, wettable powders, pastes, soluble powders, dusts and granules. Application is carried out in a customary manner, for example by watering, spraying, atomizing, broadcasting, dusting, foaming, spreading, etc. It is furthermore possible to apply the active compounds by the ultra-low volume method, or to inject the active compound preparation or the active compound itself into the soil. It is also possible to treat the seeds of the plants.

When using the active compounds according to the invention as fungicides, the application rates can be varied within a relatively wide range, depending on the kind of application. For the treatment of parts of plants, the active compound application rates are generally between 0.1 and 10 000 g/ha, preferably between 10 and 1000 g/ha. For seed dressing, the active compound application rates are generally between 0.001 and 50 g per kilogram of seed, preferably between 0.01 and 10 g per kilogram of seed. For the treatment of the soil, the active compound application rates are generally between 0.1 and 10 000 g/ha, preferably between 1 and 5 000 g/ha.

As already mentioned above, it is possible to treat all plants and their parts according to the invention. In a preferred embodiment, wild plant species and plant cultivars, or those obtained by conventional biological breeding, such as crossing or protoplast fusion, and parts thereof, are treated. In a further preferred embodiment, transgenic plants and plant cultivars obtained by genetic engineering, if appropriate in combination with conventional methods (Genetically Modified Organisms), and parts thereof, are treated. The term “parts” or “parts of plants” or “plant parts” has been explained above.

Particularly preferably, plants of the plant cultivars which are in each case commercially available or in use are treated according to the invention. Plant cultivars are to be understood as meaning plants having new properties (“traits”) and which have been obtained by conventional breeding, by mutagenesis or by recombinant DNA techniques. They can be cultivars, varieties, bio- or genotypes.

Depending on the plant species or plant cultivars, their location and growth conditions (soils, climate, vegetation period, diet), the treatment according to the invention may also result in superadditive (“synergistic”) effects. Thus, for example, reduced application rates and/or a widening of the activity spectrum and/or an increase in the activity of the substances and compositions which can be used according to the invention, better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products are possible which exceed the effects which were actually to be expected.

The transgenic plants or plant cultivars (i.e. those obtained by genetic engineering) which are preferably to be treated according to the invention include all plants which, by the genetic modification, received genetic material which imparted particularly advantageous useful properties (“traits”) to these plants. Examples of such properties are better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products. Further and particularly emphasized examples of such properties are a better defence of the plants against animal and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), maize, soya beans, potatoes, cotton, tobacco, oilseed rape and also fruit plants (with the fruits apples, pears, citrus fruits and grapes), and particular emphasis is given to maize, soya beans, potatoes, cotton, tobacco and oilseed rape. Traits that are particularly emphasized are increased defence of the plants against insects, arachnids, nematodes and slugs and snails by toxins formed in the plants, in particular those formed in the plants by the genetic material from Bacillus thuringiensis (for example by the genes CryIA(a), CryIA(b), CryIA(c), CrylIA, CrylhA, CrylIIB2, Cry9c, Cry2Ab, Cry3Bb and CrylF and also combinations thereof) (hereinbelow referred to as “Bt plants”). Traits that are also particularly emphasized are the increased defence of the plants against fungi, bacteria and viruses by systemic acquired resistance (SAR), systemin, phytoalexins, elicitors and resistance genes and correspondingly expressed proteins and toxins. Traits that are furthermore particularly emphasized are the increased tolerance of the plants to certain herbicidally active compounds, for example imidazolinones, sulphonylureas, glyphosate or phosphinotricin (for example the “PAT” gene). The genes which impart the desired traits in each case can also be present in combinations with one another in the transgenic plants. Examples of “Bt plants” which may be mentioned are maize varieties, cotton varieties, soya bean varieties and potato varieties which are sold under the trade names YIELD GARD® (for example maize, cotton, soya beans), KnockOut® (for example maize), StarLink® (for example maize), Bollgard® (cotton), Nucoton® (cotton) and NewLeaf® (potato). Examples of herbicide-tolerant plants which may be mentioned are maize varieties, cotton varieties and soya bean varieties which are sold under the trade names Roundup Ready® (tolerance to glyphosate, for example maize, cotton, soya bean), Liberty Link® (tolerance to phosphinotricin, for example oilseed rape), IMI® (tolerance to imidazolinones) and STS®V (tolerance to sulphonylureas, for example maize). Herbicide-resistant plants (plants bred in a conventional manner for herbicide tolerance) which may be mentioned also include the varieties sold under the name Clearfieldg (for example maize). Of course, these statements also apply to plant cultivars which have these genetic traits or genetic traits still to be developed, and which will be developed and/or marketed in the future.

The plants listed can be treated according to the invention in a particularly advantageous manner with the compounds of the general formula (I) or the active compound mixtures according to the invention. The preferred ranges stated above for the active compounds or mixtures also apply to the treatment of these plants. Particular emphasis is given to the treatment of plants with the compounds or mixtures specifically mentioned in the present text.

The preparation and the use of the active compounds according to the invention is illustrated by the examples below, but not limited to these examples.

### EXAMPLES

### Example 95

### 2-Methyl-N-(3-nitrophenyl)-4,5-dihydro[1,3]thiazolo[4,5-h]quinazoline-8-amine

### a) 2-Methyl-7-oxo-4,5,6,7-tetrahydrobenzothiazole

80 g (419 mmol) of 2-bromo-1,3-cyclohexanedione (Z. Chem. 1967, 7, 422) and 31.46 g (419 mmol) of thioacetamide were dissolved in 600 ml of pyridine and stirred at 50° C. overnight. The mixture was then cooled to room temperature, 600 ml of 10% strength NaCl solution were added and the mixture was extracted 3× with in each case 250 ml of dichloromethane. The organic phase was separated off, washed 2× with in each case 200 ml of 10% strength NaCl solution and dried over sodium sulphate. Removal of the solvent on a rotary evaporator gave a viscous dark-red oil which was distilled under high vacuum. At 96° C. (0.2 mbar), two fractions of 25.3 g (100% pure) and 6.2 g (90% pure), respectively, were obtained. (Total yield 44%). logP(HCOOH)=1.08

### b) 8-Formyl-2-methyl-7-oxo-4,5,6,7-tetrahydrobenzothiazole

11 g (275 mmol) of sodium hydride (as a 60% dispersion in oil) were initially charged in 400 ml of ether, and 40 ml of methanol were slowly added dropwise. After the evolution of hydrogen had ceased, 44.44 ml (550 mmol) of ethyl formate were added, followed by a solution of 18.4 g (110 mmol) of 2-methyl-7-oxo-4,5,6,7-tetrahydrobenzothiazole in 100 ml of diethyl ether. The reaction mixture was stirred at room temperature for 16 h, and 500 ml of 10% strength hydrochloric acid were then added. The organic phase was separated off and the aqueous phase was washed 3× with in each case 500 ml of ethyl acetate. The combined organic phases were dried, clarified and concentrated on a rotary evaporator. Supernatant paraffin oil was extracted with n-hexane and the product was taken up in ethyl acetate and re-concentrated. 8.5 g (77% pure; 33 mmol) of a red-brown oil are obtained as crude product (30% yield) which is used for the next step without further purification. logP(HCOOH)=1.36

### c) 2-Methyl-N-(3-nitrophenyl)-4,5-dihydro[1,3] thiazolo[4,5-h]quinazoline-8-amine

403 mg (2.06 mmol) of 8-formyl-2-methyl-7-oxo-4,5,6,7-tetrahydrobenzothiazole and 500 mg (2.06 mmol) of 3-nitrophenylguanidinium nitrate were dissolved in 5 ml of DMF, 285 mg (2.06 mmol) of K₂CO₃ were added and the mixture was stirred at 100° C. for 3 h. The mixture was then cooled to RT and poured onto water. The solid was filtered off using a Nutsch filter, washed with water and dried. This gave 106 mg (13%, 0.23 mmol, based on 85% purity) of a beige solid. logP(HCOOH)=2.79

### Example 216

### 4-(2-cyclohexyl-4-methyl-1,3-thiazol-5-yl)-N-(3-nitrophenyl)pyrimidine-2-amine

### a) 3-Dimethylamino-1-(2-cyclohexyl-4-methylthiazol-5-yl)propenone

4.2 g (18.8 mmol) of 5-acetyl-2-cyclohexyl-4-methylthiazole and 24.9 ml (188 mmol) of N,N-dimethylformamide dimethyl acetal were stirred at 100° C. for 12 h. The mixture was evaporated to dryness on a rotary evaporator and the residue was chromatographed on silica gel. This gave 5.1 g of product (81% pure). logP(HCOOH)=2.44

### b) 4-(2-Cyclohexyl-4-methyl-1,3-thiazol-5-yl)-N-(3-nitrophenyl)pyrimidine-2-amine

200 mg (0.58 mmol) of 3-dimethylamino-1-(2-cyclohexyl-4-methylthiazol-5-yl)propenone and 141 mg (0.58 mmol) of 3-nitrophenylguanidinium nitrate together with 61.6 mg (0.58 mmol) of Na₂CO₃ were stirred in 5 ml of 2-methoxyethanol at 100° C. for 18 h. The warm mixture was then filtered, the filter cake was washed with acetone and the filtrate was concentrated on a rotary evaporator. The residue was triturated with a 1:1 mixture of acetone and methyl tert-butyl ether and the resulting crystals were filtered off with suction and dried. This gave 85 mg of product (99% pure). logP(HCOOH)=4.63

### Example 874

### 5-Chloro-4-(2,4-dimethyl-1,3-thiazol-5-yl)-N-(3,4,5-trimethoxyphenyl)-pyrimidine-2-amine

### a) 2,5-Dichloro-4-(2,4-dimethyl-1,3-thiazol-5-yl)pyrimidine

Under a constant stream of argon, 4.8 g (25 mmol) of 5-broo-2,4-dimethylthiazole in 50 ml of diethyl ether are cooled to −70° C. At this temperature, 9.7 ml (25 mmol) of n-butyllithium as a 2.6 molar solution in hexane are added dropwise over a period of 30 minutes. The mixture is stirred at −70° C. for another half an hour. 3.45 g (23 mmol) of 2,5-dichloropyrimidine were added dropwise as a solution in 40 ml of ether, and the mixture is stirred for 30 minutes. The resulting suspension is warmed to 0° C. over a period of one hour and then quenched with 0.52 g (29 mmol) of water in 20 ml of THF. A solution of 5.67 g (25 mmol) of 4,5-dichloro-3,6-dioxocyclohexa-1,4-diene-1,2-dicarbonitrile (DDQ), dissolved in 100 ml of THF, is then added dropwise to the mixture. The mixture is warmed to 20° C., stirred for 15 minutes and once more cooled to 0° C. 29 ml (58 mmol) of 2 N aqueous sodium hydroxide solution are added to the solution, and the mixture is stirred at 0° C. for 5 minutes. The organic phase is separated off, dried with sodium sulphate and concentrated. Chromatographic purification on silica gel using 2-methoxy-2-methylpropane as mobile phase gives 3.9 g (63% of theory) of 2,5-dichloro-4-(2,4-dimethyl-1,3-thiazol-5-yl)pyrimidine in a purity of 97% (HPLC). logP(HCOOH)=2.47

### b) 5-Chloro-4-(2,4-dimethyl-1,3-thiazol-5-yl)-N-(3,4,5-trimethoxyphenyl)pyrimidine-2-amine

0.112 g (0.43 mmol) of 2,5-dichloro-4-(2,4-dimethyl-1,3-thiazol-5-yl)pyrimidine and 0.316 g (1.72 mmol) of 3,4,5-trimethoxyaniline in 6.9 ml (1.72 mmol) of 0.25 N hydrochloric acid are stirred in a microwave oven at 250 W, 150° C. and 15 bar for one hour. After cooling, the mixture is extracted twice with in each case 5 ml of ethyl acetate. The organic phases are combined, dried over sodium sulphate and concentrated. This gives 0.11 g (52% of theory) of 5-chloro-4-(2,4-dimethyl-1,3-thiazol-5-yl)-N-(3,4,5-trimethoxyphenyl)pyrimidine-2-amine in a purity of 82% (HPLC). logP(HCOOH)=2.82

The compounds of the formula (I) and (Ia) listed in Table 1 below were or are also obtained analogously to the methods indicated above.

TABLE 1

| Example | IUPAC Name | logP |
|---|---|---|
| 1 | 1-(3-{[2-(tert-butylamine)-4,5-dihydro[1,3]thiazole[4,5-h]quinazolin- | 2.18* |
| | 8-yl]amino}phenyl)ethanone | |
| 2 | 1-(3-{[4-(2,4-dimethyl-1,3-thiazol-5-yl)pyrimidin-2- | 2.14** |
| | yl]amino}phenyl)ethanone | |
| 3 | 1-(3-{[4-(2-amino-1,3-thiazol-5-yl)pyrimidin-2- | |
| | yl]amino}phenyl)ethanone | |
| 4 | 1-(3-{[4-(2-amino-4-methyl-1,3-thiazol-5-yl)pyrimidin-2- | 1.47* |
| | yl]amino}phenyl)ethanone | |
| 5 | 1-(3-{[4-(2-amino-4-sec-butyl-1,3-thiazol-5-yl)pyrimidin-2- | |
| | yl]amino}phenyl)ethanone | |
| 6 | 1-(3-{[4-(2-cyclohexyl-4-methyl-1,3-thiazol-5-yl)pyrimidin-2- | 4.11* |
| | yl]amino}phenyl)ethanone | |
| 7 | 1-(3-{[4-(2-cyclopropyl-4-methyl-1,3-thiazol-5-yl)pyrimidin-2- | 2.88* |
| | yl]amino}phenyl)ethanone | |
| 8 | 1-(3-{[4-(2-ethyl-1,3-thiazol-5-yl)pyrimidin-2- | |
| | yl]amino}phenyl)ethanone | |
| 9 | 1-(3-{[4-(2-ethyl-4-methyl-1,3-thiazol-5-yl)pyrimidin-2- | 2.7* |
| | yl]amino}phenyl)ethanone | |
| 10 | 1-(3-{[4-(2-isopropyl-4-methyl-1,3-thiazol-5-yl)pyrimidin-2- | 3.1* |
| | yl]amino}phenyl)ethanone | |
| 11 | 1-(3-{[4-(2-methyl-1,3-thiazol-5-yl)pyrimidin-2- | 2.57* |
| | yl]amino}phenyl)ethanone | |
| 12 | 1-(3-{[4-(4-chloro-2-methyl-1,3-thiazol-5-yl)pyrimidin-2- | |
| | yl]amino}phenyl)ethanone | |
| 13 | 1-(3-{[4-(4-cyclopropyl-2-ethyl-1,3-thiazol-5-yl)pyrimidin-2- | |
| | yl]amino}phenyl)ethanone | |
| 14 | 1-(3-{[4-(4-cyclopropyl-2-methyl-1,3-thiazol-5-yl)pyrimidin-2- | |
| | yl]amino}phenyl)ethanone | |
| 15 | 1-(3-{[4-(4-methoxy-2-methyl-1,3-thiazol-5-yl)pyrimidin-2- | |
| | yl]amino}phenyl)ethanone | |
| 16 | 1-(3-{[4-(4-methyl-2-phenyl-1,3-thiazol-5-yl)pyrimidin-2- | |
| | yl]amino}phenyl)ethanone | |
| 17 | 1-(3-{[4-(4-sec-butyl-2-ethyl-1,3-thiazol-5-yl)pyrimidin-2- | |
| | yl]amino}phenyl)ethanone | |
| 18 | 1-(3-{[4-(4-sec-butyl-2-methyl-1,3-thiazol-5-yl)pyrimidin-2- | |
| | yl]amino}phenyl)ethanone | |
| 19 | 1-[3-({2-[(2-hydroxy-1-methylethyl)amino]-4,5- | |
| | dihydro[1,3]thiazole[4,5-h]quinazolin-8-yl}amino)phenyl]ethanone | |
| 20 | 1-[3-({2-[(2-methoxy-1-methylethyl)amino]-4,5- | |
| | dihydro[1,3]thiazole[4,5-h]quinazolin-8-yl}amino)phenyl]ethanone | |
| 21 | 1-[3-({2-[(tetrahydrofuran-2-ylmethyl)amino]-4,5- | |
| | dihydro[1,3]thiazole[4,5-h]quinazolin-8-yl}amino)phenyl]ethanone | |
| 22 | 1-[3-({4-[2-(cyclopropylamine)-4-methyl-1,3-thiazol-5-yl]pyrimidin- | |
| | 2-yl}amino)phenyl]ethanone | |
| 23 | 1-[3-({4-[2-(dimethylamine)-4-(trifluoromethyl)-1,3-thiazol-5- | 1.91* |
| | yl]pyrimidin-2-yl}amino)phenyl]ethanone | |
| 24 | 1-[3-({4-[2-(methoxymethyl)-4-methyl-1,3-thiazol-5-yl]pyrimidin-2- | 2.38* |
| | yl}amino)phenyl]ethanone | |
| 25 | 1-[3-({4-[2-(tert-butylamine)-4-methyl-1,3-thiazol-5-yl]pyrimidin-2- | 2.25* |
| | yl}amino)phenyl]ethanone | |
| 26 | 1-[3-({4-[2-methyl-4-(trifluoromethyl)-1,3-thiazol-5-yl]pyrimidin-2- | 3.02* |
| | yl}amino)phenyl]ethanone | |
| 27 | 1-[3-({4-[4-methyl-2-(methylamine)-1,3-thiazol-5-yl]pyrimidin-2- | 1.62* |
| | yl}amino)phenyl]ethanone | |
| 28 | 1-[3-({4-[4-methyl-2-(methylthio)-1,3-thiazol-5-yl]pyrimidin-2- | 2.99* |
| | yl}amino)phenyl]ethanone | |
| 29 | 1-{3-[(2-amino-4,5-dihydro[1,3]thiazole[4,5-h]quinazolin-8- | |
| | yl)amino]phenyl}ethanone | |
| 30 | 1-{3-[(2-methyl-4,5-dihydro[1,3]thiazole[4,5-h]quinazolin-8- | 2.29* |
| | yl)amino]phenyl}ethanone | |
| 31 | 1-{3-[(4-{2-[(2-hydroxy-1-methylethyl)amino]-4-methyl-1,3-thiazol- | |
| | 5-yl}pyrimidin-2-yl)amino]phenyl}ethanone | |
| 32 | 1-{3-[(4-{2-[(2-methoxy-1-methylethyl)amino]-1,3-thiazol-5- | |
| | yl}pyrimidin-2-yl)amino]phenyl}ethanone | |
| 33 | 1-{3-[(4-{2-[(2-methoxy-1-methylethyl)amino]-4-methyl-1,3-thiazol- | |
| | 5-yl}pyrimidin-2-yl)amino]phenyl}ethanone | |
| 34 | 1-{3-[(4-{2-[(2-methoxyethyl)amino]-1,3-thiazol-5-yl}pyrimidin-2- | |
| | yl)amino]phenyl}ethanone | |
| 35 | 1-{3-[(4-{2-[(tetrahydrofuran-2-ylmethyl)amino]-1,3-thiazol-5- | |
| | yl}pyrimidin-2-yl)amino]phenyl}ethanone | |
| 36 | 1-{3-[(4-{4-cyclopropyl-2-[(2-methoxy-1-methylethyl)amino]-1,3- | |
| | thiazol-5-yl}pyrimidin-2-yl)amino]phenyl}ethanone | |
| 37 | 1-{3-[(4-{4-cyclopropyl-2-[(2-methoxyethyl)amino]-1,3-thiazol-5- | |
| | yl}pyrimidin-2-yl)amino]phenyl}ethanone | |
| 38 | 1-{3-[(4-{4-cyclopropyl-2-[(tetrahydrofuran-2-ylmethyl)amino]-1,3- | |
| | thiazol-5-yl}pyrimidin-2-yl)amino]phenyl}ethanone | |
| 39 | 1-{3-[(4-{4-methyl-2-[(tetrahydrofuran-2-ylmethyl)amino]-1,3- | 1.82* |
| | thiazol-5-yl}pyrimidin-2-yl)amino]phenyl}ethanone | |
| 40 | 1-{3-[(4-{4-sec-butyl-2-[(2-methoxy-1-methylethyl)amino]-1,3- | |
| | thiazol-5-yl}pyrimidin-2-yl)amino]phenyl}ethanone | |
| 41 | 1-{3-[(4-{4-sec-butyl-2-[(tetrahydrofuran-2-ylmethyl)amino]-1,3- | |
| | thiazol-5-yl}pyrimidin-2-yl)amino]phenyl}ethanone | |
| 42 | 2-({8-[(3,4,5-trimethoxyphenyl)amino]-4,5-dihydro[1,3]thiazole[4,5- | |
| | h]quinazolin-2-yl}amino)propan-1-ol | |
| 43 | 2-({8-[(3-chlorophenyl)amino]-4,5-dihydro[1,3]thiazole[4,5- | |
| | h]quinazolin-2-yl}amino)propan-1-ol | |
| 44 | 2-({8-[(3-methoxyphenyl)amino]-4,5-dihydro[1,3]thiazole[4,5- | |
| | h]quinazolin-2-yl}amino)propan-1-ol | |
| 45 | 2-({8-[(3-nitrophenyl)amino]-4,5-dihydro[1,3]thiazole[4,5- | |
| | h]quinazolin-2-yl}amino)propan-1-ol | |
| 46 | 2-({8-[(4-fluoro-3-methylphenyl)amino]-4,5-dihydro[1,3]thiazole[4,5- | |
| | h]quinazolin-2-yl}amino)propan-1-ol | |
| 47 | 2-({8-[(4-fluorophenyl)amino]-4,5-dihydro[1,3]thiazole[4,5- | |
| | h]quinazolin-2-yl}amino)propan-1-ol | |
| 48 | 2-({8-[(6-methoxypyridin-3-yl)amino]-4,5-dihydro[1,3]thiazole[4,5- | |
| | h]quinazolin-2-yl}amino)propan-1-ol | |
| 49 | 2,2,2-trifluoro-N-(5-{2-[(3,4,5-trimethoxyphenyl)amino]pyrimidin-4- | |
| | yl}-1,3-thiazol-2-yl)acetamide | |
| 50 | 2,2,2-trifluoro-N-(5-{2-[(3-hydroxyphenyl)amino]pyrimidin-4-yl}- | |
| | 1,3-thiazol-2-yl)acetamide | |
| 51 | 2,2,2-trifluoro-N-(5-{2-[(3-methoxyphenyl)amino]pyrimidin-4-yl}- | |
| | 1,3-thiazol-2-yl)acetamide | |
| 52 | 2-[(4-methyl-5-{2-[(3,4,5-trimethoxyphenyl)amino]pyrimidin-4-yl}- | |
| | 1,3-thiazol-2-yl)amino]propan-1-ol | |
| 53 | 2-[(4-methyl-5-{2-[(3-nitrophenyl)amino]pyrimidin-4-yl}-1,3-thiazol- | |
| | 2-yl)amino]propan-1-ol | |
| 54 | 2-[(5-{2-[(3-chlorophenyl)amino]pyrimidin-4-yl}-4-methyl-1,3- | |
| | thiazol-2-yl)amino]propan-1-ol | |
| 55 | 2-[(5-{2-[(3-methoxyphenyl)amino]pyrimidin-4-yl}-4-methyl-1,3- | |
| | thiazol-2-yl)amino]propan-1-ol | |
| 56 | 2-[(5-{2-[(4-fluoro-3-methylphenyl)amino]pyrimidin-4-yl}-4-methyl- | |
| | 1,3-thiazol-2-yl)amino]propan-1-ol | |
| 57 | 2-[(5-{2-[(4-fluorophenyl)amino]pyrimidin-4-yl}-4-methyl-1,3- | |
| | thiazol-2-yl)amino]propan-1-ol | |
| 58 | 2-[(5-{2-[(6-methoxypyridin-3-yl)amino]pyrimidin-4-yl}-4-methyl- | |
| | 1,3-thiazol-2-yl)amino]propan-1-ol | |
| 59 | 2-[(8-anilino-4,5-dihydro[1,3]thiazole[4,5-h]quinazolin-2- | |
| | yl)amino]propan-1-ol | |
| 60 | 2-{[5-(2-anilinopyrimidin-4-yl)-4-methyl-1,3-thiazol-2- | |
| | yl]amino}propan-1-ol | |
| 61 | 2-chloro-N1,N1-dimethyl-N4-{4-[4-methyl-2-(methylthio)-1,3- | 2.74* |
| | thiazol-5-yl]pyrimidin-2-yl}benzene-1,4-diamine | |
| 62 | 2-chloro-N4-[4-(2,4-dimethyl-1,3-thiazol-5-yl)pyrimidin-2-yl]-N1,N1- | 2.06* |
| | dimethylbenzene-1,4-diamine | |
| 63 | 2-chloro-N4-[4-(2-cyclopropyl-4-methyl-1,3-thiazol-5-yl)pyrimidin-2- | 2.59* |
| | yl]-N1,N1-dimethylbenzene-1,4-diamine | |
| 64 | 2-chloro-N4-[4-(2-isopropyl-4-methyl-1,3-thiazol-5-yl)pyrimidin-2- | 2.72* |
| | yl]-N1,N1-dimethylbenzene-1,4-diamine | |
| 65 | 2-chloro-N4-{4-[2-(methoxymethyl)-4-methyl-1,3-thiazol-5- | 2.04* |
| | yl]pyrimidin-2-yl}-N1,N1-dimethylbenzene-1,4-diamine | |
| 66 | 2-methoxyethyl-(4-{[4-(2,4-dimethyl-1,3-thiazol-5-yl)pyrimidin-2- | |
| | yl]amino}phenyl)carbamate | |
| 67 | 2-methoxyethyl-(4-{[4-(2-amino-1,3-thiazol-5-yl)pyrimidin-2- | |
| | yl]amino}phenyl)carbamate | |
| 68 | 2-methoxyethyl-(4-{[4-(2-amino-4-cyclopropyl-1,3-thiazol-5- | |
| | yl)pyrimidin-2-yl]amino}phenyl)carbamate | |
| 69 | 2-methoxyethyl-(4-{[4-(2-amino-4-methyl-1,3-thiazol-5-yl)pyrimidin- | 1.4* |
| | 2-yl]amino}phenyl)carbamate | |
| 70 | 2-methoxyethyl-(4-{[4-(2-amino-4-sec-butyl-1,3-thiazol-5- | |
| | yl)pyrimidin-2-yl]amino}phenyl)carbamate | |
| 71 | 2-methoxyethyl-(4-{[4-(2-cyclopropyl-4-methyl-1,3-thiazol-5- | 2.51* |
| | yl)pyrimidin-2-yl]amino}phenyl)carbamate | |
| 72 | 2-methoxyethyl-(4-{[4-(2-ethyl-1,3-thiazol-5-yl)pyrimidin-2- | |
| | yl]amino}phenyl)carbamate | |
| 73 | 2-methoxyethyl-(4-{[4-(2-ethyl-4-methyl-1,3-thiazol-5-yl)pyrimidin- | 2.36* |
| | 2-yl]amino}phenyl)carbamate | |
| 74 | 2-methoxyethyl-(4-{[4-(2-isopropyl-4-methyl-1,3-thiazol-5- | 2.67* |
| | yl)pyrimidin-2-yl]amino}phenyl)carbamate | |
| 75 | 2-methoxyethyl-(4-{[4-(2-methyl-1,3-thiazol-5-yl)pyrimidin-2- | |
| | yl]amino}phenyl)carbamate | |
| 76 | 2-methoxyethyl-(4-{[4-(4-cyclopropyl-2-ethyl-1,3-thiazol-5- | |
| | yl)pyrimidin-2-yl]amino}phenyl)carbamate | |
| 77 | 2-methoxyethyl-(4-{[4-(4-cyclopropyl-2-methyl-1,3-thiazol-5- | |
| | yl)pyrimidin-2-yl]amino}phenyl)carbamate | |
| 78 | 2-methoxyethyl-(4-{[4-(4-sec-butyl-2-ethyl-1,3-thiazol-5- | |
| | yl)pyrimidin-2-yl]amino}phenyl)carbamate | |
| 79 | 2-methoxyethyl-(4-{[4-(4-sec-butyl-2-methyl-1,3-thiazol-5- | |
| | yl)pyrimidin-2-yl]amino}phenyl)carbamate | |
| 80 | 2-methoxyethyl-[4-({4-[2-(acetylamine)-1,3-thiazol-5-yl]pyrimidin-2- | |
| | yl}amino)phenyl]carbamate | |
| 81 | 2-methoxyethyl-[4-({4-[2-(acetylamine)-4-cyclopropyl-1,3-thiazol-5- | |
| | yl]pyrimidin-2-yl}amino)phenyl]carbamate | |
| 82 | 2-methoxyethyl-[4-({4-[2-(acetylamine)-4-sec-butyl-1,3-thiazol-5- | |
| | yl]pyrimidin-2-yl}amino)phenyl]carbamate | |
| 83 | 2-methoxyethyl-[4-({4-[4-methyl-2-(methylamine)-1,3-thiazol-5- | |
| | yl]pyrimidin-2-yl}amino)phenyl]carbamate | |
| 84 | 2-methoxyethyl-{4-[(4-{2-[(2-methoxy-1-methylethyl)amino]-1,3- | |
| | thiazol-5-yl}pyrimidin-2-yl)amino]phenyl}carbamate | |
| 85 | 2-methoxyethyl-{4-[(4-{2-[(2-methoxyethyl)amino]-1,3-thiazol-5- | |
| | yl}pyrimidin-2-yl)amino]phenyl}carbamate | |
| 86 | 2-methoxyethyl-{4-[(4-{2-[(tetrahydrofuran-2-ylmethyl)amino]-1,3- | |
| | thiazol-5-yl}pyrimidin-2-yl)amino]phenyl}carbamate | |
| 87 | 2-methoxyethyl-{4-[(4-{2-[(trifluoroacetyl)amino]-1,3-thiazol-5- | |
| | yl}pyrimidin-2-yl)amino]phenyl}carbamate | |
| 88 | 2-methoxyethyl-{4-[(4-{4-cyclopropyl-2-[(2-methoxyethyl)amino]- | |
| | 1,3-thiazol-5-yl}pyrimidin-2-yl)amino]phenyl}carbamate | |
| 89 | 2-methoxyethyl-{4-[(4-{4-cyclopropyl-2-[(tetrahydrofuran-2- | |
| | ylmethyl)amino]-1,3-thiazol-5-yl}pyrimidin-2- | |
| | yl)amino]phenyl}carbamate | |
| 90 | 2-methoxyethyl-{4-[(4-{4-cyclopropyl-2-[(trifluoroacetyl)amino]-1,3- | |
| | thiazol-5-yl}pyrimidin-2-yl)amino]phenyl}carbamate | |
| 91 | 2-methoxyethyl-{4-[(4-{4-sec-butyl-2-[(2-methoxy-1- | |
| | methylethyl)amino]-1,3-thiazol-5-yl}pyrimidin-2- | |
| | yl)amino]phenyl}carbamate | |
| 92 | 2-methoxyethyl-{4-[(4-{4-sec-butyl-2-[(tetrahydrofuran-2- | |
| | ylmethyl)amino]-1,3-thiazol-5-yl}pyrimidin-2- | |
| | yl)amino]phenyl}carbamate | |
| 93 | 2-methoxyethyl-{4-[(4-{4-sec-butyl-2-[(trifluoroacetyl)amino]-1,3- | |
| | thiazol-5-yl}pyrimidin-2-yl)amino]phenyl}carbamate | |
| 94 | 2-methyl-N-(3,4,5-trimethoxyphenyl)-4,5-dihydro[1,3]thiazole[4,5- | 2.18* |
| | h]quinazoline-8-amine | |
| 95 | 2-methyl-N-(3-nitrophenyl)-4,5-dihydro[1,3]thiazole[4,5- | 2.79* |
| | h]quinazoline-8-amine | |
| 96 | 2-methyl-N-phenyl-4,5-dihydro[1,3]thiazole[4,5-h]quinazoline-8- | 2.48* |
| | amine | |
| 97 | 3-({2-[(2-hydroxy-1-methylethyl)amino]-4,5- | |
| | dihydro[1,3]thiazole[4,5-h]quinazolin-8-yl}amino)phenol | |
| 98 | 3-({2-[(2-methoxy-1-methylethyl)amino]-4,5- | |
| | dihydro[1,3]thiazole[4,5-h]quinazolin-8-yl}amino)phenol | |
| 99 | 3-({2-[(tetrahydrofuran-2-ylmethyl)amino]-4,5- | |
| | dihydro[1,3]thiazole[4,5-h]quinazolin-8-yl}amino)phenol | |
| 100 | 3-({4-[2-(cyclopropylamine)-4-methyl-1,3-thiazol-5-yl]pyrimidin-2- | |
| | yl}amino)phenol | |
| 101 | 3-({4-[2-(methoxymethyl)-4-methyl-1,3-thiazol-5-yl]pyrimidin-2- | 1.97* |
| | yl}amino)phenol | |
| 102 | 3-({4-[2-(tert-butylamine)-4-methyl-1,3-thiazol-5-yl]pyrimidin-2- | 1.82* |
| | yl}amino)phenol | |
| 103 | 3-({4-[2-methyl-4-(trifluoromethyl)-1,3-thiazol-5-yl]pyrimidin-2- | 2.52* |
| | yl}amino)phenol | |
| 104 | 3-({4-[4-methyl-2-(methylamine)-1,3-thiazol-5-yl]pyrimidin-2- | |
| | yl}amino)phenol | |
| 105 | 3-(4-methyl-5-{2-[(3,4,5-trimethoxyphenyl)amino]pyrimidin-4-yl}- | |
| | 1,3-thiazol-2-yl)-1,3-oxazolidin-2-one | |
| 106 | 3-(4-methyl-5-{2-[(3-nitrophenyl)amino]pyrimidin-4-yl}-1,3-thiazol- | |
| | 2-yl)-1,3-oxazolidin-2-one | |
| 107 | 3-(5-{2-[(3-acetylphenyl)amino]pyrimidin-4-yl}-4-methyl-1,3-thiazol- | |
| | 2-yl)-1,3-oxazolidin-2-one | |
| 108 | 3-(5-{2-[(3-acetylphenyl)amino]pyrimidin-4-yl}-4-methyl-1,3-thiazol- | |
| | 2-yl)-4-methyl-1,3-oxazolidin-2-one | |
| 109 | 3-(5-{2-[(3-chlorophenyl)amino]pyrimidin-4-yl}-4-methyl-1,3- | |
| | thiazol-2-yl)-1,3-oxazolidin-2-one | |
| 110 | 3-(5-{2-[(3-chlorophenyl)amino]pyrimidin-4-yl}-4-methyl-1,3- | |
| | thiazol-2-yl)-4-methyl-1,3-oxazolidin-2-one | |
| 111 | 3-(5-{2-[(3-hydroxyphenyl)amino]pyrimidin-4-yl}-4-methyl-1,3- | |
| | thiazol-2-yl)-1,3-oxazolidin-2-one | |
| 112 | 3-(5-{2-[(3-hydroxyphenyl)amino]pyrimidin-4-yl}-4-methyl-1,3- | |
| | thiazol-2-yl)-4-methyl-1,3-oxazolidin-2-one | |
| 113 | 3-(5-{2-[(3-methoxyphenyl)amino]pyrimidin-4-yl}-4-methyl-1,3- | |
| | thiazol-2-yl)-1,3-oxazolidin-2-one | |
| 114 | 3-(5-{2-[(3-methoxyphenyl)amino]pyrimidin-4-yl}-4-methyl-1,3- | |
| | thiazol-2-yl)-4-methyl-1,3-oxazolidin-2-one | |
| 115 | 3-(5-{2-[(4-fluoro-3-methylphenyl)amino]pyrimidin-4-yl}-4-methyl- | |
| | 1,3-thiazol-2-yl)-1,3-oxazolidin-2-one | |
| 116 | 3-(5-{2-[(4-fluoro-3-methylphenyl)amino]pyrimidin-4-yl}-4-methyl- | |
| | 1,3-thiazol-2-yl)-4-methyl-1,3-oxazolidin-2-one | |
| 117 | 3-(5-{2-[(4-fluorophenyl)amino]pyrimidin-4-yl}-4-methyl-1,3-thiazol- | |
| | 2-yl)-1,3-oxazolidin-2-one | |
| 118 | 3-(5-{2-[(4-fluorophenyl)amino]pyrimidin-4-yl}-4-methyl-1,3-thiazol- | |
| | 2-yl)-4-methyl-1,3-oxazolidin-2-one | |
| 119 | 3-(5-{2-[(4-hydroxyphenyl)amino]pyrimidin-4-yl}-4-methyl-1,3- | |
| | thiazol-2-yl)-1,3-oxazolidin-2-one | |
| 120 | 3-(5-{2-[(4-hydroxyphenyl)amino]pyrimidin-4-yl}-4-methyl-1,3- | |
| | thiazol-2-yl)-4-methyl-1,3-oxazolidin-2-one | |
| 121 | 3-(5-{2-[(6-methoxypyridin-3-yl)amino]pyrimidin-4-yl}-4-methyl- | |
| | 1,3-thiazol-2-yl)-1,3-oxazolidin-2-one | |
| 122 | 3-(5-{2-[(6-methoxypyridin-3-yl)amino]pyrimidin-4-yl}-4-methyl- | |
| | 1,3-thiazol-2-yl)-4-methyl-1,3-oxazolidin-2-one | |
| 123 | 3-(8-anilino-4,5-dihydro[1,3]thiazole[4,5-h]quinazolin-2-yl)-1,3- | |
| | oxazolidin-2-one | |
| 124 | 3-(8-anilino-4,5-dihydro[1,3]thiazole[4,5-h]quinazolin-2-yl)-4- | |
| | methyl-1,3-oxazolidin-2-one | |
| 125 | 3-[(2-amino-4,5-dihydro[1,3]thiazole[4,5-h]quinazolin-8- | |
| | yl)amino]phenol | |
| 126 | 3-[(2-methyl-4,5-dihydro[1,3]thiazole[4,5-h]quinazolin-8- | 1.77* |
| | yl)amino]phenol | |
| 127 | 3-[(4-{2-[(2-hydroxy-1-methylethyl)amino]-4-methyl-1,3-thiazol-5- | |
| | yl}pyrimidin-2-yl)amino]phenol | |
| 128 | 3-[(4-{2-[(2-methoxy-1-methylethyl)amino]-1,3-thiazol-5- | |
| | yl}pyrimidin-2-yl)amino]phenol | |
| 129 | 3-[(4-{2-[(2-methoxy-1-methylethyl)amino]-4-methyl-1,3-thiazol-5- | 1.53* |
| | yl}pyrimidin-2-yl)amino]phenol | |
| 130 | 3-[(4-{2-[(2-methoxyethyl)amino]-1,3-thiazol-5-yl}pyrimidin-2- | |
| | yl)amino]phenol | |
| 131 | 3-[(4-{2-[(tetrahydrofuran-2-ylmethyl)amino]-1,3-thiazol-5- | |
| | yl}pyrimidin-2-yl)amino]phenol | |
| 132 | 3-[(4-{4-cyclopropyl-2-[(2-methoxy-1-methylethyl)amino]-1,3- | |
| | thiazol-5-yl}pyrimidin-2-yl)amino]phenol | |
| 133 | 3-[(4-{4-cyclopropyl-2-[(2-methoxyethyl)amino]-1,3-thiazol-5- | |
| | yl}pyrimidin-2-yl)amino]phenol | |
| 134 | 3-[(4-{4-cyclopropyl-2-[(tetrahydrofuran-2-ylmethyl)amino]-1,3- | |
| | thiazol-5-yl}pyrimidin-2-yl)amino]phenol | |
| 135 | 3-[(4-{4-methyl-2-[(tetrahydrofuran-2-ylmethyl)amino]-1,3-thiazol-5- | |
| | yl}pyrimidin-2-yl)amino]phenol | |
| 136 | 3-[(4-{4-sec-butyl-2-[(2-methoxy-1-methylethyl)amino]-1,3-thiazol-5- | |
| | yl}pyrimidin-2-yl)amino]phenol | |
| 137 | 3-[(4-{4-sec-butyl-2-[(tetrahydrofuran-2-ylmethyl)amino]-1,3-thiazol- | |
| | 5-yl}pyrimidin-2-yl)amino]phenol | |
| 138 | 3-[5-(2-anilinopyrimidin-4-yl)-4-methyl-1,3-thiazol-2-yl]-1,3- | |
| | oxazolidin-2-one | |
| 139 | 3-[5-(2-anilinopyrimidin-4-yl)-4-methyl-1,3-thiazol-2-yl]-4-methyl- | |
| | 1,3-oxazolidin-2-one | |
| 140 | 3-{[2-(tert-butylamine)-4,5-dihydro[1,3]thiazole[4,5-h]quinazolin-8- | |
| | yl]amino}phenol | |
| 141 | 3-{[4-(2,4-dimethyl-1,3-thiazol-5-yl)pyrimidin-2-yl]amino}phenol | 1.84* |
| 142 | 3-{[4-(2-amino-1,3-thiazol-5-yl)pyrimidin-2-yl]amino}phenol | |
| 143 | 3-{[4-(2-amino-4-cyclopropyl-1,3-thiazol-5-yl)pyrimidin-2- | |
| | yl]amino}phenol | |
| 144 | 3-{[4-(2-amino-4-sec-butyl-1,3-thiazol-5-yl)pyrimidin-2- | |
| | yl]amino}phenol | |
| 145 | 3-{[4-(2-cyclohexyl-4-methyl-1,3-thiazol-5-yl)pyrimidin-2- | 3.27* |
| | yl]amino}benzoic acid | |
| 146 | 3-{[4-(2-cyclohexyl-4-methyl-1,3-thiazol-5-yl)pyrimidin-2- | 3.33* |
| | yl]amino}phenol | |
| 147 | 3-{[4-(2-cyclopropyl-4-methyl-1,3-thiazol-5-yl)pyrimidin-2- | 2.27* |
| | yl]amino}benzoic acid | |
| 148 | 3-{[4-(2-cyclopropyl-4-methyl-1,3-thiazol-5-yl)pyrimidin-2- | 2.24* |
| | yl]amino}phenol | |
| 149 | 3-{[4-(2-ethyl-1,3-thiazol-5-yl)pyrimidin-2-yl]amino}phenol | |
| 150 | 3-{[4-(2-ethyl-4-methyl-1,3-thiazol-5-yl)pyrimidin-2-yl]amino}phenol | 2.12* |
| 151 | 3-{[4-(2-isopropyl-4-methyl-1,3-thiazol-5-yl)pyrimidin-2- | 2.42* |
| | yl]amino}phenol | |
| 152 | 3-{[4-(2-methyl-1,3-thiazol-5-yl)pyrimidin-2-yl]amino}phenol | |
| 153 | 3-{[4-(4-cyclopropyl-2-ethyl-1,3-thiazol-5-yl)pyrimidin-2- | |
| | yl]amino}phenol | |
| 154 | 3-{[4-(4-cyclopropyl-2-methyl-1,3-thiazol-5-yl)pyrimidin-2- | |
| | yl]amino}phenol | |
| 155 | 3-{[4-(4-methyl-2-phenyl-1,3-thiazol-5-yl)pyrimidin-2- | |
| | yl]amino}phenol | |
| 156 | 3-{[4-(4-sec-butyl-2-ethyl-1,3-thiazol-5-yl)pyrimidin-2- | |
| | yl]amino}phenol | |
| 157 | 3-{[4-(4-sec-butyl-2-methyl-1,3-thiazol-5-yl)pyrimidin-2- | |
| | yl]amino}phenol | |
| 158 | 3-{8-[(3,4,5-trimethoxyphenyl)amino]-4,5-dihydro[1,3]thiazole[4,5- | |
| | h]quinazolin-2-yl}-1,3-oxazolidin-2-one | |
| 159 | 3-{8-[(3-acetylphenyl)amino]-4,5-dihydro[1,3]thiazole[4,5- | |
| | h]quinazolin-2-yl}-1,3-oxazolidin-2-one | |
| 160 | 3-{8-[(3-acetylphenyl)amino]-4,5-dihydro[1,3]thiazole[4,5- | |
| | h]quinazolin-2-yl}-4-methyl-1,3-oxazolidin-2-one | |
| 161 | 3-{8-[(3-chlorophenyl)amino]-4,5-dihydro[1,3]thiazole[4,5- | |
| | h]quinazolin-2-yl}-1,3-oxazolidin-2-one | |
| 162 | 3-{8-[(3-chlorophenyl)amino]-4,5-dihydro[1,3]thiazole[4,5- | |
| | h]quinazolin-2-yl}-4-methyl-1,3-oxazolidin-2-one | |
| 163 | 3-{8-[(3-hydroxyphenyl)amino]-4,5-dihydro[1,3]thiazole[4,5- | |
| | h]quinazolin-2-yl}-1,3-oxazolidin-2-one | |
| 164 | 3-{8-[(3-hydroxyphenyl)amino]-4,5-dihydro[1,3]thiazole[4,5- | |
| | h]quinazolin-2-yl}-4-methyl-1,3-oxazolidin-2-one | |
| 165 | 3-{8-[(3-methoxyphenyl)amino]-4,5-dihydro[1,3]thiazole[4,5- | |
| | h]quinazolin-2-yl}-1,3-oxazolidin-2-one | |
| 166 | 3-{8-[(3-methoxyphenyl)amino]-4,5-dihydro[1,3]thiazole[4,5- | |
| | h]quinazolin-2-yl}-4-methyl-1,3-oxazolidin-2-one | |
| 167 | 3-{8-[(3-nitrophenyl)amino]-4,5-dihydro[1,3]thiazole[4,5- | |
| | h]quinazolin-2-yl}-1,3-oxazolidin-2-one | |
| 168 | 3-{8-[(4-fluoro-3-methylphenyl)amino]-4,5-dihydro[1,3]thiazole[4,5- | |
| | h]quinazolin-2-yl}-1,3-oxazolidin-2-one | |
| 169 | 3-{8-[(4-fluoro-3-methylphenyl)amino]-4,5-dihydro[1,3]thiazole[4,5- | |
| | h]quinazolin-2-yl}-4-methyl-1,3-oxazolidin-2-one | |
| 170 | 3-{8-[(4-fluorophenyl)amino]-4,5-dihydro[1,3]thiazole[4,5- | 1.34* |
| | h]quinazolin-2-yl}-1,3-oxazolidin-2-one | |
| 171 | 3-{8-[(4-fluorophenyl)amino]-4,5-dihydro[1,3]thiazole[4,5- | |
| | h]quinazolin-2-yl}-4-methyl-1,3-oxazolidin-2-one | |
| 172 | 3-{8-[(4-hydroxyphenyl)amino]-4,5-dihydro[1,3]thiazole[4,5- | |
| | h]quinazolin-2-yl}-1,3-oxazolidin-2-one | |
| 173 | 3-{8-[(4-hydroxyphenyl)amino]-4,5-dihydro[1,3]thiazole[4,5- | |
| | h]quinazolin-2-yl}-4-methyl-1,3-oxazolidin-2-one | |
| 174 | 3-{8-[(6-methoxypyridin-3-yl)amino]-4,5-dihydro[1,3]thiazole[4,5- | |
| | h]quinazolin-2-yl}-1,3-oxazolidin-2-one | |
| 175 | 3-{8-[(6-methoxypyridin-3-yl)amino]-4,5-dihydro[1,3]thiazole[4,5- | |
| | h]quinazolin-2-yl}-4-methyl-1,3-oxazolidin-2-one | |
| 176 | 4-({2-[(2-hydroxy-1-methylethyl)amino]-4,5- | |
| | dihydro[1,3]thiazole[4,5-h]quinazolin-8-yl}amino)phenol | |
| 177 | 4-({2-[(2-methoxy-1-methylethyl)amino]-4,5- | |
| | dihydro[1,3]thiazole[4,5-h]quinazolin-8-yl}amino)phenol | |
| 178 | 4-({2-[(tetrahydrofuran-2-ylmethyl)amino]-4,5- | |
| | dihydro[1,3]thiazole[4,5-h]quinazolin-8-yl}amino)phenol | |
| 179 | 4-({4-[2-(methoxymethyl)-4-methyl-1,3-thiazol-5-yl]pyrimidin-2- | 2.57* |
| | yl}amino)benzonitrile | |
| 180 | 4-({4-[2-(tert-butylamine)-4-methyl-1,3-thiazol-5-yl]pyrimidin-2- | 1.67* |
| | yl}amino)phenol | |
| 181 | 4-({4-[4-methyl-2-(methylamine)-1,3-thiazol-5-yl]pyrimidin-2- | |
| | yl}amino)benzonitrile | |
| 182 | 4-(2,4-dimethyl-1,3-thiazol-5-yl)-N-(3,4,5- | 2.1** |
| | trimethoxyphenyl)pyrimidine-2-amine | |
| 183 | 4-(2,4-dimethyl-1,3-thiazol-5-yl)-N-(3-methoxyphenyl)pyrimidine-2- | 2.64* |
| | amine | |
| 184 | 4-(2,4-dimethyl-1,3-thiazol-5-yl)-N-(3-nitrophenyl)pyrimidine-2- | 2.61** |
| | amine | |
| 185 | 4-(2,4-dimethyl-1,3-thiazol-5-yl)-N-(4-fluoro-3- | 3.07* |
| | methylphenyl)pyrimidine-2-amine | |
| 186 | 4-(2,4-dimethyl-1,3-thiazol-5-yl)-N-(4-fluorophenyl)pyrimidine-2- | 2.65* |
| | amine | |
| 187 | 4-(2,4-dimethyl-1,3-thiazol-5-yl)-N-(6-methoxypyridin-3- | 2.09* |
| | yl)pyrimidine-2-amine | |
| 188 | 4-(2,4-dimethyl-1,3-thiazol-5-yl)-N-[3- | 3.3** |
| | (trifluoromethyl)phenyl]pyrimidine-2-amine | |
| 189 | 4-(2,4-dimethyl-1,3-thiazol-5-yl)-N-[4- | 3.56* |
| | (trifluoromethyl)phenyl]pyrimidine-2-amine | |
| 190 | 4-(2,4-dimethyl-1,3-thiazol-5-yl)-N-phenylpyrimidine-2-amine | 2.4** |
| 191 | 4-(2-amino-1,3-thiazol-5-yl)-N-(2,5-dimethoxyphenyl)pyrimidine-2- | |
| | amine | |
| 192 | 4-(2-amino-1,3-thiazol-5-yl)-N-(3,4,5-trimethoxyphenyl)pyrimidine- | |
| | 2-amine | |
| 193 | 4-(2-amino-1,3-thiazol-5-yl)-N-(3-chlorophenyl)pyrimidine-2-amine | |
| 194 | 4-(2-amino-1,3-thiazol-5-yl)-N-(3-methoxyphenyl)pyrimidine-2- | |
| | amine | |
| 195 | 4-(2-amino-4-cyclopropyl-1,3-thiazol-5-yl)-N-(2,5- | |
| | dimethoxyphenyl)pyrimidine-2-amine | |
| 196 | 4-(2-amino-4-cyclopropyl-1,3-thiazol-5-yl)-N-(3,4,5- | |
| | trimethoxyphenyl)pyrimidine-2-amine | |
| 197 | 4-(2-amino-4-cyclopropyl-1,3-thiazol-5-yl)-N-(3- | |
| | chlorophenyl)pyrimidine-2-amine | |
| 198 | 4-(2-amino-4-cyclopropyl-1,3-thiazol-5-yl)-N-(3- | |
| | methoxyphenyl)pyrimidine-2-amine | |
| 199 | 4-(2-amino-4-cyclopropyl-1,3-thiazol-5-yl)-N-phenylpyrimidine-2- | |
| | amine | |
| 200 | 4-(2-amino-4-methyl-1,3-thiazol-5-yl)-N-(2,5- | |
| | dimethoxyphenyl)pyrimidine-2-amine | |
| 201 | 4-(2-amino-4-methyl-1,3-thiazol-5-yl)-N-(3,4,5- | 1.51* |
| | trimethoxyphenyl)pyrimidine-2-amine | |
| 202 | 4-(2-amino-4-methyl-1,3-thiazol-5-yl)-N-(3,4- | 1.28* |
| | dimethoxyphenyl)pyrimidine-2-amine | |
| 203 | 4-(2-amino-4-methyl-1,3-thiazol-5-yl)-N-(3-chloro-4- | 1.63* |
| | methoxyphenyl)pyrimidine-2-amine | |
| 204 | 4-(2-amino-4-methyl-1,3-thiazol-5-yl)-N-(3-chlorophenyl)pyrimidine- | 1.78* |
| | 2-amine | |
| 205 | 4-(2-amino-4-methyl-1,3-thiazol-5-yl)-N-(3- | |
| | methoxyphenyl)pyrimidine-2-amine | |
| 206 | 4-(2-amino-4-methyl-1,3-thiazol-5-yl)-N-(3-nitrophenyl)pyrimidine-2- | 1.5** |
| | amine | |
| 207 | 4-(2-amino-4-methyl-1,3-thiazol-5-yl)-N-(4-fluoro-3- | 1.68* |
| | methylphenyl)pyrimidine-2-amine | |
| 208 | 4-(2-amino-4-methyl-1,3-thiazol-5-yl)-N-(4-fluorophenyl)pyrimidine- | 1.56* |
| | 2-amine | |
| 209 | 4-(2-amino-4-methyl-1,3-thiazol-5-yl)-N-[3- | 1.97* |
| | (trifluoromethyl)phenyl]pyrimidine-2-amine | |
| 210 | 4-(2-amino-4-methyl-1,3-thiazol-5-yl)-N-phenylpyrimidine-2-amine | 1.51* |
| 211 | 4-(2-amino-4-sec-butyl-1,3-thiazol-5-yl)-N-(3,4,5- | |
| | trimethoxyphenyl)pyrimidine-2-amine | |
| 212 | 4-(2-amino-4-sec-butyl-1,3-thiazol-5-yl)-N-(3- | |
| | chlorophenyl)pyrimidine-2-amine | |
| 213 | 4-(2-amino-4-sec-butyl-1,3-thiazol-5-yl)-N-(3- | |
| | methoxyphenyl)pyrimidine-2-amine | |
| 214 | 4-(2-amino-4-sec-butyl-1,3-thiazol-5-yl)-N-phenylpyrimidine-2-amine | |
| 215 | 4-(2-cyclohexyl-4-methyl-1,3-thiazol-5-yl)-N-(3,4,5- | 3.97* |
| | trimethoxyphenyl)pyrimidine-2-amine | |
| 216 | 4-(2-cyclohexyl-4-methyl-1,3-thiazol-5-yl)-N-(3- | 4.63* |
| | nitrophenyl)pyrimidine-2-amine | |
| 217 | 4-(2-cyclohexyl-4-methyl-1,3-thiazol-5-yl)-N-(6-methoxypyridin-3- | 3.86* |
| | yl)pyrimidine-2-amine | |
| 218 | 4-(2-cyclohexyl-4-methyl-1,3-thiazol-5-yl)-N-[4- | 5.62* |
| | (trifluoromethyl)phenyl]pyrimidine-2-amine | |
| 219 | 4-(2-cyclohexyl-4-methyl-1,3-thiazol-5-yl)-N-phenylpyrimidine-2- | 4.65* |
| | amine | |
| 220 | 4-(2-cyclopropyl-4-methyl-1,3-thiazol-5-yl)-N-(2,3-dihydro-1,4- | 2.91* |
| | benzodioxin-6-yl)pyrimidine-2-amine | |
| 221 | 4-(2-cyclopropyl-4-methyl-1,3-thiazol-5-yl)-N-(2,5- | 3.71* |
| | dimethoxyphenyl)pyrimidine-2-amine | |
| 222 | 4-(2-cyclopropyl-4-methyl-1,3-thiazol-5-yl)-N-(3,4,5- | 2.77* |
| | trimethoxyphenyl)pyrimidine-2-amine | |
| 223 | 4-(2-cyclopropyl-4-methyl-1,3-thiazol-5-yl)-N-(3,4- | 2.6* |
| | dimethoxyphenyl)pyrimidine-2-amine | |
| 224 | 4-(2-cyclopropyl-4-methyl-1,3-thiazol-5-yl)-N-(3- | 3.28* |
| | methoxyphenyl)pyrimidine-2-amine | |
| 225 | 4-(2-cyclopropyl-4-methyl-1,3-thiazol-5-yl)-N-(3- | 3.29* |
| | nitrophenyl)pyrimidine-2-amine | |
| 226 | 4-(2-cyclopropyl-4-methyl-1,3-thiazol-5-yl)-N-(4-fluoro-3- | 3.71* |
| | methylphenyl)pyrimidine-2-amine | |
| 227 | 4-(2-cyclopropyl-4-methyl-1,3-thiazol-5-yl)-N-(4- | 3.31* |
| | fluorophenyl)pyrimidine-2-amine | |
| 228 | 4-(2-cyclopropyl-4-methyl-1,3-thiazol-5-yl)-N-(6-methoxypyridin-3- | 2.53* |
| | yl)pyrimidine-2-amine | |
| 229 | 4-(2-cyclopropyl-4-methyl-1,3-thiazol-5-yl)-N-[3- | 4.04* |
| | (trifluoromethyl)phenyl]pyrimidine-2-amine | |
| 230 | 4-(2-cyclopropyl-4-methyl-1,3-thiazol-5-yl)-N-[4- | 4.2* |
| | (trifluoromethyl)phenyl]pyrimidine-2-amine | |
| 231 | 4-(2-cyclopropyl-4-methyl-1,3-thiazol-5-yl)-N-phenylpyrimidine-2- | 3.26* |
| | amine | |
| 232 | 4-(2-ethyl-1,3-thiazol-5-yl)-N-(3,4,5-trimethoxyphenyl)pyrimidine-2- | |
| | amine | |
| 233 | 4-(2-ethyl-1,3-thiazol-5-yl)-N-(3-methoxyphenyl)pyrimidine-2-amine | |
| 234 | 4-(2-ethyl-1,3-thiazol-5-yl)-N-phenylpyrimidine-2-amine | |
| 235 | 4-(2-ethyl-4-methyl-1,3-thiazol-5-yl)-N-(3,4,5- | 2.61* |
| | trimethoxyphenyl)pyrimidine-2-amine | |
| 236 | 4-(2-ethyl-4-methyl-1,3-thiazol-5-yl)-N-(3- | 3* |
| | methoxyphenyl)pyrimidine-2-amine | |
| 237 | 4-(2-ethyl-4-methyl-1,3-thiazol-5-yl)-N-(3-nitrophenyl)pyrimidine-2- | 3.14* |
| | amine | |
| 238 | 4-(2-ethyl-4-methyl-1,3-thiazol-5-yl)-N-(4-fluoro-3- | |
| | methylphenyl)pyrimidine-2-amine | |
| 239 | 4-(2-ethyl-4-methyl-1,3-thiazol-5-yl)-N-(4-fluorophenyl)pyrimidine-2- | 3.1* |
| | amine | |
| 240 | 4-(2-ethyl-4-methyl-1,3-thiazol-5-yl)-N-(6-methoxypyridin-3- | 2.33* |
| | yl)pyrimidine-2-amine | |
| 241 | 4-(2-ethyl-4-methyl-1,3-thiazol-5-yl)-N-[3- | 3.85* |
| | (trifluoromethyl)phenyl]pyrimidine-2-amine | |
| 242 | 4-(2-ethyl-4-methyl-1,3-thiazol-5-yl)-N-[4- | |
| | (trifluoromethyl)phenyl]pyrimidine-2-amine | |
| 243 | 4-(2-ethyl-4-methyl-1,3-thiazol-5-yl)-N-phenylpyrimidine-2-amine | 3.07* |
| 244 | 4-(2-isopropyl-4-methyl-1,3-thiazol-5-yl)-N-(3,4,5- | 2.99* |
| | trimethoxyphenyl)pyrimidine-2-amine | |
| 245 | 4-(2-isopropyl-4-methyl-1,3-thiazol-5-yl)-N-(3- | 3.5* |
| | methoxyphenyl)pyrimidine-2-amine | |
| 246 | 4-(2-isopropyl-4-methyl-1,3-thiazol-5-yl)-N-(3- | 3.55* |
| | nitrophenyl)pyrimidine-2-amine | |
| 247 | 4-(2-isopropyl-4-methyl-1,3-thiazol-5-yl)-N-(6-methoxypyridin-3- | 2.76* |
| | yl)pyrimidine-2-amine | |
| 248 | 4-(2-isopropyl-4-methyl-1,3-thiazol-5-yl)-N-[3- | 4.3* |
| | (trifluoromethyl)phenyl]pyrimidine-2-amine | |
| 249 | 4-(2-isopropyl-4-methyl-1,3-thiazol-5-yl)-N-[4-(4-methylpiperazin-1- | 1.71* |
| | yl)phenyl]pyrimidine-2-amine | |
| 250 | 4-(2-isopropyl-4-methyl-1,3-thiazol-5-yl)-N-[4- | |
| | (trifluoromethyl)phenyl]pyrimidine-2-amine | |
| 251 | 4-(2-isopropyl-4-methyl-1,3-thiazol-5-yl)-N-phenylpyrimidine-2- | 3.55* |
| | amine | |
| 252 | 4-(2-methyl-1,3-thiazol-5-yl)-N-(3,4,5-trimethoxyphenyl)pyrimidine- | |
| | 2-amine | |
| 253 | 4-(2-methyl-1,3-thiazol-5-yl)-N-phenylpyrimidine-2-amine | |
| 254 | 4-(4-chloro-2-methyl-1,3-thiazol-5-yl)-N-(3,4,5- | |
| | trimethoxyphenyl)pyrimidine-2-amine | |
| 255 | 4-(4-chloro-2-methyl-1,3-thiazol-5-yl)-N-(3-chlorophenyl)pyrimidine- | |
| | 2-amine | |
| 256 | 4-(4-chloro-2-methyl-1,3-thiazol-5-yl)-N-phenylpyrimidine-2-amine | |
| 257 | 4-(4-cyclopropyl-2-ethyl-1,3-thiazol-5-yl)-N-(2,5- | |
| | dimethoxyphenyl)pyrimidine-2-amine | |
| 258 | 4-(4-cyclopropyl-2-ethyl-1,3-thiazol-5-yl)-N-(3,4,5- | |
| | trimethoxyphenyl)pyrimidine-2-amine | |
| 259 | 4-(4-cyclopropyl-2-ethyl-1,3-thiazol-5-yl)-N-(3- | |
| | methoxyphenyl)pyrimidine-2-amine | |
| 260 | 4-(4-cyclopropyl-2-ethyl-1,3-thiazol-5-yl)-N-phenylpyrimidine-2- | |
| | amine | |
| 261 | 4-(4-cyclopropyl-2-methyl-1,3-thiazol-5-yl)-N-(2,5- | |
| | dimethoxyphenyl)pyrimidine-2-amine | |
| 262 | 4-(4-cyclopropyl-2-methyl-1,3-thiazol-5-yl)-N-(3,4,5- | |
| | trimethoxyphenyl)pyrimidine-2-amine | |
| 263 | 4-(4-cyclopropyl-2-methyl-1,3-thiazol-5-yl)-N-(3- | |
| | methoxyphenyl)pyrimidine-2-amine | |
| 264 | 4-(4-cyclopropyl-2-methyl-1,3-thiazol-5-yl)-N-phenylpyrimidine-2- | |
| | amine | |
| 265 | 4-(4-methoxy-2-methyl-1,3-thiazol-5-yl)-N-(3,4,5- | |
| | trimethoxyphenyl)pyrimidine-2-amine | |
| 266 | 4-(4-methoxy-2-methyl-1,3-thiazol-5-yl)-N-phenylpyrimidine-2-amine | |
| 267 | 4-(4-methyl-1,3-thiazol-5-yl)-N-[3- | 3.14* |
| | (trifluoromethyl)phenyl]pyrimidine-2-amine | |
| 268 | 4-(4-methyl-2-phenyl-1,3-thiazol-5-yl)-N-(3,4,5- | |
| | trimethoxyphenyl)pyrimidine-2-amine | |
| 269 | 4-(4-methyl-2-phenyl-1,3-thiazol-5-yl)-N-(3-nitrophenyl)pyrimidine- | |
| | 2-amine | |
| 270 | 4-(4-methyl-2-phenyl-1,3-thiazol-5-yl)-N-phenylpyrimidine-2-amine | |
| 271 | 4-(4-sec-butyl-2-ethyl-1,3-thiazol-5-yl)-N-(2,5- | |
| | dimethoxyphenyl)pyrimidine-2-amine | |
| 272 | 4-(4-sec-butyl-2-ethyl-1,3-thiazol-5-yl)-N-(3,4,5- | |
| | trimethoxyphenyl)pyrimidine-2-amine | |
| 273 | 4-(4-sec-butyl-2-ethyl-1,3-thiazol-5-yl)-N-(3- | |
| | chlorophenyl)pyrimidine-2-amine | |
| 274 | 4-(4-sec-butyl-2-ethyl-1,3-thiazol-5-yl)-N-(3- | |
| | methoxyphenyl)pyrimidine-2-amine | |
| 275 | 4-(4-sec-butyl-2-ethyl-1,3-thiazol-5-yl)-N-phenylpyrimidine-2-amine | |
| 276 | 4-(4-sec-butyl-2-methyl-1,3-thiazol-5-yl)-N-(2,5- | |
| | dimethoxyphenyl)pyrimidine-2-amine | |
| 277 | 4-(4-sec-butyl-2-methyl-1,3-thiazol-5-yl)-N-(3,4,5- | |
| | trimethoxyphenyl)pyrimidine-2-amine | |
| 278 | 4-(4-sec-butyl-2-methyl-1,3-thiazol-5-yl)-N-(3- | |
| | chlorophenyl)pyrimidine-2-amine | |
| 279 | 4-(4-sec-butyl-2-methyl-1,3-thiazol-5-yl)-N-(3- | |
| | methoxyphenyl)pyrimidine-2-amine | |
| 280 | 4-(4-sec-butyl-2-methyl-1,3-thiazol-5-yl)-N-phenylpyrimidine-2- | |
| | amine | |
| 281 | 4-[(2-methyl-4,5-dihydro[1,3]thiazole[4,5-h]quinazolin-8- | 1.41* |
| | yl)amino]phenol | |
| 282 | 4-[(2-methyl-4,5-dihydro[1,3]thiazole[4,5-h]quinazolin-8- | |
| | yl)amino]phenol | |
| 283 | 4-[(4-{2-[(2-hydroxy-1-methylethyl)amino]-4-methyl-1,3-thiazol-5- | |
| | yl}pyrimidin-2-yl)amino]phenol | |
| 284 | 4-[(4-{2-[(2-methoxy-1-methylethyl)amino]-4-methyl-1,3-thiazol-5- | 1.4* |
| | yl}pyrimidin-2-yl)amino]phenol | |
| 285 | 4-[(4-{4-methyl-2-[(tetrahydrofuran-2-ylmethyl)amino]-1,3-thiazol-5- | |
| | yl}pyrimidin-2-yl)amino]phenol | |
| 286 | 4-[2-(cyclopropylamine)-4-methyl-1,3-thiazol-5-yl]-N-(3,4,5- | |
| | trimethoxyphenyl)pyrimidine-2-amine | |
| 287 | 4-[2-(cyclopropylamine)-4-methyl-1,3-thiazol-5-yl]-N-(3- | |
| | methoxyphenyl)pyrimidine-2-amine | |
| 288 | 4-[2-(cyclopropylamine)-4-methyl-1,3-thiazol-5-yl]-N-(3- | |
| | nitrophenyl)pyrimidine-2-amine | |
| 289 | 4-[2-(cyclopropylamine)-4-methyl-1,3-thiazol-5-yl]-N-(4-fluoro-3- | |
| | methylphenyl)pyrimidine-2-amine | |
| 290 | 4-[2-(cyclopropylamine)-4-methyl-1,3-thiazol-5-yl]-N- | 1.96* |
| | phenylpyrimidine-2-amine | |
| 291 | 4-[2-(dimethylamine)-4-(trifluoromethyl)-1,3-thiazol-5-yl]-N-(3,4,5- | 3.43* |
| | trimethoxyphenyl)pyrimidine-2-amine | |
| 292 | 4-[2-(dimethylamine)-4-(trifluoromethyl)-1,3-thiazol-5-yl]-N- | 3.88* |
| | phenylpyrimidine-2-amine | |
| 293 | 4-[2-(dimethylamine)-4-methyl-1,3-thiazol-5-yl]-N-(3- | 1.98* |
| | methoxyphenyl)pyrimidine-2-amine | |
| 294 | 4-[2-(dimethylamine)-4-methyl-1,3-thiazol-5-yl]-N-(4-fluoro-3- | 2.22* |
| | methylphenyl)pyrimidine-2-amine | |
| 295 | 4-[2-(methoxymethyl)-4-methyl-1,3-thiazol-5-yl]-N-(3,4,5- | 2.36* |
| | trimethoxyphenyl)pyrimidine-2-amine | |
| 296 | 4-[2-(methoxymethyl)-4-methyl-1,3-thiazol-5-yl]-N-(3- | 2.67* |
| | methoxyphenyl)pyrimidine-2-amine | |
| 297 | 4-[2-(methoxymethyl)-4-methyl-1,3-thiazol-5-yl]-N-(3- | |
| | nitrophenyl)pyrimidine-2-amine | |
| 298 | 4-[2-(methoxymethyl)-4-methyl-1,3-thiazol-5-yl]-N-(6- | 2.13* |
| | methoxypyridin-3-yl)pyrimidine-2-amine | |
| 299 | 4-[2-(methoxymethyl)-4-methyl-1,3-thiazol-5-yl]-N-[3- | 3.41* |
| | (trifluoromethyl)phenyl]pyrimidine-2-amine | |
| 300 | 4-[2-(methoxymethyl)-4-methyl-1,3-thiazol-5-yl]-N-[4- | 3.59* |
| | (trifluoromethyl)phenyl]pyrimidine-2-amine | |
| 301 | 4-[2-(methoxymethyl)-4-methyl-1,3-thiazol-5-yl]-N- | 3.01* |
| | phenylpyrimidine-2-amine | |
| 302 | 4-[2-(tert-butylamine)-4-methyl-1,3-thiazol-5-yl]-N-(3,4,5- | |
| | trimethoxyphenyl)pyrimidine-2-amine | |
| 303 | 4-[2-(tert-butylamine)-4-methyl-1,3-thiazol-5-yl]-N-(3- | 2.99* |
| | chlorophenyl)pyrimidine-2-amine | |
| 304 | 4-[2-(tert-butylamine)-4-methyl-1,3-thiazol-5-yl]-N-(3- | 2.4* |
| | methoxyphenyl)pyrimidine-2-amine | |
| 305 | 4-[2-(tert-butylamine)-4-methyl-1,3-thiazol-5-yl]-N-(3- | 2.63* |
| | nitrophenyl)pyrimidine-2-amine | |
| 306 | 4-[2-(tert-butylamine)-4-methyl-1,3-thiazol-5-yl]-N-(4-fluoro-3- | 2.7* |
| | methylphenyl)pyrimidine-2-amine | |
| 307 | 4-[2-(tert-butylamine)-4-methyl-1,3-thiazol-5-yl]-N-(4- | 2.54* |
| | fluorophenyl)pyrimidine-2-amine | |
| 308 | 4-[2-(tert-butylamine)-4-methyl-1,3-thiazol-5-yl]-N-(6- | 2.04* |
| | methoxypyridin-3-yl)pyrimidine-2-amine | |
| 309 | 4-[2-(tert-butylamine)-4-methyl-1,3-thiazol-5-yl]-N- | 2.42* |
| | phenylpyrimidine-2-amine | |
| 310 | 4-[2-methyl-4-(trifluoromethyl)-1,3-thiazol-5-yl]-N-(3,4,5- | 3.02* |
| | trimethoxyphenyl)pyrimidine-2-amine | |
| 311 | 4-[2-methyl-4-(trifluoromethyl)-1,3-thiazol-5-yl]-N-(3- | 3.5* |
| | nitrophenyl)pyrimidine-2-amine | |
| 312 | 4-[2-methyl-4-(trifluoromethyl)-1,3-thiazol-5-yl]-N- | 3.45* |
| | phenylpyrimidine-2-amine | |
| 313 | 4-[4-methyl-2-(methylamine)-1,3-thiazol-5-yl]-N-(3,4,5- | 1.53* |
| | trimethoxyphenyl)pyrimidine-2-amine | |
| 314 | 4-[4-methyl-2-(methylamine)-1,3-thiazol-5-yl]-N-(3- | |
| | nitrophenyl)pyrimidine-2-amine | |
| 315 | 4-[4-methyl-2-(methylamine)-1,3-thiazol-5-yl]-N-phenylpyrimidine-2- | 1.61* |
| | amine | |
| 316 | 4-[4-methyl-2-(methylthio)-1,3-thiazol-5-yl]-N-(3,4,5- | 2.76* |
| | trimethoxyphenyl)pyrimidine-2-amine | |
| 317 | 4-[4-methyl-2-(methylthio)-1,3-thiazol-5-yl]-N-[4- | 4.37* |
| | (trifluoromethyl)phenyl]pyrimidine-2-amine | |
| 318 | 4-[4-methyl-2-(methylthio)-1,3-thiazol-5-yl]-N-phenylpyrimidine-2- | 3.34* |
| | amine | |
| 319 | 4-{[2-(tert-butylamine)-4,5-dihydro[1,3]thiazole[4,5-h]quinazolin-8- | |
| | yl]amino}phenol | |
| 320 | 4-{[4-(2,4-dimethyl-1,3-thiazol-5-yl)pyrimidin-2- | |
| | yl]amino}benzonitrile | |
| 321 | 4-{[4-(2,4-dimethyl-1,3-thiazol-5-yl)pyrimidin-2-yl]amino}phenol | 1.62* |
| 322 | 4-{[4-(2-amino-4-methyl-1,3-thiazol-5-yl)pyrimidin-2- | |
| | yl]amino}benzonitrile | |
| 323 | 4-{[4-(2-amino-4-methyl-1,3-thiazol-5-yl)pyrimidin-2- | 0.93* |
| | yl]amino}phenol | |
| 324 | 4-{[4-(2-cyclopropyl-4-methyl-1,3-thiazol-5-yl)pyrimidin-2- | 2.01* |
| | yl]amino}phenol | |
| 325 | 4-{[4-(2-ethyl-4-methyl-1,3-thiazol-5-yl)pyrimidin-2- | 2.16* |
| | yl]amino}benzoic acid | |
| 326 | 4-{[4-(2-ethyl-4-methyl-1,3-thiazol-5-yl)pyrimidin-2- | |
| | yl]amino}benzonitrile | |
| 327 | 4-{[4-(2-ethyl-4-methyl-1,3-thiazol-5-yl)pyrimidin-2-yl]amino}phenol | 1.88* |
| 328 | 4-{[4-(2-isopropyl-4-methyl-1,3-thiazol-5-yl)pyrimidin-2- | |
| | yl]amino}benzonitrile | |
| 329 | 4-{[4-(2-isopropyl-4-methyl-1,3-thiazol-5-yl)pyrimidin-2- | 2.18* |
| | yl]amino}phenol | |
| 330 | 4-{2-[(2-methoxy-1-methylethyl)amino]-1,3-thiazol-5-yl}-N-(3,4,5- | |
| | trimethoxyphenyl)pyrimidine-2-amine | |
| 331 | 4-{2-[(2-methoxy-1-methylethyl)amino]-1,3-thiazol-5-yl}-N-(3- | |
| | methoxyphenyl)pyrimidine-2-amine | |
| 332 | 4-{2-[(2-methoxy-1-methylethyl)amino]-4-methyl-1,3-thiazol-5-yl}- | 1.85* |
| | N-(3,4,5-trimethoxyphenyl)pyrimidine-2-amine | |
| 333 | 4-{2-[(2-methoxy-1-methylethyl)amino]-4-methyl-1,3-thiazol-5-yl}- | 2.03* |
| | N-(3-methoxyphenyl)pyrimidine-2-amine | |
| 334 | 4-{2-[(2-methoxy-1-methylethyl)amino]-4-methyl-1,3-thiazol-5-yl}- | 2.2* |
| | N-(3-nitrophenyl)pyrimidine-2-amine | |
| 335 | 4-{2-[(2-methoxy-1-methylethyl)amino]-4-methyl-1,3-thiazol-5-yl}- | Jan 65 |
| | N-(6-methoxypyridin-3-yl)pyrimidine-2-amine | |
| 336 | 4-{2-[(2-methoxy-1-methylethyl)amino]-4-methyl-1,3-thiazol-5-yl}- | |
| | N-phenylpyrimidine-2-amine | |
| 337 | 4-{2-[(2-methoxyethyl)amino]-1,3-thiazol-5-yl}-N-(3,4,5- | |
| | trimethoxyphenyl)pyrimidine-2-amine | |
| 338 | 4-{2-[(2-methoxyethyl)amino]-1,3-thiazol-5-yl}-N-(3- | |
| | methoxyphenyl)pyrimidine-2-amine | |
| 339 | 4-{2-[(2-methoxyethyl)amino]-1,3-thiazol-5-yl}-N-phenylpyrimidine- | |
| | 2-amine | |
| 340 | 4-{2-[(tetrahydrofuran-2-ylmethyl)amino]-1,3-thiazol-5-yl}-N-(3,4,5- | |
| | trimethoxyphenyl)pyrimidine-2-amine | |
| 341 | 4-{2-[cyclopropyl(methyl)amino]-4-methyl-1,3-thiazol-5-yl}-N- | 2.43* |
| | phenylpyrimidine-2-amine | |
| 342 | 4-{4-cyclopropyl-2-[(2-methoxy-1-methylethyl)amino]-1,3-thiazol-5- | |
| | yl}-N-(2,5-dimethoxyphenyl)pyrimidine-2-amine | |
| 343 | 4-{4-cyclopropyl-2-[(2-methoxy-1-methylethyl)amino]-1,3-thiazol-5- | |
| | yl}-N-(3,4,5-trimethoxyphenyl)pyrimidine-2-amine | |
| 344 | 4-{4-cyclopropyl-2-[(2-methoxy-1-methylethyl)amino]-1,3-thiazol-5- | |
| | yl}-N-(3-methoxyphenyl)pyrimidine-2-amine | |
| 345 | 4-{4-cyclopropyl-2-[(2-methoxy-1-methylethyl)amino]-1,3-thiazol-5- | |
| | yl}-N-phenylpyrimidine-2-amine | |
| 346 | 4-{4-cyclopropyl-2-[(2-methoxyethyl)amino]-1,3-thiazol-5-yl}-N- | |
| | (2,5-dimethoxyphenyl)pyrimidine-2-amine | |
| 347 | 4-{4-cyclopropyl-2-[(2-methoxyethyl)amino]-1,3-thiazol-5-yl}-N- | |
| | (3,4,5-trimethoxyphenyl)pyrimidine-2-amine | |
| 348 | 4-{4-cyclopropyl-2-[(2-methoxyethyl)amino]-1,3-thiazol-5-yl}-N-(3- | |
| | methoxyphenyl)pyrimidine-2-amine | |
| 349 | 4-{4-cyclopropyl-2-[(2-methoxyethyl)amino]-1,3-thiazol-5-yl}-N- | |
| | phenylpyrimidine-2-amine | |
| 350 | 4-{4-cyclopropyl-2-[(tetrahydrofuran-2-ylmethyl)amino]-1,3-thiazol- | |
| | 5-yl}-N-(2,5-dimethoxyphenyl)pyrimidine-2-amine | |
| 351 | 4-{4-cyclopropyl-2-[(tetrahydrofuran-2-ylmethyl)amino]-1,3-thiazol- | |
| | 5-yl}-N-(3,4,5-trimethoxyphenyl)pyrimidine-2-amine | |
| 352 | 4-{4-cyclopropyl-2-[(tetrahydrofuran-2-ylmethyl)amino]-1,3-thiazol- | |
| | 5-yl}-N-(3-methoxyphenyl)pyrimidine-2-amine | |
| 353 | 4-{4-cyclopropyl-2-[(tetrahydrofuran-2-ylmethyl)amino]-1,3-thiazol- | |
| | 5-yl}-N-phenylpyrimidine-2-amine | |
| 354 | 4-{4-methyl-2-[(tetrahydrofuran-2-ylmethyl)amino]-1,3-thiazol-5-yl}- | 1.8* |
| | N-(3,4,5-trimethoxyphenyl)pyrimidine-2-amine | |
| 355 | 4-{4-methyl-2-[(tetrahydrofuran-2-ylmethyl)amino]-1,3-thiazol-5-yl}- | 2.13* |
| | N-(3-nitrophenyl)pyrimidine-2-amine | |
| 356 | 4-{4-methyl-2-[(tetrahydrofuran-2-ylmethyl)amino]-1,3-thiazol-5-yl}- | 1.87* |
| | N-phenylpyrimidine-2-amine | |
| 357 | 4-{4-methyl-2-[methyl(phenyl)amino]-1,3-thiazol-5-yl}-N- | 3.21* |
| | phenylpyrimidine-2-amine | |
| 358 | 4-{4-sec-butyl-2-[(2-methoxy-1-methylethyl)amino]-1,3-thiazol-5-yl}- | |
| | N-(2,5-dimethoxyphenyl)pyrimidine-2-amine | |
| 359 | 4-{4-sec-butyl-2-[(2-methoxy-1-methylethyl)amino]-1,3-thiazol-5-yl}- | |
| | N-(3,4,5-trimethoxyphenyl)pyrimidine-2-amine | |
| 360 | 4-{4-sec-butyl-2-[(2-methoxy-1-methylethyl)amino]-1,3-thiazol-5-yl}- | |
| | N-(3-chlorophenyl)pyrimidine-2-amine | |
| 361 | 4-{4-sec-butyl-2-[(2-methoxy-1-methylethyl)amino]-1,3-thiazol-5-yl}- | |
| | N-(3-methoxyphenyl)pyrimidine-2-amine | |
| 362 | 4-{4-sec-butyl-2-[(2-methoxy-1-methylethyl)amino]-1,3-thiazol-5-yl}- | |
| | N-phenylpyrimidine-2-amine | |
| 363 | 4-{4-sec-butyl-2-[(2-methoxyethyl)amino]-1,3-thiazol-5-yl}-N- | |
| | phenylpyrimidine-2-amine | |
| 364 | 4-{4-sec-butyl-2-[(tetrahydrofuran-2-ylmethyl)amino]-1,3-thiazol-5- | |
| | yl}-N-(2,5-dimethoxyphenyl)pyrimidine-2-amine | |
| 365 | 4-{4-sec-butyl-2-[(tetrahydrofuran-2-ylmethyl)amino]-1,3-thiazol-5- | |
| | yl}-N-(3,4,5-trimethoxyphenyl)pyrimidine-2-amine | |
| 366 | 4-{4-sec-butyl-2-[(tetrahydrofuran-2-ylmethyl)amino]-1,3-thiazol-5- | |
| | yl}-N-(3-chlorophenyl)pyrimidine-2-amine | |
| 367 | 4-{4-sec-butyl-2-[(tetrahydrofuran-2-ylmethyl)amino]-1,3-thiazol-5- | |
| | yl}-N-(3-methoxyphenyl)pyrimidine-2-amine | |
| 368 | 4-{4-sec-butyl-2-[(tetrahydrofuran-2-ylmethyl)amino]-1,3-thiazol-5- | |
| | yl}-N-phenylpyrimidine-2-amine | |
| 369 | 4-methyl-3-(4-methyl-5-{2-[(3,4,5- | |
| | trimethoxyphenyl)amino]pyrimidin-4-yl}-1,3-thiazol-2-yl)-1,3- | |
| | oxazolidin-2-one | |
| 370 | 4-methyl-3-(4-methyl-5-{2-[(3-nitrophenyl)amino]pyrimidin-4-yl}- | |
| | 1,3-thiazol-2-yl)-1,3-oxazolidin-2-one | |
| 371 | 4-methyl-3-{8-[(3,4,5-trimethoxyphenyl)amino]-4,5- | |
| | dihydro[1,3]thiazole[4,5-h]quinazolin-2-yl}-1,3-oxazolidin-2-one | |
| 372 | 4-methyl-3-{8-[(3-nitrophenyl)amino]-4,5-dihydro[1,3]thiazole[4,5- | |
| | h]quinazolin-2-yl}-1,3-oxazolidin-2-one | |
| 373 | 5-(2-anilinopyrimidin-4-yl)-N,N,4-trimethyl-1,3-thiazole-2- | 2.72* |
| | carboxamide | |
| 374 | ethyl-3-{[4-(2-cyclopropyl-4-methyl-1,3-thiazol-5-yl)pyrimidin-2- | 3.56* |
| | yl]amino}benzoate | |
| 375 | ethyl-3-{[4-(2-isopropyl-4-methyl-1,3-thiazol-5-yl)pyrimidin-2- | 3.83* |
| | yl]amino}benzoate | |
| 376 | ethyl-4-({4-[2-(methoxymethyl)-4-methyl-1,3-thiazol-5-yl]pyrimidin- | |
| | 2-yl}amino)benzoate | |
| 377 | ethyl-4-({4-[4-methyl-2-(methylamine)-1,3-thiazol-5-yl]pyrimidin-2- | |
| | yl}amino)benzoate | |
| 378 | ethyl-4-{[4-(2,4-dimethyl-1,3-thiazol-5-yl)pyrimidin-2- | |
| | yl]amino}benzoate | |
| 379 | ethyl-4-{[4-(2-ethyl-4-methyl-1,3-thiazol-5-yl)pyrimidin-2- | |
| | yl]amino}benzoate | |
| 380 | methyl-(4-{[4-(2,4-dimethyl-1,3-thiazol-5-yl)pyrimidin-2- | 1.84** |
| | yl]amino}phenyl)carbamate | |
| 381 | methyl-(4-{[4-(2-ethyl-4-methyl-1,3-thiazol-5-yl)pyrimidin-2- | |
| | yl]amino}phenyl)carbamate | |
| 382 | methyl-(4-{[4-(2-isopropyl-4-methyl-1,3-thiazol-5-yl)pyrimidin-2- | |
| | yl]amino}phenyl)carbamate | |
| 383 | methyl-[4-({4-[4-methyl-2-(methylamine)-1,3-thiazol-5-yl]pyrimidin- | |
| | 2-yl}amino)phenyl]carbamate | |
| 384 | methyl-{4-[(2-methyl-4,5-dihydro[1,3]thiazole[4,5-h]quinazolin-8- | 1.92* |
| | yl)amino]phenyl}carbamate | |
| 385 | N-(2,3-dihydro-1,4-benzodioxin-6-yl)-2-methyl-4,5- | 2.19* |
| | dihydro[1,3]thiazole[4,5-h]quinazoline-8-amine | |
| 386 | N-(2,3-dihydro-1,4-benzodioxin-6-yl)-2-methyl-4,5- | |
| | dihydro[1,3]thiazole[4,5-h]quinazoline-8-amine | |
| 387 | N-(2,3-dihydro-1,4-benzodioxin-6-yl)-4-(2,4-dimethyl-1,3-thiazol-5- | 2.34* |
| | yl)pyrimidine-2-amine | |
| 388 | N-(2,3-dihydro-1,4-benzodioxin-6-yl)-4-(2-ethyl-4-methyl-1,3-thiazol- | 2.7* |
| | 5-yl)pyrimidine-2-amine | |
| 389 | N-(2,3-dihydro-1,4-benzodioxin-6-yl)-4-(2-isopropyl-4-methyl-1,3- | 3.16* |
| | thiazol-5-yl)pyrimidine-2-amine | |
| 390 | N-(2,3-dihydro-1,4-benzodioxin-6-yl)-4-[2-(methoxymethyl)-4- | 2.49* |
| | methyl-1,3-thiazol-5-yl]pyrimidine-2-amine | |
| 391 | N-(2,5-dimethoxyphenyl)-4-(2,4-dimethyl-1,3-thiazol-5- | 3.03* |
| | yl)pyrimidine-2-amine | |
| 392 | N-(2,5-dimethoxyphenyl)-4-(2-ethyl-1,3-thiazol-5-yl)pyrimidine-2- | |
| | amine | |
| 393 | N-(2,5-dimethoxyphenyl)-4-(2-ethyl-4-methyl-1,3-thiazol-5- | |
| | yl)pyrimidine-2-amine | |
| 394 | N-(2,5-dimethoxyphenyl)-4-(2-isopropyl-4-methyl-1,3-thiazol-5- | |
| | yl)pyrimidine-2-amine | |
| 395 | N-(2,5-dimethoxyphenyl)-4-(2-methyl-1,3-thiazol-5-yl)pyrimidine-2- | |
| | amine | |
| 396 | N-(2,5-dimethoxyphenyl)-4-[2-(methoxymethyl)-4-methyl-1,3- | |
| | thiazol-5-yl]pyrimidine-2-amine | |
| 397 | N-(2,5-dimethoxyphenyl)-4-[4-methyl-2-(methylamine)-1,3-thiazol-5- | |
| | yl]pyrimidine-2-amine | |
| 398 | N-(2,5-dimethoxyphenyl)-4-{2-[(2-methoxy-1-methylethyl)amino]- | |
| | 1,3-thiazol-5-yl}pyrimidine-2-amine | |
| 399 | N-(2,5-dimethoxyphenyl)-4-{2-[(2-methoxyethyl)amino]-1,3-thiazol- | |
| | 5-yl}pyrimidine-2-amine | |
| 400 | N-(2,5-dimethoxyphenyl)-4-{2-[(tetrahydrofuran-2-ylmethyl)amino]- | |
| | 1,3-thiazol-5-yl}pyrimidine-2-amine | |
| 401 | N-(3,4-difluorophenyl)-4-(2-isopropyl-4-methyl-1,3-thiazol-5- | 3.95* |
| | yl)pyrimidine-2-amine | |
| 402 | N-(3,4-dimethoxyphenyl)-2-methyl-4,5-dihydro[1,3]thiazole[4,5- | 1.95* |
| | h]quinazoline-8-amine | |
| 403 | N-(3,4-dimethoxyphenyl)-4-(2,4-dimethyl-1,3-thiazol-5- | 2.16* |
| | yl)pyrimidine-2-amine | |
| 404 | N-(3,4-dimethoxyphenyl)-4-[2-(methoxymethyl)-4-methyl-1,3- | 2.23* |
| | thiazol-5-yl]pyrimidine-2-amine | |
| 405 | N-(3,4-dimethoxyphenyl)-4-[4-methyl-2-(methylthio)-1,3-thiazol-5- | 2.78* |
| | yl]pyrimidine-2-amine | |
| 406 | N-(3,5-dimethoxyphenyl)-4-(2,4-dimethyl-1,3-thiazol-5- | 2.64* |
| | yl)pyrimidine-2-amine | |
| 407 | N-(3,5-dimethoxyphenyl)-4-[4-methyl-2-(methylthio)-1,3-thiazol-5- | 3.39* |
| | yl]pyrimidine-2-amine | |
| 408 | N-(3-{[2-(2-oxo-1,3-oxazolidin-3-yl)-4,5-dihydro[1,3]thiazole[4,5- | |
| | h]quinazolin-8-yl]amino}phenyl)acetamide | |
| 409 | N-(3-{[2-(2-oxo-1,3-oxazolidin-3-yl)-4,5-dihydro[1,3]thiazole[4,5- | |
| | h]quinazolin-8-yl]amino}phenyl)methanesulphonamide | |
| 410 | N-(3-{[2-(4-methyl-2-oxo-1,3-oxazolidin-3-yl)-4,5- | |
| | dihydro[1,3]thiazole[4,5-h]quinazolin-8-yl]amino}phenyl)acetamide | |
| 411 | N-(3-{[2-(4-methyl-2-oxo-1,3-oxazolidin-3-yl)-4,5- | |
| | dihydro[1,3]thiazole[4,5-h]quinazolin-8- | |
| | yl]amino}phenyl)methanesulphonamide | |
| 412 | N-(3-{[2-(tert-butylamine)-4,5-dihydro[1,3]thiazole[4,5-h]quinazolin- | |
| | 8-yl]amino}phenyl)acetamide | |
| 413 | N-(3-{[2-(tert-butylamine)-4,5-dihydro[1,3]thiazole[4,5-h]quinazolin- | |
| | 8-yl]amino}phenyl)methanesulphonamide | |
| 414 | N-(3-{[4-(2-cyclopropyl-4-methyl-1,3-thiazol-5-yl)pyrimidin-2- | 1.96* |
| | yl]amino}benzyl)acetamide | |
| 415 | N-(3-{[4-(2-ethyl-4-methyl-1,3-thiazol-5-yl)pyrimidin-2- | 2.05* |
| | yl]amino}phenyl)acetamide | |
| 416 | N-(3-chloro-4-methoxyphenyl)-4-(2,4-dimethyl-1,3-thiazol-5- | 2.88* |
| | yl)pyrimidine-2-amine | |
| 417 | N-(3-chloro-4-methoxyphenyl)-4-(2-ethyl-4-methyl-1,3-thiazol-5- | |
| | yl)pyrimidine-2-amine | |
| 418 | N-(3-chloro-4-methoxyphenyl)-4-(2-isopropyl-4-methyl-1,3-thiazol-5- | |
| | yl)pyrimidine-2-amine | |
| 419 | N-(3-chloro-4-methoxyphenyl)-4-[2-(methoxymethyl)-4-methyl-1,3- | 2.91* |
| | thiazol-5-yl]pyrimidine-2-amine | |
| 420 | N-(3-chlorophenyl)-2-methyl-4,5-dihydro[1,3]thiazole[4,5- | 3.33* |
| | h]quinazoline-8-amine | |
| 421 | N-(3-chlorophenyl)-4-(2,4-dimethyl-1,3-thiazol-5-yl)pyrimidine-2- | 3.11** |
| | amine | |
| 422 | N-(3-chlorophenyl)-4-(2-cyclopropyl-4-methyl-1,3-thiazol-5- | 3.94* |
| | yl)pyrimidine-2-amine | |
| 423 | N-(3-chlorophenyl)-4-(2-ethyl-1,3-thiazol-5-yl)pyrimidine-2-amine | |
| 424 | N-(3-chlorophenyl)-4-(2-ethyl-4-methyl-1,3-thiazol-5-yl)pyrimidine- | 3.72* |
| | 2-amine | |
| 425 | N-(3-chlorophenyl)-4-(2-isopropyl-4-methyl-1,3-thiazol-5- | 4.25* |
| | yl)pyrimidine-2-amine | |
| 426 | N-(3-chlorophenyl)-4-(2-methyl-1,3-thiazol-5-yl)pyrimidine-2-amine | |
| 427 | N-(3-chlorophenyl)-4-(4-cyclopropyl-2-ethyl-1,3-thiazol-5- | |
| | yl)pyrimidine-2-amine | |
| 428 | N-(3-chlorophenyl)-4-(4-cyclopropyl-2-methyl-1,3-thiazol-5- | |
| | yl)pyrimidine-2-amine | |
| 429 | N-(3-chlorophenyl)-4-(4-methoxy-2-methyl-1,3-thiazol-5- | |
| | yl)pyrimidine-2-amine | |
| 430 | N-(3-chlorophenyl)-4-(4-methyl-2-phenyl-1,3-thiazol-5-yl)pyrimidine- | |
| | 2-amine | |
| 431 | N-(3-chlorophenyl)-4-[2-(cyclopropylamine)-4-methyl-1,3-thiazol-5- | |
| | yl]pyrimidine-2-amine | |
| 432 | N-(3-chlorophenyl)-4-[2-(methoxymethyl)-4-methyl-1,3-thiazol-5- | 3.24* |
| | yl]pyrimidine-2-amine | |
| 433 | N-(3-chlorophenyl)-4-[2-methyl-4-(trifluoromethyl)-1,3-thiazol-5- | 4.03* |
| | yl]pyrimidine-2-amine | |
| 434 | N-(3-chlorophenyl)-4-[4-methyl-2-(methylamine)-1,3-thiazol-5- | 2.03* |
| | yl]pyrimidine-2-amine | |
| 435 | N-(3-chlorophenyl)-4-[4-methyl-2-(methylthio)-1,3-thiazol-5- | 4.08* |
| | yl]pyrimidine-2-amine | |
| 436 | N-(3-chlorophenyl)-4-{2-[(2-methoxy-1-methylethyl)amino]-1,3- | |
| | thiazol-5-yl}pyrimidine-2-amine | |
| 437 | N-(3-chlorophenyl)-4-{2-[(2-methoxy-1-methylethyl)amino]-4- | 2.47* |
| | methyl-1,3-thiazol-5-yl}pyrimidine-2-amine | |
| 438 | N-(3-chlorophenyl)-4-{2-[(2-methoxyethyl)amino]-1,3-thiazol-5- | |
| | yl}pyrimidine-2-amine | |
| 439 | N-(3-chlorophenyl)-4-{2-[(tetrahydrofuran-2-ylmethyl)amino]-1,3- | |
| | thiazol-5-yl}pyrimidine-2-amine | |
| 440 | N-(3-chlorophenyl)-4-{4-cyclopropyl-2-[(2-methoxy-1- | |
| | methylethyl)amino]-1,3-thiazol-5-yl}pyrimidine-2-amine | |
| 441 | N-(3-chlorophenyl)-4-{4-cyclopropyl-2-[(2-methoxyethyl)amino]-1,3- | |
| | thiazol-5-yl}pyrimidine-2-amine | |
| 442 | N-(3-chlorophenyl)-4-{4-cyclopropyl-2-[(tetrahydrofuran-2- | |
| | ylmethyl)amino]-1,3-thiazol-5-yl}pyrimidine-2-amine | |
| 443 | N-(3-chlorophenyl)-4-{4-methyl-2-[(tetrahydrofuran-2- | 2.36* |
| | ylmethyl)amino]-1,3-thiazol-5-yl}pyrimidine-2-amine | |
| 444 | N-(3-methoxyphenyl)-2-methyl-4,5-dihydro[1,3]thiazole[4,5- | 2.59* |
| | h]quinazoline-8-amine | |
| 445 | N-(3-methoxyphenyl)-4-(2-methyl-1,3-thiazol-5-yl)pyrimidine-2- | |
| | amine | |
| 446 | N-(3-methoxyphenyl)-4-(4-methyl-2-phenyl-1,3-thiazol-5- | |
| | yl)pyrimidine-2-amine | |
| 447 | N-(3-methoxyphenyl)-4-[2-methyl-4-(trifluoromethyl)-1,3-thiazol-5- | |
| | yl]pyrimidine-2-amine | |
| 448 | N-(3-methoxyphenyl)-4-[4-methyl-2-(methylamine)-1,3-thiazol-5- | |
| | yl]pyrimidine-2-amine | |
| 449 | N-(3-methoxyphenyl)-4-[4-methyl-2-(methylthio)-1,3-thiazol-5- | 3.33* |
| | yl]pyrimidine-2-amine | |
| 450 | N-(3-methoxyphenyl)-4-{2-[(tetrahydrofuran-2-ylmethyl)amino]-1,3- | |
| | thiazol-5-yl}pyrimidine-2-amine | |
| 451 | N-(3-methoxyphenyl)-4-{4-methyl-2-[(tetrahydrofuran-2- | 1.94* |
| | ylmethyl)amino]-1,3-thiazol-5-yl}pyrimidine-2-amine | |
| 452 | N-(4-{[2-(2-oxo-1,3-oxazolidin-3-yl)-4,5-dihydro[1,3]thiazole[4,5- | |
| | h]quinazolin-8-yl]amino}phenyl)acetamide | |
| 453 | N-(4-{[2-(2-oxo-1,3-oxazolidin-3-yl)-4,5-dihydro[1,3]thiazole[4,5- | |
| | h]quinazolin-8-yl]amino}phenyl)methanesulphonamide | |
| 454 | N-(4-{[2-(4-methyl-2-oxo-1,3-oxazolidin-3-yl)-4,5- | |
| | dihydro[1,3]thiazole[4,5-h]quinazolin-8-yl]amino}phenyl)acetamide | |
| 455 | N-(4-{[2-(4-methyl-2-oxo-1,3-oxazolidin-3-yl)-4,5- | |
| | dihydro[1,3]thiazole[4,5-h]quinazolin-8- | |
| | yl]amino}phenyl)methanesulphonamide | |
| 456 | N-(4-{[2-(tert-butylamine)-4,5-dihydro[1,3]thiazole[4,5-h]quinazolin- | 1.72* |
| | 8-yl]amino}phenyl)acetamide | |
| 457 | N-(4-{[2-(tert-butylamine)-4,5-dihydro[1,3]thiazole[4,5-h]quinazolin- | |
| | 8-yl]amino}phenyl)methanesulphonamide | |
| 458 | N-(4-{[4-(2,4-dimethyl-1,3-thiazol-5-yl)pyrimidin-2- | 1.62* |
| | yl]amino}phenyl)acetamide | |
| 459 | N-(4-{[4-(2-cyclopropyl-4-methyl-1,3-thiazol-5-yl)pyrimidin-2- | 2.0* |
| | yl]amino}phenyl)acetamide | |
| 460 | N-(4-{[4-(2-ethyl-4-methyl-1,3-thiazol-5-yl)pyrimidin-2- | 1.88* |
| | yl]amino}phenyl)acetamide | |
| 461 | N-(4-{[4-(2-isopropyl-4-methyl-1,3-thiazol-5-yl)pyrimidin-2- | 2.15* |
| | yl]amino}phenyl)acetamide | |
| 462 | N-(4-chlorophenyl)-4-(2,4-dimethyl-1,3-thiazol-5-yl)pyrimidine-2- | 3.09** |
| | amine | |
| 463 | N-(4-chlorophenyl)-4-(2-ethyl-4-methyl-1,3-thiazol-5-yl)pyrimidine- | 3.72* |
| | 2-amine | |
| 464 | N-(4-chlorophenyl)-4-(2-isopropyl-4-methyl-1,3-thiazol-5- | 4.25* |
| | yl)pyrimidine-2-amine | |
| 465 | N-(4-chlorophenyl)-4-[2-(methoxymethyl)-4-methyl-1,3-thiazol-5- | 3.29* |
| | yl]pyrimidine-2-amine | |
| 466 | N-(4-chlorophenyl)-4-[4-methyl-2-(methylamine)-1,3-thiazol-5- | |
| | yl]pyrimidine-2-amine | |
| 467 | N-(4-cyclopropyl-5-{2-[(2,5-dimethoxyphenyl)amino]pyrimidin-4- | |
| | yl}-1,3-thiazol-2-yl)-2,2,2-trifluoroacetamide | |
| 468 | N-(4-cyclopropyl-5-{2-[(2,5-dimethoxyphenyl)amino]pyrimidin-4- | |
| | yl}-1,3-thiazol-2-yl)acetamide | |
| 469 | N-(4-cyclopropyl-5-{2-[(3,4,5-trimethoxyphenyl)amino]pyrimidin-4- | |
| | yl}-1,3-thiazol-2-yl)-2,2,2-trifluoroacetamide | |
| 470 | N-(4-cyclopropyl-5-{2-[(3,4,5-trimethoxyphenyl)amino]pyrimidin-4- | |
| | yl}-1,3-thiazol-2-yl)acetamide | |
| 471 | N-(4-cyclopropyl-5-{2-[(3-hydroxyphenyl)amino]pyrimidin-4-yl}- | |
| | 1,3-thiazol-2-yl)-2,2,2-trifluoroacetamide | |
| 472 | N-(4-cyclopropyl-5-{2-[(3-hydroxyphenyl)amino]pyrimidin-4-yl}- | |
| | 1,3-thiazol-2-yl)acetamide | |
| 473 | N-(4-cyclopropyl-5-{2-[(3-methoxyphenyl)amino]pyrimidin-4-yl}- | |
| | 1,3-thiazol-2-yl)-2,2,2-trifluoroacetamide | |
| 474 | N-(4-cyclopropyl-5-{2-[(3-methoxyphenyl)amino]pyrimidin-4-yl}- | |
| | 1,3-thiazol-2-yl)acetamide | |
| 475 | N-(4-fluoro-3-methylphenyl)-2-methyl-4,5-dihydro[1,3]thiazole[4,5- | 2.94* |
| | h]quinazoline-8-amine | |
| 476 | N-(4-fluoro-3-methylphenyl)-4-(2-isopropyl-4-methyl-1,3-thiazol-5- | |
| | yl)pyrimidine-2-amine | |
| 477 | N-(4-fluoro-3-methylphenyl)-4-(4-methyl-2-phenyl-1,3-thiazol-5- | |
| | yl)pyrimidine-2-amine | |
| 478 | N-(4-fluoro-3-methylphenyl)-4-[2-(methoxymethyl)-4-methyl-1,3- | 3.14* |
| | thiazol-5-yl]pyrimidine-2-amine | |
| 479 | N-(4-fluoro-3-methylphenyl)-4-[2-methyl-4-(trifluoromethyl)-1,3- | |
| | thiazol-5-yl]pyrimidine-2-amine | |
| 480 | N-(4-fluoro-3-methylphenyl)-4-[4-methyl-2-(methylamine)-1,3- | |
| | thiazol-5-yl]pyrimidine-2-amine | |
| 481 | N-(4-fluoro-3-methylphenyl)-4-[4-methyl-2-(methylthio)-1,3-thiazol- | 3.89* |
| | 5-yl]pyrimidine-2-amine | |
| 482 | N-(4-fluoro-3-methylphenyl)-4-{2-[(2-methoxy-1- | 2.19* |
| | methylethyl)amino]-4-methyl-1,3-thiazol-5-yl}pyrimidine-2-amine | |
| 483 | N-(4-fluoro-3-methylphenyl)-4-{4-methyl-2-[(tetrahydrofuran-2- | 2.03* |
| | ylmethyl)amino]-1,3-thiazol-5-yl}pyrimidine-2-amine | |
| 484 | N-(4-fluorophenyl)-2-methyl-4,5-dihydro[1,3]thiazole[4,5- | 2.62* |
| | h]quinazoline-8-amine | |
| 485 | N-(4-fluorophenyl)-4-(2-isopropyl-4-methyl-1,3-thiazol-5- | 3.58* |
| | yl)pyrimidine-2-amine | |
| 486 | N-(4-fluorophenyl)-4-[2-(methoxymethyl)-4-methyl-1,3-thiazol-5- | 2.8* |
| | yl]pyrimidine-2-amine | |
| 487 | N-(4-fluorophenyl)-4-[4-methyl-2-(methylamine)-1,3-thiazol-5- | |
| | yl]pyrimidine-2-amine | |
| 488 | N-(4-fluorophenyl)-4-{2-[(2-methoxy-1-methylethyl)amino]-4- | 2.0* |
| | methyl-1,3-thiazol-5-yl}pyrimidine-2-amine | |
| 489 | N-(4-fluorophenyl)-4-{4-methyl-2-[(tetrahydrofuran-2- | 2.1* |
| | ylmethyl)amino]-1,3-thiazol-5-yl}pyrimidine-2-amine | |
| 490 | N-(4-methyl-5-{2-[(3,4,5-trimethoxyphenyl)amino]pyrimidin-4-yl}- | |
| | 1,3-thiazol-2-yl)acetamide | |
| 491 | N-(4-methyl-5-{2-[(3-nitrophenyl)amino]pyrimidin-4-yl}-1,3-thiazol- | |
| | 2-yl)acetamide | |
| 492 | N-(4-sec-butyl-5-{2-[(2,5-dimethoxyphenyl)amino]pyrimidin-4-yl}- | |
| | 1,3-thiazol-2-yl)-2,2,2-trifluoroacetamide | |
| 493 | N-(4-sec-butyl-5-{2-[(2,5-dimethoxyphenyl)amino]pyrimidin-4-yl}- | |
| | 1,3-thiazol-2-yl)acetamide | |
| 494 | N-(4-sec-butyl-5-{2-[(3,4,5-trimethoxyphenyl)amino]pyrimidin-4-yl}- | |
| | 1,3-thiazol-2-yl)-2,2,2-trifluoroacetamide | |
| 495 | N-(4-sec-butyl-5-{2-[(3,4,5-trimethoxyphenyl)amino]pyrimidin-4-yl}- | |
| | 1,3-thiazol-2-yl)acetamide | |
| 496 | N-(4-sec-butyl-5-{2-[(3-chlorophenyl)amino]pyrimidin-4-yl}-1,3- | |
| | thiazol-2-yl)-2,2,2-trifluoroacetamide | |
| 497 | N-(4-sec-butyl-5-{2-[(3-chlorophenyl)amino]pyrimidin-4-yl}-1,3- | |
| | thiazol-2-yl)acetamide | |
| 498 | N-(4-sec-butyl-5-{2-[(3-hydroxyphenyl)amino]pyrimidin-4-yl}-1,3- | |
| | thiazol-2-yl)-2,2,2-trifluoroacetamide | |
| 499 | N-(4-sec-butyl-5-{2-[(3-hydroxyphenyl)amino]pyrimidin-4-yl}-1,3- | |
| | thiazol-2-yl)acetamide | |
| 500 | N-(4-sec-butyl-5-{2-[(3-methoxyphenyl)amino]pyrimidin-4-yl}-1,3- | |
| | thiazol-2-yl)-2,2,2-trifluoroacetamide | |
| 501 | N-(4-sec-butyl-5-{2-[(3-methoxyphenyl)amino]pyrimidin-4-yl}-1,3- | |
| | thiazol-2-yl)acetamide | |
| 502 | N-(5-{2-[(2,5-dimethoxyphenyl)amino]pyrimidin-4-yl}-1,3-thiazol-2- | |
| | yl)-2,2,2-trifluoroacetamide | |
| 503 | N-(5-{2-[(2,5-dimethoxyphenyl)amino]pyrimidin-4-yl}-1,3-thiazol-2- | |
| | yl)acetamide | |
| 504 | N-(5-{2-[(3,4,5-trimethoxyphenyl)amino]pyrimidin-4-yl}-1,3-thiazol- | |
| | 2-yl)acetamide | |
| 505 | N-(5-{2-[(3-acetylphenyl)amino]pyrimidin-4-yl}-1,3-thiazol-2-yl)- | |
| | 2,2,2-trifluoroacetamide | |
| 506 | N-(5-{2-[(3-acetylphenyl)amino]pyrimidin-4-yl}-1,3-thiazol-2- | |
| | yl)acetamide | |
| 507 | N-(5-{2-[(3-acetylphenyl)amino]pyrimidin-4-yl}-4-cyclopropyl-1,3- | |
| | thiazol-2-yl)-2,2,2-trifluoroacetamide | |
| 508 | N-(5-{2-[(3-acetylphenyl)amino]pyrimidin-4-yl}-4-cyclopropyl-1,3- | |
| | thiazol-2-yl)acetamide | |
| 509 | N-(5-{2-[(3-acetylphenyl)amino]pyrimidin-4-yl}-4-sec-butyl-1,3- | |
| | thiazol-2-yl)-2,2,2-trifluoroacetamide | |
| 510 | N-(5-{2-[(3-acetylphenyl)amino]pyrimidin-4-yl}-4-sec-butyl-1,3- | |
| | thiazol-2-yl)acetamide | |
| 511 | N-(5-{2-[(3-chlorophenyl)amino]pyrimidin-4-yl}-1,3-thiazol-2-yl)- | |
| | 2,2,2-trifluoroacetamide | |
| 512 | N-(5-{2-[(3-chlorophenyl)amino]pyrimidin-4-yl}-1,3-thiazol-2- | |
| | yl)acetamide | |
| 513 | N-(5-{2-[(3-chlorophenyl)amino]pyrimidin-4-yl}-4-cyclopropyl-1,3- | |
| | thiazol-2-yl)-2,2,2-trifluoroacetamide | |
| 514 | N-(5-{2-[(3-chlorophenyl)amino]pyrimidin-4-yl}-4-cyclopropyl-1,3- | |
| | thiazol-2-yl)acetamide | |
| 515 | N-(5-{2-[(3-chlorophenyl)amino]pyrimidin-4-yl}-4-methyl-1,3- | 2.49** |
| | thiazol-2-yl)acetamide | |
| 516 | N-(5-{2-[(3-hydroxyphenyl)amino]pyrimidin-4-yl}-1,3-thiazol-2- | |
| | yl)acetamide | |
| 517 | N-(5-{2-[(3-hydroxyphenyl)amino]pyrimidin-4-yl}-4-methyl-1,3- | |
| | thiazol-2-yl)acetamide | |
| 518 | N-(5-{2-[(3-methoxyphenyl)amino]pyrimidin-4-yl}-1,3-thiazol-2- | |
| | yl)acetamide | |
| 519 | N-(5-{2-[(3-methoxyphenyl)amino]pyrimidin-4-yl}-4-methyl-1,3- | |
| | thiazol-2-yl)acetamide | |
| 520 | N-(5-{2-[(4-fluoro-3-methylphenyl)amino]pyrimidin-4-yl}-4-methyl- | |
| | 1,3-thiazol-2-yl)acetamide | |
| 521 | N-(6-methoxypyridin-3-yl)-2-methyl-4,5-dihydro[1,3]thiazole[4,5- | 2.01* |
| | h]quinazoline-8-amine | |
| 522 | N-(6-methoxypyridin-3-yl)-4-[4-methyl-2-(methylthio)-1,3-thiazol-5- | 2.67* |
| | yl]pyrimidine-2-amine | |
| 523 | N-(6-methoxypyridin-3-yl)-4-{4-methyl-2-[(tetrahydrofuran-2- | 1.57* |
| | ylmethyl)amino]-1,3-thiazol-5-yl}pyrimidine-2-amine | |
| 524 | N,N-dimethyl-N′-{4-[4-methyl-2-(methylthio)-1,3-thiazol-5- | 1.75* |
| | yl]pyrimidin-2-yl}benzene-1,4-diamine | |
| 525 | N-[3-({2-[(2-hydroxy-1-methylethyl)amino]-4,5- | |
| | dihydro[1,3]thiazole[4,5-h]quinazolin-8-yl}amino)phenyl]acetamide | |
| 526 | N-[3-({2-[(2-hydroxy-1-methylethyl)amino]-4,5- | |
| | dihydro[1,3]thiazole[4,5-h]quinazolin-8- | |
| | yl}amino)phenyl]methanesulphonamide | |
| 527 | N-[3-({2-[(2-methoxy-1-methylethyl)amino]-4,5- | |
| | dihydro[1,3]thiazole[4,5-h]quinazolin-8-yl}amino)phenyl]acetamide | |
| 528 | N-[3-({2-[(2-methoxy-1-methylethyl)amino]-4,5- | |
| | dihydro[1,3]thiazole[4,5-h]quinazolin-8- | |
| | yl}amino)phenyl]methanesulphonamide | |
| 529 | N-[3-({2-[(tetrahydrofuran-2-ylmethyl)amino]-4,5- | |
| | dihydro[1,3]thiazole[4,5-h]quinazolin-8-yl}amino)phenyl]acetamide | |
| 530 | N-[3-({2-[(tetrahydrofuran-2-ylmethyl)amino]-4,5- | |
| | dihydro[1,3]thiazole[4,5-h]quinazolin-8- | |
| | yl}amino)phenyl]methanesulphonamide | |
| 531 | N-[3-({4-[2-(tert-butylamine)-4-methyl-1,3-thiazol-5-yl]pyrimidin-2- | 1.57* |
| | yl}amino)phenyl]acetamide | |
| 532 | N-[3-({4-[2-(tert-butylamine)-4-methyl-1,3-thiazol-5-yl]pyrimidin-2- | 1.57* |
| | yl}amino)phenyl]methanesulphonamide | |
| 533 | N-[3-({4-[4-methyl-2-(2-oxo-1,3-oxazolidin-3-yl)-1,3-thiazol-5- | |
| | yl]pyrimidin-2-yl}amino)phenyl]acetamide | |
| 534 | N-[3-({4-[4-methyl-2-(2-oxo-1,3-oxazolidin-3-yl)-1,3-thiazol-5- | |
| | yl]pyrimidin-2-yl}amino)phenyl]methanesulphonamide | |
| 535 | N-[3-({4-[4-methyl-2-(4-methyl-2-oxo-1,3-oxazolidin-3-yl)-1,3- | |
| | thiazol-5-yl]pyrimidin-2-yl}amino)phenyl]acetamide | |
| 536 | N-[3-({4-[4-methyl-2-(4-methyl-2-oxo-1,3-oxazolidin-3-yl)-1,3- | |
| | thiazol-5-yl]pyrimidin-2-yl}amino)phenyl]methanesulphonamide | |
| 537 | N-[3-(aminomethyl)phenyl]-4-(2-cyclopropyl-4-methyl-1,3-thiazol-5- | 1.45* |
| | yl)pyrimidine-2-amine | |
| 538 | N-[4-({2-[(2-hydroxy-1-methylethyl)amino]-4,5- | |
| | dihydro[1,3]thiazole[4,5-h]quinazolin-8-yl}amino)phenyl]acetamide | |
| 539 | N-[4-({2-[(2-hydroxy-1-methylethyl)amino]-4,5- | |
| | dihydro[1,3]thiazole[4,5-h]quinazolin-8- | |
| | yl}amino)phenyl]methanesulphonamide | |
| 540 | N-[4-({2-[(2-methoxy-1-methylethyl)amino]-4,5- | |
| | dihydro[1,3]thiazole[4,5-h]quinazolin-8-yl}amino)phenyl]acetamide | |
| 541 | N-[4-({2-[(2-methoxy-1-methylethyl)amino]-4,5- | |
| | dihydro[1,3]thiazole[4,5-h]quinazolin-8- | |
| | yl}amino)phenyl]methanesulphonamide | |
| 542 | N-[4-({2-[(tetrahydrofuran-2-ylmethyl)amino]-4,5- | |
| | dihydro[1,3]thiazole[4,5-h]quinazolin-8-yl}amino)phenyl]acetamide | |
| 543 | N-[4-({2-[(tetrahydrofuran-2-ylmethyl)amino]-4,5- | |
| | dihydro[1,3]thiazole[4,5-h]quinazolin-8- | |
| | yl}amino)phenyl]methanesulphonamide | |
| 544 | N-[4-({4-[2-(methoxymethyl)-4-methyl-1,3-thiazol-5-yl]pyrimidin-2- | 1.8* |
| | yl}amino)phenyl]acetamide | |
| 545 | N-[4-({4-[2-(tert-butylamine)-4-methyl-1,3-thiazol-5-yl]pyrimidin-2- | 1.75* |
| | yl}amino)phenyl]acetamide | |
| 546 | N-[4-({4-[2-(tert-butylamine)-4-methyl-1,3-thiazol-5-yl]pyrimidin-2- | 1.83* |
| | yl}amino)phenyl]methanesulphonamide | |
| 547 | N-[4-({4-[4-methyl-2-(2-oxo-1,3-oxazolidin-3-yl)-1,3-thiazol-5- | |
| | yl]pyrimidin-2-yl}amino)phenyl]acetamide | |
| 548 | N-[4-({4-[4-methyl-2-(2-oxo-1,3-oxazolidin-3-yl)-1,3-thiazol-5- | |
| | yl]pyrimidin-2-yl}amino)phenyl]methanesulphonamide | |
| 549 | N-[4-({4-[4-methyl-2-(4-methyl-2-oxo-1,3-oxazolidin-3-yl)-1,3- | |
| | thiazol-5-yl]pyrimidin-2-yl}amino)phenyl]acetamide | |
| 550 | N-[4-({4-[4-methyl-2-(4-methyl-2-oxo-1,3-oxazolidin-3-yl)-1,3- | |
| | thiazol-5-yl]pyrimidin-2-yl}amino)phenyl]methanesulphonamide | |
| 551 | N′-[4-(2,4-dimethyl-1,3-thiazol-5-yl)pyrimidin-2-yl]-N,N- | 1.75* |
| | dimethylbenzene-1,3-diamine | |
| 552 | N′-[4-(2,4-dimethyl-1,3-thiazol-5-yl)pyrimidin-2-yl]-N,N- | 1.41* |
| | dimethylbenzene-1,4-diamine | |
| 553 | N′-[4-(2-cyclohexyl-4-methyl-1,3-thiazol-5-yl)pyrimidin-2-yl]-N,N- | 2.38* |
| | dimethylbenzene-1,4-diamine | |
| 554 | N′-[4-(2-cyclopropyl-4-methyl-1,3-thiazol-5-yl)pyrimidin-2-yl]-N,N- | 2.19* |
| | dimethylbenzene-1,3-diamine | |
| 555 | N′-[4-(2-cyclopropyl-4-methyl-1,3-thiazol-5-yl)pyrimidin-2-yl]-N,N- | 1.63* |
| | dimethylbenzene-1,4-diamine | |
| 556 | N-[5-(2-anilinopyrimidin-4-yl)-1,3-thiazol-2-yl]-2,2,2- | |
| | trifluoroacetamide | |
| 557 | N-[5-(2-anilinopyrimidin-4-yl)-1,3-thiazol-2-yl]acetamide | |
| 558 | N-[5-(2-anilinopyrimidin-4-yl)-4-cyclopropyl-1,3-thiazol-2-yl]-2,2,2- | |
| | trifluoroacetamide | |
| 559 | N-[5-(2-anilinopyrimidin-4-yl)-4-cyclopropyl-1,3-thiazol-2- | |
| | yl]acetamide | |
| 560 | N-[5-(2-anilinopyrimidin-4-yl)-4-methyl-1,3-thiazol-2-yl]acetamide | 2.09* |
| 561 | N-[5-(2-anilinopyrimidin-4-yl)-4-sec-butyl-1,3-thiazol-2-yl]-2,2,2- | |
| | trifluoroacetamide | |
| 562 | N-[5-(2-anilinopyrimidin-4-yl)-4-sec-butyl-1,3-thiazol-2-yl]acetamide | |
| 563 | N-{3-[(2-amino-4,5-dihydro[1,3]thiazole[4,5-h]quinazolin-8- | |
| | yl)amino]phenyl}acetamide | |
| 564 | N-{3-[(2-amino-4,5-dihydro[1,3]thiazole[4,5-h]quinazolin-8- | |
| | yl)amino]phenyl}methanesulphonamide | |
| 565 | N-{3-[(2-methyl-4,5-dihydro[1,3]thiazole[4,5-h]quinazolin-8- | 1.71* |
| | yl)amino]phenyl}acetamide | |
| 566 | N-{3-[(2-methyl-4,5-dihydro[1,3]thiazole[4,5-h]quinazolin-8- | |
| | yl)amino]phenyl}methanesulphonamide | |
| 567 | N-{3-[(4-{2-[(2-hydroxy-1-methylethyl)amino]-4-methyl-1,3-thiazol- | |
| | 5-yl}pyrimidin-2-yl)amino]phenyl}acetamide | |
| 568 | N-{3-[(4-{2-[(2-hydroxy-1-methylethyl)amino]-4-methyl-1,3-thiazol- | |
| | 5-yl}pyrimidin-2-yl)amino]phenyl}methanesulphonamide | |
| 569 | N-{3-[(4-{2-[(2-methoxy-1-methylethyl)amino]-4-methyl-1,3-thiazol- | 1.47* |
| | 5-yl}pyrimidin-2-yl)amino]phenyl}acetamide | |
| 570 | N-{3-[(4-{2-[(2-methoxy-1-methylethyl)amino]-4-methyl-1,3-thiazol- | 1.57* |
| | 5-yl}pyrimidin-2-yl)amino]phenyl}methanesulphonamide | |
| 571 | N-{3-[(4-{4-methyl-2-[(tetrahydrofuran-2-ylmethyl)amino]-1,3- | |
| | thiazol-5-yl}pyrimidin-2-yl)amino]phenyl}acetamide | |
| 572 | N-{3-[(4-{4-methyl-2-[(tetrahydrofuran-2-ylmethyl)amino]-1,3- | |
| | thiazol-5-yl}pyrimidin-2-yl)amino]phenyl}methanesulphonamide | |
| 573 | N-{4-[(2-amino-4,5-dihydro[1,3]thiazole[4,5-h]quinazolin-8- | |
| | yl)amino]phenyl}methanesulphonamide | |
| 574 | N-{4-[(2-methyl-4,5-dihydro[1,3]thiazole[4,5-h]quinazolin-8- | 1.56* |
| | yl)amino]phenyl}acetamide | |
| 575 | N-{4-[(2-methyl-4,5-dihydro[1,3]thiazole[4,5-h]quinazolin-8- | 1 79* |
| | yl)amino]phenyl}methanesulphonamide | |
| 576 | N-{4-[(4-{2-[(2-hydroxy-1-methylethyl)amino]-4-methyl-1,3-thiazol- | |
| | 5-yl}pyrimidin-2-yl)amino]phenyl}acetamide | |
| 577 | N-{4-[(4-{2-[(2-hydroxy-1-methylethyl)amino]-4-methyl-1,3-thiazol- | |
| | 5-yl}pyrimidin-2-yl)amino]phenyl}methanesulphonamide | |
| 578 | N-{4-[(4-{2-[(2-methoxy-1-methylethyl)amino]-4-methyl-1,3-thiazol- | 1.43* |
| | 5-yl}pyrimidin-2-yl)amino]phenyl}acetamide | |
| 579 | N-{4-[(4-{2-[(2-methoxy-1-methylethyl)amino]-4-methyl-1,3-thiazol- | |
| | 5-yl}pyrimidin-2-yl)amino]phenyl}methanesulphonamide | |
| 580 | N-{4-[(4-{4-methyl-2-[(tetrahydrofuran-2-ylmethyl)amino]-1,3- | 1.39* |
| | thiazol-5-yl}pyrimidin-2-yl)amino]phenyl}acetamide | |
| 581 | N-{4-[(4-{4-methyl-2-[(tetrahydrofuran-2-ylmethyl)amino]-1,3- | |
| | thiazol-5-yl}pyrimidin-2-yl)amino]phenyl}methanesulphonamide | |
| 582 | N2-(2-methoxy-1-methylethyl)-N8-(3,4,5-trimethoxyphenyl)-4,5- | |
| | dihydro[1,3]thiazole[4,5-h]quinazoline-2,8-diamine | |
| 583 | N2-(2-methoxy-1-methylethyl)-N8-(3-methoxyphenyl)-4,5- | |
| | dihydro[1,3]thiazole[4,5-h]quinazoline-2,8-diamine | |
| 584 | N2-(2-methoxy-1-methylethyl)-N8-(3-nitrophenyl)-4,5- | |
| | dihydro[1,3]thiazole[4,5-h]quinazoline-2,8-diamine | |
| 585 | N2-(2-methoxy-1-methylethyl)-N8-(6-methoxypyridin-3-yl)-4,5- | |
| | dihydro[1,3]thiazole[4,5-h]quinazoline-2,8-diamine | |
| 586 | N2-(2-methoxy-1-methylethyl)-N8-phenyl-4,5- | |
| | dihydro[1,3]thiazole[4,5-h]quinazoline-2,8-diamine | |
| 587 | N2-(tert-butyl)-N8-(3,4,5-trimethoxyphenyl)-4,5- | |
| | dihydro[1,3]thiazole[4,5-h]quinazoline-2,8-diamine | |
| 588 | N2-(tert-butyl)-N8-(3-chlorophenyl)-4,5-dihydro[1,3]thiazole[4,5- | |
| | h]quinazoline-2,8-diamine | |
| 589 | N2-(tert-butyl)-N8-(3-methoxyphenyl)-4,5-dihydro[1,3]thiazole[4,5- | |
| | h]quinazoline-2,8-diamine | |
| 590 | N2-(tert-butyl)-N8-(3-nitrophenyl)-4,5-dihydro[1,3]thiazole[4,5- | |
| | h]quinazoline-2,8-diamine | |
| 591 | N2-(tert-butyl)-N8-(4-fluoro-3-methylphenyl)-4,5- | |
| | dihydro[1,3]thiazole[4,5-h]quinazoline-2,8-diamine | |
| 592 | N2-(tert-butyl)-N8-(4-fluorophenyl)-4,5-dihydro[1,3]thiazole[4,5- | |
| | h]quinazoline-2,8-diamine | |
| 593 | N2-(tert-butyl)-N8-(6-methoxypyridin-3-yl)-4,5- | |
| | dihydro[1,3]thiazole[4,5-h]quinazoline-2,8-diamine | |
| 594 | N2-(tert-butyl)-N8-phenyl-4,5-dihydro[1,3]thiazole[4,5- | |
| | h]quinazoline-2,8-diamine | |
| 595 | N2-(tetrahydrofuran-2-ylmethyl)-N8-(3,4,5-trimethoxyphenyl)-4,5- | |
| | dihydro[1,3]thiazole[4,5-h]quinazoline-2,8-diamine | |
| 596 | N4-[4-(1-acetyl-2,3-dihydro-1H-indol-5-yl)pyrimidin-2-yl]-2-chloro- | |
| | N1,N1-dimethylbenzene-1,4-diamine | |
| 597 | N8-(3,4,5-trimethoxyphenyl)-4,5-dihydro[1,3]thiazole[4,5- | |
| | h]quinazoline-2,8-diamine | |
| 598 | N8-(3-chlorophenyl)-4,5-dihydro[1,3]thiazole[4,5-h]quinazoline-2,8- | |
| | diamine | |
| 599 | N8-(3-chlorophenyl)-N2-(tetrahydrofuran-2-ylmethyl)-4,5- | |
| | dihydro[1,3]thiazole[4,5-h]quinazoline-2,8-diamine | |
| 600 | N8-(3-methoxyphenyl)-4,5-dihydro[1,3]thiazole[4,5-h]quinazoline- | |
| | 2,8-diamine | |
| 601 | N8-(3-methoxyphenyl)-N2-(tetrahydrofuran-2-ylmethyl)-4,5- | |
| | dihydro[1,3]thiazole[4,5-h]quinazoline-2,8-diamine | |
| 602 | N8-(3-nitrophenyl)-4,5-dihydro[1,3]thiazole[4,5-h]quinazoline-2,8- | |
| | diamine | |
| 603 | N8-(3-nitrophenyl)-N2-(tetrahydrofuran-2-ylmethyl)-4,5- | |
| | dihydro[1,3]thiazole[4,5-h]quinazoline-2,8-diamine | |
| 604 | N8-(4-fluoro-3-methylphenyl)-4,5-dihydro[1,3]thiazole[4,5- | |
| | h]quinazoline-2,8-diamine | |
| 605 | N8-(4-fluoro-3-methylphenyl)-N2-(2-methoxy-1-methylethyl)-4,5- | |
| | dihydro[1,3]thiazole[4,5-h]quinazoline-2,8-diamine | |
| 606 | N8-(4-fluoro-3-methylphenyl)-N2-(tetrahydrofuran-2-ylmethyl)-4,5- | |
| | dihydro[1,3]thiazole[4,5-h]quinazoline-2,8-diamine | |
| 607 | N8-(4-fluorophenyl)-4,5-dihydro[1,3]thiazole[4,5-h]quinazoline-2,8- | |
| | diamine | |
| 608 | N8-(4-fluorophenyl)-N2-(2-methoxy-1-methylethyl)-4,5- | |
| | dihydro[1,3]thiazole[4,5-h]quinazoline-2,8-diamine | |
| 609 | N8-(4-fluorophenyl)-N2-(tetrahydrofuran-2-ylmethyl)-4,5- | |
| | dihydro[1,3]thiazole[4,5-h]quinazoline-2,8-diamine | |
| 610 | N8-(4-fluorophenyl)-N2,N2-dimethyl-4,5-dihydro[1,3]thiazole[4,5- | 1.78* |
| | h]quinazoline-2,8-diamine | |
| 611 | N8-(6-methoxypyridin-3-yl)-4,5-dihydro[1,3]thiazole[4,5- | |
| | h]quinazoline-2,8-diamine | |
| 612 | N8-(6-methoxypyridin-3-yl)-N2-(tetrahydrofuran-2-ylmethyl)-4,5- | |
| | dihydro[1,3]thiazole[4,5-h]quinazoline-2,8-diamine | |
| 613 | N8-phenyl-4,5-dihydro[1,3]thiazole[4,5-h]quinazoline-2,8-diamine | |
| 614 | N8-phenyl-N2-(tetrahydrofuran-2-ylmethyl)-4,5- | |
| | dihydro[1,3]thiazole[4,5-h]quinazoline-2,8-diamine | |
| 615 | N-1,3-benzodioxol-5-yl-2-methyl-4,5-dihydro[1,3]thiazole[4,5- | 2.25* |
| | h]quinazoline-8-amine | |
| 616 | N-1,3-benzodioxol-5-yl-4-(2,4-dimethyl-1,3-thiazol-5-yl)pyrimidine- | 2.44* |
| | 2-amine | |
| 617 | N-1,3-benzodioxol-5-yl-4-(2-cyclopropyl-4-methyl-1,3-thiazol-5- | 2.96* |
| | yl)pyrimidine-2-amine | |
| 618 | N-1,3-benzodioxol-5-yl-4-(2-ethyl-4-methyl-1,3-thiazol-5- | 2.77* |
| | yl)pyrimidine-2-amine | |
| 619 | N-1,3-benzodioxol-5-yl-4-(2-isopropyl-4-methyl-1,3-thiazol-5- | 3.25* |
| | yl)pyrimidine-2-amine | |
| 620 | N-1,3-benzodioxol-5-yl-4-[2-(methoxymethyl)-4-methyl-1,3-thiazol- | 2.47* |
| | 5-yl]pyrimidine-2-amine | |
| 621 | N-acetyl-N-(3-{[4-(2-cyclopropyl-4-methyl-1,3-thiazol-5- | 1.87* |
| | yl)pyrimidin-2-yl]amino}benzyl)acetamide | |
| 622 | N-acetyl-N-(3-{acetyl[4-(2-cyclopropyl-4-methyl-1,3-thiazol-5- | 2.39* |
| | yl)pyrimidin-2-yl]amino}benzyl)acetamide | |
| 623 | N-phenyl-4-{2-[(tetrahydrofuran-2-ylmethyl)amino]-1,3-thiazol-5- | |
| | yl}pyrimidine-2-amine | |
| 624 | tert-butyl-(3-{[4-(2-cyclopropyl-4-methyl-1,3-thiazol-5-yl)pyrimidin- | 2.87* |
| | 2-yl]amino}benzyl)carbamate | |
| 625 | tert-butyl-(4-{[4-(2,4-dimethyl-1,3-thiazol-5-yl)pyrimidin-2- | 2.7** |
| | yl]amino}benzyl)carbamate | |
| 626 | tert-butyl-(4-{[4-(2-amino-1,3-thiazol-5-yl)pyrimidin-2- | |
| | yl]amino}benzyl)carbamate | |
| 627 | tert-butyl-(4-{[4-(2-amino-4-cyclopropyl-1,3-thiazol-5-yl)pyrimidin- | |
| | 2-yl]amino}benzyl)carbamate | |
| 628 | tert-butyl-(4-{[4-(2-amino-4-methyl-1,3-thiazol-5-yl)pyrimidin-2- | 1.86* |
| | yl]amino}benzyl)carbamate | |
| 629 | tert-butyl-(4-{[4-(2-amino-4-sec-butyl-1,3-thiazol-5-yl)pyrimidin-2- | |
| | yl]amino}benzyl)carbamate | |
| 630 | tert-butyl-(4-{[4-(2-cyclopropyl-4-methyl-1,3-thiazol-5-yl)pyrimidin- | 3.46* |
| | 2-yl]amino}benzyl)carbamate | |
| 631 | tert-butyl-(4-{[4-(2-ethyl-1,3-thiazol-5-yl)pyrimidin-2- | |
| | yl]amino}benzyl)carbamate | |
| 632 | tert-butyl-(4-{[4-(2-ethyl-4-methyl-1,3-thiazol-5-yl)pyrimidin-2- | 3.26* |
| | yl]amino}benzyl)carbamate | |
| 633 | tert-butyl-(4-{[4-(2-isopropyl-4-methyl-1,3-thiazol-5-yl)pyrimidin-2- | 3.68* |
| | yl]amino}benzyl)carbamate | |
| 634 | tert-butyl-(4-{[4-(2-methyl-1,3-thiazol-5-yl)pyrimidin-2- | |
| | yl]amino}benzyl)carbamate | |
| 635 | tert-butyl-(4-{[4-(4-cyclopropyl-2-ethyl-1,3-thiazol-5-yl)pyrimidin-2- | |
| | yl]amino}benzyl)carbamate | |
| 636 | tert-butyl-(4-{[4-(4-cyclopropyl-2-methyl-1,3-thiazol-5-yl)pyrimidin- | |
| | 2-yl]amino}benzyl)carbamate | |
| 637 | tert-butyl-(4-{[4-(4-sec-butyl-2-ethyl-1,3-thiazol-5-yl)pyrimidin-2- | |
| | yl]amino}benzyl)carbamate | |
| 638 | tert-butyl-(4-{[4-(4-sec-butyl-2-methyl-1,3-thiazol-5-yl)pyrimidin-2- | |
| | yl]amino}benzyl)carbamate | |
| 639 | tert-butyl-[3-({4-[2-(methoxymethyl)-4-methyl-1,3-thiazol-5- | 2.94* |
| | yl]pyrimidin-2-yl}amino)benzyl]carbamate | |
| 640 | tert-butyl-[4-({4-[2-(acetylamine)-1,3-thiazol-5-yl]pyrimidin-2- | |
| | yl}amino)benzyl]carbamate | |
| 641 | tert-butyl-[4-({4-[2-(acetylamine)-4-cyclopropyl-1,3-thiazol-5- | |
| | yl]pyrimidin-2-yl}amino)benzyl]carbamate | |
| 642 | tert-butyl-[4-({4-[2-(acetylamine)-4-sec-butyl-1,3-thiazol-5- | |
| | yl]pyrimidin-2-yl}amino)benzyl]carbamate | |
| 643 | tert-butyl-[4-({4-[2-(methoxymethyl)-4-methyl-1,3-thiazol-5- | 2.99* |
| | yl]pyrimidin-2-yl}amino)benzyl]carbamate | |
| 644 | tert-butyl-{4-[(4-{2-[(2-methoxy-1-methylethyl)amino]-1,3-thiazol-5- | |
| | yl}pyrimidin-2-yl)amino]benzyl}carbamate | |
| 645 | tert-butyl-{4-[(4-{2-[(2-methoxyethyl)amino]-1,3-thiazol-5- | |
| | yl}pyrimidin-2-yl)amino]benzyl}carbamate | |
| 646 | tert-butyl-{4-[(4-{2-[(tetrahydrofuran-2-ylmethyl)amino]-1,3-thiazol- | |
| | 5-yl}pyrimidin-2-yl)amino]benzyl}carbamate | |
| 647 | tert-butyl-{4-[(4-{2-[(trifluoroacetyl)amino]-1,3-thiazol-5- | |
| | yl}pyrimidin-2-yl)amino]benzyl}carbamate | |
| 648 | tert-butyl-{4-[(4-{4-cyclopropyl-2-[(2-methoxy-1- | |
| | methylethyl)amino]-1,3-thiazol-5-yl}pyrimidin-2- | |
| | yl)amino]benzyl}carbamate | |
| 649 | tert-butyl-{4-[(4-{4-cyclopropyl-2-[(2-methoxyethyl)amino]-1,3- | |
| | thiazol-5-yl}pyrimidin-2-yl)amino]benzyl}carbamate | |
| 650 | tert-butyl-{4-[(4-{4-cyclopropyl-2-[(tetrahydrofuran-2- | |
| | ylmethyl)amino]-1,3-thiazol-5-yl}pyrimidin-2- | |
| | yl)amino]benzyl}carbamate | |
| 651 | tert-butyl-{4-[(4-{4-cyclopropyl-2-[(trifluoroacetyl)amino]-1,3- | |
| | thiazol-5-yl}pyrimidin-2-yl)amino]benzyl}carbamate | |
| 652 | tert-butyl-{4-[(4-{4-sec-butyl-2-[(2-methoxy-1-methylethyl)amino]- | |
| | 1,3-thiazol-5-yl}pyrimidin-2-yl)amino]benzyl}carbamate | |
| 653 | tert-butyl-{4-[(4-{4-sec-butyl-2-[(tetrahydrofuran-2-ylmethyl)amino]- | |
| | 1,3-thiazol-5-yl}pyrimidin-2-yl)amino]benzyl}carbamate | |
| 654 | tert-butyl-{4-[(4-{4-sec-butyl-2-[(trifluoroacetyl)amino]-1,3-thiazol-5- | |
| | yl}pyrimidin-2-yl)amino]benzyl}carbamate | |
| 655 | N2,N2-dimethyl-N8-(3,4,5-trimethoxyphenyl)-4,5- | 1.67* |
| | dihydro[1,3]thiazole[4,5-h]quinazoline-2,8-diamine | |
| 656 | methyl-(4-{[4-(2-amino-4-methyl-1,3-thiazol-5-yl)pyrimidin-2- | 1.31* |
| | yl]amino}phenyl)carbamate | |
| 657 | N-{4-[(4-{2-[(2-methoxy-1-methylethyl)amino]-4-methyl-1,3-thiazol- | 1.53* |
| | 5-yl}pyrimidin-2-yl)amino]phenyl}methanesulphonamide | |
| 658 | tert-butyl-[3-({4-[4-methyl-2-(methylamine)-1,3-thiazol-5- | 1.99* |
| | yl]pyrimidin-2-yl}amino)benzyl]carbamate | |
| 659 | 4-(2,4-dimethyl-1,3-thiazol-5-yl)-N-phenyl-1,3,5-triazine-2-amine | |
| 660 | 4-(2-ethyl-4-methyl-1,3-thiazol-5-yl)-N-phenyl-1,3,5-triazine-2-amine | |
| 661 | 4-(2-isopropyl-4-methyl-1,3-thiazol-5-yl)-N-phenyl-1,3,5-triazine-2- | |
| | amine | |
| 662 | 4-(2,4-dimethyl-1,3-thiazol-5-yl)-N-(3,4,5-trimethoxyphenyl)-1,3,5- | |
| | triazine-2-amine | |
| 663 | 4-(2-ethyl-4-methyl-1,3-thiazol-5-yl)-N-(3,4,5-trimethoxyphenyl)- | |
| | 1,3,5-triazine-2-amine | |
| 664 | 4-(2-isopropyl-4-methyl-1,3-thiazol-5-yl)-N-(3,4,5-trimethoxyphenyl)- | |
| | 1,3,5-triazine-2-amine | |
| 665 | N-(3-acetylphenyl)-N-[4-(2-cyclopropyl-4-methyl-1,3-thiazol-5- | 2.43* |
| | yl)pyrimidin-2-yl]acetamide | |
| 666 | N-[4-(2-isopropyl-4-methyl-1,3-thiazol-5-yl)pyrimidin-2-yl]-N-(3,4,5- | 2.53* |
| | trimethoxyphenyl)acetamide | |
| 667 | N-[4-(2,4-dimethyl-1,3-thiazol-5-yl)pyrimidin-2-yl]-N-(3,4,5- | 1.9* |
| | trimethoxyphenyl)acetamide | |
| 668 | N-[4-(2-ethyl-4-methyl-1,3-thiazol-5-yl)pyrimidin-2-yl]-N- | 2.33* |
| | phenylacetamide | |
| 669 | N-[4-(2-ethyl-4-methyl-1,3-thiazol-5-yl)pyrimidin-2-yl]-N-(3,4,5- | 2.19* |
| | trimethoxyphenyl)acetamide | |
| 670 | N-acetyl-N-(3-{acetyl-[4-(2-cyclopropyl-4-methyl-1,3-thiazol-5- | Feb 39 |
| | yl)pyrimidin-2-yl]amino}benzyl)acetamide | |
| 671 | N-(3-acetylphenyl)-N-[4-(2-ethyl-4-methyl-1,3-thiazol-5- | 2.23* |
| | yl)pyrimidin-2-yl]acetamide | |
| 672 | methyl-(4-{[4-(2-cyclopropyl-4-methyl-1,3-thiazol-5-yl)pyrimidin-2- | 2.51* |
| | yl]amino}phenyl)carbamate | |
| 673 | 1-{4-[(2-methyl-4,5-dihydro[1,3]thiazole[4,5-h]quinazolin-8- | 2.36* |
| | yl)amino]phenyl}ethanone | |
| 674 | 2-chloro-N4-[4-(2,4-dimethyl-1,3-thiazol-5-yl)pyrimidin-2-yl]-N1,N1- | 2* |
| | dimethylbenzene-1,4-diamine | |
| 675 | N-[4-(2,4-dimethyl-1,3-thiazol-5-yl)pyrimidin-2-yl]-N-(3,4,5- | 1.9* |
| | trimethoxyphenyl)acetamide | |
| 676 | N-(3-acetylphenyl)-N-[4-(2-cyclopropyl-4-methyl-1,3-thiazol-5- | 2.43* |
| | yl)pyrimidin-2-yl]acetamide | |
| 677 | N-(4-fluorophenyl)-4-{2-[(2-methoxyethyl)amino]-4-methyl-1,3- | 1.9* |
| | thiazol-5-yl}pyrimidine-2-amine | |
| 678 | 4-{2-[(2-methoxyethyl)amino]-4-methyl-1,3-thiazol-5-yl}-N- | 1.82* |
| | phenylpyrimidine-2-amine | |
| 679 | 4-{2-[(2-methoxyethyl)amino]-4-methyl-1,3-thiazol-5-yl}-N-(3,4,5- | 1.74* |
| | trimethoxyphenyl)pyrimidine-2-amine | |
| 680 | N-(3-chlorophenyl)-4-{2-[(2-methoxyethyl)amino]-4-methyl-1,3- | 2.28* |
| | thiazol-5-yl}pyrimidine-2-amine | |
| 681 | 4-{2-[(2-methoxyethyl)amino]-4-methyl-1,3-thiazol-5-yl}-N-(3- | 1.86* |
| | methoxyphenyl)pyrimidine-2-amine | |
| 682 | N-{3-[(4-{2-[(2-methoxyethyl)amino]-4-methyl-1,3-thiazol-5- | 1.36* |
| | yl}pyrimidin-2-yl)amino]phenyl}acetamide | |
| 683 | N-{4-[(4-{2-[(2-methoxyethyl)amino]-4-methyl-1,3-thiazol-5- | 1.35* |
| | yl}pyrimidin-2-yl)amino]phenyl}acetamide | |
| 684 | N-[3-({4-[4-methyl-2-(methylamine)-1,3-thiazol-5-yl]pyrimidin-2- | 1.26* |
| | yl}amino)phenyl]acetamide | |
| 685 | 1-[3-({4-[2-(dimethylamine)-4-methyl-1,3-thiazol-5-yl]pyrimidin-2- | 1.89* |
| | yl}amino)phenyl]ethanone | |
| 686 | 4-[2-(dimethylamine)-4-methyl-1,3-thiazol-5-yl]-N-(3- | 2.34* |
| | nitrophenyl)pyrimidine-2-amine | |
| 687 | N-(3-chlorophenyl)-4-[2-(dimethylamine)-4-methyl-1,3-thiazol-5- | 2.62* |
| | yl]pyrimidine-2-amine | |
| 688 | 4-[2-(dimethylamine)-4-methyl-1,3-thiazol-5-yl]-N-phenylpyrimidine- | 1.98* |
| | 2-amine | |
| 689 | 1-{3-[(4-{2-[(2-methoxyethyl)amino]-4-methyl-1,3-thiazol-5- | 1.69* |
| | yl}pyrimidin-2-yl)amino]phenyl}ethanone | |
| 690 | 3-[(4-{2-[(2-methoxyethyl)amino]-4-methyl-1,3-thiazol-5- | 1.38* |
| | yl}pyrimidin-2-yl)amino]phenol | |
| 691 | N-{4-[(4-{2-[(2-methoxyethyl)amino]-4-methyl-1,3-thiazol-5- | 1.39* |
| | yl}pyrimidin-2-yl)amino]phenyl}methanesulphonamide | |
| 692 | 4-[2-(dimethylamine)-4-(trifluoromethyl)-1,3-thiazol-5-yl]-N-(4- | 4.43* |
| | fluoro-3-methylphenyl)pyrimidine-2-amine | |
| 693 | methyl-[4-({4-[2-methyl-4-(trifluoromethyl)-1,3-thiazol-5- | 2.7* |
| | yl]pyrimidin-2-yl}amino)phenyl]carbamate | |
| 694 | methyl-[4-({4-[2-(dimethylamine)-4-(trifluoromethyl)-1,3-thiazol-5- | 3.04* |
| | yl]pyrimidin-2-yl}amino)phenyl]carbamate | |
| 695 | N-(3-chlorophenyl)-4-[2-(dimethylamine)-4-(trifluoromethyl)-1,3- | 4.51* |
| | thiazol-5-yl]pyrimidine-2-amine | |
| 696 | 4-({4-[2-methyl-4-(trifluoromethyl)-1,3-thiazol-5-yl]pyrimidin-2- | 2.39* |
| | yl}amino)phenol | |
| 697 | 4-({4-[2-(dimethylamine)-4-(trifluoromethyl)-1,3-thiazol-5- | 2.64* |
| | yl]pyrimidin-2-yl}amino)phenol | |
| 698 | N-[4-({4-[2-methyl-4-(trifluoromethyl)-1,3-thiazol-5-yl]pyrimidin-2- | 2.26* |
| | yl}amino)phenyl]acetamide | |
| 699 | N-[4-({4-[2-(dimethylamine)-4-(trifluoromethyl)-1,3-thiazol-5- | 2.58* |
| | yl]pyrimidin-2-yl}amino)phenyl]acetamide | |
| 700 | 2-chloro-N1,N1-dimethyl-N4-{4-[2-methyl-4-(trifluoromethyl)-1,3- | 2.88* |
| | thiazol-5-yl]pyrimidin-2-yl}benzene-1,4-diamine | |
| 701 | 2-chloro-N4-{4-[2-(dimethylamine)-4-(trifluoromethyl)-1,3-thiazol-5- | 3.46* |
| | yl]pyrimidin-2-yl}-N1,N1-dimethylbenzene-1,4-diamine | |
| 702 | 3-({4-[2-(dimethylamine)-4-(trifluoromethyl)-1,3-thiazol-5- | 2.84* |
| | yl]pyrimidin-2-yl}amino)phenol | |
| 703 | N-1,3-benzodioxol-5-yl-4-[2-methyl-4-(trifluoromethyl)-1,3-thiazol-5- | 3.21* |
| | yl]pyrimidine-2-amine | |
| 704 | N-1,3-benzodioxol-5-yl-4-[2-(dimethylamine)-4-(trifluoromethyl)-1,3- | 3.63* |
| | thiazol-5-yl]pyrimidine-2-amine | |
| 705 | N-(3,4-difluorophenyl)-4-[2-methyl-4-(trifluoromethyl)-1,3-thiazol-5- | 3.76* |
| | yl]pyrimidine-2-amine | |
| 706 | N-(3,5-dimethoxyphenyl)-4-[2-methyl-4-(trifluoromethyl)-1,3-thiazol- | 3.41* |
| | 5-yl]pyrimidine-2-amine | |
| 707 | N-(3,5-dimethoxyphenyl)-4-[2-(dimethylamine)-4-(trifluoromethyl)- | 3.85* |
| | 1,3-thiazol-5-yl]pyrimidine-2-amine | |
| 708 | 4-[4-(difluoromethyl)-2-methyl-1,3-thiazol-5-yl]-N-(3,4,5- | 2.61* |
| | trimethoxyphenyl)pyrimidine-2-amine | |
| 709 | 1-[3-({4-[4-(difluoromethyl)-2-methyl-1,3-thiazol-5-yl]pyrimidin-2- | 2.64* |
| | yl}amino)phenyl]ethanone | |
| 710 | 4-[4-(difluoromethyl)-2-methyl-1,3-thiazol-5-yl]-N-phenylpyrimidine- | 2.97* |
| | 2-amine | |
| 711 | 4-{[4-(2-ethyl-4-methyl-1,3-thiazol-5-yl)pyrimidin-2-yl]amino}-N-(2- | 2.09* |
| | methoxyethyl)benzamide | |
| 712 | methyl-[3-({4-[4-methyl-2-(methylthio)-1,3-thiazol-5-yl]pyrimidin-2- | 2.16* |
| | yl}amino)benzyl]carbamate | |
| 713 | 4-(4-methyl-2-pyridin-3-yl-1,3-thiazol-5-yl)-N-(3,4,5- | 2.56* |
| | trimethoxyphenyl)pyrimidine-2-amine | |
| 714 | 4-(2-ethyl-4-methyl-1,3-thiazol-5-yl)-N-pyridin-3-ylpyrimidine-2- | 1.35* |
| | amine | |
| 715 | 4-(4-methyl-2-pyridin-3-yl-1,3-thiazol-5-yl)-N-phenylpyrimidine-2- | 3.04* |
| | amine | |
| 716 | 4-(2,4-dimethyl-1,3-thiazol-5-yl)-N-pyridin-3-ylpyrimidine-2-amine | 0.98* |
| 717 | 1-(4-{[4-(2-ethyl-4-methyl-1,3-thiazol-5-yl)pyrimidin-2- | 2.6* |
| | yl]amino}phenyl)ethanone | |
| 718 | N-(3,4-dimethylphenyl)-4-(2-ethyl-4-methyl-1,3-thiazol-5- | 3.71* |
| | yl)pyrimidine-2-amine | |
| 719 | N-(3,5-dimethoxyphenyl)-4-{4-methyl-2-[(tetrahydrofuran-2- | 2.02* |
| | ylmethyl)amino]-1,3-thiazol-5-yl}pyrimidine-2-amine | |
| 720 | 1-{4-[(4-{4-methyl-2-[(tetrahydrofuran-2-ylmethyl)amino]-1,3- | 1.6* |
| | thiazol-5-yl}pyrimidin-2-yl)amino]phenyl}pyrrolidin-2-one | |
| 721 | N-[6-(2,2-dimethylpropoxy)pyridin-3-yl]-4-(2-ethyl-4-methyl-1,3- | 4.23* |
| | thiazol-5-yl)pyrimidine-2-amine | |
| 722 | N-(8-anilino-4,5-dihydro[1,3]thiazole[4,5-h]quinazolin-2- | 1.83* |
| | yl)acetamide | |
| 723 | N-{3-[(2-acetamido-4,5-dihydro[1,3]thiazole[4,5-h]quinazolin-8- | 1.38* |
| | yl)amino]phenyl}acetamide | |
| 724 | N-[8-({3-[(methylsulphonyl)amino]phenyl}amino)-4,5- | 1.54* |
| | dihydro[1,3]thiazole[4,5-h]quinazolin-2-yl]acetamide | |
| 725 | 4-{2-[(3-methoxypropyl)amino]-4-methyl-1,3-thiazol-5-yl}-N- | 1.81* |
| | phenylpyrimidine-2-amine | |
| 726 | N-(3-chlorophenyl)-4-{2-[(3-methoxypropyl)amino]-4-methyl-1,3- | 2.28* |
| | thiazol-5-yl}pyrimidine-2-amine | |
| 727 | 1-{4-[(2-methyl-4,5-dihydro[1,3]thiazole[4,5-h]quinazolin-8- | 1.94* |
| | yl)amino]phenyl}pyrrolidin-2-one | |
| 728 | 1-{4-[(4-{2-[(2-methoxy-1-methylethyl)amino]-4-methyl-1,3-thiazol- | 1.64* |
| | 5-yl}pyrimidin-2-yl)amino]phenyl}pyrrolidin-2-one | |
| 729 | 1-{4-[(4-{2-[(2-methoxy-1-methylethyl)amino]-4-methyl-1,3-thiazol- | 1.92* |
| | 5-yl}pyrimidin-2-yl)amino]phenyl}ethanone | |
| 730 | N-{8-[(3,4,5-trimethoxyphenyl)amino]-4,5-dihydro[1,3]thiazole[4,5- | 1.76* |
| | h]quinazolin-2-yl}acetamide | |
| 731 | 4-(4-methyl-2-morpholin-4-yl-1,3-thiazol-5-yl)-N-phenylpyrimidine- | 2.25* |
| | 2-amine | |
| 732 | methyl-[4-(2-ethyl-4-methyl-1,3-thiazol-5-yl)pyrimidin-2- | 2.59* |
| | yl]phenylcarbamate | |
| 733 | 1-(4-{[4-(2-ethyl-4-methyl-1,3-thiazol-5-yl)pyrimidin-2- | 3.5* |
| | yl]amino}phenyl)butan-1-one | |
| 734 | 1-(3-{[4-(4-methyl-2-pyridin-3-yl-1,3-thiazol-5-yl)pyrimidin-2- | 2.56* |
| | yl]amino}phenyl)ethanone | |
| 735 | 4-(4-methyl-2-morpholin-4-yl-1,3-thiazol-5-yl)-N-(3,4,5- | 2.07* |
| | trimethoxyphenyl)pyrimidine-2-amine | |
| 736 | 1-(3-{[4-(4-methyl-2-morpholin-4-yl-1,3-thiazol-5-yl)pyrimidin-2- | 2.16* |
| | yl]amino}phenyl)ethanone | |
| 737 | N-(4-{2-[(2-methoxy-1-methylethyl)amino]-4-methyl-1,3-thiazol-5- | 1.68* |
| | yl}pyrimidin-2-yl)benzene-1,4-diamine | |
| 738 | 1-{3-[(4-{2-[(3-methoxypropyl)amino]-4-methyl-1,3-thiazol-5- | 1.75* |
| | yl}pyrimidin-2-yl)amino]phenyl}ethanone | |
| 739 | 4-[2-(tert-butylamine)-4-methyl-1,3-thiazol-5-yl]-N-(3,5- | 2.44* |
| | dimethoxyphenyl)pyrimidine-2-amine | |
| 740 | 4-[2-(tert-butylamine)-4-methyl-1,3-thiazol-5-yl]-N-(2,3-dihydro-1,4- | 2.11* |
| | benzodioxin-6-yl)pyrimidine-2-amine | |
| 741 | 1-[4-({4-[2-(tert-butylamine)-4-methyl-1,3-thiazol-5-yl]pyrimidin-2- | 1.96* |
| | yl}amino)phenyl]pyrrolidin-2-one | |
| 742 | 1-[4-({4-[2-(tert-butylamine)-4-methyl-1,3-thiazol-5-yl]pyrimidin-2- | 2.35* |
| | yl}amino)phenyl]ethanone | |
| 743 | 4-[2-(tert-butylamine)-4-methyl-1,3-thiazol-5-yl]-N-(3,4- | 2* |
| | dimethoxyphenyl)pyrimidine-2-amine | |
| 744 | N-1,3-benzodioxol-5-yl-4-[2-(tert-butylamine)-4-methyl-1,3-thiazol-5- | 2.14* |
| | yl]pyrimidine-2-amine | |
| 745 | N-(3-chlorophenyl)-4-(4-methyl-2-morpholin-4-yl-1,3-thiazol-5- | 2.99* |
| | yl)pyrimidine-2-amine | |
| 746 | 4-({4-[4-methyl-2-(methylamine)-1,3-thiazol-5-yl]pyrimidin-2- | 1.11* |
| | yl}amino)phenol | |
| 747 | 4-(2-ethyl-4-methyl-1,3-thiazol-5-yl)-N-(5-methylpyridin-3- | 1.41* |
| | yl)pyrimidine-2-amine | |
| 748 | 1-(4-{[4-(2-ethyl-4-methyl-1,3-thiazol-5-yl)pyrimidin-2- | 2.24* |
| | yl]amino}phenyl)pyrrolidin-2-one | |
| 749 | 1-(4-{[4-(2,4-dimethyl-1,3-thiazol-5-yl)pyrimidin-2- | 1.99* |
| | yl]amino}phenyl)pyrrolidin-2-one | |
| 750 | N-(4-fluoro-3-methylphenyl)-4-{2-[(2-methoxyethyl)amino]-4- | 2.04* |
| | methyl-1,3-thiazol-5-yl}pyrimidine-2-amine | |
| 751 | 1-{4-[(4-{2-[(2-methoxyethyl)amino]-4-methyl-1,3-thiazol-5- | 1.72* |
| | yl}pyrimidin-2-yl)amino]phenyl}ethanone | |
| 752 | 1-{4-[(4-{2-[(2-methoxyethyl)amino]-4-methyl-1,3-thiazol-5- | 1.51* |
| | yl}pyrimidin-2-yl)amino]phenyl}pyrrolidin-2-one | |
| 753 | 1-{3-[(2-methyl-4,5-dihydro[1,3]thiazole[4,5-h]quinazolin-8- | 2.01* |
| | yl)amino]phenyl}pyrrolidin-2-one | |
| 754 | 4-({4-[4-methyl-2-(methylamine)-1,3-thiazol-5-yl]pyrimidin-2- | 1.73* |
| | yl}amino)phenylacetate | |
| 755 | N-(3,4-difluorophenyl)-4-(2-ethyl-4-methyl-1,3-thiazol-5- | 3.44* |
| | yl)pyrimidine-2-amine | |
| 756 | N-(3,4-difluorophenyl)-4-(2,4-dimethyl-1,3-thiazol-5-yl)pyrimidine-2- | 2.99* |
| | amine | |
| 757 | N-(3-chlorophenyl)-4-[4-(difluoromethyl)-2-methyl-1,3-thiazol-5- | 1.41* |
| | yl]pyrimidine-2-amine | |
| 758 | tert-butyl-[4-({4-[4-(difluoromethyl)-2-methyl-1,3-thiazol-5- | 3.2* |
| | yl]pyrimidin-2-yl}amino)benzyl]carbamate | |
| 759 | N-(2,4-difluorophenyl)-4-(2,4-dimethyl-1,3-thiazol-5-yl)pyrimidine-2- | 2.8* |
| | amine | |
| 760 | N-(2,4-difluorophenyl)-4-(2-ethyl-4-methyl-1,3-thiazol-5- | 3.27* |
| | yl)pyrimidine-2-amine | |
| 761 | 1-[4-({4-[2-(dimethylamine)-4-methyl-1,3-thiazol-5-yl]pyrimidin-2- | 1.6* |
| | yl}amino)phenyl]pyrrolidin-2-one | |
| 762 | N-[3-({4-[2-(dimethylamine)-4-methyl-1,3-thiazol-5-yl]pyrimidin-2- | 1.42* |
| | yl}amino)phenyl]acetamide | |
| 763 | 4-({4-[2-(methoxymethyl)-4-methyl-1,3-thiazol-5-yl]pyrimidin-2- | 1.8* |
| | yl}amino)phenol | |
| 764 | N-(3,5-dimethoxyphenyl)-4-[2-(methoxymethyl)-4-methyl-1,3- | 2.73* |
| | thiazol-5-yl]pyrimidine-2-amine | |
| 765 | 3-methoxypropyl-4-({4-[2-(methoxymethyl)-4-methyl-1,3-thiazol-5- | 2.63* |
| | yl]pyrimidin-2-yl}amino)benzoate | |
| 766 | 4-(4-methyl-2-propyl-1,3-thiazol-5-yl)-N-phenylpyrimidine-2-amine | 3.49* |
| 767 | 1-(3-{[4-(4-methyl-2-propyl-1,3-thiazol-5-yl)pyrimidin-2- | 3.04* |
| | yl]amino}phenyl)ethanone | |
| 768 | 4-(4-methyl-2-propyl-1,3-thiazol-5-yl)-N-(3,4,5- | 2.93* |
| | trimethoxyphenyl)pyrimidine-2-amine | |
| 769 | 1-[4-({4-[2-(dimethylamine)-4-(trifluoromethyl)-1,3-thiazol-5- | 3.44* |
| | yl]pyrimidin-2-yl}amino)phenyl]ethanone | |
| 770 | 1-[4-({4-[2-(dimethylamine)-4-(trifluoromethyl)-1,3-thiazol-5- | 2.99* |
| | yl]pyrimidin-2-yl}amino)phenyl]pyrrolidin-2-one | |
| 771 | 1-[3-({4-[2-(tert-butylamine)-4-methyl-1,3-thiazol-5-yl]pyrimidin-2- | 1.93* |
| | yl}amino)phenyl]pyrrolidin-2-one | |
| 772 | 1-{3-[(4-{2-[(2-methoxy-1-methylethyl)amino]-4-methyl-1,3-thiazol- | 1.68* |
| | 5-yl}pyrimidin-2-yl)amino]phenyl}pyrrolidin-2-one | |
| 773 | N8-(4-fluorophenyl)-N2-(2-methoxyethyl)-N2-methyl-4,5- | 1.94* |
| | dihydro[1,3]thiazole[4,5-h]quinazoline-2,8-diamine | |
| 774 | 4-[2-(dimethylamine)-4-methyl-1,3-thiazol-5-yl]-N-(2,2,3,3- | 3.6* |
| | tetrafluoro-2,3-dihydro-1,4-benzodioxin-6-yl)pyrimidine-2-amine | |
| 775 | 1-[3-({4-[2-(dimethylamine)-4-(trifluoromethyl)-1,3-thiazol-5- | 3.07* |
| | yl]pyrimidin-2-yl}amino)phenyl]pyrrolidin-2-one | |
| 776 | N2-(3-methoxypropyl)-N2-methyl-N8-(3,4,5-trimethoxyphenyl)-4,5- | 1.89* |
| | dihydro[1,3]thiazole[4,5-h]quinazoline-2,8-diamine | |
| 777 | 4-[2-(dimethylamine)-4-(trifluoromethyl)-1,3-thiazol-5-yl]-N-(4- | 3.96* |
| | fluorophenyl)pyrimidine-2-amine | |
| 778 | 4-[2-(dimethylamine)-4-(trifluoromethyl)-1,3-thiazol-5-yl]-N-(4- | 4.88* |
| | iodophenyl)pyrimidine-2-amine | |
| 779 | N-(2,2-difluoro-1,3-benzodioxol-5-yl)-4-[2-(dimethylamine)-4- | 4.72* |
| | (trifluoromethyl)-1,3-thiazol-5-yl]pyrimidine-2-amine | |
| 780 | 4-[2-(dimethylamine)-4-(trifluoromethyl)-1,3-thiazol-5-yl]-N-(2,2,3,3- | 5.28* |
| | tetrafluoro-2,3-dihydro-1,4-benzodioxin-6-yl)pyrimidine-2-amine | |
| 781 | 4-{2-[(2-methoxyethyl)amino]-4-methyl-1,3-thiazol-5-yl}-N-(3- | 1.97* |
| | nitrophenyl)pyrimidine-2-amine | |
| 782 | 1-[3-({4-[2-(dimethylamine)-4-methyl-1,3-thiazol-5-yl]pyrimidin-2- | 1.65* |
| | yl}amino)phenyl]pyrrolidin-2-one | |
| 783 | 4-[2-(dimethylamine)-4-(trifluoromethyl)-1,3-thiazol-5-yl]-N-(6- | 3.17* |
| | methoxypyridin-3-yl)pyrimidine-2-amine | |
| 784 | 4-{2-[(2-methoxyethyl)amino]-4-methyl-1,3-thiazol-5-yl}-N-(6- | 1.45* |
| | methoxypyridin-3-yl)pyrimidine-2-amine | |
| 785 | 4-[2-(dimethylamine)-4-methyl-1,3-thiazol-5-yl]-N-(6- | 1.55* |
| | methoxypyridin-3-yl)pyrimidine-2-amine | |
| 786 | 4-(2-phenyl-1,3-thiazol-5-yl)-N-(3,4,5-trimethoxyphenyl)pyrimidine- | 3.37* |
| | 2-amine | |
| 787 | N-phenyl-4-(2-phenyl-1,3-thiazol-5-yl)pyrimidine-2-amine | 3.97* |
| 788 | 4-(2,4-dimethyl-1,3-thiazol-5-yl)-N-(4-methoxyphenyl)pyrimidine-2- | 2.34* |
| | amine | |
| 789 | 4-(2-ethyl-4-methyl-1,3-thiazol-5-yl)-N-(4- | 2.78* |
| | methoxyphenyl)pyrimidine-2-amine | |
| 790 | 5-{[4-(2,4-dimethyl-1,3-thiazol-5-yl)pyrimidin-2-yl]amino}-2- | 1.79* |
| | methoxyphenol | |
| 791 | 5-{[4-(2-ethyl-4-methyl-1,3-thiazol-5-yl)pyrimidin-2-yl]amino}-2- | 2.09* |
| | methoxyphenol | |
| 792 | 4-[2-(4-chlorophenyl)-1,3-thiazol-5-yl]-N-(3,4,5- | 4.12* |
| | trimethoxyphenyl)pyrimidine-2-amine | |
| 793 | 4-[2-(4-chlorophenyl)-1,3-thiazol-5-yl]-N-phenylpyrimidine-2-amine | 4.85* |
| 794 | N-(2,4-dichlorophenyl)-4-(2-ethyl-4-methyl-1,3-thiazol-5- | 4.9* |
| | yl)pyrimidine-2-amine | |
| 795 | N-(3-chlorophenyl)-4-[2-(4-chlorophenyl)-1,3-thiazol-5- | 5.57* |
| | yl]pyrimidine-2-amine | |
| 796 | ethyl-(4-{[4-(2-isopropyl-4-methyl-1,3-thiazol-5-yl)pyrimidin-2- | 2.18* |
| | yl]amino}phenyl)carbamate | |
| 797 | N5-(4-fluorophenyl)-N2,N2-dimethyl-8H- | 1.56* |
| | [1,3]thiazole[4′,5′:4,5]cyclopenta[1,2-d]pyrimidine-2,5-diamine | |
| 798 | N-(4-fluorophenyl)-4-[2-(isobutylamine)-4-methyl-1,3-thiazol-5- | 2.36* |
| | yl]pyrimidine-2-amine | |
| 799 | 4-[2-(isobutylamine)-4-methyl-1,3-thiazol-5-yl]-N-(4-methyl-3- | 2.68* |
| | nitrophenyl)pyrimidine-2-amine | |
| 800 | N-(3,4-dimethoxyphenyl)-4-{2-[(2-methoxyethyl)amino]-4-methyl- | 1.55* |
| | 1,3-thiazol-5-yl}pyrimidine-2-amine | |
| 801 | N-(3,5-dimethoxyphenyl)-4-{2-[(2-methoxyethyl)amino]-4-methyl- | 1.96* |
| | 1,3-thiazol-5-yl}pyrimidine-2-amine | |
| 802 | N-1,3-benzodioxol-5-yl-4-{2-[(2-methoxyethyl)amino]-4-methyl-1,3- | 1.65* |
| | thiazol-5-yl}pyrimidine-2-amine | |
| 803 | N-(2,3-dihydro-1,4-benzodioxin-6-yl)-4-{2-[(2-methoxyethyl)amino]- | 1.63* |
| | 4-methyl-1,3-thiazol-5-yl}pyrimidine-2-amine | |
| 804 | N-(3-chloro-4-methoxyphenyl)-4-{2-[(2-methoxyethyl)amino]-4- | 1.96* |
| | methyl-1,3-thiazol-5-yl}pyrimidine-2-amine | |
| 805 | 2-chloro-N4-(4-{2-[(2-methoxyethyl)amino]-4-methyl-1,3-thiazol-5- | 1.57* |
| | yl}pyrimidin-2-yl)-N1,N1-dimethylbenzene-1,4-diamine | |
| 806 | 4-{2-[(2-methoxyethyl)amino]-4-methyl-1,3-thiazol-5-yl}-N-(4- | 2.17* |
| | methyl-3-nitrophenyl)pyrimidine-2-amine | |
| 807 | 4-[(4-{2-[(2-methoxyethyl)amino]-4-methyl-1,3-thiazol-5- | 1.9* |
| | yl}pyrimidin-2-yl)amino]benzonitrile | |
| 808 | 4-[(4-{2-[(2-methoxyethyl)amino]-4-methyl-1,3-thiazol-5- | 1.22* |
| | yl}pyrimidin-2-yl)amino]phenol | |
| 809 | N-(2,4-difluorophenyl)-4-{4-methyl-2-[(tetrahydrofuran-2- | 2.09* |
| | ylmethyl)amino]-1,3-thiazol-5-yl}pyrimidine-2-amine | |
| 810 | N-(4-methyl-3-nitrophenyl)-4-{4-methyl-2-[(tetrahydrofuran-2- | 2.33* |
| | ylmethyl)amino]-1,3-thiazol-5-yl}pyrimidine-2-amine | |
| 811 | 4-({4-[2-(tert-butylamine)-4-methyl-1,3-thiazol-5-yl]pyrimidin-2- | 2.63* |
| | yl}amino)benzonitrile | |
| 812 | 4-[2-(tert-butylamine)-4-methyl-1,3-thiazol-5-yl]-N-(3-chloro-4- | 2.52* |
| | methoxyphenyl)pyrimidine-2-amine | |
| 813 | N4-{4-[2-(tert-butylamine)-4-methyl-1,3-thiazol-5-yl]pyrimidin-2-yl}- | 2.09* |
| | 2-chloro-N1,N1-dimethylbenzene-1,4-diamine | |
| 814 | 4-[2-(tert-butylamine)-4-methyl-1,3-thiazol-5-yl]-N-(2,4- | 2.71* |
| | difluorophenyl)pyrimidine-2-amine | |
| 815 | 4-[2-(tert-butylamine)-4-methyl-1,3-thiazol-5-yl]-N-(4-methyl-3- | 2.88* |
| | nitrophenyl)pyrimidine-2-amine | |
| 816 | 1-{4-[(4-{4-methyl-2-[(tetrahydrofuran-2-ylmethyl)amino]-1,3- | 1.83* |
| | thiazol-5-yl}pyrimidin-2-yl)amino]phenyl}ethanone | |
| 817 | N-(3,4-dimethoxyphenyl)-4-{4-methyl-2-[(tetrahydrofuran-2- | 1.65* |
| | ylmethyl)amino]-1,3-thiazol-5-yl}pyrimidine-2-amine | |
| 818 | N-(3-{[4-(2-isopropyl-4-methyl-1,3-thiazol-5-yl)pyrimidin-2- | 2.26* |
| | yl]amino}phenyl)acetamide | |
| 819 | N-(3,4-dimethoxyphenyl)-4-(2-isopropyl-4-methyl-1,3-thiazol-5- | 2.85* |
| | yl)pyrimidine-2-amine | |
| 820 | 1-(4-{[4-(2-isopropyl-4-methyl-1,3-thiazol-5-yl)pyrimidin-2- | 3.06* |
| | yl]amino}phenyl)ethanone | |
| 821 | 4-(2-isopropyl-4-methyl-1,3-thiazol-5-yl)-N-(4- | 3.25* |
| | methoxyphenyl)pyrimidine-2-amine | |
| 822 | 4-[2-(isobutylamine)-4-methyl-1,3-thiazol-5-yl]-N-phenylpyrimidine- | 2.2* |
| | 2-amine | |
| 823 | 4-[2-(isobutylamine)-4-methyl-1,3-thiazol-5-yl]-N-(3,4,5- | 2.06* |
| | trimethoxyphenyl)pyrimidine-2-amine | |
| 824 | 1-[3-({4-[2-(isobutylamine)-4-methyl-1,3-thiazol-5-yl]pyrimidin-2- | 2.08* |
| | yl}amino)phenyl]ethanone | |
| 825 | N-(3-chlorophenyl)-4-[2-(isobutylamine)-4-methyl-1,3-thiazol-5- | 2.74* |
| | yl]pyrimidine-2-amine | |
| 826 | 4-[2-(isobutylamine)-4-methyl-1,3-thiazol-5-yl]-N-(3- | 2.22* |
| | methoxyphenyl)pyrimidine-2-amine | |
| 827 | N-[3-({4-[2-(isobutylamine)-4-methyl-1,3-thiazol-5-yl]pyrimidin-2- | 1.63* |
| | yl}amino)phenyl]acetamide | |
| 828 | 1-[4-({4-[2-(isobutylamine)-4-methyl-1,3-thiazol-5-yl]pyrimidin-2- | 2.13* |
| | yl}amino)phenyl]ethanone | |
| 829 | N-(4-fluoro-3-methylphenyl)-4-[2-(isobutylamine)-4-methyl-1,3- | 2.55* |
| | thiazol-5-yl]pyrimidine-2-amine | |
| 830 | 4-[2-(isobutylamine)-4-methyl-1,3-thiazol-5-yl]-N-(6-methoxypyridin- | 1.87* |
| | 3-yl)pyrimidine-2-amine | |
| 831 | 1-(4-{[4-(2-isopropyl-4-methyl-1,3-thiazol-5-yl)pyrimidin-2- | 2.56* |
| | yl]amino}phenyl)pyrrolidin-2-one | |
| 832 | N-(2,4-difluorophenyl)-4-{2-[(2-methoxyethyl)amino]-4-methyl-1,3- | 1.99* |
| | thiazol-5-yl}pyrimidine-2-amine | |
| 833 | N-[4-({4-[2-(isobutylamine)-4-methyl-1,3-thiazol-5-yl]pyrimidin-2- | 1.58* |
| | yl}amino)phenyl]acetamide | |
| 834 | 1-[4-({4-[2-(isobutylamine)-4-methyl-1,3-thiazol-5-yl]pyrimidin-2- | 1.82* |
| | yl}amino)phenyl]pyrrolidin-2-one | |
| 835 | 4-[2-(butylamine)-4-methyl-1,3-thiazol-5-yl]-N-(4- | 2.32* |
| | fluorophenyl)pyrimidine-2-amine | |
| 836 | 4-[2-(butylamine)-4-methyl-1,3-thiazol-5-yl]-N-phenylpyrimidine-2- | 2.2* |
| | amine | |
| 837 | 4-[2-(butylamine)-4-methyl-1,3-thiazol-5-yl]-N-(3,4,5- | 2.04* |
| | trimethoxyphenyl)pyrimidine-2-amine | |
| 838 | 4-[2-(butylamine)-4-methyl-1,3-thiazol-5-yl]-N-(3- | 2.46* |
| | nitrophenyl)pyrimidine-2-amine | |
| 839 | 1-[3-({4-[2-(butylamine)-4-methyl-1,3-thiazol-5-yl]pyrimidin-2- | 2.07* |
| | yl}amino)phenyl]ethanone | |
| 840 | 4-[2-(butylamine)-4-methyl-1,3-thiazol-5-yl]-N-(3- | 2.75* |
| | chlorophenyl)pyrimidine-2-amine | |
| 841 | 4-[2-(butylamine)-4-methyl-1,3-thiazol-5-yl]-N-(3- | 2.23* |
| | methoxyphenyl)pyrimidine-2-amine | |
| 842 | N-[3-({4-[2-(butylamine)-4-methyl-1,3-thiazol-5-yl]pyrimidin-2- | 1.65* |
| | yl}amino)phenyl]acetamide | |
| 843 | N-[4-({4-[2-(butylamine)-4-methyl-1,3-thiazol-5-yl]pyrimidin-2- | 1.59* |
| | yl}amino)phenyl]acetamide | |
| 844 | 1-[4-({4-[2-(butylamine)-4-methyl-1,3-thiazol-5-yl]pyrimidin-2- | 2.2* |
| | yl}amino)phenyl]ethanone | |
| 845 | 1-[4-({4-[2-(butylamine)-4-methyl-1,3-thiazol-5-yl]pyrimidin-2- | 1.8* |
| | yl}amino)phenyl]pyrrolidin-2-one | |
| 846 | 4-[2-(butylamine)-4-methyl-1,3-thiazol-5-yl]-N-(4-fluoro-3- | 2.67* |
| | methylphenyl)pyrimidine-2-amine | |
| 847 | 4-[2-(butylamine)-4-methyl-1,3-thiazol-5-yl]-N-(6-methoxypyridin-3- | 1.85* |
| | yl)pyrimidine-2-amine | |
| 848 | N-[3-(4-acetylpiperazin-1-yl)-4-fluorophenyl]-4-(2-ethyl-4-methyl- | 2.42* |
| | 1,3-thiazol-5-yl)pyrimidine-2-amine | |
| 849 | 4-[2-(dimethylamine)-4-methyl-1,3-thiazol-5-yl]-N-(4- | 1.99* |
| | fluorophenyl)pyrimidine-2-amine | |
| 850 | 4-[2-(dimethylamine)-4-methyl-1,3-thiazol-5-yl]-N-(3,4,5- | 1.77* |
| | trimethoxyphenyl)pyrimidine-2-amine | |
| 851 | N-(4-fluorophenyl)-4-{2-[(3-methoxypropyl)amino]-4-methyl-1,3- | 1.92* |
| | thiazol-5-yl}pyrimidine-2-amine | |
| 852 | N-(3-methoxyphenyl)-4-{2-[(3-methoxypropyl)amino]-4-methyl-1,3- | 1.89* |
| | thiazol-5-yl}pyrimidine-2-amine | |
| 853 | N-{3-[(4-{2-[(3-methoxypropyl)amino]-4-methyl-1,3-thiazol-5- | 1.44* |
| | yl}pyrimidin-2-yl)amino]phenyl}acetamide | |
| 854 | N-{4-[(4-{2-[(3-methoxypropyl)amino]-4-methyl-1,3-thiazol-5- | 1.4* |
| | yl}pyrimidin-2-yl)amino]phenyl}acetamide | |
| 855 | 1-{4-[(4-{2-[(3-methoxypropyl)amino]-4-methyl-1,3-thiazol-5- | 1.81* |
| | yl}pyrimidin-2-yl)amino]phenyl}ethanone | |
| 856 | 1-{4-[(4-{2-[(3-methoxypropyl)amino]-4-methyl-1,3-thiazol-5- | 1.57* |
| | yl}pyrimidin-2-yl)amino]phenyl}pyrrolidin-2-one | |
| 857 | N-(4-fluoro-3-methylphenyl)-4-{2-[(3-methoxypropyl)amino]-4- | 2.11* |
| | methyl-1,3-thiazol-5-yl}pyrimidine-2-amine | |
| 858 | 4-{2-[(3-methoxypropyl)amino]-4-methyl-1,3-thiazol-5-yl}-N-(6- | 1.57* |
| | methoxypyridin-3-yl)pyrimidine-2-amine | |
| 859 | 4-[2-(dimethylamine)-4-(trifluoromethyl)-1,3-thiazol-5-yl]-N-(3- | 3.99* |
| | nitrophenyl)pyrimidine-2-amine | |
| 860 | N-[3-({4-[2-(dimethylamine)-4-(trifluoromethyl)-1,3-thiazol-5- | 2.65* |
| | yl]pyrimidin-2-yl}amino)phenyl]acetamide | |
| 861 | 1-{3-[(4-{2-[(3-methoxypropyl)amino]-4-methyl-1,3-thiazol-5- | 1.62* |
| | yl}pyrimidin-2-yl)amino]phenyl}pyrrolidin-2-one | |
| 862 | N-[4-({4-[2-(dimethylamine)-4-methyl-1,3-thiazol-5-yl]pyrimidin-2- | 1.33* |
| | yl}amino)phenyl]acetamide | |
| 863 | 4-(2-ethyl-4-methyl-1,3-thiazol-5-yl)-N-(4-fluoro-3- | 2.99* |
| | methoxyphenyl)pyrimidine-2-amine | |
| 864 | 4-[(4-{2-[(2-methoxy-1-methylethyl)amino]-4-methyl-1,3-thiazol-5- | 1.49* |
| | yl}pyrimidin-2-yl)amino]benzenesulphonamide | |
| 865 | 4-{2-[(3-methoxypropyl)amino]-4-methyl-1,3-thiazol-5-yl}-N-(3- | 2.03* |
| | nitrophenyl)pyrimidine-2-amine | |
| 866 | 4-{2-[(3-methoxypropyl)amino]-4-methyl-1,3-thiazol-5-yl}-N-(3,4,5- | 1.73* |
| | trimethoxyphenyl)pyrimidine-2-amine | |
| 867 | 1-[4-({4-[2-(dimethylamine)-4-methyl-1,3-thiazol-5-yl]pyrimidin-2- | 1.9* |
| | yl}amino)phenyl]ethanone | |
| 868 | 4-[2-(isobutylamine)-4-methyl-1,3-thiazol-5-yl]-N-(3- | 2.47* |
| | nitrophenyl)pyrimidine-2-amine | |
| 869 | N2,N2-dimethyl-N5-phenyl-8H-[1,3]thiazole[4′,5′:4,5]cyclopenta[1,2- | 1.49* |
| | d]pyrimidin-2,5-diamine | |
| 870 | 1-(4-{[2-(dimethylamine)-8H-[1,3]thiazole[4′,5′:4,5]cyclopenta[1,2- | 1.37* |
| | d]pyrimidin-5-yl]amino}phenyl)pyrrolidin-2-one | |
| 871 | 4-[2-(tert-butylamine)-4-methyl-1,3-thiazol-5-yl]-N-(6-chloropyridin- | 2.48* |
| | 3-yl)pyrimidine-2-amine | |
| 872 | N-(6-chloropyridin-3-yl)-4-{2-[(2-methoxyethyl)amino]-4-methyl-1,3- | 1.71* |
| | thiazol-5-yl}pyrimidine-2-amine | |
| 873 | 5-chloro-4-(2,4-dimethyl-1,3-thiazol-5-yl)-N-phenylpyrimidine-2- | 3.26* |
| | amine | |
| 874 | 5-chloro-4-(2,4-dimethyl-1,3-thiazol-5-yl)-N-(3,4,5- | 2.82* |
| | trimethoxyphenyl)pyrimidine-2-amine | |
| 875 | 5-chloro-N-(3-chlorophenyl)-4-(2,4-dimethyl-1,3-thiazol-5- | 3.86* |
| | yl)pyrimidine-2-amine | |
| 876 | 5-chloro-4-(2-ethyl-4-methyl-1,3-thiazol-5-yl)-N-phenylpyrimidine-2- | 3.78* |
| | amine | |
| 877 | 5-chloro-N-(4-chlorophenyl)-4-(2-ethyl-4-methyl-1,3-thiazol-5- | 4.48* |
| | yl)pyrimidine-2-amine | |
| 878 | 5-chloro-N-(2-chlorophenyl)-4-(2-ethyl-4-methyl-1,3-thiazol-5- | 4.62* |
| | yl)pyrimidine-2-amine | |
| 879 | N′-[4-(2-ethyl-4-methyl-1,3-thiazol-5-yl)pyrimidin-2-yl]-N,N- | 1.56* |
| | dimethylbenzene-1,4-diamine | |
| 880 | N′-[4-(2-ethyl-4-methyl-1,3-thiazol-5-yl)pyrimidin-2-yl]-N,N- | 1.99* |
| | dimethylbenzene-1,3-diamine | |
| 881 | 2-methoxyethyl-4-{[4-(2-ethyl-4-methyl-1,3-thiazol-5-yl)pyrimidin-2- | 2.91* |
| | yl]amino}benzoate | |
| 882 | 4-(2-tert-butyl-4-methyl-1,3-thiazol-5-yl)-N-phenylpyrimidine-2- | 4.24* |
| | amine | |
| 883 | 4-(2-tert-butyl-4-methyl-1,3-thiazol-5-yl)-N-(4- | 4.18* |
| | fluorophenyl)pyrimidine-2-amine | |
| 884 | 4-(2-tert-butyl-4-methyl-1,3-thiazol-5-yl)-N-(3- | 4.82* |
| | chlorophenyl)pyrimidine-2-amine | |
| 885 | 1-(4-{[4-(2-tert-butyl-4-methyl-1,3-thiazol-5-yl)pyrimidin-2- | 4.22* |
| | yl]amino}phenyl)ethanone | |
| 886 | 4-(2-tert-butyl-4-methyl-1,3-thiazol-5-yl)-N-(6-methoxypyridin-3- | 3.42* |
| | yl)pyrimidine-2-amine | |
| 887 | 1-(3-{[4-(2-tert-butyl-4-methyl-1,3-thiazol-5-yl)pyrimidin-2- | 3.66* |
| | yl]amino}phenyl)ethanone | |
| 888 | 4-(2-tert-butyl-4-methyl-1,3-thiazol-5-yl)-N-(3,4,5- | 3.48* |
| | trimethoxyphenyl)pyrimidine-2-amine | |
| 889 | 6-[(4-{2-[(2-methoxy-1-methylethyl)amino]-4-methyl-1,3-thiazol-5- | 1.65* |
| | yl}pyrimidin-2-yl)amino]-2-benzofuran-1(3H)-one | |
| 890 | 6-[(4-{2-[(2-methoxyethyl)amino]4-methyl-1,3-thiazol-5- | 1.53* |
| | yl}pyrimidin-2-yl)amino]-2-benzofuran-1(3H)-one | |
| 891 | 2-[(5-{2-[(4-fluorophenyl)amino]pyrimidin-4-yl}-4-methyl-1,3- | 1.5* |
| | thiazol-2-yl)amino]ethanol | |
| 892 | 6-({4-[2-(tert-butylamine)-4-methyl-1,3-thiazol-5-yl]pyrimidin-2- | 2* |
| | yl}amino)-2-benzofuran-1(3H)-one | |
| 893 | 4-[2-(tert-butylamine)-4-methyl-1,3-thiazol-5-yl]-N-(3,4- | 2.79* |
| | dimethylphenyl)pyrimidine-2-amine | |
| 894 | 4-[2-(tert-butylamine)-4-methyl-1,3-thiazol-5-yl]-N-(3,4- | 2.96* |
| | difluorophenyl)pyrimidine-2-amine | |
| 895 | 4-[2-(tert-butylamine)-4-methyl-1,3-thiazol-5-yl]-N-(4- | 3.13* |
| | chlorophenyl)pyrimidine-2-amine | |
| 896 | N-(3,4-dimethylphenyl)-4-{2-[(2-methoxyethyl)amino]-4-methyl-1,3- | 2.08* |
| | thiazol-5-yl}pyrimidine-2-amine | |
| 897 | N-(3,4-difluorophenyl)-4-{2-[(2-methoxyethyl)amino]-4-methyl-1,3- | 2.15* |
| | thiazol-5-yl}pyrimidine-2-amine | |
| 898 | N-(4-chlorophenyl)-4-{2-[(2-methoxyethyl)amino]-4-methyl-1,3- | 2.24* |
| | thiazol-5-yl}pyrimidine-2-amine | |
| 899 | N-(4-fluorophenyl)-4-[2-(2-methoxyethoxy)-4-methyl-1,3-thiazol-5- | 3.12* |
| | yl]pyrimidine-2-amine | |
| 900 | N2-tert-butyl-N8-(4-fluorophenyl)-4,5-dihydro[1,3]thiazole[4,5- | 2.22* |
| | h]quinazoline-2,8-diamine | |
| 901 | 4-[2-(2-methoxyethoxy)-4-methyl-1,3-thiazol-5-yl]-N- | 3.01* |
| | phenylpyrimidine-2-amine | |
| 902 | 5-bromo-4-(2-ethyl-4-methyl-1,3-thiazol-5-yl)-N-phenylpyrimidine-2- | 3.82* |
| | amine | |
| 903 | 5-chloro-4-(2-isopropyl-4-methyl-1,3-thiazol-5-yl)-N- | 4.27* |
| | phenylpyrimidine-2-amine | |
| 904 | 5-bromo-4-(2-isopropyl-4-methyl-1,3-thiazol-5-yl)-N- | 4.32* |
| | phenylpyrimidine-2-amine | |
| 905 | 5-bromo-4-(2-ethyl-4-methyl-1,3-thiazol-5-yl)-N-(3,4,5- | 3.29* |
| | trimethoxyphenyl)pyrimidine-2-amine | |
| 906 | 5-bromo-4-(2,4-dimethyl-1,3-thiazol-5-yl)-N-(3,4,5- | 2.85* |
| | trimethoxyphenyl)pyrimidine-2-amine | |
| 907 | 5-chloro-4-(2-ethyl-4-methyl-1,3-thiazol-5-yl)-N-(3,4,5- | 3.2* |
| | trimethoxyphenyl)pyrimidine-2-amine | |
| 908 | 5-bromo-4-(2-isopropyl-4-methyl-1,3-thiazol-5-yl)-N-(3,4,5- | 3.65* |
| | trimethoxyphenyl)pyrimidine-2-amine | |
| 909 | 5-bromo-4-(2,4-dimethyl-1,3-thiazol-5-yl)-N-phenylpyrimidine-2- | 3.3* |
| | amine | |
| 910 | 4-[2-(2-methoxyethoxy)-4-methyl-1,3-thiazol-5-yl]-N-(3,4,5- | 2.53* |
| | trimethoxyphenyl)pyrimidine-2-amine | |
| 911 | 4-[2-(2-methoxyethoxy)-4-methyl-1,3-thiazol-5-yl]-N-(3- | 3.08* |
| | nitrophenyl)pyrimidine-2-amine | |
| 912 | 1-[3-({4-[2-(2-methoxyethoxy)-4-methyl-1,3-thiazol-5-yl]pyrimidin-2- | 2.64* |
| | yl}amino)phenyl]ethanone | |
| 913 | N-(3-chlorophenyl)-4-[2-(2-methoxyethoxy)-4-methyl-1,3-thiazol-5- | 3.57* |
| | yl]pyrimidine-2-amine | |
| 914 | 4-[2-(2-methoxyethoxy)-4-methyl-1,3-thiazol-5-yl]-N-(3- | 2.92* |
| | methoxyphenyl)pyrimidine-2-amine | |
| 915 | 4-[2-(2-methoxyethoxy)-4-methyl-1,3-thiazol-5-yl]-N-(6- | 2.34* |
| | methoxypyridin-3-yl)pyrimidine-2-amine | |
| 916 | 1-[4-({4-[2-(2-methoxyethoxy)-4-methyl-1,3-thiazol-5-yl]pyrimidin-2- | 2.58* |
| | yl}amino)phenyl]ethanone | |
| 917 | 1-[4-({4-[2-(2-methoxyethoxy)-4-methyl-1,3-thiazol-5-yl]pyrimidin-2- | 2.23* |
| | yl}amino)phenyl]pyrrolidin-2-one | |
| 918 | N2-tert-butyl-N8-phenyl-4,5-dihydro[1,3]thiazole[4,5-h]quinazoline- | 2.15* |
| | 2,8-diamine | |
| 919 | 4-(2-isopropyl-4-methyl-1,3-thiazol-5-yl)-N-pyridin-3-ylpyrimidine-2- | 1.51* |
| | amine | |
| 920 | 5-bromo-N-(3-chlorophenyl)-4-(2-ethyl-4-methyl-1,3-thiazol-5- | 4.36* |
| | yl)pyrimidine-2-amine | |
| 921 | 4-{[5-bromo-4-(2-ethyl-4-methyl-1,3-thiazol-5-yl)pyrimidin-2- | 3.36* |
| | yl]amino}-N,N-dimethylbenzenesulphonamide | |
| 922 | 5-bromo-N-(3-chlorophenyl)-4-(2-isopropyl-4-methyl-1,3-thiazol-5- | 4.9* |
| | yl)pyrimidine-2-amine | |
| 923 | N2-tert-butyl-N8-(3-chlorophenyl)-4,5-dihydro[1,3]thiazole[4,5- | 2.99* |
| | h]quinazoline-2,8-diamine | |
| 924 | 1-(4-{[2-(tert-butylamine)-4,5-dihydro[1,3]thiazole[4,5-h]quinazolin- | 2.47* |
| | 8-yl]amino}phenyl)ethanone | |
| 925 | 1-(4-{[2-(tert-butylamine)-4,5-dihydro[1,3]thiazole[4,5-h]quinazolin- | 1.91* |
| | 8-yl]amino}phenyl)pyrrolidin-2-one | |
| 926 | N2-tert-butyl-N8-(6-methoxypyridin-3-yl)-4,5- | 1.95* |
| | dihydro[1,3]thiazole[4,5-h]quinazoline-2,8-diamine | |

^{a}The logP values were determined in accordance with EEC Directive 79/831 Annex V.A8 by HPLC (High Performance Liquid Chromatography) on reversed-phase columns (C 18) using the methods below:
*Temperature: 40° C.; mobile phase: 0.1% aqueous formic acid and acetonitrile; linear gradient from 10% acetonitrile to 95% acetonitrile.
**Temperature: 43° C.; mobile phase: 0.1% aqueous phosphoric acid and acetonitrile; linear gradient from 10% acetonitrile to 95% acetonitrile.

Calibration was in each case carried out using unbranched alkan-2-ones (3 to 16 carbon atoms) with known logP values (determination of the logP values by the retention times using linear interpolation between two specific alkanones).

The lambda max values were in each case determined in the maxima of the chromatographic signals using the UV spectra between 190 nm and 400 nm.

### USE EXAMPLES

### Example A

### Leptosphaeria Nodorum Test (Wheat)/Protective

| | |
|---|---|
| Solvent: | 50 parts by weight of N,N-dimethylacetamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with water to the desired concentration.

To test for protective activity, young plants are sprayed with the active compound preparation at the stated application rate. After the spray coating has dried on, the plants are sprayed with a spore suspension of Leptosphaeria nodorum. The plants remain in an incubation cabin at 20° C. and 100% relative atmospheric humidity for 48 hours.

The plants are placed in a greenhouse at a temperature of about 20° C. and a relative atmospheric humidity of 80%.

Evaluation is carried out 10 days after the inoculation. 0% means an efficacy which corresponds to that of the control, whereas an efficacy of 100% means that no infection is observed.

In this test, the following compounds according to the invention show, at an active compound concentration of 1000 ppm, an efficacy of 70% or more:

7, 62, 73, 151, 182, 190, 240, 244, 247, 251, 461, 742, 759

### Example B

### Fusarium graminearum Test (Barley)/Protective

| | |
|---|---|
| Solvent: | 50 parts by weight of N,N-dimethylacetamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with water to the desired concentration.

To test for protective activity, young plants are sprayed with the active compound preparation at the stated application rate. After the spray coating has dried on, the plants are sprayed with a conidia suspension of Fusarium graminearum.

The plants are placed in a greenhouse under translucent incubation hoods at a temperature of about 22° C. and a relative atmospheric humidity of 100%. Evaluation is carried out 4 days after the inoculation. 0% means an efficacy which corresponds to that of the control, whereas an efficacy of 100% means that no infection is observed.

In this test, the following compounds according to the invention show, at an active compound concentration of 1000 ppm, an efficacy of 70% or more:

9, 151, 182, 190, 222, 235, 240, 243, 244, 247, 251, 375, 461

### Example C

### Sphaerotheca Test (Cucumber)/Protective

| | |
|---|---|
| Solvents: | 24.5 parts by weight of acetone |
| | 24.5 parts by weight of dimethylacetamide |
| Emulsifier: | 1 part by weight of alkylaryl polyglycol ether |

To produce a suitable preparation of active compound, 1 part by weight of active compound is mixed with the stated amounts of solvents and emulsifier, and the concentrate is diluted with water to the desired concentration.

To test for protective activity, young plants are sprayed with the active compound preparation at the stated application rate. After the spray coating has dried on, the plants are inoculated with an aqueous spore suspension of Sphaerothecafuliginea. The plants are then placed in a greenhouse at about 23° C. and a relative atmospheric humidity of about 70%.

Evaluation is carried out 7 days after the inoculation. 0% means an efficacy which corresponds to that of the control, whereas an efficacy of 100% means that no infection is observed.

In this test, the compounds according to the invention of the formulae below show, at an active compound concentration of 100 ppm, an efficacy of 70% or more:

10, 244

### Example D

### Botrytis Test (Bean)/Protective

| | |
|---|---|
| Solvents: | 24.5 parts by weight of acetone |
| | 24.5 parts by weight of dimethylacetamide |
| Emulsifier: | 1 part by weight of alkylaryl polyglycol ether |

To produce a suitable preparation of active compound, 1 part by weight of active compound is mixed with the stated amounts of solvents and emulsifier, and the concentrate is diluted with water to the desired concentration.

To test for protective activity, young plants are sprayed with the active compound preparation at the stated application rate. After the spray coating has dried on, 2 small pieces of agar colonized by Botrytis cinerea are placed onto each leaf. The inoculated plants are placed in a dark chamber at about 20° C. and 100% relative atmospheric humidity.

The size of the infected areas on the leaves is evaluated 2 days after the inoculation. 0% means an efficacy which corresponds to that of the control, whereas an efficacy of 100% means that no infection is observed.

In this test, the compounds according to the invention of the formulae below show, at an active compound concentration of 100 ppm, an efficacy of 70% or more:

201, 210, 244, 742, 758, 759, 780

### Example E

### Phytophthora Test (Tomato)/Protective

| | |
|---|---|
| Solvent: | 49 parts by weight of N,N-dimethylformamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with water to the desired concentration.

To test for protective activity, young tomato plants are sprayed with the active compound preparation at the stated application rate. 1 day after the treatment, the plants are inoculated with a spore suspension of Phytophthora infestans and are then allowed to stand at 100% relative humidity and 20° C. for 24 h. The plants are then placed in a climatized chamber at about 96% relative atmospheric humidity and a temperature of about 20° C.

Evaluation is carried out 7 days after the inoculation. 0% means an efficacy which corresponds to that of the control, whereas an efficacy of 100% means that no infection is observed.

In this test, the compounds according to the invention of the formulae below show, at an active compound concentration of 500 ppm, an efficacy of 70% or more:

69, 73, 182, 186, 201, 203, 210, 235, 239, 243, 247, 251, 321, 324, 327, 329, 460, 628, 630

### Example F

### Pyricularia Test (Rice)/Protective

| | |
|---|---|
| Solvent: | 28.5 parts by weight of acetone |
| Emulsifier: | 1.5 parts by weight of alkylaryl polyglycol ether |

To produce a suitable preparation of active compound, 1 part by weight of active compound is mixed with the stated amount of solvent, and the concentrate is diluted with water and the stated amount of emulsifier to the desired concentration.

To test for protective activity, young rice plants are sprayed with the active compound preparation at the stated application rate. 1 day after the treatment, the plants are inoculated with an aqueous spore suspension of Pyricularia oryzae. The plants are then placed in a greenhouse at 100% relative atmospheric humidity and 25° C.

Evaluation is carried out 7 days after the inoculation. 0% means an efficacy which corresponds to that of the control, whereas an efficacy of 100% means that no infection is observed.

In this test, the compounds according to the invention of the example numbers below showed, at an active compound concentration of 500 ppm, an efficacy of 80% or more:

9, 235, 243

### Example G

### Rhizoctonia Test (Rice)/Protective

| | |
|---|---|
| Solvent: | 28.5 parts by weight of acetone |
| Emulsifier: | 1.5 parts by weight of alkylaryl polyglycol ether |

To produce a suitable preparation of active compound, 1 part by weight of active compound is mixed with the stated amount of solvent, and the concentrate is diluted with water and the stated amount of emulsifier to the desired concentration.

To test for protective activity, young rice plants are sprayed with the active compound preparation at the stated application rate. 1 day after the treatment, the plants are inoculated with Hyphae of Rhizoctonia solani. The plants are then placed in a greenhouse at 100% relative atmospheric humidity and 25° C.

Evaluation is carried out 4 days after the inoculation. 0% means an efficacy which corresponds to that of the control, whereas an efficacy of 100% means that no infection is observed. In this test, the compounds according to the invention of the example numbers below showed, at an active compound concentration of 500 ppm, an efficacy of 80% or more:

9, 235, 243, 244

### Example H

### Cochliobolus Test (Rice)/Protective

| | |
|---|---|
| Solvent: | 28.5 parts by weight of acetone |
| Emulsifier: | 1.5 parts by weight of alkylaryl polyglycol ether |

To produce a suitable preparation of active compound, 1 part by weight of active compound is mixed with the stated amount of solvent, and the concentrate is diluted with water and the stated amount of emulsifier to the desired concentration.

To test for protective activity, young rice plants are sprayed with the active compound preparation at the stated application rate. 1 day after the treatment, the plants are inoculated with an aqueous spore suspension of Cochliobolus miyabeanus. The plants are then placed in a greenhouse at 100% relative atmospheric humidity and 25° C.

Evaluation is carried out 7 days after the inoculation. 0% means an efficacy which corresponds to that of the control, whereas an efficacy of 100% means that no infection is observed.

In this test, the compounds according to the invention of the example numbers below showed, at an active compound concentration of 500 ppm, an efficacy of 80% or more:

235, 243, 244

1. A fungicide comprising a compound of the formula (I) 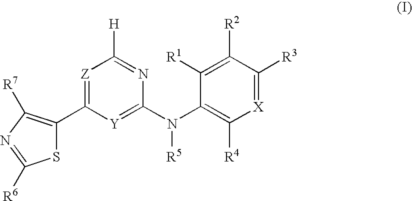
in which the symbols are as defined below:
R¹ to R⁴ independently of one another are hydrogen, halogen, cyano, hydroxyl, nitro, OR¹, SR¹¹, SOR¹, SO₂R¹¹, SO₂N(R¹¹)₂, C═OR¹¹NR¹¹COOR²N(R¹¹)₂ NR¹¹COR¹¹, NR¹¹COR¹¹, NR¹¹SO₂R¹¹, OCON(R¹¹)₂, OC═OR¹¹, CON(R¹¹)₂, COOR¹¹, (CH₂)ₘOR¹¹, (CH₂)ₘSR¹¹, (CH₂)ₘN(R¹¹)₂, (CH₂)ₘCOOR¹², (CH₂)ₘNR¹¹COOR¹¹, unsubstituted or substituted C₁-C₈-alkyl, C₁-C₈-haloalkyl, C₃-C₈-cycloalkyl; where m=1-8
or in each case two adjacent radicals R¹, R² or R², R³ or R³, R³ or
R⁸, R⁴ together form an unsubstituted or substituted heterocyclic five- or six-membered ring,
R⁵ is hydrogen, unsubstituted or substituted C₁-C₈-alkyl, unsubstituted or substituted C₁-C₄-alkylC(═O), C₁-C₄-alkylOC(═O), unsubstituted or substituted C₁-C₄-alkoxy(C₁-C₄)alkyl, unsubstituted or substituted C₂-C₆-alkenyl, unsubstituted or substituted C₂-C₆-alkynyl, C₁-C₆-alkylsulphinyl, C₁-C₆-alkylsulphonyl, C₃-C₈-cycloalkyl; C₁-C₆-haloalkyl, C₁-C₄-haloalkylsulphinyl, C₁-C₄-haloalkylsulphonyl, halo-C₁-C₄-alkoxy-C₁-C₄-alkyl, C₃-C₈-halocycloalkyl having in each case 1 to 9 fluorine, chlorine and/or bromine atoms; formyl, formyl-C₁-C₃-alkyl, (C₁-C₃-alkyl)-carbonyl-C₁-C₃-alkyl, (C₁-C₃-alkoxy)carbonyl-C₁-C₃-alkyl; halo-(C₁-C₃-alkyl)carbonyl-C₁-C₃-alkyl, halo-(C₁-C₃-alkoxy)carbonyl-C₁-C₃-alkyl having in each case 1 to 13 fluorine, chlorine and/or bromine atoms; (C₁-C₈-alkyl)carbonyl, (C₁-C₈-alkoxy)carbonyl, (C₁-C₈-alkylthio)carbonyl, (C₁-C₄-alkoxy-C₁-C₄-alkyl)carbonyl, (C₃-C₆-alkenyloxy)carbonyl, (C₃-C₆-alkynyloxy)carbonyl, (C₃-C₈-cycloalkyl)carbonyl; (C₁-C₆-haloalkyl)carbonyl, (C₁-C₆-haloalkylthio)carbonyl, (C₁-C₆-haloalkoxy)carbonyl, (C₃-C₆-haloalkenyloxy)carbonyl, (C₃-C₆-haloalkynyloxy)carbonyl, (halo-C₁-C₄-alkoxy-C₁-C₄-alkyl)carbonyl, (C₃-C₈-halocycloalkyl)carbonyl having in each case 1 to 9 fluorine, chlorine and/or bromine atoms, or —CH₂—C═C—R^{1-A}, —CH₂—CH═CH—R^{1-A}, —CH═C═CH—R^{1-A}, —C(═O)C(═O)R², —CONR³R⁴, —CH₂NR⁵R⁶, C₁-C₄-trialkylsilyl or C₁-C₄-dialkylmonophenylsilyl,
R^{1-A} represents hydrogen, C₁-C₆-alkyl, C₁-C₆-haloalkyl, C₂-C₆-alkenyl, C₂-C₆-alkynyl, C₃-C₇-cycloalkyl, (C₁-C₄-alkoxy)carbonyl, (C₃-C₆-alkenyloxy)carbonyl, (C₃-C₆-alkynyloxy)carbonyl or cyano,
X is nitrogen or CR³,
Y is nitrogen or CR⁹,
Z is nitrogen or CR¹⁰,
R⁶ is hydrogen, halogen, cyano, hydroxyl, OR¹¹, SR¹¹, COR¹¹, CO₂R¹¹, unsubstituted or substituted C₁-C₈-alkyl, C₁-C₈-haloalkyl, C₁-C₄-trialkylsilyl, N(R¹¹)₂, NR¹¹COR¹¹, NR¹¹COR¹², NR¹¹SOR¹¹, CON(R¹¹)₂, C₃-C₈-cycloalkyl, aryl, hetaryl; where n=0-2,
R⁷ is hydrogen, halogen, cyano, hydroxyl, amino, N(R¹¹)₂, nitro, OR¹¹, SR¹¹, unsubstituted or substituted C₁-C₈-alkyl, unsubstituted or substituted C₃-C₆-cycloalkyl, C₁-C₄-trialkylsilyl, COOR¹¹, CON(R¹¹)₂, COR¹¹or
R⁷ and R¹⁰ together form a saturated or unsaturated bridge of the following structure: 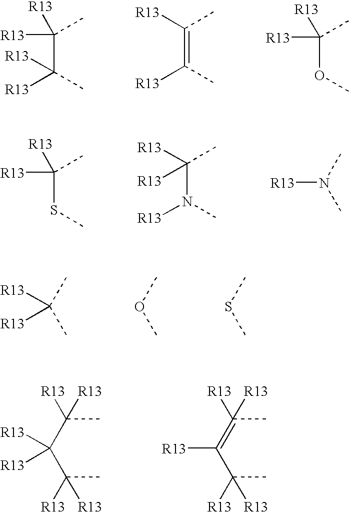
where R¹³=independently of one another hydrogen, halogen, unsubstituted or substituted C₁-C₃-alkyl, unsubstituted or substituted C₁-C₃-alkoxy or
two geminal radicals R¹³ represent doubly attached oxygen or sulphur,
R⁸ is hydrogen, halogen, cyano, hydroxyl, nitro, OR¹¹, SR¹¹, SOR¹¹, SO₂R¹¹, SO₂N(R¹¹)₂, C═OR¹¹, NR¹¹COOR¹², N(R¹¹)₂, NR¹¹COR¹¹, NR¹¹COR¹²NR¹¹SO₂R¹¹, OCON(R¹¹)₂, OC═OR¹¹, CON(R¹¹)₂, COOR¹¹, (CH₂)ₘOR¹¹, (CH₂)ₘSR¹¹, (CH₂)ₘN(R¹¹)₂, (CH₂)ₘCOOR¹², (CH₂)ₘNR¹¹COOR¹¹, unsubstituted or substituted C₁-C₈-alkyl, C₁-C₈-haloalkyl, C₃-C₈-cycloalkyl; where m=1-8,
R⁹ is hydrogen, halogen, N(R¹¹)₂, cyano, hydroxyl, OR¹¹, SR¹¹, COR¹¹, unsubstituted or substituted C₁-C₈-alkyl or unsubstituted or substituted C₃-C₈-cycloalkyl,
R¹⁰ is hydrogen, halogen, cyano, hydroxyl, amino, N(R¹¹)₂, nitro, OR¹¹, SR¹¹, unsubstituted or substituted C₁-C₈-alkyl, C₁-C₄-trialkylsilyl, COOR¹¹, CON(R¹¹)₂,
R¹¹ are identical or different and are hydrogen, unsubstituted or substituted C₁-C₈-alkyl, C₁-C₈-haloalkyl, C₁-C₄-trialkylsilyl, aryl, or
if two radicals R¹¹ are attached to one nitrogen atom, two radicals R¹¹ may form a 3- to 7-membered unsubstituted or substituted saturated or unsaturated cycle which may contain up to five further heteroatoms selected from the group consisting of N, O and S, where two oxygen atoms are not adjacent, or
if two radicals R¹¹ are adjacent in the grouping NR¹¹COR¹¹, two radicals R¹¹ may form a 3- to 7-membered unsubstituted or substituted saturated or unsaturated cycle which may contain up to five further heteroatoms selected from the group consisting of N, O and S, where two oxygen atoms are not adjacent,
R¹² are identical or different and are unsubstituted or substituted C₁-C₈-alkyl, C₁-C₈-haloalkyl, (CH₂)ₚOR¹¹, C₁-C₄-trialkylsilyl; where p=1-4
And/or an agrochemically active salt thereof.

2. A fungicide of claim 1, wherein:
R¹ to R⁴ independently of one another are hydrogen, fluorine, chlorine, bromine, iodine, cyano, hydroxyl, nitro, OR¹¹, O-Me, O-Et, O—Pr, O-iPr, O-Bu, O-secBu, O-isoBu, O-tBu, O—(CH₂)₂OH, O—(CH₂)₂OCH₃, O—(CH₂)₃OH, O—(CH₂)₃OCH₃, OCF₃, SR¹¹, SH, S-Me, S-Et, S—Pr, S-iPr, S-Bu, S-secBu, S-isoBu, S-tBu, SCF₃, SOR¹¹, SO-Me, SO-Et, SO—Pr, SO-iPr, SO-Bu, SO-secBu, SO-isoBu, SO-tBu, SO₂R¹¹, SO₂-Me, SO₂-Et, SO₂—Pr, SO₂-iPr, SO₂—Bu, SO₂-secBu, SO₂-isoBu, SO₂-tBu, SON(R¹¹)₂, SONHMe, SONMe₂, SONHEt, SONEt₂, SONHPr, SONPr₂, SONHBu, SONBu₂, SONHCF₃, SON(CF₃)₂, SO₂N(R¹¹)₂, SO₂NHMe, SO₂NMe₂, SO₂NEt₂, SO₂NHEt, SO₂NPr₂, SO₂NHPr, SO₂NHCF₃, SO₂N(CF₃)₂, COR¹¹, COMe, COEt, COPr, COiPr, COBu, COsecBu, CO-isoBu, COtBu, COCF₃, NR¹¹CO₂R¹¹, NR¹¹CO₂Me, NR¹¹CO₂Et, NR¹¹CO₂Pr, NR¹¹CO₂iPr, NR¹¹CO₂Bu, NR¹¹CO₂secBu, NR¹¹CO₂ isoBu, NR¹¹CO₂tBu, NR¹¹COR¹¹, NHCOMe, NHCOEt, NHCOPr, NHCOiPr, NHCOBu, NHCOiBu, NHCOsecBu, NHCOisoBu, NHCOtBu, NHCO(CH₂)₂OH, NHCO(CH₂)₂OCH₃, NHCO(CH₂)₃OH, NHCO(CH₂)₃OCH₃, NR¹¹COMe, NR¹¹COEt, NR¹¹COPr, NR¹¹COiPr, NR¹¹COBu, NR¹¹COsecBu, NR¹¹COtBu, NR¹¹CO(CH₂)₂OH, NR¹¹CO(CH₂)₂OCH₃, NR¹¹CO(CH₂)₃OH, NR¹¹CO(CH₂)₃OCH₃, N(R¹¹)₂, NMe₂, NEt₂, NHMe, NH₂, NHtBu, NHEt, NHPr, NHiPr, NHBu, NHiBu, NHsecBu, piperazin-1-yl, 4-methylpiperazin-1-yl, morpholin-1-yl, NR¹¹SO₂R¹², NHSOR¹¹, NR¹¹SOR¹¹, NHSOMe, NHSO₂Me, NHSOEt, NHSO₂Et, NMeSOMe, NHSO₂R¹¹, NR¹¹SO₂R¹¹, NMeSO₂Me, NMeSOEt, NMeSO₂Et, NHSOCF₃, NHSO₂CF₃, OCON(R¹¹)₂, OCONHMe, OCONHEt, OCONHPr, OCONHiPr, OCONHBu, OCONHsecBu, OCONHisoBu, OCONHtBu, OCONMe₂, OCONEt₂, OCONPr₂, OCONiPr₂, OCONBu₂, OCONsecBu₂, OCONisoBu₂, OCONHtBu₂, OCOR¹¹, OCOMe, OCOEt, OCOPr, OCOiPr, OCOBu, OCOsecBu, OCOisoBu, OCOtBu, CON(R¹¹)₂, CONHEt, CONEt₂, CONHMe, CONMe₂, CONHPr, CONPr₂, CONHBu, CONHsecBu, CONHisoBu, CONHtBu, CONHCH(CH₃)CH₂OH, CONHCH(CH₃)CH₂OCH₃, CONHCH(C₂H₅)CH₂OH, CONHCH(C₂H₅)CH₂OCH₃, CONH(CH₂)₂OCH₃, CONH(CH₂)₂OH, CONH(CH₂)₃OCH₃, CONH(CH₂)₃OH, CONR¹¹CH(CH₃)CH₂OH, CONR¹¹CH(CH₃)CH₂OCH₃, CONR¹¹CH(C₂H₅)CH₂OH, CONR¹¹CH(C₂H₅)CH₂OCH₃, CONR¹¹(CH₂)₂OCH₃, CONR¹¹(CH₂)₂OH, CONR¹¹(CH₂)₃OCH₃, CONR¹¹(CH₂)₃OH, CO₂R¹¹, CO₂Me, CO₂Et, CO₂Pr, CO₂iPr, CO₂Bu, CO₂secBu, CO₂ isoBu, CO₂tBu, CO₂(CH₂)₂OH, CO₂(CH₂)₂OCH₃, CO₂(CH₂)₃OH, CO₂(CH₂)₃OCH₃, (CH₂)ₘOR¹¹, CH₂OH, (CH₂)₂OH, (CH₂)₃OH, (CH₂)₄OH, CH₂OMe, (CH₂)₂OMe, (CH₂)₃OMe, (CH₂)₄OMe, (CH₂)ₘSR¹¹, CH₂SH, (CH₂)₂SH, (CH₂)₃SH, (CH₂)₄SH, CH₂SMe, (CH₂)₂SMe, (CH₂)₃SMe, (CH₂)₄SMe, (CH₂)ₘN(R¹¹)₂, CH₂NH₂, CH₂NAc₂, CH₂N(COCF₃)₂, CH₂NHAc, CH₂NHCOCF₃, (CH₂)₂NH₂, (CH₂)₃NH₂, (CH₂)₄NH₂, CH₂NMe₂, (CH₂)₂NHMe, (CH₂)₂NMe₂, (CH₂)₃NHMe, (CH₂)₃NMe₂, (CH₂)₄NHMe, (CH₂)₄NMe₂, (CH₂)ₘCOOR¹², CH₂COOMe, (CH₂)₂COOMe, (CH₂)₃COOMe, CH₂COOEt, (CH₂)₂COOEt, (CH₂)₃COOEt, CH₂COOPr, (CH₂)₂COOPr, (CH₂)₃COOPr, CH₂COOiPr, (CH₂)₂COOiPr, (CH₂)₃COOiPr, CH₂COOtBu, (CH₂)₂COOtBu, (CH₂)₃COOtBu, CH₂COO(CH₂)₂OH CH₂COO(CH₂)₂OCH₃, CH₂COO(CH₂)₃OH, CH₂COO(CH₂)₃OCH₃, CH₂NHCOOR¹¹, CH₂NR¹¹COOR¹¹, CH₂NHCOOMe, CH₂NHCOOtBu, CH₂NHCOOEt, CH₂NHCOOPr, CH₂NHCOOiPr, CH₂NHCOOBu, CH₂NHCOOtBu, CH₂NHCOOsecBu, CH₂NHCOOisoBu, CH₂NR¹¹COOEt, CH₂NR¹¹COOPr, CH₂NR¹¹COOiPr, CH₂NR¹¹COOBu, CH₂NR¹¹COOtBu, CH₂NR¹¹COOsecBu, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; cyclopropyl, cyclobutyl, cyclopentyl, cyclohexyl; CF₃, CF₂H, CCl₃, C₂F₅, C₃F₇, CF(CF₃)₂,
or in each case two adjacent radicals R¹, R² or R², R³ or R³, R³ or R⁸, R⁴ together form a heterocyclic five- or six-membered ring which is unsubstituted or substituted by 1 to 4 halogen atoms or by 1 to 4 C₁-C₃-alkyl groups and which contains in each case up to two oxygen atoms,
R⁵ is hydrogen, methyl, ethyl, propyl, 1-methylethyl, butyl, 1-methylpropyl, 2-methylpropyl, 1,1-dimethylethyl, CF₃, CF₂H, CCl₃, C₂F₅, C₃F₇, CF(CF₃)₂;
acetyl, C₂H₅C(═O), C₃H₇C(═O), C₄H₉C(═O), CF₃C(═O), C₂F₅C(═O), CH₃C(═O), C₂H₅OC (═O), C₃H₇C(═O), C₃H₇C(═O), C₄H₉C(═O), CF₃C(═O), CCl₃C(═O), C₂F₅C(═O), CH₂OCH₃; C₂H₄OCH₃, CH═CH₂, CH₂CH═CH₂, C═CH, CH₂C═CH, SOCH₃, SOC₂H₅, SOC₃H₇ SO₂CH₃, SO₂C₂H₅, SO₂C₃H₇, cyclopropyl, cyclobutyl, cyclopentyl, cyclohexyl; CH₂OCF₃; C₂H₄OCF₃, C═ONH₂, SiMe₃, SiMe₂tBu, SiMe₂Ph,
X is nitrogen or CR⁸,
Y is nitrogen or CR⁹,
Z is nitrogen or CR¹⁰,
where either Y and Z are nitrogen,
or Y is nitrogen and Z is CR¹⁰,
or Y is CR⁹ and Z is nitrogen,
R⁶ is hydrogen, fluorine, chlorine, bromine, iodine, cyano, hydroxyl, OR¹¹, O-Me, O-Et, O—Pr, O-iPr, O-Bu, O-secBu, O-isoBu, O-tBu, OCH(CH₃)CH₂OH, OCH(CH₃)CH₂OCH₃, OCH(C₂H₅)CH₂OH, OCH(C₂H₅)CH₂OCH₃, O(CH₂)₂OCH₃, O(CH₂)₂OH, O(CH₂)₃OCH₃, O(CH₂)₃OH, OCF₃, SR¹¹, S-Me, S-Et, S—Pr, S-iPr, S-Bu, S-secBu, S-isoBu, S-tBu, SCF₃, SOR¹¹, SO-Me, SO-Et, SO—Pr, SO-iPr, SO-Bu, SO-secBu, SO-isoBu, SO-tBu, SO₂R¹¹, SO₂-Me, SO₂-Et, SO₂—Pr, SO₂-iPr, SO₂—Bu, SO₂-secBu, SO₂-isoBu, SO₂-tBu, COR¹¹, COMe, COEt, COPr, COiPr, COBu, COsecBu, COisoBu, COtBu, COCF₃, CO₂R¹¹, CO₂Me, CO₂Et, CO₂Pr, CO₂iPr, CO₂Bu, CO₂secBu, CO₂ isoBu, CO₂tBu, CO₂CH(CH₃)CH₂OH, CO₂CH(CH₃)CH₂OCH₃, CO₂CH(C₂H₅)CH₂OH, CO₂CH(C₂H₅)CH₂OCH₃, CO₂(CH₂)₂OCH₃, CO₂(CH₂)₂OH, CO₂(CH₂)₃OCH₃, CO₂(CH₂)₃OH, NR¹¹CO₂R¹¹, NR¹¹CO₂Me, NR¹¹CO₂Et, NR¹¹CO₂Pr, NR¹¹CO₂iPr, NR¹¹CO₂Bu, NR¹¹CO₂secBu, NR¹¹CO₂ isoBu, NR¹¹CO₂tBu, NR¹¹COR¹¹, NR¹¹COMe, NR¹¹COEt, NR¹¹COPr, NR¹¹COiPr, NR¹¹COBu, NR¹¹COsecBu, NR¹¹COisoBu, NR¹¹COtBu, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethyl-butyl, 1,1,2-trimethylpropyl, 1,2,2-trimethylpropyl, 1-ethyl-1-methylpropyl and 1-ethyl-2-methylpropyl; CH₂OCH₃, (CH₂)₂OCH₃, CH(CH₃)CH₂OCH₃, (CH₂)₂OH, CH(CH₃)CH₂OH, CF₃, CF₂H, CCl₃, C₂F₅, C₃F₇, CF(CF₃)₂
SiMe₃, SiMe₂tBu, SiMe₂Ph, NHR¹¹, N(R¹¹)₂, NMe₂, NEt₂, NHMe, NH₂, NHtBu, NHEt, NHPr, NHiPr, NHBu, NHiBu, NHsecBu, piperazin-1-yl, 4-methylpiperazin-1-yl, morpholin-1-yl, NHCH(CH₃)CH₂OH, NHCH(CH₃)CH₂OCH₃, NHCH(C₂H₅)CH₂OH, NHCH(C₂H₅)CH₂OCH₃, NH(CH₂)₂OCH₃, NH(CH₂)₂OH, NH(CH₂)₃OCH₃, NH(CH₂)₃OH, NHAc, NR¹¹CH(CH₃)CH₂OH, NR¹¹CH(CH₃)CH₂OCH₃, NR¹¹CH(C₂H₅)CH₂OH, NR¹¹CH(C₂H₅)CH₂OCH₃, NR¹¹(CH₂)₂OCH₃, NR¹¹(CH₂)₂OH, NR¹¹(CH₂)₃OCH₃, NR¹¹(CH₂)₃OH, NR¹¹Ac, NHCOEt, NHCOPr, NHCOiPr, NHCOBu, NHCOsecBu, NHCOisoBu, NHCOtBu, NHCOCF₃, NHCOC₂F₅, N(Me)tBu, N(Me)Et, N(Me)Pr, N(Me)iPr, N(Me)Bu, N(Me)secBu, N(Me)isoBu, N(CH₃)CH(CH₃)CH₂OH, N(CH₃)CH(CH₃)CH₂OCH₃, N(CH₃)CH(C₂H₅)CH₂OH, N(CH₃)CH(C₂H₅)CH₂OCH₃, N(CH₃)(CH₂)₂OCH₃, N(CH₃)(CH₂)₂OH, N(CH₃)(CH₂)₃OCH₃, N(CH₃)(CH₂)₃OH, NHCOR¹¹, NR¹¹COR¹¹, NMeAc, NMeCOEt, NMeCOPr, NMeCOiPr, NMeCOBu, NMeCOsecBu, NMeCOisoBu, NMeCOtBu, NMeCOCF₃, NMeCOC₂F₅, NHCO₂R¹¹, NR¹¹CO₂R¹¹, NHCO₂Me, NHCO₂Et, NHCO₂Pr, NHCO₂iPr, NHCO₂Bu, NHCO₂secBu, NHCO₂ isoBu, NHCO₂tBu, NHCO₂CF₃, NHCO₂C₂F₅, NHCO₂CH(CH₃)CH₂OH, NHCO₂CH(CH₃)CH₂OCH₃, NHCO₂CH(C₂H₅)CH₂OH, NHCO₂CH(C₂H₆)CH₂OCH₃, NHCO₂(CH₂)₂OCH₃, NHCO₂(CH₂)₂OH, NHCO₂(CH₂)₃OCH₃, NHCO₂(CH₂)₃OH, NMeCO₂Me, NMeCO₂Et, NMeCO₂Pr, NMeCO₂iPr, NMeCO₂Bu, NMeCO₂secBu, NMeCO₂ isoBu, NMeCO₂tBu, NMeCO₂CF₃, NMeCO₂C₂F₅, NEtCO₂Me, NEtCO₂Et, NEtCO₂Pr, NEtCO₂iPr, NEtCO₂Bu, NEtCO₂secBu, NEtCO₂ isoBu, NEtCO₂tBu, NEtCO₂CF₃, NEtCO₂C₂F₅, SON(R¹¹)₂, SONHMe, SONMe₂, SONHEt, SONEt₂, SONHPr, SONPr₂, SONHBu, SONBu₂, SONHCF₃, SON(CF₃)₂, SO₂N(R¹¹)₂, SO₂NHMe, SO₂NMe₂, SO₂NEt₂, SO₂NHEt, SO₂NPr₂, SO₂NHPr, SO₂NHCF₃, SO₂N(CF₃)₂, SO₂NHCH(CH₃)CH₂OH, SO₂NHCH(CH₃)CH₂OCH₃, SO₂NHCH(C₂H₅)CH₂OH, SO₂NHCH(C₂H₅)CH₂OCH₃, SO₂NH(CH₂)₂OCH₃, SO₂NH(CH₂)₂OH, SO₂NH(CH₂)₃OCH₃, SO₂NH(CH₂)₃OH, NHSOR¹¹, NR¹¹SOR¹¹, NHSOMe, NHSO₂Me, NHSOEt, NHSO₂Et, NMeSOMe, NHSO₂R¹¹, NR¹¹SO₂R¹¹, NMeSO₂Me, NMeSOEt, NMeSO₂Et, NHSOCF₃, NHSO₂CF₃, CON(R¹¹)₂, CONH₂, CONHMe, CONMe₂, CONHEt, CONEt₂, CO-morpholine, CO-piperidine, CO-piperazine, CO-(4-methylpiperazine), CONHCH(CH₃)CH₂OH, CONHCH(CH₃)CH₂OCH₃, CONHCH(C₂H₅)CH₂OH, CONHCH(C₂H₅)CH₂OCH₃, CONH(CH₂)₂OCH₃, CONH(CH₂)₂OH, CONH(CH₂)₃OCH₃, CONH(CH₂)₃OH, cyclopropyl, cyclopentyl, cyclohexyl, phenyl, pyridin-4-yl, pyridin-3-yl, pyridin-2-yl, thien-2-yl, thien-3-yl, furan-2-yl, furan-3-yl, N-1,3-oxazolidin-2-one, N-4-methyl-1,3-oxazolidin-2-one, N-4,4-dimethyl-1,3-oxazolidin-2-one, N-4,5-dimethyl-1,3-oxazolidin-2-one, N-5,5-dimethyl-1,3-oxazolidin-2-one, N-4-ethyl-1,3-oxazolidin-2-one, N-4,4-ethyl-1,3-oxazolidin-2-one, NHCH₂-tetrahydrofuran-2-yl,
R⁷ is hydrogen, fluorine, chlorine, bromine, iodine, cyano, hydroxyl, amino, N(R¹¹)₂, nitro, OR¹¹, SR¹¹, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; CF₃, CF₂H, CCl₃, C₂F₅, C₃F₇, CF(CF₃)₂, cyclopropyl, cyclobutyl, cyclopentyl, cyclohexyl, SiMe₃, COOR¹¹, CON(R¹¹)₂, COR¹¹, or
R⁷ and R¹⁰ together form a saturated or unsaturated bridge of the following structure: 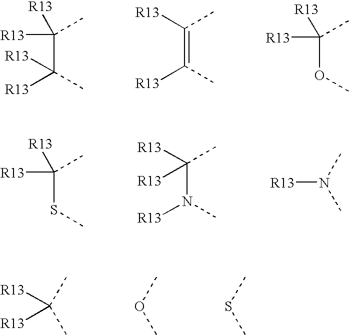
where R¹³=independently of one another hydrogen, fluorine, chlorine, bromine, iodine, methyl, ethyl, propyl, 1-methylethyl, CF₂H, CF₃, C₂F₅, OCH₃, OC₂H₅, OCF₃, OC₂F₅ or
two geminal radicals R¹³ represent doubly attached oxygen or sulphur,
R⁸ is hydrogen, fluorine, chlorine, bromine, iodine, cyano, hydroxyl, nitro, OR¹¹, O-Me, O-Et, O—Pr, O-iPr, O-Bu, O-secBu, O-isoBu, O-tBu, O—(CH₂)₂OH, O—(CH₂)₂OCH₃, O—(CH₂)₃OH, O—(CH₂)₃OCH₃, OCF₃, SR¹¹, SH, S-Me, S-Et, S—Pr, S-iPr, S-Bu, S-secBu, S-isoBu, S-tBu, SCF₃, SOR¹¹, SO-Me, SO-Et, SO—Pr, SO-iPr, SO-Bu, SO-secBu, SO-isoBu, SO-tBu, SO₂R¹¹, SO₂-Me, SO₂-Et, SO₂—Pr, SO₂-iPr, SO₂—Bu, SO₂-secBu, SO₂-isoBu, SO₂-tBu, SON(R¹¹)₂, SONHMe, SONMe₂, SONHEt, SONEt₂, SONHPr, SONPr₂, SONHBu, SONBu₂, SONHCF₃, SON(CF₃)₂, SO₂N(R¹¹)₂, SO₂NHMe, SO₂NMe₂, SO₂NEt₂, SO₂NHEt, SO₂NPr₂, SO₂NHPr, SO₂NHCF₃, SO₂N(CF₃)₂, COR¹¹, COMe, COEt, COPr, COiPr, COBu, COsecBu, COisoBu, COtBu, COCF₃, NR¹¹CO₂R¹¹, NR¹¹CO₂Me, NR¹¹CO₂Et, NR¹¹CO₂Pr, NR¹¹CO₂iPr, NR¹¹CO₂Bu, NR¹¹CO₂secBu, NR¹¹CO₂ isoBu, NR¹¹CO₂tBu, NR¹¹COR¹¹, NHCOMe, NHCOEt, NHCOPr, NHCOiPr, NHCOBu, NHCOiBu, NHCOsecBu, NHCOisoBu, NHCOtBu, NHCO(CH₂)₂OH, NHCO(CH₂)₂OCH₃, NHCO(CH₂)₃OH, NHCO(CH₂)₃OCH₃, NR¹¹COMe, NR¹¹COEt, NR¹¹COPr, NR¹¹COiPr, NR¹¹COBu, NR¹¹COsecBu, NR¹¹COisoBu, NR¹¹COtBu, NR¹¹CO(CH₂)₂OH, NR¹¹CO(CH₂)₂OCH₃, NR¹¹CO(CH₂)₃OH, NR¹¹CO(CH₂)₃OCH₃, N(R¹¹)₂, NMe₂, NEt₂, NHMe, NH₂, NHtBu, NHEt, NHPr, NHiPr, NHBu, NHsecBu, NHisoBu, 4-methylpiperazin-1-yl, piperazin-1-yl, morpholin-1-yl, NR¹¹SO₂R¹², NHSOR¹¹, NR¹¹SOR¹¹, NHSOMe, NHSO₂Me, NHSOEt, NHSO₂Et, NMeSOMe, NHSO₂R¹¹, NR¹¹SO₂R¹¹, NMeSO₂Me, NMeSOEt, NMeSO₂Et, NHSOCF₃, NHSO₂CF₃, OCON(R¹¹)₂, OCONHMe, OCONHEt, OCONHPr, OCONHiPr, OCONHBu, OCONHsecBu, OCONHisoBu, OCONHtBu, OCONMe₂, OCONEt₂, OCONPr₂, OCONiPr₂, OCONBu₂, OCONsecBu₂, OCONisoBu₂, OCONtBu₂, OCOR¹¹, OCOMe, OCOEt, OCOPr, OCOiPr, OCOBu, OCOsecBu, OCOisoBu, OCOtBu, CON(R¹¹)₂, CONHEt, CONEt₂, CONHMe, CONMe₂, CONHPr, CONPr₂, CONHBu, OCONHBu, CONHtBu, CONHCH(CH₃)CH₂OH, CONHCH(CH₃)CH₂OCH₃, CONHCH(C₂H₅)CH₂OH, CONHCH(C₂H₅)CH₂OCH₃, CONH(CH₂)₂OCH₃, CONH(CH₂)₂OH, CONH(CH₂)₃OCH₃, CONH(CH₂)₃OH, CONR¹¹CH(CH₃)CH₂OH, CONR¹¹CH(CH₃)CH₂OCH₃, CONR¹¹CH(C₂H₅)CH₂OH, CONR¹¹CH(C₂H₅)CH₂OCH₃, CONR¹¹(CH₂)₂OCH₃, CONR¹¹(CH₂)₂OH, CONR¹¹(CH₂)₃OCH₃, CONR¹¹(CH₂)₃OH, CO₂R¹¹, CO₂Me, CO₂Et, CO₂Pr, CO₂iPr, CO₂Bu, CO₂secBu, CO₂ isoBu, CO₂tBu, CO₂(CH₂)₂OH, CO₂(CH₂)₂OCH₃, CO₂(CH₂)₃OH, CO₂(CH₂)₃OCH₃, (CH₂)ₘOR¹¹, CH₂OH, (CH₂)₂OH, (CH₂)₃OH, (CH₂)₄OH, CH₂OMe, (CH₂)₂OMe, (CH₂)₃OMe, (CH₂)₄OMe, (CH₂)ₘSR¹¹, CH₂SH, (CH₂)₂SH, (CH₂)₃SH, (CH₂)₄SH, CH₂SMe, (CH₂)₂SMe, (CH₂)₃SMe, (CH₂)₄SMe, (CH₂)ₘN(R¹¹)₂, CH₂NH₂, CH₂NAC₂, CH₂N(COCF₃)₂, CH₂NHAc, CH₂NHCOCF₃, (CH₂)₂NH₂, (CH₂)₃NH₂, (CH₂)₄NH₂, CH₂NMe₂, (CH₂)₂NHMe, (CH₂)₂NMe₂, (CH₂)₃NHMe, (CH₂)₃NMe₂, (CH₂)₄NHMe, (CH₂)₄NMe₂, (CH₂)ₘCOOR¹², CH₂COOMe, (CH₂)₂COOMe, (CH₂)₃COOMe, CH₂COOEt, (CH₂)₂COOEt, (CH₂)₃COOEt, CH₂COOPr, (CH₂)₂COOPr, (CH₂)₃COOPr, CH₂COOiPr, (CH₂)₂COOiPr, (CH₂)₃COOiPr, CH₂COOtBu, (CH₂)₂COOtBu, (CH₂)₃COOtBu, CH₂COO(CH₂)₂OH, CH₂COO(CH₂)₂OCH₃, CH₂COO(CH₂)₃OH, CH₂COO(CH₂)₃OCH₃, CH₂NHCOOR¹¹, CH₂NR¹¹COOR¹¹, CH₂NHCOOMe, CH₂NHCOOtBu, CH₂NHCOOEt, CH₂NHCOOPr, CH₂NHCOOiPr, CH₂NHCOOBu, CH₂NHCOOtBu, CH₂NHCOOsecBu, CH₂NHCOOisoBu, CH₂NR¹¹COOEt, CH₂NR¹¹COOPr, CH₂NR¹¹COOiPr, CH₂NR¹¹COOBu, CH₂NR¹¹COOtBu, CH₂NR¹¹COOsecBu, CH₂NR¹¹COOisoBu, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methyl-pentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; cyclopropyl, cyclobutyl, cyclopentyl, cyclohexyl; CF₃, CF₂H, CCl₃, C₂F₅, C₃F₇, CF(CF₃)₂,
R⁹ is hydrogen, fluorine, chlorine, bromine, iodine, N(R¹¹)₂, NMe₂, NEt₂, NHMe, NH₂, NHtBu, NHEt, NHPr, NHiPr, NHBu, NHsecBu, NHisoBu, 4-methylpiperazin-1-yl, piperazin-1-yl, morpholin-1-yl, cyano, hydroxyl, OR¹¹, O-Me, O-Et, O—Pr, O-iPr, O-Bu, O-secBu, O-isoBu, O-tBu, O—(CH₂)₂OH, O—(CH₂)₂OCH₃, O—(CH₂)₃OH, O—(CH₂)₃OCH₃, OCF₃, SR¹¹, SH, S-Me, S-Et, S—Pr, S-iPr, S-Bu, S-secBu, S-isoBu, S-tBu, SCF₃, COR¹¹, COMe, COEt, COPr, COiPr, COBu, COsecBu, COisoBu, COtBu, COCF₃, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethyl-butyl, 1,1,2-trimethylpropyl, 1,2,2-trimethylpropyl, 1-ethyl-1-methylpropyl and 1-ethyl-2-methylpropyl; CF₃, CF₂H, CCl₃, C₂F₅, C₃F₇, CF(CF₃)₂, cyclopropyl, cyclobutyl, cyclopentyl, cyclohexyl,
R¹⁰ is hydrogen, fluorine, chlorine, bromine, iodine, cyano, hydroxyl, amino, N(R¹¹)₂, nitro, OR¹¹, SR¹¹, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; CF₃, CF₂H, CCl₃, C₂F₅, C₃F₇, CF(CF₃)₂, cyclopropyl, cyclobutyl, cyclopentyl, cyclohexyl, SiMe₃, COOR¹¹, CON(R¹¹)₂,
R¹¹ are identical or different and are hydrogen, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, CH₂CH₂OMe, CH(CH₃)CH₂OMe, CH₂CH(CH₃)OMe, CH₂CH₂OEt, CH(CH₃)CH₂OEt, CH₂CH(CH₃)OEt, CF₃, CF₂H, CCl₃, C₂F₅, C₃F₇, CF(CF₃)₂, cyclopropyl, cyclobutyl, cyclopentyl, cyclohexyl, SiMe₃, phenyl or
if two radicals R¹¹ are attached to one nitrogen atom, two radicals R¹¹ may form a 3- to 7-membered, unsubstituted or substituted saturated or unsaturated cycle which may contain up to five further heteroatoms from the group consisting of N, O and S, where two oxygen atoms are not adjacent, or
if two radicals R¹¹ are adjacent in the grouping NR¹¹COR¹¹, two radicals R¹¹ may form a 3- to 7-membered unsubstituted or substituted saturated or unsaturated cycle which may contain up to five further heteroatoms selected from the group consisting of N, O and S, where two oxygen atoms are not adjacent,
R¹² is methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; CF₃, CF₂H, CCl₃, C₂F₅, C₃F₇, CF(CF₃)₂, cyclopropyl, cyclobutyl, cyclopentyl, cyclohexyl, CH₂OR¹¹, (CH₂)₂OR¹¹, (CH₂)₃OR¹¹, (CH₂)₄OR¹¹, SiMe₃
And/or an agrochemically active salt thereof.

3. A fungicide of claim 1, wherein:
R¹ to R⁴ independently of one another are hydrogen, fluorine, chlorine, bromine, iodine, cyano, hydroxyl, nitro, OR¹¹, O-Me, O-Et, O—Pr, O-iPr, O-Bu, O-secBu, O-isoBu, O-tBu, O—(CH₂)₂OH, O—(CH₂)₂OCH₃, O—(CH₂)₃OH, O—(CH₂)₃OCH₃, OCF₃, SR¹¹, SH, S-Me, S-Et, S—Pr, S-iPr, S-Bu, S-secBu, S-isoBu, S-tBu, SCF₃, SOR¹¹, SO-Me, SO-Et, SO—Pr, SO-iPr, SO-Bu, SO-secBu, SO-isoBu, SO-tBu, SO₂R¹¹, SO₂-Me, SO₂-Et, SO₂—Pr, SO₂-iPr, SO₂—Bu, SO₂-secBu, SO₂-isoBu, SO₂-tBu, SON(R¹¹)₂, SONHMe, SONMe₂, SONHEt, SONEt₂, SONHPr, SONPr₂, SONHBu, SONBu₂, SONHCF₃, SON(CF₃)₂, SO₂N(R¹¹)₂, SO₂NHMe, SO₂NMe₂, SO₂NEt₂, SO₂NHEt, SO₂NPr₂, SO₂NHPr, SO₂NHCF₃, SO₂N(CF₃)₂, COR¹¹, COMe, COEt, COPr, COiPr, COBu, COsecBu, CO-isoBu, COtBu, COCF₃, NR¹¹CO₂R¹¹, NR¹¹CO₂Me, NR¹¹CO₂Et, NR¹¹CO₂Pr, NR¹¹CO₂iPr, NR¹¹CO₂Bu, NR¹¹CO₂secBu, NR¹¹CO₂ isoBu, NR¹¹CO₂tBu, NR¹¹COR¹¹, NHCOMe, NHCOEt, NHCOPr, NHCOiPr, NHCOBu, NHCOiBu, NHCOsecBu, NHCOisoBu, NHCOtBu, NHCO(CH₂)₂OH, NHCO(CH₂)₂OCH₃, NHCO(CH₂)₃OH, NHCO(CH₂)₃OCH₃, NR¹¹COMe, NR¹¹COEt, NR¹¹COPr, NR¹¹COiPr, NR¹¹COBu, NR¹¹COsecBu, NR¹¹COtBu, NR¹¹CO(CH₂)₂OH, NR¹¹CO(CH₂)₂OCH₃, NR¹¹CO(CH₂)₃OH, NR¹¹CO(CH₂)₃OCH₃, N(R¹¹)₂, NMe₂, NEt₂, NHMe, NH₂, NHtBu, NHEt, NHPr, NHiPr, NHBu, NHiBu, NHsecBu, piperazin-1-yl, 4-methylpiperazin-1-yl, morpholin-1-yl, NR¹¹SO₂R¹², NHSOR¹¹, NR¹¹SOR¹¹NHSOMe, NHSO₂Me, NHSOEt, NHSO₂Et, NMeSOMe, NHSO₂R¹¹, NR¹¹SO₂R¹¹, NMeSO₂Me, NMeSOEt, NMeSO₂Et, NHSOCF₃, NHSO₂CF₃, OCON(R¹¹)₂, OCONHMe, OCONHEt, OCONHPr, OCONHiPr, OCONHBu, OCONHsecBu, OCONHisoBu, OCONHtBu, OCONMe₂, OCONEt₂, OCONPr₂, OCONiPr₂, OCONBu₂, OCONsecBu₂, OCONisoBu₂, OCONHtBu₂, OCOR¹¹, OCOMe, OCOEt, OCOPr, OCOiPr, OC9Bu, OCOsecBu, OCOisoBu, OCOtBu, CON(R¹¹)₂, CONHEt, CONEt₂, CONHMe, CONMe₂, CONHPr, CONPr₂, CONHBu, CONHsecBu, CONHisoBu, CONHtBu, CONHCH(CH₃)CH₂OH, CONHCH(CH₃)CH₂OCH₃, CONHCH(C₂H₅)CH₂OH, CONHCH(C₂H₅)CH₂OCH₃, CONH(CH₂)₂OCH₃, CONH(CH₂)₂OH, CONH(CH₂)₃OCH₃, CONH(CH₂)₃OH, CONR¹¹CH(CH₃)CH₂OH, CONR¹¹CH(CH₃)CH₂OCH₃, CONR¹¹CH(C₂H₅)CH₂OH, CONR¹¹CH(C₂H₅)CH₂OCH₃, CONR¹¹(CH₂)₂OCH₃, CONR¹¹(CH₂)₂OH, CONR¹¹(CH₂)₃OCH₃, CONR¹¹(CH₂)₃OH, CO₂R¹¹, CO₂Me, CO₂Et, CO₂Pr, CO₂iPr, CO₂Bu, CO₂secBu, CO₂ isoBu, CO₂tBu, CO₂(CH₂)₂OH, CO₂(CH₂)₂OCH₃, CO₂(CH₂)₃OH, CO₂(CH₂)₃OCH₃, (CH₂)ₘOR¹¹CH₂OH, (CH₂)₂OH, (CH₂)₃OH, (CH₂)₄OH, CH₂OMe, (CH₂)₂OMe, (CH₂)₃OMe, (CH₂)₄OMe, (CH₂)ₘSR¹¹, CH₂SH, (CH₂)₂SH, (CH₂)₃SH, (CH₂)₄SH, CH₂SMe, (CH₂)₂SMe, (CH₂)₃SMe, (CH₂)₄SMe, (CH₂)ₘN(R¹¹)₂, CH₂NH₂, CH₂NAc₂, CH₂N(COCF₃)₂, CH₂NHAc, CH₂NHCOCF₃, (CH₂)₂NH₂, (CH₂)₃NH₂, (CH₂)₄NH₂, CH₂NMe₂, (CH₂)₂NHMe, (CH₂)₂NMe₂, (CH₂)₃NHMe, (CH₂)₃NMe₂, (CH₂)₄NHMe, (CH₂)₄NMe₂, (CH₂)ₘCOOR¹², CH₂COOMe, (CH₂)₂COOMe, (CH₂)₃COOMe, CH₂COOEt, (CH₂)₂COOEt, (CH₂)₃COOEt, CH₂COOPr, (CH₂)₂COOPr, (CH₂)₃COOPr, CH₂COOiPr, (CH₂)₂COOiPr, (CH₂)₃COOiPr, CH₂COOtBu, (CH₂)₂COOtBu, (CH₂)₃COOtBu, CH₂COO(CH₂)₂OH CH₂COO(CH₂)₂OCH₃, CH₂COO(CH₂)₃OH, CH₂COO(CH₂)₃OCH₃, CH₂NHCOOR¹¹, CH₂NR¹¹COOR¹¹, CH₂NHCOOMe, CH₂NHCOOtBu, CH₂NHCOOEt, CH₂NHCOOPr, CH₂NHCOOiPr, CH₂NHCOOBu, CH₂NHCOOtBu, CH₂NHCOOsecBu, CH₂NHCOOisoBu, CH₂NR¹¹COOEt, CH₂NR¹¹COOPr, CH₂NR¹¹COOiPr, CH₂NR¹¹COOBu, CH₂NR¹¹COOtBu, CH₂NR¹¹COOsecBu, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; cyclopropyl, cyclobutyl, cyclopentyl, cyclohexyl; CF₃, CF₂H, CCl₃, C₂F₅, C₃F₇, CF(CF₃)₂,
or in each case two adjacent radicals R¹, R² or R², R³ or R³, R³ or R⁸, R⁴ together form a 2,3-dihydro-1,4-dioxin, a 2,2,3,3-tetrafluoro-2,3-dihydro-1,4-dioxin, a 1,3-dioxole or a 2,2-difluoro-1,3-dioxole ring,
R⁵ is hydrogen, acetyl, trifluoroacetyl,
X is nitrogen or CR⁸,
Y is nitrogen,
Z is nitrogen or CR¹⁰,
R⁶ is hydrogen, fluorine, chlorine, bromine, iodine, cyano, hydroxyl, OR¹¹, O-Me, O-Et, O—Pr, O-iPr, O-Bu, O-secBu, O-isoBu, O-tBu, OCH(CH₃)CH₂OH, OCH(CH₃)CH₂OCH₃, OCH(C₂H₅)CH₂OH, OCH(C₂H₅)CH₂OCH₃, O(CH₂)₂OCH₃, O(CH₂)₂OH, O(CH₂)₃OCH₃, O(CH₂)₃OH, OCF₃, SR¹¹, S-Me, S-Et, S—Pr, S-iPr, S-Bu, S-secBu, S-isoBu, S-tBu, SCF₃, SOR¹¹, SO-Me, SO-Et, SO—Pr, SO-iPr, SO-Bu, SO-secBu, SO-isoBu, SO-tBu, SO₂R¹¹, SO₂-Me, SO₂-Et, SO₂—Pr, SO₂-iPr, SO₂—Bu, SO₂-secBu, SO₂-isoBu, SO₂-tBu, COR¹¹, COMe, COEt, COPr, COiPr, COBu, COsecBu, COisoBu, COtBu, COCF₃, CO₂R¹¹, CO₂Me, CO₂Et, CO₂Pr, CO₂iPr, CO₂Bu, CO₂secBu, CO₂ isoBu, CO₂tBu, CO₂CH(CH₃)CH₂OH, CO₂CH(CH₃)CH₂OCH₃, CO₂CH(C₂H₅)CH₂OH, CO₂CH(C₂H₅)CH₂OCH₃, CO₂(CH₂)₂OCH₃, CO₂(CH₂)₂OH, CO₂(CH₂)₃OCH₃, CO₂(CH₂)₃OH, NR¹¹CO₂R¹¹, NR¹¹CO₂Me, NR¹¹CO₂Et, NR¹¹CO₂Pr, NR¹¹CO₂iPr, NR¹¹CO₂Bu, NR¹¹CO₂secBu, NR¹¹CO₂ isoBu, NR¹¹CO₂tBu, NR¹¹COR¹¹, NR¹¹COMe, NR¹¹COEt, NR¹¹COPr, NR¹¹COiPr, NR¹¹COBu, NR¹¹COsecBu, NR¹¹COisoBu, NR¹¹COtBu, Me, Et, Pr, iPr, Bu, secBu, isoBu, tBu, CH₂OCH₃, (CH₂)₂OCH₃, CH(CH₃)CH₂OCH₃, (CH₂)₂OH, CH(CH₃)CH₂OH, CF₃, CF₂H, CCl₃, C₂F₅, C₃F₇, CF(CF₃)₂,
SiMe₃, SiMe₂tBu, SiMe₂Ph, NHR¹¹, N(R¹¹)₂, NMe₂, NEt₂, NHMe, NH₂, NHtBu, NHEt, NHPr, NHiPr, NHBu, NHsecBu, NHisoBu, NHCH(CH₃)CH₂OH, NHCH(CH₃)CH₂OCH₃, NHCH(C₂H₅)CH₂OH, NHCH(C₂H₅)CH₂OCH₃, NH(CH₂)₂OCH₃, NH(CH₂)₂OH, NH(CH₂)₃OCH₃, NH(CH₂)₃OH, NHAc, NR¹¹CH(CH₃)CH₂OH, NR¹¹CH(CH₃)CH₂OCH₃, NR¹¹CH(C₂H₅)CH₂OH, NR¹¹CH(C₂H₅)CH₂OCH₃, NR¹¹(CH₂)₂OCH₃, NR¹¹(CH₂)₂OH, NR¹¹(CH₂)₃OCH₃, NR¹¹(CH₂)₃OH, NR¹¹Ac, NHCOEt, NHCOPr, NHCOiPr, NHCOBu, NHCOsecBu, NHCOisoBu, NHCOtBu, NHCOCF₃, NHCOC₂F₅, N(Me)tBu, N(Me)Et, N(Me)Pr, N(Me)iPr, N(Me)Bu, N(Me)secBu, N(Me)isoBu, N(CH₃)CH(CH₃)CH₂OH, N(CH₃)CH(CH₃)CH₂OCH₃, N(CH₃)CH(C₂H₅)CH₂OH, N(CH₃)CH(C₂H₅)CH₂OCH₃, N(CH₃)(CH₂)₂OCH₃, N(CH₃)(CH₂)₂OH, N(CH₃)(CH₂)₃OCH₃, N(CH₃)(CH₂)₃OH, NHCOR¹¹, NR¹¹COR¹¹, NMeAc, NMeCOEt, NMeCOPr, NMeCOiPr, NMeCOBu, NMeCOsecBu, NMeCOisoBu, NMeCOtBu, NMeCOCF₃, NMeCOC₂F₅, NHCO₂R¹¹, NR¹¹CO₂R¹¹, NHCO₂Me, NHCO₂Et, NHCO₂Pr, NHCO₂iPr, NHCO₂Bu, NHCO₂secBu, NHCO₂ isoBu, NHCO₂tBu, NHCO₂CF₃, NHCO₂C₂F₅, NHCO₂CH(CH₃)CH₂OH, NHCO₂CH(CH₃)CH₂OCH₃, NHCO₂CH(C₂H₅)CH₂OH, NHCO₂CH(C₂H₅)CH₂OCH₃, NHCO₂(CH₂)₂OCH₃, NHCO₂(CH₂)₂OH, NHCO₂(CH₂)₃OCH₃, NHCO₂(CH₂)₃OH, NMeCO₂Me, NMeCO₂Et, NMeCO₂Pr, NMeCO₂iPr, NMeCO₂Bu, NMeCO₂secBu, NMeCO₂ isoBu, NMeCO₂tBu, NMeCO₂CF₃, NMeCO₂C₂F₅, NEtCO₂Me, NEtCO₂Et, NEtCO₂Pr, NEtCO₂iPr, NEtCO₂Bu, NEtCO₂secBu, NEtCO₂ isoBu, NEtCO₂tBu, NEtCO₂CF₃, NEtCO₂C₂F₅, SON(R¹¹)₂, SONHMe, SONMe₂, SONHEt, SONEt₂, SONHPr, SONPr₂, SONHBu, SONBu₂, SONHCF₃, SON(CF₃)₂, SO₂N(R¹¹)₂, SO₂NHMe, SO₂NMe₂, SO₂NEt₂, SO₂NHEt, SO₂NPr₂, SO₂NHPr, SO₂NHCF₃, SO₂N(CF₃)₂, SO₂NHCH(CH₃)CH₂OH, SO₂NHCH(CH₃)CH₂OCH₃, SO₂NHCH(C₂H₅)CH₂OH, SO₂NHCH(C₂H₅)CH₂OCH₃, SO₂NH(CH₂)₂OCH₃, SO₂NH(CH₂)₂OH, SO₂NH(CH₂)₃OCH₃, SO₂NH(CH₂)₃OH, NHSOR¹¹, NR¹¹SOR¹¹, NHSOMe, NHSO₂Me, NHSOEt, NHSO₂Et, NMeSOMe, NHSO₂R¹¹, NR¹¹SO₂R¹¹, NMeSO₂Me, NMeSOEt, NMeSO₂Et, NHSOCF₃, NHSO₂CF₃, CON(R¹¹)₂, CONH₂, CONHMe, CONMe₂, CONHEt, CONEt₂, CO-morpholine, CO-piperidine, CO-piperazine, CO-(4-methylpiperazine), CONHCH(CH₃)CH₂OH, CONHCH(CH₃)CH₂OCH₃, CONHCH(C₂H₅)CH₂OH, CONHCH(C₂H₅)CH₂OCH₃, CONH(CH₂)₂OCH₃, CONH(CH₂)₂OH, CONH(CH₂)₃OCH₃, CONH(CH₂)₃OH, cyclopropyl, cyclopentyl, cyclohexyl, phenyl, pyridin-4-yl, pyridin-3-yl, pyridin-2-yl, thien-2-yl, thien-3-yl, furan-2-yl, furan-3-yl, N-1,3-oxazolidin-2-one, N-4-methyl-1,3-oxazolidin-2-one, N-4,4-dimethyl-1,3-oxazolidin-2-one, N-4,5-dimethyl-1,3-oxazolidin-2-one, N-5,5-dimethyl-1,3-oxazolidin-2-one, N-4-ethyl-1,3-oxazolidin-2-one, N-4,4-ethyl-1,3-oxazolidin-2-one, NHCH₂-tetrahydrofuran-2-yl,
R⁷ is hydrogen, fluorine, chlorine, bromine, iodine, cyano, hydroxyl, amino, N(R¹¹)₂, NMe₂, NEt₂, NHMe, NH₂, NHtBu, NHEt, NHPr, NHiPr, NHBu, NHiBu, NHsecBu, piperazin-1-yl, 4-methylpiperazin-1-yl, morpholin-1-yl, nitro, OR¹¹, O-Me, O-Et, O—Pr, O-iPr, O-Bu, O-secBu, O-isoBu, O-tBu, OCH(CH₃)CH₂OH, OCH(CH₃)CH₂OCH₃, OCH(C₂H₅)CH₂OH, OCH(C₂H₅)CH₂OCH₃, O(CH₂)₂OCH₃, O(CH₂)₂OH, O(CH₂)₃OCH₃, O(CH₂)₃OH, OCF₃, SR¹¹, S-Me, S-Et, S—Pr, S-iPr, S-Bu, S-secBu, S-isoBu, S-tBu, SCF₃, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methyl-pentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; CF₃, CF₂H, CCl₃, C₂F₅, C₃F₇, CF(CF₃)₂, cyclopropyl, cyclobutyl, cyclopentyl, cyclohexyl, SiMe₃, COOR¹¹, CO₂Me, CO₂Et, CO₂Pr, CO₂iPr, CO₂Bu, CO₂secBu, CO₂ isoBu, CO₂tBu, CON(R¹¹)₂, CONH₂, CONHMe, CONMe₂, CONHEt, CONEt₂, CO-morpholine, CO-piperidine, CO-piperazine, CO-(4-methylpiperazine), CONHCH(CH₃)CH₂OH, CONHCH(CH₃)CH₂OCH₃, CONHCH(C₂H₅)CH₂OH, CONHCH(C₂H₅)CH₂OCH₃, CONH(CH₂)₂OCH₃, CONH(CH₂)₂OH, CONH(CH₂)₃OCH₃, CONH(CH₂)₃OH, COR¹¹, COMe, COEt, COPr, COiPr, COBu, COsecBu, COisoBu, COtBu, COF₃ or
R⁷ and R¹⁰ together form a saturated or unsaturated bridge of the following structure: 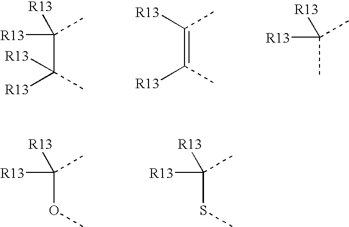
where R¹³=independently of one another hydrogen, fluorine, chlorine, bromine, iodine, methyl, ethyl, propyl, 1-methylethyl, CF₂H, CF₃, C₂F₅, OCH₃; OC₂H₅, OCF₃, OC₂F₆ or
two geminal radicals R¹³ represent doubly attached oxygen or sulphur,
R⁸ is hydrogen, fluorine, chlorine, bromine, iodine, cyano, hydroxyl, nitro, OR¹¹, O-Me, O-Et, O—Pr, O-iPr, O-Bu, O-secBu, O-isoBu, O-tBu, O—(CH₂)₂OH, O—(CH₂)₂OCH₃, O—(CH₂)₃OH, O—(CH₂)₃OCH₃, OCF₃, SR¹¹, SH, S-Me, S-Et, S—Pr, S-iPr, S-Bu, S-secBu, S-isoBu, S-tBu, SCF₃, SOR¹¹, SO-Me, SO-Et, SO—Pr, SO-iPr, SO-Bu, SO-secBu, SO-isoBu, SO-tBu, SO₂R¹¹, SO₂-Me, SO₂-Et, SO₂—Pr, SO₂-iPr, SO₂—Bu, SO₂-secBu, SO₂-isoBu, SO₂-tBu, SON(R¹¹)₂, SONHMe, SONMe₂, SONHEt, SONEt₂, SONHPr, SONPr₂, SONHBu, SONBu₂, SONHCF₃, SON(CF₃)₂, SO₂N(R¹¹)₂, SO₂NHMe, SO₂NMe₂, SO₂NEt₂, SO₂NHEt, SO₂NPr₂, SO₂NHPr, SO₂NHCF₃, SO₂N(CF₃)₂, COR¹¹, COMe, COEt, COPr, COiPr, COBu, COsecBu, COisoBu, COtBu, COCF₃, NR¹¹CO₂R¹¹, NR¹¹CO₂Me, NR¹¹CO₂Et, NR¹¹CO₂Pr, NR¹¹CO₂iPr, NR¹¹CO₂Bu, NR¹¹CO₂secBu, NR¹¹CO₂ isoBu, NR¹¹CO₂tBu, NR¹¹COR¹¹, NHCOMe, NHCOEt, NHCOPr, NHCOiPr, NHCOBu, NHCOiBu, NHCOsecBu, NHCOisoBu, NHCOtBu, NHCO(CH₂)₂OH, NHCO(CH₂)₂OCH₃, NHCO(CH₂)₃OH, NHCO(CH₂)₃OCH₃, NR¹¹COMe, NR¹¹COEt, NR¹¹COPr, NR¹¹COiPr, NR¹¹COBu, NR¹¹COsecBu, NR¹¹COisoBu, NR¹¹COtBu, NR¹¹CO(CH₂)₂OH, NR¹¹CO(CH₂)₂OCH₃, NR¹¹CO(CH₂)₃OH, NR¹¹CO(CH₂)₃OCH₃, N(R¹¹)₂, NMe₂, NEt₂, NHMe, NH₂, NHtBu, NHEt, NHPr, NHiPr, NHBu, NHsecBu, NHisoBu, 4-methylpiperazin-1-yl, piperazin-1-yl, morpholin-1-yl, NR¹¹SO₂R¹², NHSOR¹¹, NR¹¹SOR¹¹, NHSOMe, NHSO₂Me, NHSOEt, NHSO₂Et, NMeSOMe, NHSO₂R¹¹, NR¹¹SO₂R¹¹, NMeSO₂Me, NMeSOEt, NMeSO₂Et, NHSOCF₃, NHSO₂CF₃, OCON(R¹¹)₂, OCONHMe, OCONHEt, OCONHPr, OCONHPr, OCONHBu, OCONHsecBu, OCONHisoBu, OCONHtBu, OCONMe₂, OCONEt₂, OCONPr₂, OCONiPr₂, OCONBu₂, OCONsecBu₂, OCONisoBu₂, OCONtBu₂, OCOR¹¹, OCOMe, OCOEt, OCOPr, OCOiPr, OCOBu, OCOsecBu, OCOisoBu, OCOtBu, CON(R¹¹)₂, CONHEt, CONEt₂, CONHMe, CONMe₂, CONHPr, CONPr₂, CONHBu, OCONHBu, CONHtBu, CONHCH(CH₃)CH₂OH, CONHCH(CH₃)CH₂OCH₃, CONHCH(C₂H₅)CH₂OH, CONHCH(C₂H₅)CH₂OCH₃, CONH(CH₂)₂OCH₃, CONH(CH₂)₂OH, CONH(CH₂)₃OCH₃, CONH(CH₂)₃OH, CONR¹¹CH(CH₃)CH₂OH, CONR¹¹CH(CH₃)CH₂OCH₃, CONR¹¹CH(C₂H₅)CH₂OH, CONR¹¹CH(C₂H₅)CH₂OCH₃, CONR¹¹(CH₂)₂OCH₃, CONR¹¹(CH₂)₂OH, CONR¹¹(CH₂)₃OCH₃, CONR¹¹(CH₂)₃OH, CO₂R¹¹, CO₂Me, CO₂Et, CO₂Pr, CO₂iPr, CO₂Bu, CO₂secBu, CO₂ isoBu, CO₂tBu, CO₂(CH₂)₂OH, CO₂(CH₂)₂OCH₃, CO₂(CH₂)₃OH, CO₂(CH₂)₃OCH₃, (CH₂)ₘOR¹¹, CH₂OH, (CH₂)₂OH, (CH₂)₃OH, (CH₂)₄OH, CH₂OMe, (CH₂)₂OMe, (CH₂)₃OMe, (CH₂)₄OMe, (CH₂)ₘSR¹¹, CH₂SH, (CH₂)₂SH, (CH₂)₃SH, (CH₂)₄SH, CH₂SMe, (CH₂)₂SMe, (CH₂)₃SMe, (CH₂)₄SMe, (CH₂)ₘN(R¹¹)₂, CH₂NH₂, CH₂NAc₂, CH₂N(COCF₃)₂, CH₂NHAc, CH₂NHCOCF₃, (CH₂)₂NH₂, (CH₂)₃NH₂, (CH₂)₄NH₂, CH₂NMe₂, (CH₂)₂NHMe, (CH₂)₂NMe₂, (CH₂)₃NHMe, (CH₂)₃NMe₂, (CH₂)₄NHMe, (CH₂)₄NMe₂, (CH₂)ₘCOOR¹², CH₂COOMe, (CH₂)₂COOMe, (CH₂)₃COOMe, CH₂COOEt, (CH₂)₂COOEt, (CH₂)₃COOEt, CH₂COOPr, (CH₂)₂COOPr, (CH₂)₃COOPr, CH₂COOiPr, (CH₂)₂COOiPr, (CH₂)₃COOiPr, CH₂COOtBu, (CH₂)₂COOtBu, (CH₂)₃COOtBu, CH₂COO(CH₂)₂OH, CH₂COO(CH₂)₂OCH₃, CH₂COO(CH₂)₃OH, CH₂COO(CH₂)₃OCH₃, CH₂NHCOOR¹¹, CH₂NR¹¹COOR¹¹, CH₂NHCOOMe, CH₂NHCOOtBu, CH₂NHCOOEt, CH₂NHCOOPr, CH₂NHCOOiPr, CH₂NHCOOBu, CH₂NHCOOtBu, CH₂NHCOOsecBu, CH₂NHCOOisoBu, CH₂NR¹¹COOEt, CH₂NR¹¹COOPr, CH₂NR¹¹COOiPr, CH₂NR¹¹COOBu, CH₂NR¹¹COOtBu, CH₂NR¹¹COOsecBu, CH₂NR¹¹COOisoBu, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methyl-pentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; cyclopropyl, cyclobutyl, cyclopentyl, cyclohexyl; CF₃, CF₂H, CCl₃, C₂F₅, C₃F₇, CF(CF₃)₂,
R¹⁰ is hydrogen, fluorine, chlorine, bromine, iodine, cyano, hydroxyl, amino, N(R¹¹)₂, NMe₂, NEt₂, NHMe, NH₂, NHtBu, NHEt, NHPr, NHiPr, NHBu, NHsecBu, NHisoBu, 4-methylpiperazin-1-yl, piperazin-1-yl, morpholin-1-yl, nitro, OR¹¹, O-Me, O-Et, O—Pr, O-iPr, O-Bu, O-secBu, O-isoBu, O-tBu, OCH(CH₃)CH₂OH, OCH(CH₃)CH₂OCH₃, OCH(C₂H₅)CH₂OH, OCH(C₂H₅)CH₂OCH₃, O(CH₂)₂OCH₃, O(CH₂)₂OH, O(CH₂)₃OCH₃, O(CH₂)₃OH, OCF₃, SR¹¹, S-Me, S-Et, S—Pr, S-iPr, S-Bu, S-secBu, S-isoBu, S-tBu, SCF₃, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methyl-pentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; CF₃, CF₂H, CCl₃, C₂F₅, C₃F₇, CF(CF₃)₂, cyclopropyl, cyclobutyl, cyclopentyl, cyclohexyl, SiMe₃, COOR¹¹, CO₂Me, CO₂Et, CO₂Pr, CO₂iPr, CO₂Bu, CO₂secBu, CO₂ isoBu, CO₂tBu, CON(R¹¹)₂ CONH₂, CONHMe, CONMe₂, CONHEt, CONEt₂, CO-morpholine, CO-piperidine, CO-piperazine, CO-(4-methylpiperazine), CONHCH(CH₃)CH₂OH, CONHCH(CH₃)CH₂OCH₃, CONHCH(C₂H₅)CH₂OH, CONHCH(C₂H₅)CH₂OCH₃, CONH(CH₂)₂OCH₃, CONH(CH₂)₂OH, CONH(CH₂)₃OCH₃, CONH(CH₂)₃OH,
R¹¹ are identical or different and are hydrogen, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, CH₂CH₂OMe, CH(CH₃)CH₂OMe, CH₂CH(CH₃)OMe, CH₂CH₂OEt, CH(CH₃)CH₂OEt, CH₂CH(CH₃)OEt, CF₃, CF₂H, CCl₃, C₂F₅, C₃F₇, CF(CF₃)₂, cyclopropyl, cyclobutyl, cyclopentyl, cyclohexyl, SiMe₃, phenyl or
if two radicals R¹¹ are attached to one nitrogen atom, these radicals together may represent piperidin-1-yl, piperazin-1-yl, 4-methylpiperazin-1-yl or morpholin-1-yl, or
if two radicals R¹¹ are adjacent in the grouping NR¹¹COR¹¹, two radicals R¹¹ may form a 3- to 7-membered, unsubstituted or substituted saturated or unsaturated cycle which may contain up to five further heteroatoms selected from the group consisting of N, O and S, where two oxygen atoms are not adjacent,
R¹² is methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; CF₃, CF₂H, CCl₃, C₂F₅, C₃F₇, CF(CF₃)₂, cyclopropyl, cyclobutyl, cyclopentyl, cyclohexyl, CH₂OR¹¹, (CH₂)₂OR¹¹, (CH₂)₃OR¹¹, (CH₂)₄OR¹¹, SiMe₃
And/or an agrochemically active salts thereof as fungicides.

4. A fungicide of claim 1, wherein:
R¹ to R⁴ independently of one another are hydrogen, fluorine, chlorine, cyano, hydroxyl, nitro, OMe, CF₃, COCH₃, COOCH₃, COOH, N(Me)₂, NHCOCH₃, NHCOCF₃, NHSO₂CH₃, NHCOOCH₃, NHCOO(CH₂CH₂)OCH₃, 4-N-methylpiperazin-1-yl, CH₂NH₂, CH₂NHCOOCH₃, CH₂NHCOOtBu, methyl,
or in each case two adjacent radicals R¹, R² or R², R³ or R³, R⁸ or R⁸, R⁴ together form a 2,3-dihydro-1,4-dioxin or a 1,3-dioxole ring,
R⁵ is hydrogen, COCH₃,
X is nitrogen or CR³,
Y is nitrogen,
Z is nitrogen or CR¹⁰,
R⁶ is hydrogen, S-Me, NHCOMe, NHCOCF₃, NMe₂, NHMe, NH₂, NHtBu, NHCH(CH₃)CH₂OH, NHCH(CH₃)CH₂OCH₃, CONMe₂, cyclopropyl, cyclohexyl, phenyl, N-1,3-oxazolidin-2-one, N-4-methyl-1,3-oxazolidin-2-one, NHCH₂-tetrahydrofuran-2-yl,
R⁷ is hydrogen, methyl, 1-methylpropyl, CF₃, CF₂H, cyclopropyl, or
R⁷ and R¹⁰ together form a saturated or unsaturated bridge of the following structure: 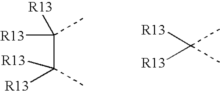
where R¹³=hydrogen,
R⁸ is hydrogen, fluorine, chlorine, cyano, hydroxyl, nitro, OMe, CF₃, COCH₃, COOCH₃, COOH, N(Me)₂, NHCOCH₃, NHCOCF₃, NHSO₂CH₃, NHCOOCH₃, NHCOO(CH₂CH₂)OCH₃, 4-N-methylpiperazin-1-yl, CH₂NH₂, CH₂NHCOOCH₃, CH₂NHCOOtBu, methyl,
R¹⁰ is hydrogen or
R⁷ and R¹⁰ together form a saturated or unsaturated bridge of the following structure: 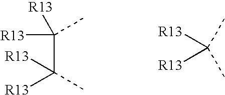
where R¹³=hydrogen
and/or an agrochemically active salt thereof.

5. A fungicide of claim 1, wherein:
R¹ to R⁴ independently of one another are hydrogen, fluorine, chlorine, iodine, cyano, hydroxyl, nitro, OMe, OCH₂tBu, OCOCH₃, SO₂NH₂, SO₂N(CH₃)₂, COCH₃, COCH₂CH₂CH₃, COOCH₂CH₂OCH₃, COOCH₂CH₂CH₂OCH₃, CONHCH₂CH₂OCH₃, NH₂, N(Me)₂, NHCOCH₃, NHSO₂CH₃, NHCOOCH₃, NHCOOCH₂CH₃, NHCOOtBu, NHCOOCH₂CH₂OCH₃, 4-N-acetylpiperazin-1-yl, N-pyrrolidin-2-on-1-yl, CH₂NHCOOCH₃, CH₂NHCOOtBu, methyl, CF₃
or in each case two adjacent radicals R¹, R² or R², R³ or R³, R⁸ or R⁸, R⁴ together form a 2,3-dihydrofuran-2-one, 2,3-dihydro-1,4-dioxin, a 2,2,3,3-tetrafluoro-2,3-dihydro-1,4-dioxin, a 1,3-dioxole or a 2,2-difluoro-1,3-dioxole ring,
R⁵ is hydrogen, COCH₃, COCF₃, COOCH₃,
X is nitrogen or CR³,
R⁸ is hydrogen, O-Me,
Y is nitrogen,
Z is CR¹⁰,
R¹⁰ is hydrogen, fluorine, chlorine, bromine, cyano, CF₃ or
R⁷ and R¹⁰ together form a CH₂ or a CH₂CH₂ bridge,
R⁶ is methyl, ethyl, isopropyl, cyclopropyl, phenyl, 4-chlorophenyl, 3-pyridyl, CH₂OCH₃, O(CH₂)₂OCH₃, S-Me, NH₂, NHMe, NMe₂, NHtBu, NHBu, NHiBu, N-morpholinyl, NH(CH₂)₂OH, NH(CH₂)₂OCH₃, NH(CH₂)₃OCH₃, NHCH(CH₃)CH₂OCH₃, NCH₃(CH₂)₂OCH₃, NCH₃(CH₂)₃OCH₃, NHCH₂-tetrahydrofuran-2-yl, N-1,3-oxazolidin-2-one,
R⁷ is hydrogen, methyl, CF₃, CF₂H, or
R⁷ and R¹⁰ together form a CH₂ or a CH₂CH₂ bridge And/or an also agrochemically active salt thereof.

6. A fungicide of claim 1, wherein:
R¹ to R⁴ independently of one another are hydrogen, fluorine, chlorine, iodine, cyano, hydroxyl, nitro, OMe, OCH₂tBu, OCOCH₃, SO₂NH₂, SO₂N(CH₃)₂, COCH₃, COCH₂CH₂CH₃, COOCH₂CH₂OCH₃,
COOCH₂CH₂CH₂OCH₃, CONHCH₂CH₂OCH₃, NH₂, N(Me)₂, NHCOCH₃, NHSO₂CH₃, NHCOOCH₃, NHCOOCH₂CH₃, NHCOOtBu, NHCOOCH₂CH₂OCH₃, CH₂NHCOOCH₃, CH₂NHCOOtBu, methyl, CF₃
or in each case two adjacent radicals R¹, R² or R², R³ or R³, R⁸ or R⁸, R⁴ together form a 2,3-dihydro-1,4-dioxin, a 2,2,3,3-tetrafluoro-2,3-dihydro-1,4-dioxin, a 1,3-dioxole or 2,2-difluoro-1,3-dioxole ring,
R⁵ is hydrogen, COCH₃, COCF₃, COOCH₃,
X is nitrogen or CR⁸,
R⁸ is hydrogen, O-Me,
Y is nitrogen,
Z is CR¹⁰,
R¹⁰ is hydrogen, fluorine, chlorine, bromine, cyano, CF₃ or
R⁷ and R¹⁰ together form a CH₂— or a CH₂CH₂ bridge,
R⁶ is methyl, ethyl, isopropyl, cyclopropyl, phenyl, 3-pyridyl, CH₂OCH₃, O(CH₂)₂OCH₃, S-Me, NH₂, NHMe, NMe₂, NHtBu, NHBu, NHiBu, N-morpholinyl, NH(CH₂)₂OH, NH(CH₂)₂OCH₃, NH(CH₂)₃OCH₃, NHCH(CH₃)CH₂OCH₃, NCH₃(CH₂)₂OCH₃, NCH₃(CH₂)₃OCH₃, NHCH₂-tetrahydrofuran-2-yl, N-1,3-oxazolidin-2-one,
R⁷ is hydrogen, methyl, CF₃, CF₂H, or
R⁷ and R¹⁰ together form a CH₂ or a CH₂CH₂ bridge
And/or an agrochemically active salt thereof.

7. Composition for controlling unwanted microorganisms, comprising at least one fungicide according to claim 1 and optionally an extender and/or surfactant.

8. Composition according to claim 7, comprising at least one further agrochemically active compound.

9. Method for controlling unwanted microorganisms, comprising applying a fungicide according to claim 1 to unwanted microorganisms and/or a habitat thereof.

10. Process for preparing a composition for controlling unwanted microorganisms, comprising mixing a fungicide of claim 1 with an extender and/or surfactant.

11. A compound of the formula Ia 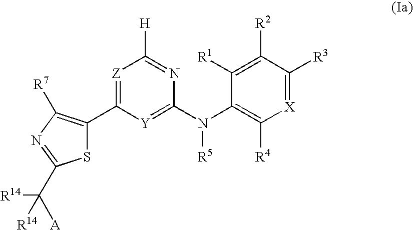
in which the symbols are as defined below:
R¹ to R⁴ independently of one another are hydrogen, halogen, cyano, hydroxyl, nitro, OR¹¹, SR¹¹, SOR¹¹, SO₂R¹¹, SO₂N(R¹¹)₂, C═OR¹¹, NR¹¹COOR¹², N(R¹¹)₂, NR¹¹COR¹¹, NR¹¹COR¹¹, NR¹¹SO₂R¹¹, OCON(R¹¹)₂, OC═OR¹¹, CON(R¹¹)₂, COOR¹¹, (CH₂)ₘOR¹¹, (CH₂)ₘSR¹¹, (CH₂)ₘN(R¹¹)₂, (CH₂)ₘCOOR¹², (CH₂)ₘNR¹¹COOR¹¹, unsubstituted or substituted C₁-C₈-alkyl, C₁-C₈-haloalkyl, C₃-C₈-cycloalkyl; where m=1-8
or in each case two adjacent radicals R¹, R² or R², R³ or R³, R⁸ or R⁸, R⁴ together form an unsubstituted or substituted heterocyclic five- or six-membered ring,
R⁵ is hydrogen, unsubstituted or substituted C₁-C₈-alkyl, unsubstituted or substituted C₁-C₄-alkylC(═O), C₁-C₄-alkylOC(═O), unsubstituted or substituted C₁-C₄-alkoxy(C₁-C₄)alkyl, unsubstituted or substituted C₂-C₆-alkenyl, unsubstituted or substituted C₂-C₆-alkynyl, C₁-C₆-alkylsulphinyl, C₁-C₆-alkylsulphonyl, C₃-C₈-cycloalkyl; C₁-C₆-haloalkyl, C₁-C₄-haloalkylsulphinyl, C₁-C₄-haloalkylsulphonyl, halo-C₁-C₄-alkoxy-C₁-C₄-alkyl, C₃-C₈-halocycloalkyl having in each case 1 to 9 fluorine, chlorine and/or bromine atoms; formyl, formyl-C₁-C₃-alkyl, (C₁-C₃-alkyl)-carbonyl-C₁-C₃-alkyl, (C₁-C₃-alkoxy)carbonyl-C₁-C₃-alkyl; halo-(C₁-C₃-alkyl)carbonyl-C₁-C₃-alkyl, halo-(C₁-C₃-alkoxy)carbonyl-C₁-C₃-alkyl having in each case 1 to 13 fluorine, chlorine and/or bromine atoms; (C₁-C₈-alkyl)carbonyl, (C₁-C₈-alkoxy)carbonyl, (C₁-C₈-alkylthio)carbonyl, (C₁-C₄-alkoxy-C₁-C₄-alkyl)carbonyl, (C₃-C₆-alkenyloxy)carbonyl, (C₃-C₆-alkynyloxy)carbonyl, (C₃-C₈-cycloalkyl)carbonyl; (C₁-C₆-haloalkyl)carbonyl, (C₁-C₆-haloalkylthio)carbonyl, (C₁-C₆-haloalkoxy)carbonyl, (C₃-C₆-haloalkenyloxy)carbonyl, (C₃-C₆-haloalkynyloxy)carbonyl, (halo-C₁-C₄-alkoxy-C₁-C₄-alkyl)carbonyl, (C₃-C₈-halocycloalkyl)carbonyl having in each case 1 to 9 fluorine, chlorine and/or bromine atoms, or —CH₂—C≡C—R^{1-A}, —CH₂—CH═CH—R^{1-A}, —CH═C═CH—R^{1-A}, —C(═O)C(═O)R², CONR³R⁴, —CH₂NR⁵R⁶, C₁-C₄-trialkylsilyl or C₁-C₄-dialkylmonophenylsilyl,
R^{1-A} represents hydrogen, C₁-C₆-alkyl, C₁-C₆-haloalkyl, C₂-C₆-alkenyl, C₂-C₆-alkynyl, C₃-C₇-cycloalkyl, (C₁-C₄-alkoxy)carbonyl, (C₃-C₆-alkenyloxy)carbonyl, (C₃-C₆-alkynyloxy)carbonyl or cyano,
X is nitrogen or CR⁸,
Y is nitrogen or CR⁹,
Z is nitrogen or CR¹⁰,
R⁷ is hydrogen, halogen, cyano, hydroxyl, amino, N(R¹¹)₂, nitro, OR¹¹, SR¹¹, unsubstituted or substituted C₁-C₈-alkyl, unsubstituted or substituted C₃-C₆-cycloalkyl, C₁-C₄-trialkylsilyl, unsubstituted or substituted aryl or hetaryl, COOR¹¹, CON(R¹¹)₂, COR¹¹ or
R⁷ and R¹⁰ together form a saturated or unsaturated bridge of the following structure: 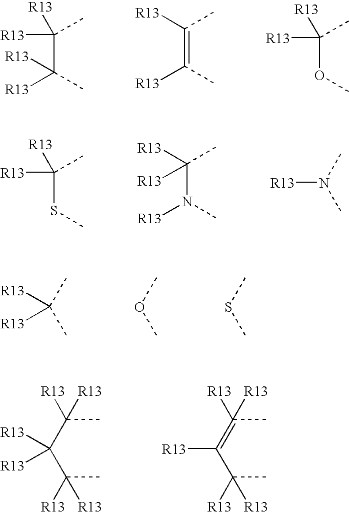
where R¹³=independently of one another hydrogen, halogen, unsubstituted or substituted C₁-C₃-alkyl, unsubstituted or substituted C₁-C₃-alkoxy or
two geminal radicals R¹³ represent doubly attached oxygen or sulphur,
R⁸ is hydrogen, halogen, cyano, hydroxyl, nitro, OR¹¹, SR¹¹, SOR¹¹, SO₂R¹¹SO₂N(R¹¹)₂, C═OR¹¹, NR¹¹COOR¹², N(R¹¹)₂, NR¹¹COR¹¹, NR¹¹COR¹², NR¹¹SO₂R¹¹, OCON(R¹¹)₂, OC═OR¹¹, CON(R¹¹)₂, COOR¹¹, (CH₂)ₘOR¹¹, (CH₂)ₘSR¹¹, (CH₂)ₘN(R¹¹)₂, (CH₂)ₘCOOR¹², (CH₂)ₘNR¹¹COOR¹¹, unsubstituted or substituted C₁-C₈-alkyl, C₁-C₈-haloalkyl, C₃-C₈-cycloalkyl; where m=1-8,
R⁹ is hydrogen, halogen, N(R¹¹)₂, cyano, hydroxyl, OR¹¹, SR¹¹, COR¹¹, unsubstituted or substituted C₁-C₈-alkyl or unsubstituted or substituted C₃-C₈-cycloalkyl,
R¹⁰ is hydrogen, halogen, cyano, hydroxyl, amino, N(R¹¹)₂, nitro, OR¹¹, SR¹¹, unsubstituted or substituted C₁-C₈-alkyl, C₁-C₄-trialkylsilyl, COOR¹¹, CON(R¹¹)₂,
R¹¹ are identical or different and are hydrogen, unsubstituted or substituted C₁-C₈-alkyl, C₁-C₈-haloalkyl, C₁-C₄-trialkylsilyl, aryl, or
if two radicals R¹¹ are attached to one nitrogen atom, two radicals R¹¹ may form a 3- to 7-membered unsubstituted or substituted saturated or unsaturated cycle which may contain up to five further heteroatoms selected from the group consisting of N, O and S, where two oxygen atoms are not adjacent, or
if two radicals R¹¹ are adjacent in the grouping NR¹¹COR¹¹, two radicals R¹¹ may form a 3- to 7-membered unsubstituted or substituted saturated or unsaturated cycle which may contain up to five further heteroatoms selected from the group consisting of N, O and S, where two oxygen atoms are not adjacent,
R¹² are identical or different and are unsubstituted or substituted C₁-C₈-alkyl, C₁-C₈-haloalkyl, (CH₂)ₜOR¹¹, C₁-C₄-trialkylsilyl; where t=1-4.
R¹⁴ independently of one another are hydrogen, halogen, cyano, hydroxyl, OR¹¹, SR¹¹, COR¹¹, unsubstituted or substituted C₁-C₈-alkyl, C₁-C₈-haloalkyl, C₁-C₄-trialkylsilyl or unsubstituted or substituted C₃-C₈-cycloalkyl,
A is OR¹⁵, SR¹⁵, N(R¹⁵)₂ or C(R¹⁶)₃,
R¹⁵ independently of one another are COR¹⁷, unsubstituted or substituted C₂-C₈-alkyl, C₁-C₈-haloalkyl, unsubstituted or substituted C₃-C₈-cycloalkyl, (CH₂)ᵤOR¹¹; where u=1-4,
R¹⁶ independently of one another are hydrogen, halogen, cyano, hydroxyl, OR¹¹, SR¹¹, COR¹¹, N(R¹¹)₂, unsubstituted or substituted C₁-C₈-alkyl, C₁-C₈-haloalkyl, or unsubstituted or substituted C₃-C₈-cycloalkyl, (CH₂)ᵤOR¹¹; where u=1-4; or
in each case two radicals R¹⁶ represent doubly attached oxygen or doubly attached sulphur, or
in each case two radicals R¹⁴, R¹⁴ or R¹⁵, R¹⁵ or R¹⁶, R¹⁶ or R¹⁴, R¹⁶ together form a saturated or unsaturated carbocyclic 3- to 7-membered ring or an optionally substituted 4-pyridyl,
R¹⁷ is unsubstituted or substituted C₁-C₃-alkyl or C₁-C₃-haloalkyl
And/or an agrochemically active salt thereof.

12. A Compound of claim 11, wherein:
R¹ to R⁴ independently of one another are hydrogen, fluorine, chlorine, bromine, iodine, cyano, hydroxyl, nitro, OR¹¹, O-Me, O-Et, O—Pr, O-iPr, O-Bu, O-secBu, O-isoBu, O-tBu, O—(CH₂)₂OH, O—(CH₂)₂OCH₃, O—(CH₂)₃OH, O—(CH₂)₃OCH₃, OCF₃, SR¹¹, SH, S-Me, S-Et, S—Pr, S-iPr, S-Bu, S-secBu, S-isoBu, S-tBu, SCF₃, SOR¹¹, SO-Me, SO-Et, SO—Pr, SO-iPr, SO-Bu, SO-secBu, SO-isoBu, SO-tBu, SO₂R¹¹, SO₂-Me, SO₂-Et, SO₂—Pr, SO₂-iPr, SO₂—Bu, SO₂-secBu, SO₂-isoBu, SO₂-tBu, SON(R¹¹)₂, SONHMe, SONMe₂, SONHEt, SONEt₂, SONHPr, SONPr₂, SONHBu, SONBu₂, SONHCF₃, SON(CF₃)₂, SO₂N(R¹¹)₂, SO₂NHMe, SO₂NMe₂, SO₂NEt₂, SO₂NHEt, SO₂NPr₂, SO₂NHPr, SO₂NHCF₃, SO₂N(CF₃)₂, COR¹¹, COMe, COEt, COPr, COiPr, COBu, COsecBu, CO-isoBu, COtBu, COCF₃, NR¹¹CO₂R¹¹, NR¹¹CO₂Me, NR¹¹CO₂Et, NR¹¹CO₂Pr, NR¹¹CO₂iPr, NR¹¹CO₂Bu, NR¹¹CO₂secBu, NR¹¹CO₂ isoBu, NR¹¹CO₂tBu, NR¹¹COR¹¹, NHCOMe, NHCOEt, NHCOPr, NHCOiPr, NHCOBu, NHCOiBu, NHCOsecBu, NHCOisoBu, NHCOtBu, NHCO(CH₂)₂OH, NHCO(CH₂)₂OCH₃, NHCO(CH₂)₃OH, NHCO(CH₂)₃OCH₃, NR¹¹COMe, NR¹¹COEt, NR¹¹COPr, NR¹¹COiPr, NR¹¹COBu, NR¹¹COsecBu, NR¹¹COtBu, NR¹¹CO(CH₂)₂OH, NR¹¹CO(CH₂)₂OCH₃, NR¹¹CO(CH₂)₃OH, NR¹¹CO(CH₂)₃OCH₃, N(R¹¹)₂, NMe₂, NEt₂, NHMe, NH₂, NHtBu, NHEt, NHPr, NHiPr, NHBu, NHiBu, NHsecBu, piperazin-1-yl, 4-methylpiperazin-1-yl, morpholin-1-yl, NR¹¹SO₂R¹², NHSOR¹¹, NR¹¹SOR¹¹, NHSOMe, NHSO₂Me, NHSOEt, NHSO₂Et, NMeSOMe, NHSO₂R¹¹, NR¹¹SO₂R¹¹, NMeSO₂Me, NMeSOEt, NMeSO₂Et, NHSOCF₃, NHSO₂CF₃, OCON(R¹¹)₂, OCONHMe, OCONHEt, OCONHPr, OCONHiPr, OCONHBu, OCONHsecBu, OCONHisoBu, OCONHtBu, OCONMe₂, OCONEt₂, OCONPr₂, OCONiPr₂, OCONBu2, OCONsecBu₂, OCONisoBu₂, OCONHtBu₂, OCOR¹¹, OCOMe, OCOEt, OCOPr, OCOiPr, OCOBu, OCOsecBu, OCOisoBu, OCOtBu, CON(R¹¹)₂, CONHEt, CONEt₂, CONHMe, CONMe₂, CONHPr, CONPr₂, CONHBu, CONHsecBu, CONHisoBu, CONHtBu, CONHCH(CH₃)CH₂OH, CONHCH(CH₃)CH₂OCH₃, CONHCH(C₂H₅)CH₂OH, CONHCH(C₂H₅)CH₂OCH₃, CONH(CH₂)₂OCH₃, CONH(CH₂)₂OH, CONH(CH₂)₃OCH₃, CONH(CH₂)₃OH, CONR¹¹CH(CH₃)CH₂OH, CONR¹¹CH(CH₃)CH₂OCH₃, CONR¹¹CH(C₂H₅)CH₂OH, CONR¹¹CH(C₂H₅)CH₂OCH₃, CONR¹¹(CH₂)₂OCH₃, CONR¹¹(CH₂)₂OH, CONR¹¹(CH₂)₃OCH₃, CONR¹¹(CH₂)₃OH, CO₂R¹¹, CO₂Me, CO₂Et, CO₂Pr, CO₂iPr, CO₂Bu, CO₂secBu, CO₂ isoBu, CO₂tBu, CO₂(CH₂)₂OH, CO₂(CH₂)₂OCH₃, CO₂(CH₂)₃OH, CO₂(CH₂)₃OCH₃, (CH₂)ₘOR¹¹, CH₂OH, (CH₂)₂OH, (CH₂)₃OH, (CH₂)₄OH, CH₂OMe, (CH₂)₂OMe, (CH₂)₃OMe, (CH₂)₄OMe, (CH₂)ₘSR¹¹, CH₂SH, (CH₂)₂SH, (CH₂)₃SH, (CH₂)₄SH, CH₂SMe, (CH₂)₂SMe, (CH₂)₃SMe, (CH₂)₄SMe, (CH₂)ₘN(R¹¹)₂, CH₂NH₂, CH₂NAc₂, CH₂N(COCF₃)₂, CH₂NHAc, CH₂NHCOCF₃, (CH₂)₂NH₂, (CH₂)₃NH₂, (CH₂)₄NH₂, CH₂NMe₂, (CH₂)₂NHMe, (CH₂)₂NMe₂, (CH₂)₃NHMe, (CH₂)₃NMe₂, (CH₂)₄NHMe, (CH₂)₄NMe₂, (CH₂)ₘCOOR¹², CH₂COOMe, (CH₂)₂COOMe, (CH₂)₃COOMe, CH₂COOEt, (CH₂)₂COOEt, (CH₂)₃COOEt, CH₂COOPr, (CH₂)₂COOPr, (CH₂)₃COOPr, CH₂COOiPr, (CH₂)₂COOiPr, (CH₂)₃COOiPr, CH₂COOtBu, (CH₂)₂COOtBu, (CH₂)₃COOtBu, CH₂COO(CH₂)₂OH CH₂COO(CH₂)₂OCH₃, CH₂COO(CH₂)₃OH, CH₂COO(CH₂)₃OCH₃, CH₂NHCOOR¹¹, CH₂NR¹¹CH₂R¹¹, CH₂NHCOOMe, CH₂NHCOOtBu, CH₂NHCOOEt, CH₂NHCOOPr, CH₂NHCOOiPr, CH₂NHCOOBu, CH₂NHCOOtBu, CH₂NHCOOsecBu, CH₂NHCOOisoBu, CH₂NR¹¹COOEt, CH₂NR¹¹COOPr, CH₂ NH₂NHR¹¹COOiPr, CH₂NR¹¹COOBu, CH₂NR¹¹COOtBu, CH₂NR¹¹COOsecBu, methyl, ethyl, propyl, 1-methylethyl, butyl, 1-methylpropyl, 2-methylpropyl, 1,1-dimethylethyl, pentyl, 1-methylbutyl, 2-methylpbutyl, 3-methylpbutyl, 2,2-dimethylpropyl, 1-ethylpropyl, hexyl, 1,12-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; cyclopropyl, cyclobutyl, cyclopentyl, cyclohexyl; CF₃, CF₂H, CCl₃, C₂F₅, C₃F₇, CF(CF₃)₂
or in each case two adjacent radicals R¹, R² or R², R³ or R³, R⁸ or R⁸, R⁴ together form a heterocyclic five- or six-membered ring which is unsubstituted or substituted by 1 to 4 halogen atoms or by 1 to 4 C₁-C₃-alkyl groups and which contains in each case up to two oxygen atoms,
R⁵ is hydrogen, methyl, ethyl, propyl, 1-methylethyl, butyl, 1-methylpropyl, 2-methylpropyl, 1,1-dimethylethyl, CF₃, CF₂H, CCl₃, C₂F₅, C₃F₇, CF(CF₃)₂,
acetyl, C₂H₅C(═O), C₃H₇C(═O), C₄H₉C(═O), CF₃C(═O), C₂F₅C(═O), CH₃C(═O), C₂H₅OC (═O), C₃H₇C(═O), C₃H₇C(═O), C₄H₉C(═O), CF₃C(═O), CCl₃C(═O), C₂F₅C(═O), CH₂OCH₃; C₂H₄OCH₃, CH═CH₂, CH₂CH═CH₂, C≡CH, CH₂C≡CH, SOCH₃, SOC₂H₅, SOC₃H₇ SO₂CH₃, SO₂C₂H₅, SO₂C₃H₇, cyclopropyl, cyclobutyl, cyclopentyl, cyclohexyl; CH₂OCF₃; C₂H₄OCF₃, C═ONH₂, SiMe₃, SiMe₂tBu, SiMe₂Ph,
X is nitrogen or CR³,
Y is nitrogen or CR⁹,
Z is nitrogen or CR¹⁰,
where either Y and Z are nitrogen,
or Y is nitrogen and Z is CR¹⁰,
or Y is CR⁹ and Z is nitrogen,
R⁷ is hydrogen, fluorine, chlorine, bromine, iodine, cyano, hydroxyl, amino, N(R¹¹)₂, nitro, OR¹¹, SR¹¹, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; CF₃, CF₂H, CCl₃, C₂F₅, C₃F₇, CF(CF₃)₂, cyclopropyl, cyclobutyl, cyclopentyl, cyclohexyl, phenyl, p-Cl-phenyl, p-F-phenyl, p-Br-phenyl, p-I-phenyl, p-methoxyphenyl, p-trifluoromethoxy phenyl, 2-pyridyl, 3-pyridyl, 4-pyridyl, 2-thienyl, 3-thienyl, SiMe₃, COOR¹¹, CON(R¹¹)₂, COR¹¹, or
R⁷ and R¹⁰ together form a saturated or unsaturated bridge of the following structure: 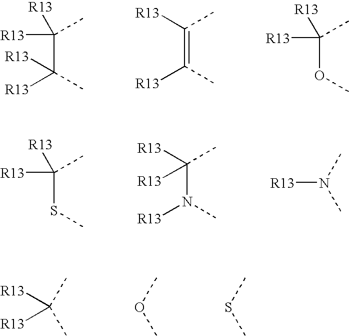
where R¹³=independently of one another hydrogen, fluorine, chlorine, bromine, iodine, methyl, ethyl, propyl, 1-methylethyl, CF₂H, CF₃, C₂F₅, OCH₃, OC₂H₅, OCF₃, OC₂F₅ or
two geminal radicals R¹³ represent doubly attached oxygen or sulphur,
R⁸ is hydrogen, fluorine, chlorine, bromine, iodine, cyano, hydroxyl, nitro, OR¹¹, O-Me, O-Et, O—Pr, O-iPr, O-Bu, O-secBu, O-isoBu, O-tBu, O—(CH₂)₂OH, O—(CH₂)₂OCH₃, O—(CH₂)₃OH, O—(CH₂)₃OCH₃, OCF₃, SR¹¹, SH, S-Me, S-Et, S—Pr, S-iPr, S-Bu, S-secBu, S-isoBu, S-tBu, SCF₃, SOR¹¹, SO-Me, SO-Et, SO—Pr, SO-iPr, SO-Bu, SO-secBu, SO-isoBu, SO-tBu, SO₂R¹¹, SO₂-Me, SO₂-Et, SO₂—Pr, SO₂-iPr, SO₂—Bu, SO₂-secBu, SO₂-isoBu, SO₂-tBu, SON(R¹¹)₂, SONHMe, SONMe₂, SONHEt, SONEt₂, SONHPr, SONPr₂, SONHBu, SONBu₂, SONHCF₃, SON(CF₃)₂, SO₂N(R¹¹)₂, SO₂NHMe, SO₂NMe₂, SO₂NEt₂, SO₂NHEt, SO₂NPr₂, SO₂NHPr, SO₂NHCF₃, SO₂N(CF₃)₂, COR¹¹, COMe, COEt, COPr, COiPr, COBu, COsecBu, COisoBu, COtBu, COCF₃, NR¹¹CO₂R¹¹, NR¹¹CO₂Me, NR¹¹CO₂Et, NR¹¹CO₂Pr, NR¹¹CO₂iPr, NR¹¹CO₂Bu, NR¹¹CO₂secBu, NR¹¹CO₂ isoBu, NR¹¹CO₂tBu, NR¹¹COR¹¹, NHCOMe, NHCOEt, NHCOPr, NHCOiPr, NHCOBu, NHCOiBu, NHCOsecBu, NHCOisoBu, NHCOtBu, NHCO(CH₂)₂OH, NHCO(CH₂)₂OCH₃, NHCO(CH₂)₃OH, NHCO(CH₂)₃OCH₃, NR¹¹COMe, NR¹¹COEt, NR¹¹COPr, NR¹¹COiPr, NR¹¹COBu, NR¹¹COsecBu, NR¹¹COisoBu, NR¹¹COtBu, NR¹¹CO(CH₂)₂OH, NR¹¹CO(CH₂)₂OCH₃, NR¹¹CO(CH₂)₃OH, NR¹¹CO(CH₂)₃OCH₃, N(R¹¹)₂, NMe₂, NEt₂, NHMe, NH₂, NHtBu, NHEt, NHPr, NHiPr, NHBu, NHsecBu, NHisoBu, 4-methylpiperazin-1-yl, piperazin-1-yl, morpholin-1-yl, NR¹¹SO₂R¹², NHSOR¹¹, NR¹¹SOR¹¹, NHSOMe, NHSO₂Me, NHSOEt, NHSO₂Et, NMeSOMe, NHSO₂R¹¹NR¹¹SO₂R¹¹, NMeSO₂Me, NMeSOEt, NMeSO₂Et, NHSOCF₃, NHSO₂CF₃, OCON(R¹¹)₂, OCONHMe, OCONHEt, OCONHPr, OCONHiPr, OCONHBu, OCONHsecBu, OCONHisoBu, OCONHtBu, OCONMe₂, OCONEt₂, OCONPr₂, OCONiPr₂, OCONBu₂, OCONsecBu₂, OCONisoBu₂, OCONtBu₂, OCOR¹¹, OCOMe, OCOEt, OCOPr, OCOiPr, OCOBu, OCOsecBu, OCOisoBu, OCOtBu, CON(R¹¹)₂, CONHEt, CONEt₂, CONHMe, CONMe₂, CONHPr, CONPr₂, CONHBu, OCONHBu, CONHtBu, CONHCH(CH₃)CH₂OH, CONHCH(CH₃)CH₂OCH₃, CONHCH(C₂H₅)CH₂OH, CONHCH(C₂H₅)CH₂OCH₃, CONH(CH₂)₂OCH₃, CONH(CH₂)₂OH, CONH(CH₂)₃OCH₃, CONH(CH₂)₃OH, CONR¹¹CH(CH₃)CH₂OH, CONR¹¹CH(CH₃)CH₂OCH₃, CONR¹¹CH(C₂H₅)CH₂OH, CONR¹¹CH(C₂H₅)CH₂OCH₃, CONR¹¹(CH₂)₂OCH₃, CONR¹¹(CH₂)₂OH, CONR¹¹(CH₂)₃OCH₃, CONR¹¹(CH₂)₃OH, CO₂R¹¹, CO₂Me, CO₂Et, CO₂Pr, CO₂iPr, CO₂Bu, CO₂secBu, CO₂ isoBu, CO₂tBu, CO₂(CH₂)₂OH, CO₂(CH₂)₂OCH₃, CO₂(CH₂)₃OH, CO₂(CH₂)₃OCH₃, (CH₂)ₘOR¹¹CH₂OH, (CH₂)₂OH, (CH₂)₃OH, (CH₂)₄OH, CH₂OMe, (CH₂)₂OMe, (CH₂)₃OMe, (CH₂)₄OMe, (CH₂)ₘSR¹¹, CH₂SH, (CH₂)₂SH, (CH₂)₃SH, (CH₂)₄SH, CH₂SMe, (CH₂)₂SMe, (CH₂)₃SMe, (CH₂)₄SMe, (CH₂)ₘN(R¹¹)₂, CH₂NH₂, CH₂NAC₂, CH₂N(COCF₃)₂, CH₂NHAc, CH₂NHCOCF₃, (CH₂)₂NH₂, (CH₂)₃NH₂, (CH₂)₄NH₂, CH₂NMe₂, (CH₂)₂NHMe, (CH₂)₂NMe₂, (CH₂)₃NHMe, (CH₂)₃NMe₂, (CH₂)₄NHMe, (CH₂)₄NMe₂, (CH₂)ₘCOOR¹², CH₂COOMe, (CH₂)₂COOMe, (CH₂)₃COOMe, CH₂COOEt, (CH₂)₂COOEt, (CH₂)₃COOEt, CH₂COOPr, (CH₂)₂COOPr, (CH₂)₃COOPr, CH₂COOiPr, (CH₂)₂COOiPr, (CH₂)₃COOiPr, CH₂COOtBu, (CH₂)₂COOtBu, (CH₂)₃COOtBu, CH₂COO(CH₂)₂OH, CH₂COO(CH₂)₂OCH₃, CH₂COO(CH₂)₃OH, CH₂COO(CH₂)₃OCH₃, CH₂NHCOOR¹¹, CH₂NR¹¹COOR¹¹, CH₂NHCOOMe, CH₂NHCOOtBu, CH₂NHCOOEt, CH₂NHCOOPr, CH₂NHCOOiPr, CH₂NHCOOBu, CH₂NHCOOtBu, CH₂NHCOOsecBu, CH₂NHCOOisoBu, CH₂NR¹¹COOEt, CH₂NR¹¹COOPr, CH₂NR¹¹COOiPr, CH₂NR¹¹COOBu, CH₂NR¹¹COOtBu, CH₂NR¹¹COOsecBu, CH₂NR¹¹ COOisoBu, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methyl-pentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; cyclopropyl, cyclobutyl, cyclopentyl, cyclohexyl; CF₃, CF₂H, CCl₃, C₂F₅, C₃F₇, CF(CF₃)₂,
R⁹ is hydrogen, fluorine, chlorine, bromine, iodine, N(R¹¹)₂, NMe₂, NEt₂, NHMe, NH₂, NHtBu, NHEt, NHPr, NHiPr, NHBu, NHsecBu, NHisoBu, 4-methylpiperazin-1-yl, piperazin-1-yl, morpholin-1-yl, cyano, hydroxyl, OR¹¹, O-Me, O-Et, O—Pr, O-iPr, O-Bu, O-secBu, O-isoBu, O-tBu, O—(CH₂)₂OH, O—(CH₂)₂OCH₃, O—(CH₂)₃OH, O—(CH₂)₃OCH₃, OCF₃, SR¹¹, SH, S-Me, S-Et, S—Pr, S-iPr, S-Bu, S-secBu, S-isoBu, S-tBu, SCF₃, COR¹¹, COMe, COEt, COPr, CO/Pr, COBu, COsecBu, COisoBu, COtBu, COCF₃, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethyl-butyl, 1,1,2-trimethylpropyl, 1,2,2-trimethylpropyl, 1-ethyl-1-methylpropyl and 1-ethyl-2-methylpropyl; CF₃, CF₂H, CCl₃, C₂F₅, C₃F₇, CF(CF₃)₂, cyclopropyl, cyclobutyl, cyclopentyl, cyclohexyl,
R¹⁰ is hydrogen, fluorine, chlorine, bromine, iodine, cyano, hydroxyl, amino, N(R¹¹)₂, nitro, OR¹¹, SR¹¹, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; CF₃, CF₂H, CCl₃, C₂F₅, C₃F₇, CF(CF₃)₂, cyclopropyl, cyclobutyl, cyclopentyl, cyclohexyl, SiMe₃, COOR¹¹, CON(R¹¹)₂,
R¹¹ are identical or different and are hydrogen, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, CH₂CH₂OMe, CH(CH₃)CH₂OMe, CH₂CH(CH₃)OMe, CH₂CH₂OEt, CH(CH₃)CH₂OEt, CH₂CH(CH₃)OEt, CF₃, CF₂H, CCl₃, C₂F₅, C₃F₇, CF(CF₃)₂, cyclopropyl, cyclobutyl, cyclopentyl, cyclohexyl, SiMe₃, COOR¹¹, CON(R¹¹)₂, phenyl or
if two radicals R¹¹ are attached to one nitrogen atom, two radicals R¹¹ may form a 3- to 7-membered, unsubstituted or substituted saturated or unsaturated cycle which may contain up to five further heteroatoms from the group consisting of N, O and S, where two oxygen atoms are not adjacent, or
if two radicals R¹¹ are adjacent in the grouping NR¹¹COR¹¹, two radicals R¹¹ may form a 3- to 7-membered unsubstituted or substituted saturated or unsaturated cycle which may contain up to five further heteroatoms selected from the group consisting of N, O and S, where two oxygen atoms are not adjacent,
R¹² is methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; CF₃, CF₂H, CCl₃, C₂F₅, C₃F₇, CF(CF₃)₂, cyclopropyl, cyclobutyl, cyclopentyl, cyclohexyl, CH₂OR¹¹, (CH₂)₂OR¹¹, (CH₂)₃OR¹¹, (CH₂)₄OR¹¹, SiMe₃,
R¹⁴ independently of one another are hydrogen, fluorine, chlorine, bromine, iodine, cyano, hydroxyl, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; CF₃, CF₂H, CCl₃, C₂F₅, C₃F₇, CF(CF₃)₂, orcyclopropyl, cyclohexyl, cyclopentyl, SiMe₃,
A is OR¹¹, SR¹⁵, N(R¹⁵)₂ or C(R¹⁶)₃,
R¹⁵ independently of one another are COR¹⁷, COCH₃, COCF₃, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, CH₂CH₂OMe, CH(CH₃)CH₂OMe, CH₂CH(CH₃)OMe, CH₂CH₂OEt, CH(CH₃)CH₂OEt, CH₂CH(CH₃)OEt, CF₃, CF₂H, CCl₃, C₂F₅, C₃F₇, CF(CF₃)₂, cyclopropyl, cyclobutyl, cyclohexyl, CH₂OR¹¹, (CH₂)₂OR¹¹, (CH₂)₃OR¹¹, (CH₂)₄OR¹¹,
R¹⁶ independently of one another are hydrogen, halogen, cyano, hydroxyl, OR¹¹, SR¹¹, COR¹¹, N(R¹¹)₂, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; CF₃, CF₂H, CCl₃, C₂F₅, C₃F₇, CF(CF₃)₂, cyclopropyl, cyclobutyl, cyclopentyl, cyclohexyl, CH₂OR¹¹, (CH₂)₂OR¹¹, (CH₂)₃OR¹¹, (CH₂)₄OR¹¹, SiMe₃ or
in each case two radicals R¹⁶ represent doubly attached oxygen or doubly attached sulphur, or
in each case two radicals R¹⁴, R¹⁴ or R¹⁵, R¹⁵ or R¹⁶, R¹⁶ or R¹⁴, R¹⁵ or R¹⁴, R¹⁶ together form a cyclopentyl, cyclohexyl, cyclopentenyl, cyclohexenyl, cyclopentadienyl, cyclohexadienyl, 4-pyridyl,
R¹⁷ is methyl, ethyl, propyl, 1-methylethyl, CF₃, CF₂H, CCl₃, C₂F₅, C₃F₇, CF(CF₃)₂
And/or an agrochemically active salt thereof.

13. A Compound of claim 11, wherein:
R¹ to R⁴ independently of one another are hydrogen, fluorine, chlorine, bromine, iodine, cyano, hydroxyl, nitro, OR¹¹, O-Me, O-Et, O—Pr, O-iPr, O-Bu, O-secBu, O-isoBu, O-tBu, O—(CH₂)₂OH, O—(CH₂)₂OCH₃, O—(CH₂)₃OH, O—(CH₂)₃OCH₃, OCF₃, SR¹¹, SH, S-Me, S-Et, S—Pr, S-iPr, S-Bu, S-secBu, S-isoBu, S-tBu, SCF₃, SOR¹¹, SO-Me, SO-Et, SO—Pr, SO-iPr, SO-Bu, SO-secBu, SO-isoBu, SO-tBu, SO₂R¹¹, SO₂-Me, SO₂-Et, SO₂—Pr, SO₂-iPr, SO₂—Bu, SO₂-secBu, SO₂-isoBu, SO₂-tBu, SON(R¹¹)₂, SONHMe, SONMe₂, SONHEt, SONEt₂, SONHPr, SONPr₂, SONHBu, SONBu₂, SONHCF₃, SON(CF₃)₂, SO₂N(R¹¹)₂, SO₂NHMe, SO₂NMe₂, SO₂NEt₂, SO₂NHEt, SO₂NPr₂, SO₂NHPr, SO₂NHCF₃, SO₂N(CF₃)₂, COR¹¹, COMe, COEt, COPr, COiPr, COBu, COsecBu, CO-isoBu, COtBu, COCF₃, NR¹¹CO₂R¹¹, NR¹¹CO₂Me, NR¹¹CO₂Et, NR¹¹CO₂Pr, NR¹¹CO₂iPr, NR¹¹CO₂Bu, NR¹¹CO₂secBu, NR¹¹CO₂ isoBu, NR¹¹CO₂tBu, NR¹¹COR¹¹, NHCOMe, NHCOEt, NHCOPr, NHCOiPr, NHCOBu, NHCOiBu, NHCOsecBu, NHCOisoBu, NHCOtBu, NHCO(CH₂)₂OH, NHCO(CH₂)₂OCH₃, NHCO(CH₂)₃OH, NHCO(CH₂)₃OCH₃, NR¹¹COMe, NR¹¹COEt, NR¹¹COPr, NR¹¹COiPr, NR¹¹COBu, NR¹¹COsecBu, NR¹¹COtBu, NR¹¹CO(CH₂)₂OH, NR¹¹CO(CH₂)₂OCH₃, NR¹¹CO(CH₂)₃OH, NR¹¹CO(CH₂)₃OCH₃, N(R¹¹)₂, NMe₂, NEt₂, NHMe, NH₂, NHtBu, NHEt, NHPr, NHiPr, NHBu, NHiBu, NHsecBu, piperazin-1-yl, 4-methylpiperazin-1-yl, morpholin-1-yl, NR¹¹SO₂R¹², NHSOR¹¹, NR¹¹SOR¹¹, NHSOMe, NHSO₂Me, NHSOEt, NHSO₂Et, NMeSOMe, NHSO₂R¹¹, NR¹¹SO₂R¹¹, NMeSO₂Me, NMeSOEt, NMeSO₂Et, NHSOCF₃, NHSO₂CF₃, OCON(R¹¹)₂, OCONHMe, OCONHEt, OCONHPr, OCONHiPr, OCONHBu, OCONHsecBu, OCONHisoBu, OCONHtBu, OCONMe₂, OCONEt₂, OCONPr₂, OCONiPr₂, OCONBu₂, OCONsecBu₂, OCONisoBu₂, OCONHtBu₂, OCOR¹¹, OCOMe, OCOEt, OCOPr, OCOiPr, OCOBu, OCOsecBu, OCOisoBu, OCOtBu, CON(R¹¹)₂, CONHEt, CONEt₂, CONHMe, CONMe₂, CONHPr, CONPr₂, CONHBu, CONHsecBu, CONHisoBu, CONHtBu, CONHCH(CH₃)CH₂OH, CONHCH(CH₃)CH₂OCH₃, CONHCH(C₂H₅)CH₂OH, CONHCH(C₂H₅)CH₂OCH₃, CONH(CH₂)₂OCH₃, CONH(CH₂)₂OH, CONH(CH₂)₃OCH₃, CONH(CH₂)₃OH, CONR¹¹CH(CH₃)CH₂OH, CONR¹¹CH(CH₃)CH₂OCH₃, CONR¹¹CH(C₂H₅)CH₂OH, CONR¹¹CH(C₂H₅)CH₂OCH₃, CONR¹¹(CH₂)₂OCH₃, CONR¹¹(CH₂)₂OH, CONR¹¹(CH₂)₃OCH₃, CONR¹¹(CH₂)₃OH, CO₂R¹¹, CO₂Me, CO₂Et, CO₂Pr, CO₂iPr, CO₂Bu, CO₂secBu, CO₂ isoBu, CO₂tBu, CO₂(CH₂)₂OH, CO₂(CH₂)₂OCH₃, CO₂(CH₂)₃OH, CO₂(CH₂)₃OCH₃, (CH₂)ₘOR¹¹, CH₂OH, (CH₂)₂OH, (CH₂)₃OH, (CH₂)₄OH, CH₂OMe, (CH₂)₂OMe, (CH₂)₃OMe, (CH₂)₄OMe, (CH₂)ₘSR¹¹, CH₂SH, (CH₂)₂SH, (CH₂)₃SH, (CH₂)₄SH, CH₂SMe, (CH₂)₂SMe, (CH₂)₃SMe, (CH₂)₄SMe, (CH₂)ₘN(R¹¹)₂, CH₂NH₂, CH₂NAc₂, CH₂N(COCF₃)₂, CH₂NHAc, CH₂NHCOCF₃, (CH₂)₂NH₂, (CH₂)₃NH₂, (CH₂)₄NH₂, CH₂NMe₂, (CH₂)₂NHMe, (CH₂)₂NMe₂, (CH₂)₃NHMe, (CH₂)₃NMe₂, (CH₂)₄NHMe, (CH₂)₄NMe₂, (CH₂)ₘCOOR¹², CH₂COOMe, (CH₂)₂COOMe, (CH₂)₃COOMe, CH₂COOEt, (CH₂)₂COOEt, (CH₂)₃COOEt, CH₂COOPr, (CH₂)₂COOPr, (CH₂)₃COOPr, CH₂COOiPr, (CH₂)₂COOiPr, (CH₂)₃COOiPr, CH₂COOtBu, (CH₂)₂COOtBu, (CH₂)₃COOtBu, CH₂COO(CH₂)₂OH CH₂COO(CH₂)₂OCH₃, CH₂COO(CH₂)₃OH, CH₂COO(CH₂)₃OCH₃, CH₂NHCOOR¹¹, CH₂NR¹¹COOR¹¹, CH₂NHCOOMe, CH₂NHCOOtBu, CH₂NHCOOEt, CH₂NHCOOPr, CH₂NHCOOiPr, CH₂NHCOOBu, CH₂NHCOOtBu, CH₂NHCOOsecBu, CH₂NHCOOisoBu, CH₂NR¹¹COOEt, CH₂NR¹¹COOPr, CH₂NR¹¹COOiPr, CH₂NR¹¹COOBu, CH₂NR¹¹COOtBu, CH₂NR¹¹COOsecBu, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; cyclopropyl, cyclobutyl, cyclopentyl, cyclohexyl; CF₃, CF₂H, CCl₃, C₂F₅, C₃F₇, CF(CF₃)₂,
or in each case two adjacent radicals R¹, R² or R², R³ or R³, R³ or R⁸, R⁴ together form a 2,3-dihydro-1,4-dioxin, a 2,2,3,3-tetrafluoro-2,3-dihydro-1,4-dioxin, a 1,3-dioxole or a 2,2-difluoro-1,3-dioxole ring,
R⁵ is hydrogen, acetyl, trifluoroacetyl,
X is nitrogen or CR³,
Y is nitrogen,
Z is nitrogen or CR¹⁰,
R⁷ is hydrogen, fluorine, chlorine, bromine, iodine, cyano, hydroxyl, amino, N(R¹¹)₂, nitro, OR¹¹, SR¹¹, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; CF₃, CF₂H, CCl₃, C₂F₅, C₃F₇, CF(CF₃)₂, cyclopropyl, cyclobutyl, cyclopentyl, cyclohexyl, phenyl, p-Cl-phenyl, p-F-phenyl, p-Br-phenyl, p-I-phenyl, p-methoxyphenyl, p-trifluoromethoxyphenyl, 2-pyridyl, 3-pyridyl, 4-pyridyl, 2-thienyl, 3-thienyl, SiMe₃, COOR¹¹, CO₂Me, CO₂Et, CO₂Pr, CO₂iPr, CO₂Bu, CO₂secBu, CO₂ isoBu, CO₂tBu, CO₂(CH₂)₂OH, CO₂(CH₂)₂OCH₃, CO₂(CH₂)₃OH, CO₂(CH₂)₃OCH₃, CON(R¹¹)₂, CONHEt, CONEt₂, CONHMe, CONMe₂, CONHPr, CONPr₂, CONHBu, CONHsecBu, CONHisoBu, CONHtBu, CONHCH(CH₃)CH₂OH, CONHCH(CH₃)CH₂OCH₃, CONHCH(C₂H₅)CH₂OH, CONHCH(C₂H₅)CH₂OCH₃, CONH(CH₂)₂OCH₃, CONH(CH₂)₂OH, CONH(CH₂)₃OCH₃, CONH(CH₂)₃OH, CONR¹¹CH(CH₃)CH₂OH, COR¹¹COMe, COEt, COPr, CO/Pr, COBu, COsecBu, CO-isoBu, COtBu, COCF₃ or
R⁷ and R¹⁰ together form a saturated or unsaturated bridge of the following structure: 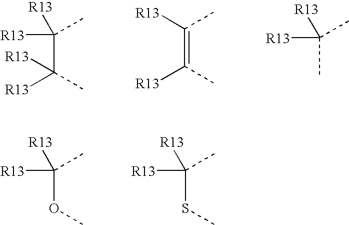
where R¹³=independently of one another hydrogen, fluorine, chlorine, bromine, iodine, methyl, ethyl, propyl, 1-methylethyl, CF₂H, CF₃, C₂F₅, OCH₃; OC₂H₅, OCF₃, OC₂F₅ or
two geminal radicals R¹³ represent doubly attached oxygen or sulphur,
R⁸ is hydrogen, fluorine, chlorine, bromine, iodine, cyano, hydroxyl, nitro, OR¹¹, O-Me, O-Et, O—Pr, O-iPr, O-Bu, O-secBu, O-isoBu, O-tBu, O—(CH₂)₂OH, O—(CH₂)₂OCH₃, O—(CH₂)₃OH, O—(CH₂)₃OCH₃, OCF₃, SR¹¹, SH, S-Me, S-Et, S—Pr, S-iPr, S-Bu, S-secBu, S-isoBu, S-tBu, SCF₃, SOR¹¹, SO-Me, SO-Et, SO—Pr, SO-iPr, SO-Bu, SO-secBu, SO-isoBu, SO-tBu, SO₂R¹¹, SO₂-Me, SO₂-Et, SO₂—Pr, SO₂-iPr, SO₂—Bu, SO₂-secBu, SO₂-isoBu, SO₂-tBu, SON(R¹¹)₂, SONHMe, SONMe₂, SONHEt, SONEt₂, SONHPr, SONPr₂, SONHBu, SONBu₂, SONHCF₃, SON(CF₃)₂, SO₂N(R¹¹)₂, SO₂NHMe, SO₂NMe₂, SO₂NEt₂, SO₂NHEt, SO₂NPr₂, SO₂NHPr, SO₂NHCF₃, SO₂N(CF₃)₂, COR¹¹, COMe, COEt, COPr, COiPr, COBu, COsecBu, COisoBu, COtBu, COCF₃, NR¹¹CO₂R¹¹, NR¹¹CO₂Me, NR¹¹CO₂Et, NR¹¹CO₂Pr, NR¹¹CO₂iPr, NR¹¹CO₂Bu, NR¹¹CO₂secBu, NR¹¹CO₂ isoBu, NR¹¹CO₂tBu, NR¹¹COR¹¹, NHCOMe, NHCOEt, NHCOPr, NHCOiPr, NHCOBu, NHCOiBu, NHCOsecBu, NHCOisoBu, NHCOtBu, NHCO(CH₂)₂OH, NHCO(CH₂)₂OCH₃, NHCO(CH₂)₃OH, NHCO(CH₂)₃OCH₃, NR¹¹COMe, NR¹¹COEt, NR¹¹COPr, NR¹¹COiPr, NR¹¹COBu, NR¹¹COsecBu, NR¹¹COisoBu, NR¹¹COtBu, NR¹¹CO(CH₂)₂OH, NR¹¹CO(CH₂)₂OCH₃, NR¹¹CO(CH₂)₃OH, NR¹¹CO(CH₂)₃OCH₃, N(R¹¹)₂, NMe₂, NEt₂, NHMe, NH₂, NHtBu, NHEt, NHPr, NHiPr, NHBu, NHsecBu, NHisoBu, 4-methylpiperazin-1-yl, piperazin-1-yl, morpholin-1-yl, NR¹¹SO₂R¹², NHSOR¹¹, NR¹¹SOR¹¹, NHSOMe, NHSO₂Me, NHSOEt, NHSO₂Et, NMeSOMe, NHSO₂R¹¹, NR¹¹SO₂R¹¹, NMeSO₂Me, NMeSOEt, NMeSO₂Et, NHSOCF₃, NHSO₂CF₃, OCON(R¹¹)₂, OCONHMe, OCONHEt, OCONHPr, OCONHiPr, OCONHBu, OCONHsecBu, OCONHisoBu, OCONHtBu, OCONMe₂, OCONEt₂, OCONPr₂, OCONiPr₂, OCONBu₂, OCONsecBu₂, OCONisoBu₂, OCONtBu₂, OCOR¹¹, OCOMe, OCOEt, OCOPr, OCOiPr, OCOBu, OCOsecBu, OCOisoBu, OCOtBu, CON(R¹¹)₂, CONHEt, CONEt₂, CONHMe, CONMe₂, CONHPr, CONPr₂, CONHBu, OCONHBu, CONHtBu, CONHCH(CH₃)CH₂OH, CONHCH(CH₃)CH₂OCH₃, CONHCH(C₂H₅)CH₂OH, CONHCH(C₂H₅)CH₂OCH₃, CONH(CH₂)₂OCH₃, CONH(CH₂)₂OH, CONH(CH₂)₃OCH₃, CONH(CH₂)₃OH, CONR¹¹CH(CH₃)CH₂OH, CONR¹¹CH(CH₃)CH₂OCH₃, CONR¹¹CH(C₂H₅)CH₂OH, CONR¹¹CH(C₂H₅)CH₂OCH₃, CONR¹¹(CH₂)₂OCH₃, CONR¹¹(CH₂)₂OH, CONR¹¹(CH₂)₃OCH₃, CONR¹¹(CH₂)₃OH, CO₂R¹¹, CO₂Me, CO₂Et, CO₂Pr, CO₂iPr, CO₂Bu, CO₂secBu, CO₂ isoBu, CO₂tBu, CO₂(CH₂)₂OH, CO₂(CH₂)₂OCH₃, CO₂(CH₂)₃OH, CO₂(CH₂)₃OCH₃, (CH₂)ₘOR¹¹, CH₂OH, (CH₂)₂OH, (CH₂)₃OH, (CH₂)₄OH, CH₂OMe, (CH₂)₂OMe, (CH₂)₃OMe, (CH₂)₄OMe, (CH₂)ₘSR¹¹, CH₂SH, (CH₂)₂SH, (CH₂)₃SH, (CH₂)₄SH, CH₂SMe, (CH₂)₂SMe, (CH₂)₃SMe, (CH₂)₄SMe, (CH₂)ₘN(R¹¹)₂, CH₂NH₂, CH₂NAc₂, CH₂N(COCF₃)₂, CH₂NHAc, CH₂NHCOCF₃, (CH₂)₂NH₂, (CH₂)₃NH₂, (CH₂)₄NH₂, CH₂NMe₂, (CH₂)₂NHMe, (CH₂)₂NMe₂, (CH₂)₃NHMe, (CH₂)₃NMe₂, (CH₂)₄NHMe, (CH₂)₄NMe₂, (CH₂)ₘCOOR¹², CH₂COOMe, (CH₂)₂COOMe, (CH₂)₃COOMe, CH₂COOEt, (CH₂)₂COOEt, (CH₂)₃COOEt, CH₂COOPr, (CH₂)₂COOPr, (CH₂)₃COOPr, CH₂COOiPr, (CH₂)₂COOiPr, (CH₂)₃COOiPr, CH₂COOtBu, (CH₂)₂COOtBu, (CH₂)₃COOtBu, CH₂COO(CH₂)₂OH, CH₂COO(CH₂)₂OCH₃, CH₂COO(CH₂)₃OH, CH₂COO(CH₂)₃OCH₃, CH₂NHCOOR¹¹, CH₂NR¹¹COOR¹¹, CH₂NHCOOMe, CH₂NHCOOtBu, CH₂NHCOOEt, CH₂NHCOOPr, CH₂NHCOOiPr, CH₂NHCOOBu, CH₂NHCOOtBu, CH₂NHCOOsecBu, CH₂NHCOOisoBu, CH₂NR¹¹COOEt, CH₂NR¹¹COOPr, CH₂NR¹¹COOiPr, CH₂NR¹¹COOBu, CH₂NR¹¹COOtBu, CH₂NR¹¹COOsecBu, CH₂NR¹¹COOisoBu, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methyl-pentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; cyclopropyl, cyclobutyl, cyclopentyl, cyclohexyl; CF₃, CF₂H, CCl₃, C₂F₅, C₃F₇, CF(CF₃)₂,
R¹⁰ is hydrogen, fluorine, chlorine, bromine, iodine, cyano, hydroxyl, amino, N(R¹¹)₂, NMe₂, NEt₂, NHMe, NH₂, NHtBu, NHEt, NHPr, NHiPr, NHBu, NHsecBu, NHisoBu, 4-methylpiperazin-1-yl, piperazin-1-yl, morpholin-1-yl, nitro, OR¹¹, O-Me, O-Et, O—Pr, O-iPr, O-Bu, O-secBu, O-isoBu, O-tBu, OCH(CH₃)CH₂OH, OCH(CH₃)CH₂OCH₃, OCH(C₂H₅)CH₂OH, OCH(C₂H₅)CH₂OCH₃, O(CH₂)₂OCH₃, O(CH₂)₂OH, O(CH₂)₃OCH₃, O(CH₂)₃OH, OCF₃, SR¹¹, S-Me, S-Et, S—Pr, S-iPr, S-Bu, S-secBu, S-isoBu, S-tBu, SCF₃, methyl, ethyl, propyl, 1-methylethyl, butyl, 1-methylpropyl, 2-methylpropyl, 1,1-dimethylethyl, pentyl, 1-methyl butyl, 2-methylbutyl, 3-methylbutyl, 2,2-dimethylpropyl, 1-ethylpropyl, hexyl, 1,1-dimethylpropyl, 1,2-dimethylpropyl, 1-methyl-pentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; CF₃, CF₂H, CCl₃, C₂F₅, C₃F₇, CF(CF₃)₂, cyclopropyl, cyclobutyl, cyclopentyl, cyclohexyl, SiMe₃, COOR¹¹, CO₂Me, CO₂Et, CO₂Pr, CO₂iPr, CO₂Bu, CO₂secBu, CO₂ isoBu, CO₂tBu, CON(R¹¹)₂, CONH₂, CONHMe, CONMe₂, CONHEt, CONEt₂, CO— morpholine, CO-piperidine, CO-piperazine, CO-(4-methylpiperazine), CONHCH(CH₃)CH₂OH, CONHCH(CH₃)CH₂OCH₃, CONHCH(C₂H₅)CH₂OH, CONHCH(C₂H₅)CH₂OCH₃, CONH(CH₂)₂OCH₃, CONH(CH₂)₂OH, CONH(CH₂)₃OCH₃, CONH(CH₂)₃OH,
R¹¹ are identical or different and are hydrogen, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, CH₂CH₂OMe, CH(CH₃)CH₂OMe, CH₂CH(CH₃)OMe, CH₂CH₂OEt, CH(CH₃)CH₂OEt, CH₂CH(CH₃)OEt, CF₃, CF₂H, CCl₃, C₂F₅, C₃F₇, CF(CF₃)₂, cyclopropyl, cyclobutyl, cyclopentyl, cyclohexyl, SiMe₃, COOR¹¹, CON(R¹¹)₂, phenyl or
if two radicals R¹¹ are attached to one nitrogen atom, these radicals together may represent piperidin-1-yl, piperazin-1-yl, 4-methylpiperazin-1-yl or morpholin-1-yl, or
if two radicals R¹¹ are adjacent in the grouping NR¹¹COR¹¹, two radicals R¹¹ may form a 3- to 7-membered, unsubstituted or substituted saturated or unsaturated cycle which may contain up to five further heteroatoms selected from the group consisting of N, O and S, where two oxygen atoms are not adjacent,
R¹² is methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; CF₃, CF₂H, CCl₃, C₂F₅, C₃F₇, CF(CF₃)₂, cyclopropyl, cyclobutyl, cyclopentyl, cyclohexyl, CH₂OR¹¹, (CH₂)₂OR¹¹, (CH₂)₃OR¹¹, (CH₂)₄OR¹¹, SiMe₃,
R¹⁴ independently of one another are hydrogen, fluorine, chlorine, bromine, iodine, cyano, hydroxyl, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; CF₃, CF₂H, CCl₃, C₂F₅, C₃F₇, CF(CF₃)₂, or cyclopropyl, cyclohexyl, cyclopentyl, SiMe₃,
A is OR¹⁵, N(R¹⁵)₂ or C(R¹⁶)₃,
R¹⁵ independently of one another are COR¹⁷, COCH₃, COCF₃, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, CH₂CH₂OMe, CH(CH₃)CH₂OMe, CH₂CH(CH₃)OMe, CH₂CH₂OEt, CH(CH₃)CH₂OEt, CH₂CH(CH₃)OEt, CF₃, CF₂H, CCl₃, C₂F₅, C₃F₇, CF(CF₃)₂, cyclopropyl, cyclobutyl, cyclohexyl, CH₂OR¹¹, (CH₂)₂OR¹¹, (CH₂)₃OR¹¹, (CH₂)₄OR¹¹,
R¹⁶ independently of one another are hydrogen, halogen, cyano, hydroxyl, OR¹¹, SR¹¹, COR¹¹, N(R¹¹)₂, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; CF₃, CF₂H, CCl₃, C₂F₅, C₃F₇, CF(CF₃)₂, cyclopropyl, cyclobutyl, cyclopentyl, cyclohexyl, CH₂OR¹¹, (CH₂)₂OR¹¹, (CH₂)₃OR¹¹, (CH₂)₄OR¹¹, SiMe₃ or
in each case two radicals R¹⁶ represent doubly attached oxygen or doubly attached sulphur, or
in each case two radicals R¹⁴, R¹⁴ or R¹⁵, R¹⁵ or R¹⁶, R¹⁶ or R¹⁴, R¹⁵ or R¹⁴, R¹⁶ together form a cyclopentyl, cyclohexyl, cyclopentenyl, cyclohexenyl, cyclopentadienyl, cyclohexadienyl, 4-pyridyl,
R¹⁷ is methyl, ethyl, propyl, 1-methylethyl, CF₃, CF₂H, CCl₃, C₂F₅, C₃F₇, CF(CF₃)₂
And/or an agrochemically active salt thereof.

14. A Compound of claim 11, wherein:
R¹ to R⁴ independently of one another are hydrogen, fluorine, chlorine, cyano, hydroxyl, nitro, OMe, CF₃, COCH₃, COOCH₃, COOH, N(Me)₂, NHCOCH₃, NHCOCF₃, NHSO₂CH₃, NHCOOCH₃, NHCOO(CH₂CH₂)OCH₃, 4-N-methylpiperazin-1-yl, CH₂NH₂, CH₂NHCOOCH₃, CH₂NHCOOtBu, methyl,
or in each case two adjacent radicals R¹, R² or R², R³ or R³, R⁸ or R⁸, R⁴ together form a 2,3-dihydro-1,4-dioxin or a 1,3-dioxole ring,
R⁵ is hydrogen, COCH₃,
X is nitrogen or CR³,
Y is nitrogen,
Z is nitrogen or CR¹⁰,
R⁷ is hydrogen, methyl, 1-methylpropyl, CF₃, CF₂H, cyclopropyl, or
R⁷ and R¹⁰ together form a saturated or unsaturated bridge of the following structure: 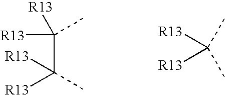
where R¹³=hydrogen,
R³ is hydrogen, fluorine, chlorine, cyano, hydroxyl, nitro, OMe, CF₃, COCH₃, COOCH₃, COOH, N(Me)₂, NHCOCH₃, NHCOCF₃, NHSO₂CH₃, NHCOOCH₃, NHCOO(CH₂CH₂)OCH₃, 4-N-methylpiperazin-1-yl, CH₂NH₂, CH₂NHCOOCH₃, CH₂NHCOOtBu, methyl,
R¹⁰ is hydrogen or
R⁷ and R¹⁰ together form a saturated or unsaturated bridge of the following structure: 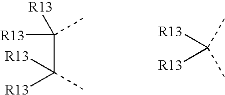
where R¹³=hydrogen,
R¹¹ are identical or different and are hydrogen, methyl, ethyl, CH(CH₃)CH₂OMe, CH₂CH(CH₃)OMe, CH₂CH₂OEt, CH(CH₃)CH₂OEt, CH₂CH(CH₃)OEt, cyclopropyl, cyclohexyl, or
if two radicals R¹¹ are attached to one nitrogen atom, these radicals together may represent 4-methylpiperazin-1-yl,
R¹² is methyl, ethyl, propyl, 1-methylethyl, CF₃, cyclopropyl, cyclohexyl,
R¹⁴ independently of one another are hydrogen, methyl, cyclopropyl,
A is OR¹⁵ or C(R¹⁶)₃,
R¹⁵ is ethyl,
R¹⁶ is hydrogen,
R¹⁷ is methyl, ethyl, propyl, 1-methylethyl, CF₃, CF₂H, CCl₃, C₂F₅, C₃F₇, CF(CF₃)₂.

15. A Compound of claim 11,
R¹ to R⁴ independently of one another are hydrogen, fluorine, chlorine, iodine, cyano, hydroxyl, nitro, OMe, OCH₂tBu, OCOCH₃, SO₂NH₂, SO₂N(CH₃)₂, COCH₃, COCH₂CH₂CH₃, COOCH₂CH₂OCH₃, COOCH₂CH₂CH₂OCH₃, CONHCH₂CH₂OCH₃, NH₂, N(Me)₂, NHCOCH₃, NHSO₂CH₃, NHCOOCH₃, NHCOOCH₂CH₃, NHCOOtBu, NHCOOCH₂CH₂OCH₃, 4-N-acetylpiperazin-1-yl, N-pyrrolidin-2-on-1-yl, CH₂NHCOOCH₃, CH₂NHCOOtBu, methyl, CF₃
or in each case two adjacent radicals R¹, R² or R², R³ or R³, R⁸ or R⁸, R⁴ together form a 2,3-dihydrofuran-2-one, 2,3-dihydro-1,4-dioxin, a 2,2,3,3-tetrafluoro-2,3-dihydro-1,4-dioxin, a 1,3-dioxole or a 2,2-difluoro-1,3-dioxole ring,
R⁵ is hydrogen, COCH₃, COCF₃, COOCH₃,
X is nitrogen or CR⁸,
R⁸ is hydrogen, O-Me,
Y is nitrogen,
Z is CR¹⁰,
R¹⁰ is hydrogen, fluorine, chlorine, bromine, cyano, CF₃ or
R⁷ and R¹⁰ together form a CH₂ or a CH₂CH₂ bridge,
R⁷ is hydrogen, methyl, CF₃, CF₂H, or
R⁷ and R¹⁰ together form a CH₂ or a CH₂CH₂ bridge,
R¹¹ are identical or different and are hydrogen, methyl, ethyl, CH(CH₃)CH₂OMe, CH₂CH(CH₃)OMe, CH₂CH₂OEt, CH(CH₃)CH₂OEt, CH₂CH(CH₃)OEt, cyclopropyl, cyclohexyl, or
if two radicals R¹¹ are attached to one nitrogen atom, these radicals together may represent 4-methylpiperazin-1-yl,
R¹² is methyl, ethyl, propyl, 1-methylethyl, CF₃, cyclopropyl, cyclohexyl,
R¹⁴ independently of one another are hydrogen, methyl, cyclopropyl,
A is OR¹⁵ or C(R¹⁶)₃,
R¹⁵ is ethyl,
R¹⁶ is hydrogen,
R¹⁷ is methyl, ethyl, propyl, 1-methylethyl, CF₃, CF₂H, CCl₃, C₂F₅, C₃F₇, CF(CF₃)₂.

16. A Compound of claim 11, wherein:
R¹ to R⁴ independently of one another are hydrogen, fluorine, chlorine, iodine, cyano, hydroxyl, nitro, OMe, OCH₂tBu, OCOCH₃, SO₂NH₂, SO₂N(CH₃)₂, COCH₃, COCH₂CH₂CH₃, COOCH₂CH₂OCH₃, COOCH₂CH₂CH₂OCH₃, CONHCH₂CH₂OCH₃, NH₂, N(Me)₂, NHCOCH₃, NHSO₂CH₃, NHCOOCH₃, NHCOOCH₂CH₃, NHCOOtBu, NHCOOCH₂CH₂OCH₃, CH₂NHCOOCH₃, CH₂NHCOOtBu, methyl, CF₃
or in each case two adjacent radicals R¹, R² or R², R³ or R³, R³ or R⁸, R⁴ together form a 2,3-dihydro-1,4-dioxin, a 2,2,3,3-tetrafluoro-2,3-dihydro-1,4-dioxin, a 1,3-dioxole or 2,2-difluoro-1,3-dioxole ring,
R⁵ is hydrogen, COCH₃, COCF₃, COOCH₃,
X is nitrogen or CR³,
R⁸ is hydrogen, O-Me,
Y is nitrogen,
Z is CR¹⁰,
R¹⁰ is hydrogen, fluorine, chlorine, bromine, cyano, CF₃ or
R⁷ and R¹⁰ together form a CH₂ or a CH₂CH₂ bridge,
R⁶ is ethyl, isopropyl, CH₂OCH₃, cyclopropyl, 3-pyridyl,
R⁷ is hydrogen, methyl, CF₃, CF₂H, or
R⁷ and R¹⁰ together form a CH₂ or a CH₂CH₂ bridge,
R¹¹ are identical or different and are hydrogen, methyl, ethyl, CH(CH₃)CH₂OMe, CH₂CH(CH₃)OMe, CH₂CH₂OEt, CH(CH₃)CH₂OEt, CH₂CH(CH₃)OEt, cyclopropyl, cyclohexyl, or,
if two radicals R¹¹ are attached to one nitrogen atom, these radicals together may represent 4-methylpiperazin-1-yl,
R¹²-methyl, ethyl, propyl, 1-methylethyl, CF₃, cyclopropyl, cyclohexyl,
R¹⁴ independently of one another are hydrogen, methyl, cyclopropyl,
A is OR¹¹ or C(R¹⁶)₃,
R¹⁵ is ethyl,
R¹⁶ is hydrogen,
R¹⁷ is methyl, ethyl, propyl, 1-methylethyl, CF₃, CF₂H, CCl₃, C₂F₅, C₃F₇, CF(CF₃)₂.

17. Composition for controlling unwanted microorganisms, comprising at least one compound according to claim 11 and an extender and/or surfactant.

18. Composition according to claim 17, comprising at least one further agrochemically active compound.

19. A method for controlling unwanted microorganisms comprising using a compound according to claim 11

20. Method for controlling unwanted microorganisms comprising applying a compound according to claim 12 to the unwanted microorganisms and/or to a habitat thereof.

21. Process for preparing a composition for controlling unwanted microorganisms, comprising mixing a compound according to claim 11 with an extender and/or surfactant.

22. Process for preparing a the compound of the formula (Id) 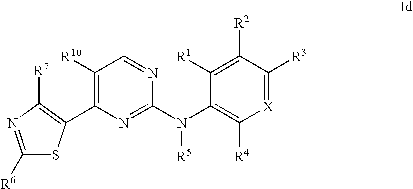
R¹ to R⁴ independently of one another are hydrogen, halogen, cyano, hydroxyl, nitro, OR¹¹, SR¹¹, SOR¹¹, SO₂R¹¹, SO₂N(R¹¹)₂, C═OR¹¹, NR¹¹COOR¹², N(R¹¹)₂, NR¹¹COR¹¹, NR¹¹COR¹², NR¹¹SO₂R¹², OCON(R¹¹)₂, OC═OR¹¹, CON(R¹¹)₂, COOR¹¹, (CH₂)ₘOR¹¹, (CH₂)ₘSR¹¹, (CH₂)ₘ(R¹¹)₂, (CH₂)ₘCOOR¹², (CH₂)ₘNR¹¹COOR¹¹, unsubstituted or substituted C₁-C₈-alkyl, C₁-C₈-haloalkyl, C₃-C₈-cycloalkyl; where m=1-8
or in each case two adjacent radicals R¹, R² or R², R³ or R³, R⁸ or R⁸, R⁴ together form an unsubstituted or substituted heterocyclic five- or six-membered ring,
R⁵ is hydrogen, unsubstituted or substituted C₁-C₈-alkyl, unsubstituted or substituted C₁-C₄-alkylC(═O), C₁-C₁₄-alkylOC(═O), unsubstituted or substituted C₁-C₄alkoxy(C₁-C₄)alkyl, unsubstituted or substituted C₉-C₆-alkenyl, unsubstituted or substituted C₂-C₆-alkynyl, C₁-C₆-alkylsulphinyl, C₁-C₆-alkylsulphonyl, C₃-C₈-cycloalkyl; C₁-C₆-haloalkyl, C₁-C₄-haloalkylsulphinyl, C₁-C₄-haloalkylsulphonyl, halo-C₁-C₄-alkoxy-C₁-C₄-alkyl, C₃-C₈-halocycloalkyl having in each case 1 to 9 fluorine, chlorine and/or bromine atoms; formyl, formyl-C₁-C₃-alkyl, (C₁-C₃-alkyl)-carbonyl-C₁₋₃alkyl, (C₁-C₃-alkoxy)carbonyl-C₁₋₃-alkyl; halo-(C₁-C₃-alkyl)carbonyl, (C₁-C₃-alkyl, halo-(C₁-C₃-alkoxy)carbonyl-C₁-C₃-alkyl having in each case 1 to 13 fluorine, chlorine and/or bromine atoms: (C₁-C₈-alkyl)carbonyl, (C₁-C₈-alkoxy)carbonyl, (C₁-C₈-alkylthio)carbonyl, (C₁-C₄-alkoxy-C₁₋₄-alkyl)carbonyl, (C₃-C₆-alkenyloxy)carbonyl. (C₃₋₆-alkynyloxy)carbonyl, (C₃-C₈-cycloalkyl)carbonyl: (C₁-C₆-haloalkyl)carbonyl, (C₁-C₆-haloalkylthio)carbonyl, (C₁-C₆-haloalkoxy)carbonyl, (C₃-C₆-haloalkenyloxy)carbonyl, (C₃-C₆-haloalkynyloxy)carbonyl, (halo-C₁-C₄-alkoxy-C₁-C₄-alkyl)carbonyl, (C₃-C₈-halocycloalkyl)carbonyl having in each case 1 to 9 fluorine, chlorine and/or bromine atoms, or —CH₂—C≡C—R^{1-A}, —CH₂—CH═CH—R^{1-A}, —CH═C═CH—R^{1-A}, —C(═O)C(═O)R², —CONR³R⁴, —CH₂NR⁶, C₁-C₄-trialkylsilyl or C₁-C₄-dialkylmonophenylsilyl,
R^{1-A} represents hydrogen, C₁₋₆-alkyl, C₁-C₆-haloalkyl, C₂-C₆-alkenyl, C₂-C₆-alkynyl, C₃-C₇-cycloalkyl, (C₁-C₄-alkoxy)carbonyl, (C₃-C₆-alkenyloxy)carbonyl, (C₃-C₆-alkynyloxy)carbonyl or cyano,
X is nitrogen or CR⁸.
Y is nitrogen or CR⁹,
Z is nitrogen or CR¹⁰,
R⁶ is hydrogen, halogen, cyano, hydroxyl, OR¹¹, SR¹¹, COR¹¹, CO₂R¹¹, unsubstituted or substituted C₁-C₈-alkyl, C₁-C₈-haloalkyl C₁-C₄-trialkylsilyl, N(R¹¹)₂, NR¹¹COR¹¹, NR¹¹COR¹², NR¹¹SOₙR¹¹, CON(R¹¹)₂, C₃-C₈-cycloalkyl, aryl, hetaryl; where n=0-2,
R⁷ is hydrogen, halogen, cyano, hydroxyl, amino, N(R¹¹)₂, nitro, OR¹¹, SR¹¹, unsubstituted or substituted C₁-C₈-alkyl, unsubstituted or substituted C₃-C₆-cycloalkyl, C₁-C₄-trialkylsilyl, COOR¹¹, CON(R¹¹), COR¹¹ or
R⁷ and R¹⁰ together form a saturated or unsaturated bridge of the following structure: 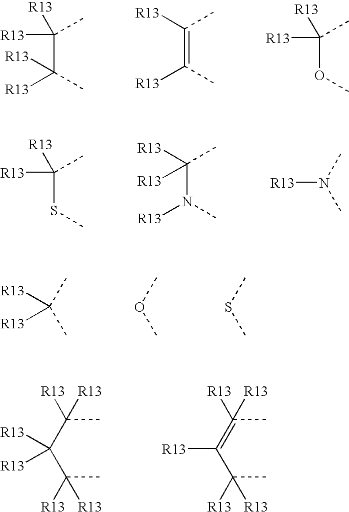
where R¹³=independently of one another hydrogen, halogen, unsubstituted or substituted C₁-C₃-alkyl, unsubstituted or substituted C₁-C₃-alkoxy or
two geminal radicals R¹³ represent doubly attached oxygen or sulphur,
R⁸ is hydrogen, halogen, cyano, hydroxyl, nitro, OR¹¹, SR¹¹, SOR¹¹, SO₂R¹¹, SO₂N(R¹¹)₂, C═OR¹¹, NR¹¹COOR¹², N(R¹¹)₂, NR¹¹COR¹¹, NR¹¹COR¹², NR¹¹SO₂R¹², OCON(R¹¹)₂, OC═OR¹¹, CON(R¹¹)₂, COOR¹¹, (CH₂)ₘOR¹¹(CH₂)ₘSR¹¹, (CH₂)ₘN(R¹¹)₂, (CH₂)ₘCOOR¹², (CH₂)ₘNR¹¹COOR¹¹, unsubstituted or substituted C₁-C₈-alkyl, C₁-C₈-haloalkyl, C₃-C₈-cycloalkyl; where m=1-8,
R⁹ is hydrogen, halogen, N(R¹¹)₂, cyano, hydroxyl, OR¹¹, SR¹¹, COR¹¹, unsubstituted or substituted C₁-C₈-alkyl or unsubstituted or substituted C₃-C₈-cycloalkyl,
R¹⁰ is hydrogen, halogen, cyano, hydroxyl, amino, N(R¹¹)₂, nitro, OR¹¹, SR¹¹, unsubstituted or substituted C₁-C₈-alkyl, C₁-C₄-trialkylsilyl, COOR¹¹, CON(R¹¹)₂,
R¹¹ are identical or different and are hydrogen, unsubstituted or substituted C₁-C₈-alkyl, C₁-C₈-haloalkyl, C₁-C₄-trialkylsilyl, aryl, or
if two radicals R¹¹ are attached to one nitrogen atom, two radicals R¹¹ may form a 3- to 7-membered unsubstituted or substituted saturated or unsaturated cycle which may contain up to five further heteroatoms selected from the group consisting of N, O and S, where two oxygen atoms are not adjacent, or
if two radicals R¹¹ are adjacent in the grouping NR¹¹COR¹¹, two radicals R¹¹ may form a 3- to 7-membered unsubstituted or substituted saturated or unsaturated cycle which may contain up to five further heteroatoms selected from the group consisting of N, O and S, where two oxygen atoms are not adjacent,
R¹² are identical or different and are unsubstituted or substituted C₁-C₈-alkyl, C₁-C₈-haloalkyl, (CH₂)ₚOR¹¹, C₁-C₄-trialkylsilyl: where p=1-4
Said process comprising:
a) metallizing a halothiazole of the formula XIV are metallized using a metal or metal organyl in the presence of a diluent, 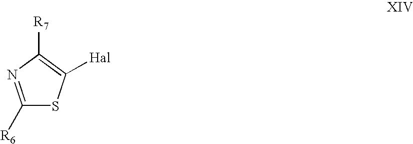
b) the resulting metal compound is reacted with a chloropyrimidine of the formula XV, if appropriate in the presence of a reaction auxiliary and in the presence of a diluent, 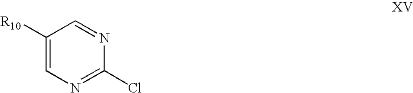
c) the resulting dihydropyrimidine is reacted in the presence of an oxidizing agent and, if appropriate, in the presence of a reaction auxiliary and in the presence of a diluent to give pyrimidines of the formula XVI 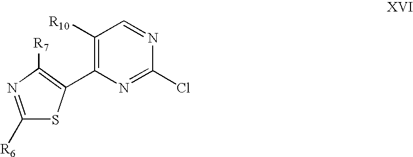
and
d) the resulting pyrimidine of the formula XVI is reacted with an amine of the formula XVII to give a compound of the formula Id, if appropriate in the presence of a reaction auxiliary, if appropriate in the presence of a metal catalyst and in the presence of a diluent 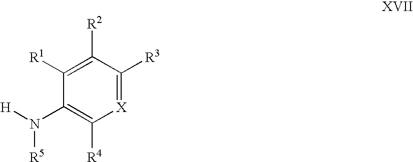
where R⁶ may also be a group C(R¹⁴)₂A in which R¹⁴ and A are as defined above.

23. An Intermediates of the formula XVI 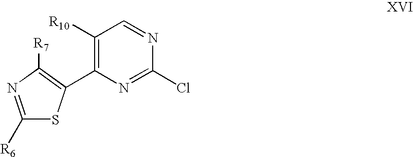
R¹ to R⁴ independently of one another are hydrogen, halogen, cyano, hydroxyl, nitro, OR¹¹, SR¹¹, SOR¹¹, SO₂R¹¹, SO₂N(R¹¹)₂, C═OR¹¹, NR¹¹COOR¹², N(R¹¹)₂, NR¹¹COR¹¹, NR¹¹COR¹², NR¹¹SO₂R¹², OCON(R¹¹)₂, OC═OR¹¹, CON(R¹¹)₂, COOR¹¹, (CH₂)ₘOR¹¹, (CH₂)ₘSR¹¹, (CH₂)ₘN(NR¹¹⁾₂, (CH₂)ₘCOOR¹², (CH₂)ₘNR¹¹COOR¹¹, unsubstituted or substituted C₁-C₈-alkyl, C₁-C₈-haloalkyl, C₃-C₈-cycloalkyl; where m=1-8
or in each case two adjacent radicals R¹, R² or R², R³ or R³, R⁸ or R⁸, R⁴ together form an unsubstituted or substituted heterocyclic five- or six-membered ring,
R⁵ is hydrogen, unsubstituted or substituted C₁₋₈ alkyl, unsubstituted or substituted C₁-C₄-alkylC(═O), C₁-C₄-alkylOC(═O), unsubstituted or substituted C₁-C₄-alkoxy(C₁-C₄)alkyl, unsubstituted or substituted C₂-C₆-alkenyl, unsubstituted or substituted C₂-C₆-alkynyl, C₁-C₆-alkylsulphinyl, C₁-C₆-alkylsulphonyl, C₃-C₈-cycloalkyl; C₁-C₆-haloalkyl, C₁-C₄-haloalkylsulphinyl, C₁-C₄-haloalkylsulphonyl, halo-C₁-C₄-alkoxy-C₁-C₄-alkyl, C₃-C₈-halocycloalkyl having in each case 1 to 9 fluorine-chlorine and/or bromine atoms; formyl, formyl-C₁-C₃-alkyl, (C₁-C₃-alkyl)carbonyl-C₁-C₃-alkyl, halo-(C₁-C₃-alkoxy)carbonyl-C₁-C₃-alkyl having in each case 1 to 13 fluorine, chlorine and/or bromine atoms; (C₁-C₈-alkyl)carbonyl, (C₁-C₈-alkoxy)carbonyl, (C₁-C₈-alkylthio)carbonyl, (C₁-C₄-alkoxy-C₁-C₄-alkyl)carbonyl, (C₃-C₆-alkenyloxy)carbonyl, (C₃₋₆-alkynyloxy)carbonyl, (C₃-C₈-cycloalkyl)carbonyl; (C₁-C₆-haloalkyl)carbonyl, (C₁-C₆-haloalkylthio)carbonyl, (C₁-C₆-haloalkoxy)carbonyl (C₃-C₆-haloalkenyloxy)carbonyl, (C₃-C₆-haloalkynyloxy)carbonyl, (halo-C₁-C₄-alkoxy-C₁-C₄-alkyl)carbonyl, (C₃-C₈-halocycloalkyl)carbonyl having in each case 1 to 9 fluorine, chlorine and/or bromine atoms, or —CH₂—C≡C—R^{1-A}, —CH₂—CH═CH—R^{1-A}, —CH═C═CH—R^{1-A}, —C(═O)C(═O)R², —CONR³R⁴, —CH₂NR⁵R⁶, C₁-C₄-trialkylsilyl or C₁-C₄-dialkylmonophenylsilyl,
R^{1-A} represents hydrogen, C₁-C₆-alkyl, C₁-C₆-haloalkyl, C₂-C₆-alkenyl, C₂-C₆-alkynyl, C₃-C₇-cycloalkyl. (C₁-C₄-alkoxy)carbonyl, (C₃-C₆-alkenyloxy)carbonyl, (C₃-C₆-alkynyloxy)carbonyl or cyano,
X is nitrogen or CR⁸,
Y is nitrogen or CR⁹,
Z is nitrogen or CR¹⁰,
R⁶ is hydrogen, halogen, cyano, hydroxyl, OR¹¹, SR¹¹, COR¹¹, CO₂R¹¹, unsubstituted or substituted C₁-C₈-alkyl, C₁-C₈-haloalkyl, C₁-C₄-trialkylsilyl, N(R¹¹)₂, NR¹¹COR¹¹, NR¹¹COR¹², NR¹¹SOR¹¹, CON(R¹¹)₂, C₃-C₈-cycloalkyl, aryl, hetaryl; where n=0-2,
R⁷ is hydrogen, halogen, cyano, hydroxyl, amino, N(R¹¹)₂, nitro, OR¹¹, SR¹¹, unsubstituted or substituted C₁-C₈-alkyl, unsubstituted or substituted C₃-C₆-cycloalkyl, C₁-C₄-trialkylsilyl, COOR¹¹, CON(R¹¹)₂ COR¹¹ or
R⁷ and R¹⁰ together form a saturated or unsaturated bridge of the following structure: 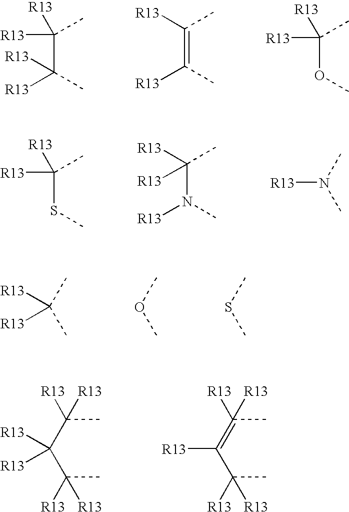
where R¹³=independently of one another hydrogen, halogen, unsubstituted or substituted C₁-C₃-alkyl, unsubstituted or substituted C₁-C₃-alkoxy or
two geminal radicals R¹³ represent doubly attached oxygen or sulphur,
R⁸ is hydrogen, halogen, cyano, hydroxyl, nitro, OR¹¹, SR¹¹, SOR¹¹, SOR¹¹, SO₂N(R¹¹)₅, C═OR¹¹, NR¹¹COOR¹², N(R¹¹)₂, NR¹¹COR¹¹, NR¹¹COR¹², NR¹¹SO₂R¹², OCON(R¹¹)₂, OC═OR¹¹, CON(R¹¹)₂, COOR¹¹, (CH₂)ₘCOOR¹¹, (CH₂)ₘSR¹¹, (CH₂)ₘN(R¹¹)₂, (CH₂)ₘCOOR¹², (CH₂)ₘNR¹¹COOR¹¹, unsubstituted or substituted C₁-C₈-alkyl, C₁-C₈-haloalkyl, C₃-C₈-cycloalkyl; where m=1-8,
R⁹ is hydrogen, halogen, N(R¹¹)₂, cyano, hydroxyl, OR¹¹, SR¹¹, COR¹¹, unsubstituted or substituted C₁-C₈-alkyl or unsubstituted or substituted C₃-C₈-cycloalkyl,
R¹⁰ is hydrogen, halogen, cyano, hydroxyl, amino, N(R¹¹)₂, nitro, OR¹¹, SR¹¹, unsubstituted or substituted C₁-C₈-alkyl, C₁-C₄-trialkylsilyl, COOR¹¹, CON(R¹¹)₂,
R¹¹ are identical or different and are hydrogen, unsubstituted or substituted C₁-C₈-alkyl, C₁-C₈-haloalkyl, C₁-C₄-trialkylsilyl, aryl, or
if two radicals R¹¹ are attached to one nitrogen atom, two radicals R¹¹ may form a 3- to 7-membered unsubstituted or substituted saturated or unsaturated cycle which may contain up to five further heteroatoms selected from the group consisting of N, O and S, where two oxygen atoms are not adjacent, or
if two radicals R¹¹ are adjacent in the grouping NR¹¹COR¹¹, two radicals R¹¹ may form a 3- to 7-membered unsubstituted or substituted saturated or unsaturated cycle which may contain up to five further heteroatoms selected from the group consisting of N, O and S, where two oxygen atoms are not adjacent,
R¹² are identical or different and are unsubstituted or substituted C₁-C₈-alkyl, C₁-C₈-haloalkyl, (CH₂)ₚOR¹¹, C₁-C₄-trialkylsilyl; where p=1-4
R⁶ may also be a group C(R¹⁴)₂A in which R¹⁴ and A are as defined above.
